SEQUENCE LISTING (758 sequences)

SEQ ID NO: 1 - DNA, 44 bp, synthetic construct
  misc_feature 1..44: note = Synthetic probe
  source 1..44: mol_type = other DNA
actggtaacccagacatgatcggttataactaccaccacgctga

SEQ ID NO: 2 - DNA, 44 bp, synthetic construct
  misc_feature 1..44: note = Synthetic probe
  source 1..44: mol_type = other DNA
actccctgtgggtgagcttaatggtataactaccaccacgctgg

SEQ ID NO: 3 - DNA, 51 bp, synthetic construct
  misc_feature 1..51: note = Synthetic probe
  source 1..51: mol_type = other DNA
catttggtcattggttcaagacgagttcctggcaattaattgtaaaaggtg

SEQ ID NO: 4 - DNA, 51 bp, synthetic construct
  misc_feature 1..51: note = Synthetic probe
  source 1..51: mol_type = other DNA
ctggtccgttgtggtccttctaacgttcctggcaattaattgtaaaaggta

SEQ ID NO: 5 - DNA, 26 bp, synthetic construct
  misc_feature 1..26: note = Synthetic probe
  source 1..26: mol_type = other DNA
ctaaaccatgtgtcaaaatcagcatg

SEQ ID NO: 6 - DNA, 28 bp, synthetic construct
  misc_feature 1..28: note = Synthetic probe
  source 1..28: mol_type = other DNA
aacaggaaactgtataattaccgatatc

SEQ ID NO: 7 - DNA, 20 bp, synthetic construct
  misc_feature 1..20: note = Synthetic oligonucleotide
  source 1..20: mol_type = other DNA
tataactaccaccacgctgr

SEQ ID NO: 8 - DNA, 26 bp, synthetic construct
  misc_feature 1..26: note = Synthetic oligonucleotide
  source 1..26: mol_type = other DNA
ctaaaccatgtgtcaaaatcagcatg

SEQ ID NO: 9 - DNA, 27 bp, synthetic construct
  misc_feature 1..27: note = Synthetic oligonucleotide
  source 1..27: mol_type = other DNA
gttcctggcaattaattgtaaaaggtr

SEQ ID NO: 10 - DNA, 28 bp, synthetic construct
  misc_feature 1..28: note = Synthetic oligonucleotide
  source 1..28: mol_type = other DNA
aacaggaaactgtataattaccgatatc

SEQ ID NO: 11 - DNA, 21 bp, synthetic construct
  misc_feature 1..21: note = Synthetic primer
  source 1..21: mol_type = other DNA
aggctattgatggtggtgtca

SEQ ID NO: 12 - DNA, 20 bp, synthetic construct
  misc_feature 1..20: note = Synthetic primer
  source 1..20: mol_type = other DNA
gcacgacaaaacccacttct

SEQ ID NO: 13 - DNA, 20 bp, synthetic construct
  misc_feature 1..20: note = Synthetic primer
  source 1..20: mol_type = other DNA
cgtggatgaggctggttcta

SEQ ID NO: 14 - DNA, 20 bp, synthetic construct
  misc_feature 1..20: note = Synthetic primer
  source 1..20: mol_type = other DNA
tttcgctgattttggggtcc

SEQ ID NO: 15 - DNA, 46 bp, synthetic construct
  misc_feature 1..46: note = Synthetic probe
  source 1..46: mol_type = other DNA
ctaatagctcctgtgccctcgtatgggacttctgtgcagttaacat

SEQ ID NO: 16 - DNA, 46 bp, synthetic construct
  misc_feature 1..46: note = Synthetic probe
  source 1..46: mol_type = other DNA
aatccgtcgactagcctgagaattgggacttctgtgcagttaacac

SEQ ID NO: 17 - DNA, 23 bp, synthetic construct
  misc_feature 1..23: note = Synthetic probe
  source 1..23: mol_type = other DNA
cctgataaagaacagcaacctgg

SEQ ID NO: 18 - DNA, 22 bp, synthetic construct
  misc_feature 1..22: note = Synthetic oligonucleotide
  source 1..22: mol_type = other DNA
gggacttctgtgcagttaacay

SEQ ID NO: 19 - DNA, 23 bp, synthetic construct
  misc_feature 1..23: note = Synthetic oligonucleotide
  source 1..23: mol_type = other DNA
cctgataaagaacagcaacctgg

SEQ ID NO: 20 - DNA, 20 bp, synthetic construct
  misc_feature 1..20: note = Synthetic primer
  source 1..20: mol_type = other DNA
acactactgatgctgtccgt

SEQ ID NO: 21 - DNA, 20 bp, synthetic construct
  misc_feature 1..20: note = Synthetic primer
  source 1..20: mol_type = other DNA
gcccctattaaacagcctgc

SEQ ID NO: 22 - DNA, 44 bp, synthetic construct
  misc_feature 1..44: note = Synthetic probe
  source 1..44: mol_type = other DNA
gtggcaacagaaatcaggtggtgacgaaatgcaccccgcattac

SEQ ID NO: 23 - DNA, 21 bp, synthetic construct
  misc_feature 1..21: note = Synthetic probe
  source 1..21: mol_type = other DNA
gtttggtggaccctcagattc

SEQ ID NO: 24 - DNA, 45 bp, synthetic construct
  misc_feature 1..45: note = Synthetic probe
  source 1..45: mol_type = other DNA
gtggcaacagaaatcaggtggtgagaatctgagggtccaccaaac

SEQ ID NO: 25 - DNA, 45 bp, synthetic construct
  misc_feature 1..45: note = Synthetic probe
  source 1..45: mol_type = other DNA
tagatgccgctgcaggtatggaaagaatctgagggtccaccaaac

SEQ ID NO: 26 - DNA, 20 bp, synthetic construct
  misc_feature 1..20: note = Synthetic probe
  source 1..20: mol_type = other DNA
gtaatgcggggtgcatttcg

SEQ ID NO: 27 - DNA, 45 bp, synthetic construct
  misc_feature 1..45: note = Synthetic probe
  source 1..45: mol_type = other DNA
tagatgccgctgcaggtatggaaagcaaattgcacaatttgcccc

SEQ ID NO: 28 - DNA, 18 bp, synthetic construct
  misc_feature 1..18: note = Synthetic probe
  source 1..18: mol_type = other DNA
cagcgcttcagcgttctt

SEQ ID NO: 29 - DNA, 41 bp, synthetic construct
  misc_feature 1..41: note = Synthetic probe
  source 1..41: mol_type = other DNA
tagatgccgctgcaggtatggaaagaacgctgaagcgctgg

SEQ ID NO: 30 - DNA, 21 bp, synthetic construct
  misc_feature 1..21: note = Synthetic probe
  source 1..21: mol_type = other DNA
gggcaaattgtgcaatttgcg

SEQ ID NO: 31 - DNA, 45 bp, synthetic construct
  misc_feature 1..45: note = Synthetic probe
  source 1..45: mol_type = other DNA
agaggactgctaaaggtttgtaggccaaaagaycacattggcacc

SEQ ID NO: 32 - DNA, 22 bp, synthetic construct
  misc_feature 1..22: note = Synthetic probe
  source 1..22: mol_type = other DNA
cgcaatcctgctaacaatgctg

SEQ ID NO: 33 - DNA, 45 bp, synthetic construct
  misc_feature 1..45: note = Synthetic probe
  source 1..45: mol_type = other DNA
agaggactgctaaaggtttgtagggcattgttagcaggattgcgg

SEQ ID NO: 34 - DNA, 22 bp, synthetic construct
  misc_feature 1..22: note = Synthetic probe
  source 1..22: mol_type = other DNA
gtgccaatgtgrtcttttggtg

SEQ ID NO: 35 - DNA, 42 bp, synthetic construct
  misc_feature 1..42: note = Synthetic probe
  source 1..42: mol_type = other DNA
cgtaccattgaatctggagaccttgagcgggttctgacctga

SEQ ID NO: 36 - DNA, 16 bp, synthetic construct
  misc_feature 1..16: note = Synthetic probe
  source 1..16: mol_type = other DNA
aggctctgcgcggact

SEQ ID NO: 37 - DNA, 41 bp, synthetic construct
  misc_feature 1..41: note = Synthetic probe
  source 1..41: mol_type = other DNA
cgtaccattgaatctggagaccttcaagtccgcgcagagcc

SEQ ID NO: 38 - DNA, 18 bp, synthetic construct
  misc_feature 1..18: note = Synthetic probe
  source 1..18: mol_type = other DNA
ttcaggtcagaacccgct

SEQ ID NO: 39 - DNA, 20 bp, synthetic construct
  misc_feature 1..20: note = Synthetic oligonucleotide
  source 1..20: mol_type = other DNA
cgaaatgcaccccgcattac

SEQ ID NO: 40 - DNA, 21 bp, synthetic construct
  misc_feature 1..21: note = Synthetic oligonucleotide
  source 1..21: mol_type = other DNA
gtttggtggaccctcagattc

SEQ ID NO: 41 - DNA, 21 bp, synthetic construct
  misc_feature 1..21: note = Synthetic oligonucleotide
  source 1..21: mol_type = other DNA
gaatctgagggtccaccaaac

SEQ ID NO: 42 - DNA, 20 bp, synthetic construct
  misc_feature 1..20: note = Synthetic oligonucleotide
  source 1..20: mol_type = other DNA
gtaatgcggggtgcatttcg

SEQ ID NO: 43 - DNA, 21 bp, synthetic construct
  misc_feature 1..21: note = Synthetic oligonucleotide
  source 1..21: mol_type = other DNA
gcaaattgcacaatttgcccc

SEQ ID NO: 44 - DNA, 18 bp, synthetic construct
  misc_feature 1..18: note = Synthetic oligonucleotide
  source 1..18: mol_type = other DNA
cagcgcttcagcgttctt

SEQ ID NO: 45 - DNA, 17 bp, synthetic construct
  misc_feature 1..17: note = Synthetic oligonucleotide
  source 1..17: mol_type = other DNA
gaacgctgaagcgctgg

SEQ ID NO: 46 - DNA, 21 bp, synthetic construct
  misc_feature 1..21: note = Synthetic oligonucleotide
  source 1..21: mol_type = other DNA
gggcaaattgtgcaatttgcg

SEQ ID NO: 47 - DNA, 21 bp, synthetic construct
  misc_feature 1..21: note = Synthetic oligonucleotide
  source 1..21: mol_type = other DNA
ccaaaagaycacattggcacc

SEQ ID NO: 48 - DNA, 22 bp, synthetic construct
  misc_feature 1..22: note = Synthetic oligonucleotide
  source 1..22: mol_type = other DNA
cgcaatcctgctaacaatgctg

SEQ ID NO: 49 - DNA, 21 bp, synthetic construct
  misc_feature 1..21: note = Synthetic oligonucleotide
  source 1..21: mol_type = other DNA
gcattgttagcaggattgcgg

SEQ ID NO: 50 - DNA, 22 bp, synthetic construct
  misc_feature 1..22: note = Synthetic oligonucleotide
  source 1..22: mol_type = other DNA
gtgccaatgtgrtcttttggtg

SEQ ID NO: 51 - DNA, 18 bp, synthetic construct
  misc_feature 1..18: note = Synthetic oligonucleotide
  source 1..18: mol_type = other DNA
gagcgggttctgacctga

SEQ ID NO: 52 - DNA, 16 bp, synthetic construct
  misc_feature 1..16: note = Synthetic oligonucleotide
  source 1..16: mol_type = other DNA
aggctctgcgcggact

SEQ ID NO: 53 - DNA, 17 bp, synthetic construct
  misc_feature 1..17: note = Synthetic oligonucleotide
  source 1..17: mol_type = other DNA
caagtccgcgcagagcc

SEQ ID NO: 54 - DNA, 18 bp, synthetic construct
  misc_feature 1..18: note = Synthetic oligonucleotide
  source 1..18: mol_type = other DNA
ttcaggtcagaacccgct

SEQ ID NO: 55 - DNA, 44 bp, synthetic construct
  misc_feature 1..44: note = Synthetic primer
  source 1..44: mol_type = other DNA
aatccgtcgactagcctgagaattgaccccaaaatcagcgaaat

SEQ ID NO: 56 - DNA, 24 bp, synthetic construct
  misc_feature 1..24: note = Synthetic primer
  source 1..24: mol_type = other DNA
tctggttactgccagttgaatctg

SEQ ID NO: 57 - DNA, 24 bp, synthetic construct
  misc_feature 1..24: note = Synthetic probe
  source 1..24: mol_type = other DNA
accccgcattacgtttggtggacc

SEQ ID NO: 58 - DNA, 44 bp, synthetic construct
  misc_feature 1..44: note = Synthetic primer
  source 1..44: mol_type = other DNA
agaggactgctaaaggtttgtaggttacaaacattggccgcaaa

SEQ ID NO: 59 - DNA, 18 bp, synthetic construct
  misc_feature 1..18: note = Synthetic primer
  source 1..18: mol_type = other DNA
gcgcgacattccgaagaa

SEQ ID NO: 60 - DNA, 23 bp, synthetic construct
  misc_feature 1..23: note = Synthetic probe
  source 1..23: mol_type = other DNA
acaatttgcccccagcgcttcag

SEQ ID NO: 61 - DNA, 46 bp, synthetic construct
  misc_feature 1..46: note = Synthetic primer
  source 1..46: mol_type = other DNA
cgtaccattgaatctggagaccttgggagccttgaatacaccaaaa

SEQ ID NO: 62 - DNA, 21 bp, synthetic construct
  misc_feature 1..21: note = Synthetic primer
  source 1..21: mol_type = other DNA
tgtagcacgattgcagcattg

SEQ ID NO: 63 - DNA, 24 bp, synthetic construct
  misc_feature 1..24: note = Synthetic probe
  source 1..24: mol_type = other DNA
aycacattggcacccgcaatcctg

SEQ ID NO: 64 - DNA, 43 bp, synthetic construct
  misc_feature 1..43: note = Synthetic primer
  source 1..43: mol_type = other DNA
actggtaacccagacatgatcggtagatttggacctgcgagcg

SEQ ID NO: 65 - DNA, 20 bp, synthetic construct
  misc_feature 1..20: note = Synthetic primer
  source 1..20: mol_type = other DNA
gagcggctgtctccacaagt

SEQ ID NO: 66 - DNA, 23 bp, synthetic construct
  misc_feature 1..23: note = Synthetic probe
  source 1..23: mol_type = other DNA
ttctgacctgaaggctctgcgcg

SEQ ID NO: 67 - DNA, 20 bp, synthetic construct
  misc_feature 1..20: note = Synthetic primer
  source 1..20: mol_type = other DNA
gaccccaaaatcagcgaaat

SEQ ID NO: 68 - DNA, 24 bp, synthetic construct
  misc_feature 1..24: note = Synthetic primer
  source 1..24: mol_type = other DNA
tctggttactgccagttgaatctg

SEQ ID NO: 69 - DNA, 20 bp, synthetic construct
  misc_feature 1..20: note = Synthetic primer
  source 1..20: mol_type = other DNA
ttacaaacattggccgcaaa

SEQ ID NO: 70 - DNA, 18 bp, synthetic construct
  misc_feature 1..18: note = Synthetic primer
  source 1..18: mol_type = other DNA
gcgcgacattccgaagaa

SEQ ID NO: 71 - DNA, 22 bp, synthetic construct
  misc_feature 1..22: note = Synthetic primer
  source 1..22: mol_type = other DNA
gggagccttgaatacaccaaaa

SEQ ID NO: 72 - DNA, 21 bp, synthetic construct
  misc_feature 1..21: note = Synthetic primer
  source 1..21: mol_type = other DNA
tgtagcacgattgcagcattg

SEQ ID NO: 73 - DNA, 19 bp, synthetic construct
  misc_feature 1..19: note = Synthetic primer
  source 1..19: mol_type = other DNA
agatttggacctgcgagcg

SEQ ID NO: 74 - DNA, 20 bp, synthetic construct
  misc_feature 1..20: note = Synthetic primer
  source 1..20: mol_type = other DNA
gagcggctgtctccacaagt

SEQ ID NO: 75 - DNA, 49 bp, synthetic construct
  misc_feature 1..49: note = Synthetic probe
  source 1..49: mol_type = other DNA
actccctgtgggtgagcttaatgggttacttggttccatgctatacatg

SEQ ID NO: 76 - DNA, 48 bp, synthetic construct
  misc_feature 1..48: note = Synthetic probe
  source 1..48: mol_type = other DNA
actggtaacccagacatgatcggtccaatgttacttggttccatgcta

SEQ ID NO: 77 - DNA, 23 bp, synthetic construct
  misc_feature 1..23: note = Synthetic probe
  source 1..23: mol_type = other DNA
tctctgggaccaatggtactaag

SEQ ID NO: 78 - DNA, 46 bp, synthetic construct
  misc_feature 1..46: note = Synthetic probe
  source 1..46: mol_type = other DNA
actccctgtgggtgagcttaatggccattggtcccagagacatgta

SEQ ID NO: 79 - DNA, 47 bp, synthetic construct
  misc_feature 1..47: note = Synthetic probe
  source 1..47: mol_type = other DNA
actggtaacccagacatgatcggtcttagtaccattggtcccagaga

SEQ ID NO: 80 - DNA, 24 bp, synthetic construct
  misc_feature 1..24: note = Synthetic probe
  source 1..24: mol_type = other DNA
tagcatggaaccaagtaacattgg

SEQ ID NO: 81 - DNA, 48 bp, synthetic construct
  misc_feature 1..48: note = Synthetic probe
  source 1..48: mol_type = other DNA
catttggtcattggttcaagacgaaatcatatggtttccaacccacta

SEQ ID NO: 82 - DNA, 48 bp, synthetic construct
  misc_feature 1..48: note = Synthetic probe
  source 1..48: mol_type = other DNA
ctggtccgttgtggtccttctaacaatcatatggtttccaacccactt

SEQ ID NO: 83 - DNA, 23 bp, synthetic construct
  misc_feature 1..23: note = Synthetic probe
  source 1..23: mol_type = other DNA
atggtgttggttaccaaccatac

SEQ ID NO: 84 - DNA, 47 bp, synthetic construct
  misc_feature 1..47: note = Synthetic probe
  source 1..47: mol_type = other DNA
catttggtcattggttcaagacgatatggttggtaaccaacaccatt

SEQ ID NO: 85 - DNA, 47 bp, synthetic construct
  misc_feature 1..47: note = Synthetic probe
  source 1..47: mol_type = other DNA
ctggtccgttgtggtccttctaactatggttggtaaccaacaccata

SEQ ID NO: 86 - DNA, 24 bp, synthetic construct
  misc_feature 1..24: note = Synthetic probe
  source 1..24: mol_type = other DNA
agtgggttggaaaccatatgattg

SEQ ID NO: 87 - DNA, 52 bp, synthetic construct
  misc_feature 1..52: note = Synthetic probe
  source 1..52: mol_type = other DNA
gtggcaacagaaatcaggtggtgactagttatcagactcagactaattctcc

SEQ ID NO: 88 - DNA, 52 bp, synthetic construct
  misc_feature 1..52: note = Synthetic probe
  source 1..52: mol_type = other DNA
agaggactgctaaaggtttgtaggctagttatcagactcagactaattctca

SEQ ID NO: 89 - DNA, 15 bp, synthetic construct
  misc_feature 1..15: note = Synthetic probe
  source 1..15: mol_type = other DNA
tcggcgggcacgtag

SEQ ID NO: 90 - DNA, 40 bp, synthetic construct
  misc_feature 1..40: note = Synthetic probe
  source 1..40: mol_type = other DNA
gtggcaacagaaatcaggtggtgactacgtgcccgccgag

SEQ ID NO: 91 - DNA, 40 bp, synthetic construct
  misc_feature 1..40: note = Synthetic probe
  source 1..40: mol_type = other DNA
agaggactgctaaaggtttgtaggctacgtgcccgccgat

SEQ ID NO: 92 - DNA, 28 bp, synthetic construct
  misc_feature 1..28: note = Synthetic probe
  source 1..28: mol_type = other DNA
gagaattagtctgagtctgataactagc

SEQ ID NO: 93 - DNA, 54 bp, synthetic construct
  misc_feature 1..54: note = Synthetic probe
  source 1..54: mol_type = other DNA
ctggtccgttgtggtccttctaacggaaaacagggtaatttcaaaaatcttagg

SEQ ID NO: 94 - DNA, 54 bp, synthetic construct
  misc_feature 1..54: note = Synthetic probe
  source 1..54: mol_type = other DNA
catttggtcattggttcaagacgaggaaaacagggtaatttcaaaaatcttagt

SEQ ID NO: 95 - DNA, 40 bp, synthetic construct
  misc_feature 1..40: note = Synthetic probe
  source 1..40: mol_type = other DNA
gaatttgtgtttaagaatattgatggttattttaaaatat

SEQ ID NO: 96 - DNA, 59 bp, synthetic construct
  misc_feature 1..59: note = Synthetic probe
  source 1..59: mol_type = other DNA
ctggtccgttgtggtccttctaactaaaataaccatcaatattcttaaacacaaattcc

SEQ ID NO: 97 - DNA, 59 bp, synthetic construct
  misc_feature 1..59: note = Synthetic probe
  source 1..59: mol_type = other DNA
catttggtcattggttcaagacgataaaataaccatcaatattcttaaacacaaattca

SEQ ID NO: 98 - DNA, 30 bp, synthetic construct
  misc_feature 1..30: note = Synthetic probe
  source 1..30: mol_type = other DNA
ctaagatttttgaaattaccctgttttcct

SEQ ID NO: 99 - DNA, 47 bp, synthetic construct
  misc_feature 1..47: note = Synthetic probe
  source 1..47: mol_type = other DNA
aatccgtcgactagcctgagaattacgcctattaatttagtgcgtga

SEQ ID NO: 100 - DNA, 47 bp, synthetic construct
  misc_feature 1..47: note = Synthetic probe
  source 1..47: mol_type = other DNA
ctaatagctcctgtgccctcgtatacgcctattaatttagtgcgtgg

SEQ ID NO: 101 - DNA, 20 bp, synthetic construct
  misc_feature 1..20: note = Synthetic probe
  source 1..20: mol_type = other DNA
tctccctcagggtttttcgg

SEQ ID NO: 102 - DNA, 45 bp, synthetic construct
  misc_feature 1..45: note = Synthetic probe
  source 1..45: mol_type = other DNA
aatccgtcgactagcctgagaattccgaaaaaccctgagggagat

SEQ ID NO: 103 - DNA, 45 bp, synthetic construct
  misc_feature 1..45: note = Synthetic probe
  source 1..45: mol_type = other DNA
ctaatagctcctgtgccctcgtatccgaaaaaccctgagggagac

SEQ ID NO: 104 - DNA, 23 bp, synthetic construct
  misc_feature 1..23: note = Synthetic probe
  source 1..23: mol_type = other DNA
cacgcactaaattaataggcgtg

SEQ ID NO: 105 - DNA, 55 bp, synthetic construct
  misc_feature 1..55: note = Synthetic probe
  source 1..55: mol_type = other DNA
agaggactgctaaaggtttgtaggattctaaggttggtggtaattataattacct

SEQ ID NO: 106 - DNA, 55 bp, synthetic construct
  misc_feature 1..55: note = Synthetic probe
  source 1..55: mol_type = other DNA
gtggcaacagaaatcaggtggtgaattctaaggttggtggtaattataattaccg

SEQ ID NO: 107 - DNA, 32 bp, synthetic construct
  misc_feature 1..32: note = Synthetic probe
  source 1..32: mol_type = other DNA
gtatagattgtttaggaagtctaatctcaaac

SEQ ID NO: 108 - DNA, 57 bp, synthetic construct
  misc_feature 1..57: note = Synthetic probe
  source 1..57: mol_type = other DNA
agaggactgctaaaggtttgtagggtttgagattagacttcctaaacaatctataca

SEQ ID NO: 109 - DNA, 57 bp, synthetic construct
  misc_feature 1..57: note = Synthetic probe
  source 1..57: mol_type = other DNA
gtggcaacagaaatcaggtggtgagtttgagattagacttcctaaacaatctatacc

SEQ ID NO: 110 - DNA, 30 bp, synthetic construct
  misc_feature 1..30: note = Synthetic probe
  source 1..30: mol_type = other DNA
ggtaattataattaccaccaaccttagaat

SEQ ID NO: 111 - DNA, 46 bp, synthetic construct
  misc_feature 1..46: note = Synthetic probe
  source 1..46: mol_type = other DNA
actggtaacccagacatgatcggtagcacaccttgtaatggtgttg

SEQ ID NO: 112 - DNA, 46 bp, synthetic construct
  misc_feature 1..46: note = Synthetic probe
  source 1..46: mol_type = other DNA
actccctgtgggtgagcttaatggagcacaccttgtaatggtgtta

SEQ ID NO: 113 - DNA, 31 bp, synthetic construct
  misc_feature 1..31: note = Synthetic probe
  source 1..31: mol_type = other DNA
aaggttttaattgttactttcctttacaatc

SEQ ID NO: 114 - DNA, 57 bp, synthetic construct
  misc_feature 1..57: note = Synthetic probe
  source 1..57: mol_type = other DNA
actggtaacccagacatgatcggttgattgtaaaggaaagtaacaattaaaaccttc

SEQ ID NO: 115 - DNA, 57 bp, synthetic construct
  misc_feature 1..57: note = Synthetic probe
  source 1..57: mol_type = other DNA
actccctgtgggtgagcttaatggtgattgtaaaggaaagtaacaattaaaaccttt

SEQ ID NO: 116 - DNA, 22 bp, synthetic construct
  misc_feature 1..22: note = Synthetic probe
  source 1..22: mol_type = other DNA
aacaccattacaaggtgtgcta

SEQ ID NO: 117 - DNA, 47 bp, synthetic construct
  misc_feature 1..47: note = Synthetic probe
  source 1..47: mol_type = other DNA
tagatgccgctgcaggtatggaaagcctacactatgtcacttggtgc

SEQ ID NO: 118 - DNA, 47 bp, synthetic construct
  misc_feature 1..47: note = Synthetic probe
  source 1..47: mol_type = other DNA
cgtaccattgaatctggagaccttgcctacactatgtcacttggtgt

SEQ ID NO: 119 - DNA, 31 bp, synthetic construct
  misc_feature 1..31: note = Synthetic probe
  source 1..31: mol_type = other DNA
agaaaattcagttgcttactctaataactct

SEQ ID NO: 120 - DNA, 56 bp, synthetic construct
  misc_feature 1..56: note = Synthetic probe
  source 1..56: mol_type = other DNA
tagatgccgctgcaggtatggaaaagagttattagagtaagcaactgaattttctg

SEQ ID NO: 121 - DNA, 56 bp, synthetic construct
  misc_feature 1..56: note = Synthetic probe
  source 1..56: mol_type = other DNA
cgtaccattgaatctggagaccttagagttattagagtaagcaactgaattttcta

SEQ ID NO: 122 - DNA, 22 bp, synthetic construct
  misc_feature 1..22: note = Synthetic probe
  source 1..22: mol_type = other DNA
caccaagtgacatagtgtaggc

SEQ ID NO: 123 - DNA, 45 bp, synthetic construct
  misc_feature 1..45: note = Synthetic probe
  source 1..45: mol_type = other DNA
tagatgccgctgcaggtatggaaagctccagggcaaactggaamg

SEQ ID NO: 124 - DNA, 45 bp, synthetic construct
  misc_feature 1..45: note = Synthetic probe
  source 1..45: mol_type = other DNA
cgtaccattgaatctggagaccttgctccagggcaaactggaamt

SEQ ID NO: 125 - DNA, 38 bp, synthetic construct
  misc_feature 1..38: note = Synthetic probe
  source 1..38: mol_type = other DNA
attgctgattataattataaattaccagatgattttac

SEQ ID NO: 126 - DNA, 63 bp, synthetic construct
  misc_feature 1..63: note = Synthetic probe
  source 1..63: mol_type = other DNA
tagatgccgctgcaggtatggaaagtaaaatcatctggtaatttataattataatcagcaatc

SEQ ID NO: 127 - DNA, 63 bp, synthetic construct
  misc_feature 1..63: note = Synthetic probe
  source 1..63: mol_type = other DNA
cgtaccattgaatctggagaccttgtaaaatcatctggtaatttataattataatcagcaata

SEQ ID NO: 128 - DNA, 20 bp, synthetic construct
  misc_feature 1..20: note = Synthetic probe
  source 1..20: mol_type = other DNA
kttccagtttgccctggagc

SEQ ID NO: 129 - DNA, 44 bp, synthetic construct
  misc_feature 1..44: note = Synthetic probe
  source 1..44: mol_type = other DNA
ctggtccgttgtggtccttctaacgctccagggcaaactggaaa

SEQ ID NO: 130 - DNA, 44 bp, synthetic construct
  misc_feature 1..44: note = Synthetic probe
  source 1..44: mol_type = other DNA
catttggtcattggttcaagacgagctccagggcaaactggaac

SEQ ID NO: 131 - DNA, 39 bp, synthetic construct
  misc_feature 1..39: note = Synthetic probe
  source 1..39: mol_type = other DNA
kattgctgattataattataaattaccagatgattttac

SEQ ID NO: 132 - DNA, 59 bp, synthetic construct
  misc_feature 1..59: note = Synthetic probe
  source 1..59: mol_type = other DNA
ctggtccgttgtggtccttctaacatcatctggtaatttataattataatcagcaatmt

SEQ ID NO: 133 - DNA, 59 bp, synthetic construct
  misc_feature 1..59: note = Synthetic probe
  source 1..59: mol_type = other DNA
catttggtcattggttcaagacgaatcatctggtaatttataattataatcagcaatmg

SEQ ID NO: 134 - DNA, 19 bp, synthetic construct
  misc_feature 1..19: note = Synthetic probe
  source 1..19: mol_type = other DNA
ttccagtttgccctggagc

SEQ ID NO: 135 - DNA, 75 bp, unidentified
  misc_feature 1..75: note = WT template strand
  source 1..75: mol_type = other DNA
cctttcttttccaatgttacttggttccatgctatacatgtctctgggaccaatggtactaagaggtttgataac

SEQ ID NO: 136 - DNA, 75 bp, synthetic construct
  misc_feature 1..75: note = Synthetic oligonucleotide
  source 1..75: mol_type = other DNA
acctttcttttccaatgttacttggttccatgctatctctgggaccaatggtactaagaggtttgataaccctgt

SEQ ID NO: 137 - DNA, 75 bp, unidentified
  misc_feature 1..75: note = WT cDNA strand
  source 1..75: mol_type = other DNA
ttatcaaacctcttagtaccattggtcccagagacatgtatagcatggaaccaagtaacattggaaaagaaaggt

SEQ ID NO: 138 - DNA, 75 bp, synthetic construct
  misc_feature 1..75: note = Synthetic oligonucleotide
  source 1..75: mol_type = other DNA
agggttatcaaacctcttagtaccattggtcccagagatagcatggaaccaagtaacattggaaaagaaaggtaa

SEQ ID NO: 139 - DNA, 75 bp, synthetic construct
  misc_feature 1..75: note = Synthetic oligonucleotide
  source 1..75: mol_type = other DNA
ttactttcctttacaatcatatggtttccaacccactaatggtgttggttaccaaccatacagagtagtagtact

SEQ ID NO: 140 - DNA, 75 bp, synthetic construct
  misc_feature 1..75: note = Synthetic oligonucleotide
  source 1..75: mol_type = other DNA
ttactttcctttacaatcatatggtttccaacccacttatggtgttggttaccaaccatacagagtagtagtact

SEQ ID NO: 141 - DNA, 75 bp, synthetic construct
  misc_feature 1..75: note = Synthetic oligonucleotide
  source 1..75: mol_type = other DNA
agtactactactctgtatggttggtaaccaacaccattagtgggttggaaaccatatgattgtaaaggaaagtaa

SEQ ID NO: 142 - DNA, 75 bp, synthetic construct
  misc_feature 1..75: note = Synthetic oligonucleotide
  source 1..75: mol_type = other DNA
agtactactactctgtatggttggtaaccaacaccataagtgggttggaaaccatatgattgtaaaggaaagtaa

SEQ ID NO: 143 - DNA, 75 bp, synthetic construct
  misc_feature 1..75: note = Synthetic oligonucleotide
  source 1..75: mol_type = other DNA
aggtatatgcgctagttatcagactcagactaattctcctcggcgggcacgtagtgtagctagtcaatccatcat

SEQ ID NO: 144 - DNA, 75 bp, synthetic construct
  misc_feature 1..75: note = Synthetic oligonucleotide
  source 1..75: mol_type = other DNA
aggtatatgcgctagttatcagactcagactaattctcatcggcgggcacgtagtgtagctagtcaatccatcat

SEQ ID NO: 145 - DNA, 75 bp, synthetic construct
  misc_feature 1..75: note = Synthetic oligonucleotide
  source 1..75: mol_type = other DNA
atgatggattgactagctacactacgtgcccgccgaggagaattagtctgagtctgataactagcgcatatacct

SEQ ID NO: 146 - DNA, 75 bp, synthetic construct
  misc_feature 1..75: note = Synthetic oligonucleotide
  source 1..75: mol_type = other DNA
atgatggattgactagctacactacgtgcccgccgatgagaattagtctgagtctgataactagcgcatatacct

SEQ ID NO: 147 - DNA, 78 bp, synthetic construct
  misc_feature 1..78: note = Synthetic oligonucleotide
  source 1..78: mol_type = other DNA
accttgaaggaaaacagggtaatttcaaaaatcttagggaatttgtgtttaagaatattgatggttattttaaaatat

SEQ ID NO: 148 - DNA, 78 bp, synthetic construct
  misc_feature 1..78: note = Synthetic oligonucleotide
  source 1..78: mol_type = other DNA
accttgaaggaaaacagggtaatttcaaaaatcttagtgaatttgtgtttaagaatattgatggttattttaaaatat

SEQ ID NO: 149 - DNA, 75 bp, synthetic construct
  misc_feature 1..75: note = Synthetic oligonucleotide
  source 1..75: mol_type = other DNA
ttttaaaataaccatcaatattcttaaacacaaattccctaagatttttgaaattaccctgttttccttcaaggt

SEQ ID NO: 150 - DNA, 75 bp, synthetic construct
  misc_feature 1..75: note = Synthetic oligonucleotide
  source 1..75: mol_type = other DNA
ttttaaaataaccatcaatattcttaaacacaaattcactaagatttttgaaattaccctgttttccttcaaggt

SEQ ID NO: 151 - DNA, 75 bp, synthetic construct
  misc_feature 1..75: note = Synthetic oligonucleotide
  source 1..75: mol_type = other DNA
atatattctaagcacacgcctattaatttagtgcgtgatctccctcagggtttttcggctttagaaccattggta

SEQ ID NO: 152 - DNA, 75 bp, synthetic construct
  misc_feature 1..75: note = Synthetic oligonucleotide
  source 1..75: mol_type = other DNA
atatattctaagcacacgcctattaatttagtgcgtggtctccctcagggtttttcggctttagaaccattggta

SEQ ID NO: 153 - DNA, 75 bp, synthetic construct
  misc_feature 1..75: note = Synthetic oligonucleotide
  source 1..75: mol_type = other DNA
taccaatggttctaaagccgaaaaaccctgagggagatcacgcactaaattaataggcgtgtgcttagaatatat

SEQ ID NO: 154 - DNA, 75 bp, synthetic construct
  misc_feature 1..75: note = Synthetic oligonucleotide
  source 1..75: mol_type = other DNA
taccaatggttctaaagccgaaaaaccctgagggagaccacgcactaaattaataggcgtgtgcttagaatatat

SEQ ID NO: 155 - DNA, 75 bp, synthetic construct
  misc_feature 1..75: note = Synthetic oligonucleotide
  source 1..75: mol_type = other DNA
aatcttgattctaaggttggtggtaattataattacctgtatagattgtttaggaagtctaatctcaaacctttt

SEQ ID NO: 156 - DNA, 75 bp, synthetic construct
  misc_feature 1..75: note = Synthetic oligonucleotide
  source 1..75: mol_type = other DNA
aatcttgattctaaggttggtggtaattataattaccggtatagattgtttaggaagtctaatctcaaacctttt

SEQ ID NO: 157 - DNA, 75 bp, synthetic construct
  misc_feature 1..75: note = Synthetic oligonucleotide
  source 1..75: mol_type = other DNA
aaaaggtttgagattagacttcctaaacaatctatacaggtaattataattaccaccaaccttagaatcaagatt

SEQ ID NO: 158 - DNA, 75 bp, synthetic construct
  misc_feature 1..75: note = Synthetic oligonucleotide
  source 1..75: mol_type = other DNA
aaaaggtttgagattagacttcctaaacaatctataccggtaattataattaccaccaaccttagaatcaagatt

SEQ ID NO: 159 - DNA, 76 bp, synthetic construct
  misc_feature 1..76: note = Synthetic oligonucleotide
  source 1..76: mol_type = other DNA
aatctatcaggccggtagcacaccttgtaatggtgttgaaggttttaattgttactttcctttacaatcatatggt

SEQ ID NO: 160 - DNA, 76 bp, synthetic construct
  misc_feature 1..76: note = Synthetic oligonucleotide
  source 1..76: mol_type = other DNA
aatctatcaggccggtagcacaccttgtaatggtgttaaaggttttaattgttactttcctttacaatcatatggt

SEQ ID NO: 161 - DNA, 75 bp, synthetic construct
  misc_feature 1..75: note = Synthetic oligonucleotide
  source 1..75: mol_type = other DNA
ccatatgattgtaaaggaaagtaacaattaaaaccttcaacaccattacaaggtgtgctaccggcctgatagatt

SEQ ID NO: 162 - DNA, 75 bp, synthetic construct
  misc_feature 1..75: note = Synthetic oligonucleotide
  source 1..75: mol_type = other DNA
ccatatgattgtaaaggaaagtaacaattaaaacctttaacaccattacaaggtgtgctaccggcctgatagatt

SEQ ID NO: 163 - DNA, 75 bp, synthetic construct
  misc_feature 1..75: note = Synthetic oligonucleotide
  source 1..75: mol_type = other DNA
agtcaatccatcattgcctacactatgtcacttggtgcagaaaattcagttgcttactctaataactctattgcc

SEQ ID NO: 164 - DNA, 75 bp, synthetic construct
  misc_feature 1..75: note = Synthetic oligonucleotide
  source 1..75: mol_type = other DNA
agtcaatccatcattgcctacactatgtcacttggtgtagaaaattcagttgcttactctaataactctattgcc

SEQ ID NO: 165 - DNA, 75 bp, synthetic construct
  misc_feature 1..75: note = Synthetic oligonucleotide
  source 1..75: mol_type = other DNA
ggcaatagagttattagagtaagcaactgaattttctgcaccaagtgacatagtgtaggcaatgatggattgact

SEQ ID NO: 166 - DNA, 75 bp, synthetic construct
  misc_feature 1..75: note = Synthetic oligonucleotide
  source 1..75: mol_type = other DNA
ggcaatagagttattagagtaagcaactgaattttctacaccaagtgacatagtgtaggcaatgatggattgact

SEQ ID NO: 167 - DNA, 75 bp, synthetic construct
  misc_feature 1..75: note = Synthetic oligonucleotide
  source 1..75: mol_type = other DNA
atgaagtcagacaaatcgctccagggcaaactggaamgattgctgattataattataaattaccagatgatttta

SEQ ID NO: 168 - DNA, 75 bp, synthetic construct
  misc_feature 1..75: note = Synthetic oligonucleotide
  source 1..75: mol_type = other DNA
atgaagtcagacaaatcgctccagggcaaactggaamtattgctgattataattataaattaccagatgatttta

SEQ ID NO: 169 - DNA, 75 bp, synthetic construct
  misc_feature 1..75: note = Synthetic oligonucleotide
  source 1..75: mol_type = other DNA
taaaatcatctggtaatttataattataatcagcaatckttccagtttgccctggagcgatttgtctgacttcat

SEQ ID NO: 170 - DNA, 75 bp, synthetic construct
  misc_feature 1..75: note = Synthetic oligonucleotide
  source 1..75: mol_type = other DNA
taaaatcatctggtaatttataattataatcagcaatakttccagtttgccctggagcgatttgtctgacttcat

SEQ ID NO: 171 - DNA, 75 bp, synthetic construct
  misc_feature 1..75: note = Synthetic oligonucleotide
  source 1..75: mol_type = other DNA
gatgaagtcagacaaatcgctccagggcaaactggaaakattgctgattataattataaattaccagatgatttt

SEQ ID NO: 172 - DNA, 75 bp, synthetic construct
  misc_feature 1..75: note = Synthetic oligonucleotide
  source 1..75: mol_type = other DNA
gatgaagtcagacaaatcgctccagggcaaactggaackattgctgattataattataaattaccagatgatttt

SEQ ID NO: 173 - DNA, 75 bp, synthetic construct
  misc_feature 1..75: note = Synthetic oligonucleotide
  source 1..75: mol_type = other DNA
aaaatcatctggtaatttataattataatcagcaatmtttccagtttgccctggagcgatttgtctgacttcatc

SEQ ID NO: 174 - DNA, 75 bp, synthetic construct
  misc_feature 1..75: note = Synthetic oligonucleotide
  source 1..75: mol_type = other DNA
aaaatcatctggtaatttataattataatcagcaatmgttccagtttgccctggagcgatttgtctgacttcatc

SEQ ID NO: 175 - DNA, 25 bp, synthetic construct
  misc_feature 1..25: note = Synthetic oligonucleotide
  source 1..25: mol_type = other DNA
gttacttggttccatgctatacatg

SEQ ID NO: 176 - DNA, 24 bp, synthetic construct
  misc_feature 1..24: note = Synthetic oligonucleotide
  source 1..24: mol_type = other DNA
ccaatgttacttggttccatgcta

SEQ ID NO: 177 - DNA, 23 bp, synthetic construct
  misc_feature 1..23: note = Synthetic oligonucleotide
  source 1..23: mol_type = other DNA
tctctgggaccaatggtactaag

SEQ ID NO: 178 - DNA, 22 bp, synthetic construct
  misc_feature 1..22: note = Synthetic oligonucleotide
  source 1..22: mol_type = other DNA
ccattggtcccagagacatgta

SEQ ID NO: 179 - DNA, 23 bp, synthetic construct
  misc_feature 1..23: note = Synthetic oligonucleotide
  source 1..23: mol_type = other DNA
cttagtaccattggtcccagaga

SEQ ID NO: 180 - DNA, 24 bp, synthetic construct
  misc_feature 1..24: note = Synthetic oligonucleotide
  source 1..24: mol_type = other DNA
tagcatggaaccaagtaacattgg

SEQ ID NO: 181 - DNA, 24 bp, synthetic construct
  misc_feature 1..24: note = Synthetic oligonucleotide
  source 1..24: mol_type = other DNA
aatcatatggtttccaacccacty

SEQ ID NO: 182 - DNA, 23 bp, synthetic construct
  misc_feature 1..23: note = Synthetic oligonucleotide
  source 1..23: mol_type = other DNA
atggtgttggttaccaaccatac

SEQ ID NO: 183 - DNA, 23 bp, synthetic construct
  misc_feature 1..23: note = Synthetic oligonucleotide
  source 1..23: mol_type = other DNA
tatggttggtaaccaacaccaty

SEQ ID NO: 184 - DNA, 24 bp, synthetic construct
  misc_feature 1..24: note = Synthetic oligonucleotide
  source 1..24: mol_type = other DNA
agtgggttggaaaccatatgattg

SEQ ID NO: 185 - DNA, 28 bp, synthetic construct
  misc_feature 1..28: note = Synthetic oligonucleotide
  source 1..28: mol_type = other DNA
ctagttatcagactcagactaattctcm

SEQ ID NO: 186 - DNA, 15 bp, synthetic construct
  misc_feature 1..15: note = Synthetic oligonucleotide
  source 1..15: mol_type = other DNA
tcggcgggcacgtag

SEQ ID NO: 187 - DNA, 16 bp, synthetic construct
  misc_feature 1..16: note = Synthetic oligonucleotide
  source 1..16: mol_type = other DNA
ctacgtgcccgccgab

SEQ ID NO: 188 - DNA, 28 bp, synthetic construct
  misc_feature 1..28: note = Synthetic oligonucleotide
  source 1..28: mol_type = other DNA
gagaattagtctgagtctgataactagc

SEQ ID NO: 189 - DNA, 30 bp, synthetic construct
  misc_feature 1..30: note = Synthetic oligonucleotide
  source 1..30: mol_type = other DNA
ggaaaacagggtaatttcaaaaatcttagk

SEQ ID NO: 190 - DNA, 40 bp, synthetic construct; identical to SEQ ID NO: 95
  misc_feature 1..40: note = Synthetic oligonucleotide
  source 1..40: mol_type = other DNA

SEQ ID NO: 191 - DNA, 35 bp, synthetic construct
  misc_feature 1..35: note = Synthetic oligonucleotide
  source 1..35: mol_type = other DNA
taaaataaccatcaatattcttaaacacaaattcm

SEQ ID NO: 192 - DNA, 30 bp, synthetic construct; identical to SEQ ID NO: 98
  misc_feature 1..30: note = Synthetic oligonucleotide
  source 1..30: mol_type = other DNA

SEQ ID NO: 193 - DNA, 23 bp, synthetic construct
  misc_feature 1..23: note = Synthetic oligonucleotide
  source 1..23: mol_type = other DNA
acgcctattaatttagtgcgtgr

SEQ ID NO: 194 - DNA, 20 bp, synthetic construct
  misc_feature 1..20: note = Synthetic oligonucleotide
  source 1..20: mol_type = other DNA
tctccctcagggtttttcgg

SEQ ID NO: 195 - DNA, 21 bp, synthetic construct
  misc_feature 1..21: note = Synthetic oligonucleotide
  source 1..21: mol_type = other DNA
ccgaaaaaccctgagggagay

SEQ ID NO: 196 - DNA, 23 bp, synthetic construct
  misc_feature 1..23: note = Synthetic oligonucleotide
  source 1..23: mol_type = other DNA
cacgcactaaattaataggcgtg

SEQ ID NO: 197 - DNA, 31 bp, synthetic construct
  misc_feature 1..31: note = Synthetic oligonucleotide
  source 1..31: mol_type = other DNA
attctaaggttggtggtaattataattacck

SEQ ID NO: 198 - DNA, 32 bp, synthetic construct; identical to SEQ ID NO: 107
  misc_feature 1..32: note = Synthetic oligonucleotide
  source 1..32: mol_type = other DNA

SEQ ID NO: 199 - DNA, 33 bp, synthetic construct
  misc_feature 1..33: note = Synthetic oligonucleotide
  source 1..33: mol_type = other DNA
gtttgagattagacttcctaaacaatctatacm

SEQ ID NO: 200 - DNA, 30 bp, synthetic construct; identical to SEQ ID NO: 110
  misc_feature 1..30: note = Synthetic oligonucleotide
  source 1..30: mol_type = other DNA

SEQ ID NO: 201 - DNA, 22 bp, synthetic construct
  misc_feature 1..22: note = Synthetic oligonucleotide
  source 1..22: mol_type = other DNA
agcacaccttgtaatggtgttr

SEQ ID NO: 202 - DNA, 31 bp, synthetic construct; identical to SEQ ID NO: 113
  misc_feature 1..31: note = Synthetic oligonucleotide
  source 1..31: mol_type = other DNA

SEQ ID NO: 203 - DNA, 33 bp, synthetic construct
  misc_feature 1..33: note = Synthetic oligonucleotide
  source 1..33: mol_type = other DNA
tgattgtaaaggaaagtaacaattaaaacctty

SEQ ID NO: 204 - DNA, 22 bp, synthetic construct
  misc_feature 1..22: note = Synthetic oligonucleotide
  source 1..22: mol_type = other DNA
aacaccattacaaggtgtgcta

SEQ ID NO: 205 - DNA, 23 bp, synthetic construct
  misc_feature 1..23: note = Synthetic oligonucleotide
  source 1..23: mol_type = other DNA
gcctacactatgtcacttggtgy

SEQ ID NO: 206 - DNA, 31 bp, synthetic construct; identical to SEQ ID NO: 119
  misc_feature 1..31: note = Synthetic oligonucleotide
  source 1..31: mol_type = other DNA

SEQ ID NO: 207 - DNA, 32 bp, synthetic construct
  misc_feature 1..32: note = Synthetic oligonucleotide
  source 1..32: mol_type = other DNA
agagttattagagtaagcaactgaattttctr

SEQ ID NO: 208 - DNA, 22 bp, synthetic construct
  misc_feature 1..22: note = Synthetic oligonucleotide
  source 1..22: mol_type = other DNA
caccaagtgacatagtgtaggc

SEQ ID NO: 209 - DNA, 21 bp, synthetic construct
  misc_feature 1..21: note = Synthetic oligonucleotide
  source 1..21: mol_type = other DNA
gctccagggcaaactggaamk

SEQ ID NO: 210 - DNA, 38 bp, synthetic construct; identical to SEQ ID NO: 125
  misc_feature 1..38: note = Synthetic oligonucleotide
  source 1..38: mol_type = other DNA

SEQ ID NO: 211 - DNA, 39 bp, synthetic construct
  misc_feature 1..39: note = Synthetic oligonucleotide
  source 1..39: mol_type = other DNA
gtaaaatcatctggtaatttataattataatcagcaatm

SEQ ID NO: 212 - DNA, 20 bp, synthetic construct
  misc_feature 1..20: note = Synthetic oligonucleotide
  source 1..20: mol_type = other DNA
kttccagtttgccctggagc

SEQ ID NO: 213 - DNA, 20 bp, synthetic construct
  misc_feature 1..20: note = Synthetic oligonucleotide
  source 1..20: mol_type = other DNA
gctccagggcaaactggaam

SEQ ID NO: 214 - DNA, 39 bp, synthetic construct; identical to SEQ ID NO: 131
  misc_feature 1..39: note = Synthetic oligonucleotide
  source 1..39: mol_type = other DNA

SEQ ID NO: 215 - DNA, 35 bp, synthetic construct
  misc_feature 1..35: note = Synthetic oligonucleotide
  source 1..35: mol_type = other DNA
atcatctggtaatttataattataatcagcaatmk

SEQ ID NO: 216 - DNA, 19 bp, synthetic construct
  misc_feature 1..19: note = Synthetic oligonucleotide
  source 1..19: mol_type = other DNA
ttccagtttgccctggagc

SEQ ID NO: 217 - DNA, 22 bp, synthetic construct
  misc_feature 1..22: note = Synthetic primer
  source 1..22: mol_type = other DNA
acgtggtgtttattaccctgac

SEQ ID NO: 218 - DNA, 22 bp, synthetic construct
  misc_feature 1..22: note = Synthetic primer
  source 1..22: mol_type = other DNA
gctgcattcagttgaatcacca

SEQ ID NO: 219 - DNA, 25 bp, synthetic construct
  misc_feature 1..25: note = Synthetic primer
  source 1..25: mol_type = other DNA
ttccaatgttacttggttccatgct

SEQ ID NO: 220 - DNA, 30 bp, synthetic construct
  misc_feature 1..30: note = Synthetic primer
  source 1..30: mol_type = other DNA
gggtatggcaatagagttattagagtaagc

SEQ ID NO: 221 - DNA, 27 bp, synthetic construct
  misc_feature 1..27: note = Synthetic primer
  source 1..27: mol_type = other DNA
acattcaactcaggacttgttcttacc

SEQ ID NO: 222 - DNA, 25 bp, synthetic construct
  misc_feature 1..25: note = Synthetic primer
  source 1..25: mol_type = other DNA
gctgtccaacctgaagaagaatcac

SEQ ID NO: 223 - DNA, 25 bp, synthetic construct
  misc_feature 1..25: note = Synthetic primer
  source 1..25: mol_type = other DNA
caggaagagaatcagcaactgtgtt

SEQ ID NO: 224 - DNA, 27 bp, synthetic construct
  misc_feature 1..27: note = Synthetic primer
  source 1..27: mol_type = other DNA
tgtcaagaatctcaagtgtctgtggat

SEQ ID NO: 225 - DNA, 26 bp, synthetic construct
  misc_feature 1..26: note = Synthetic primer
  source 1..26: mol_type = other DNA
atcaacttactcctacttggcgtgtt

SEQ ID NO: 226 - DNA, 26 bp, synthetic construct
  misc_feature 1..26: note = Synthetic primer
  source 1..26: mol_type = other DNA
tctagcagcaatatcaccaaggcaat

SEQ ID NO: 227 - DNA, 19 bp, synthetic construct
  misc_feature 1..19: note = Synthetic probe
  source 1..19: mol_type = other DNA
tccctcagggtttttcggc

SEQ ID NO: 228 - DNA, 19 bp, synthetic construct
  misc_feature 1..19: note = Synthetic probe
  source 1..19: mol_type = other DNA
tccctcagggtttttcggt

SEQ ID NO: 229 - DNA, 25 bp, synthetic construct
  misc_feature 1..25: note = Synthetic probe
  source 1..25: mol_type = other DNA
tttagaaccattggtagatttgcca

SEQ ID NO: 230 - DNA, 25 bp, synthetic construct
  misc_feature 1..25: note = Synthetic probe
  source 1..25: mol_type = other DNA
ggcaaatctaccaatggttctaaag

SEQ ID NO: 231 - DNA, 25 bp, synthetic construct
  misc_feature 1..25: note = Synthetic probe
  source 1..25: mol_type = other DNA
ggcaaatctaccaatggttctaaaa

SEQ ID NO: 232 - DNA, 20 bp, synthetic construct
  misc_feature 1..20: note = Synthetic probe
  source 1..20: mol_type = other DNA
ccgaaaaaccctgagggaga

SEQ ID NO: 233 - DNA, 18 bp, synthetic construct
  misc_feature 1..18: note = Synthetic probe
  source 1..18: mol_type = other DNA
cggtgatgctgctcttgc

SEQ ID NO: 234 - DNA, 18 bp, synthetic construct
  misc_feature 1..18: note = Synthetic probe
  source 1..18: mol_type = other DNA
cggtgatgctgctcttgt

SEQ ID NO: 235 - DNA, 22 bp, synthetic construct
  misc_feature 1..22: note = Synthetic probe
  source 1..22: mol_type = other DNA
tttgctgctgcttgacagattg

SEQ ID NO: 236 - DNA, 23 bp, synthetic construct
  misc_feature 1..23: note = Synthetic probe
  source 1..23: mol_type = other DNA
caatctgtcaagcagcagcaaag

SEQ ID NO: 237 - DNA, 23 bp, synthetic construct
  misc_feature 1..23: note = Synthetic probe
  source 1..23: mol_type = other DNA
caatctgtcaagcagcagcaaaa

SEQ ID NO: 238 - DNA, 18 bp, synthetic construct
  misc_feature 1..18: note = Synthetic probe
  source 1..18: mol_type = other DNA
caagagcagcatcaccgc

SEQ ID NO: 239 - DNA, 27 bp, synthetic construct
  misc_feature 1..27: note = Synthetic probe
  source 1..27: mol_type = other DNA
tgaattctcgtaactacatagcacaag

SEQ ID NO: 240 - DNA, 27 bp, synthetic construct
  misc_feature 1..27: note = Synthetic probe
  source 1..27: mol_type = other DNA
tgaattctcgtaactacatagcacaat

SEQ ID NO: 241 - DNA, 30 bp, synthetic construct
  misc_feature 1..30: note = Synthetic probe
  source 1..30: mol_type = other DNA
tagatgtagttaactttaatctcacatagc

SEQ ID NO: 242 - DNA, 31 bp, synthetic construct
  misc_feature 1..31: note = Synthetic probe
  source 1..31: mol_type = other DNA
gctatgtgagattaaagttaactacatctac

SEQ ID NO: 243 - DNA, 31 bp, synthetic construct
  misc_feature 1..31: note = Synthetic probe
  source 1..31: mol_type = other DNA
gctatgtgagattaaagttaactacatctaa

SEQ ID NO: 244 - DNA, 26 bp, synthetic construct
  misc_feature 1..26: note = Synthetic probe
  source 1..26: mol_type = other DNA
ttgtgctatgtagttacgagaattca

SEQ ID NO: 245 - DNA, 31 bp, synthetic construct
  misc_feature 1..31: note = Synthetic probe
  source 1..31: mol_type = other DNA
ttgaaattaaattggcaaagaaatttgacac

SEQ ID NO: 246 - DNA, 31 bp, synthetic construct
  misc_feature 1..31: note = Synthetic probe
  source 1..31: mol_type = other DNA
ttgaaattaaattggcaaagaaatttgacat

SEQ ID NO: 247 - DNA, 26 bp, synthetic construct
  misc_feature 1..26: note = Synthetic probe
  source 1..26: mol_type = other DNA
cttcaatggggaatgtccaaattttg

SEQ ID NO: 248 - DNA, 26 bp, synthetic construct
  misc_feature 1..26: note = Synthetic probe
  source 1..26: mol_type = other DNA
aaaatttggacattccccattgaagg

SEQ ID NO: 249 - DNA, 26 bp, synthetic construct
  misc_feature 1..26: note = Synthetic probe
  source 1..26: mol_type = other DNA
aaaatttggacattccccattgaaga

SEQ ID NO: 250 - DNA, 31 bp, synthetic construct
  misc_feature 1..31: note = Synthetic probe
  source 1..31: mol_type = other DNA
tgtcaaatttctttgccaatttaatttcaaa

SEQ ID NO: 251 - DNA, 24 bp, synthetic construct
  misc_feature 1..24: note = Synthetic probe
  source 1..24: mol_type = other DNA
gttgcacttcttgctgtttttcag

SEQ ID NO: 252 - DNA, 24 bp, synthetic construct
  misc_feature 1..24: note = Synthetic probe
  source 1..24: mol_type = other DNA
gttgcacttcttgctgtttttcat

SEQ ID NO: 253 - DNA, 23 bp, synthetic construct
  misc_feature 1..23: note = Synthetic probe
  source 1..23: mol_type = other DNA
agcgcttccaaaatcataaccct

SEQ ID NO: 254 - DNA, 23 bp, synthetic construct
  misc_feature 1..23: note = Synthetic probe
  source 1..23: mol_type = other DNA
gggttatgattttggaagcgctc

SEQ ID NO: 255 - DNA, 23 bp, synthetic construct
  misc_feature 1..23: note = Synthetic probe
  source 1..23: mol_type = other DNA
gggttatgattttggaagcgcta

SEQ ID NO: 256 - DNA, 23 bp, synthetic construct
  misc_feature 1..23: note = Synthetic probe
  source 1..23: mol_type = other DNA
tgaaaaacagcaagaagtgcaac

SEQ ID NO: 257 - DNA, 29 bp, synthetic construct
  misc_feature 1..29: note = Synthetic probe
  source 1..29: mol_type = other DNA
ttttgtaatgatccatttttgggtgttta

SEQ ID NO: 258 - DNA, 28 bp, synthetic construct
  misc_feature 1..28: note = Synthetic probe
  source 1..28: mol_type = other DNA
aattttgtaatgatccatttttgggtgt

SEQ ID NO: 259 - DNA, 27 bp, synthetic construct
  misc_feature 1..27: note = Synthetic probe
  source 1..27: mol_type = other DNA
ttaccacaaaaacaacaaaagttggat

SEQ ID NO: 260 - DNA, 28 bp, synthetic construct
  misc_feature 1..28: note = Synthetic probe
  source 1..28: mol_type = other DNA
ccaacttttgttgtttttgtggtaataa

SEQ ID NO: 261 - DNA, 27 bp, synthetic construct
  misc_feature 1..27: note = Synthetic probe
  source 1..27: mol_type = other DNA
atccaacttttgttgtttttgtggtaa

SEQ ID NO: 262 - DNA, 28 bp, synthetic construct
  misc_feature 1..28: note = Synthetic probe
  source 1..28: mol_type = other DNA
acacccaaaaatggatcattacaaaatt

SEQ ID NO: 263 - DNA, 24 bp, synthetic construct
  misc_feature 1..24: note = Synthetic probe
  source 1..24: mol_type = other DNA
ccaacaatttggcagagacattgc

SEQ ID NO: 264 - DNA, 24 bp, synthetic construct
  misc_feature 1..24: note = Synthetic probe
  source 1..24: mol_type = other DNA
ccaacaatttggcagagacattga

SEQ ID NO: 265 - DNA, 21 bp, synthetic construct
  misc_feature 1..21: note = Synthetic probe
  source 1..21: mol_type = other DNA
tgacactactgatgctgtccg

SEQ ID NO: 266 - DNA, 22 bp, synthetic construct
  misc_feature 1..22: note = Synthetic probe
  source 1..22: mol_type = other DNA
cggacagcatcagtagtgtcag

SEQ ID NO: 267 - DNA, 22 bp, synthetic construct
  misc_feature 1..22: note = Synthetic probe
  source 1..22: mol_type = other DNA
cggacagcatcagtagtgtcat

SEQ ID NO: 268 - DNA, 24 bp, synthetic construct
  misc_feature 1..24: note = Synthetic probe
  source 1..24: mol_type = other DNA
caatgtctctgccaaattgttgga

SEQ ID NO: 269 - DNA, 26 bp, synthetic construct
  misc_feature 1..26: note = Synthetic probe
  source 1..26: mol_type = other DNA
tctaataactctattgccatacccac

SEQ ID NO: 270 - DNA, 26 bp, synthetic construct
  misc_feature 1..26: note = Synthetic probe
  source 1..26: mol_type = other DNA
tctaataactctattgccatacccat

SEQ ID NO: 271 - DNA, 32 bp, synthetic construct
  misc_feature 1..32: note = Synthetic probe
  source 1..32: mol_type = other DNA
aaattttactattagtgttaccacagaaattc

SEQ ID NO: 272 - DNA, 33 bp, synthetic construct
  misc_feature 1..33: note = Synthetic probe
  source 1..33: mol_type = other DNA
gaatttctgtggtaacactaatagtaaaatttg

SEQ ID NO: 273 - DNA, 33 bp, synthetic construct
  misc_feature 1..33: note = Synthetic probe
  source 1..33: mol_type = other DNA
gaatttctgtggtaacactaatagtaaaattta

SEQ ID NO: 274 - DNA, 29 bp, synthetic construct
  misc_feature 1..29: note = Synthetic probe
  source 1..29: mol_type = other DNA
tgggtatggcaatagagttattagagtaa

SEQ ID NO: 275 - DNA, 31 bp, synthetic construct
  misc_feature 1..31: note = Synthetic probe
  source 1..31: mol_type = other DNA
gcaatttcaagtgttttaaatgatatccttt

SEQ ID NO: 276 - DNA, 31 bp, synthetic construct
  misc_feature 1..31: note = Synthetic probe
  source 1..31: mol_type = other DNA
gcaatttcaagtgttttaaatgatatccttg

SEQ ID NO: 277 - DNA, 22 bp, synthetic construct
  misc_feature 1..22: note = Synthetic probe
  source 1..22: mol_type = other DNA
cacgtcttgacaaagttgaggc

SEQ ID NO: 278 - DNA, 23 bp, synthetic construct
  misc_feature 1..23: note = Synthetic probe
  source 1..23: mol_type = other DNA
gcctcaactttgtcaagacgtga

SEQ ID NO: 279 - DNA, 23 bp, synthetic construct
  misc_feature 1..23: note = Synthetic probe
  source 1..23: mol_type = other DNA
gcctcaactttgtcaagacgtgc

SEQ ID NO: 280 - DNA, 31 bp, synthetic construct
  misc_feature 1..31: note = Synthetic probe
  source 1..31: mol_type = other DNA
aaggatatcatttaaaacacttgaaattgca

SEQ ID NO: 281 - DNA, 32 bp, synthetic construct
  misc_feature 1..32: note = Synthetic probe
  source 1..32: mol_type = other DNA
gaatttttatgaaccacaaatcattactacag

SEQ ID NO: 282 - DNA, 32 bp, synthetic construct
  misc_feature 1..32: note = Synthetic probe
  source 1..32: mol_type = other DNA
gaatttttatgaaccacaaatcattactacac

SEQ ID NO: 283 - DNA, 24 bp, synthetic construct
  misc_feature 1..24: note = Synthetic probe
  source 1..24: mol_type = other DNA
acaacacatttgtgtctggtaact

SEQ ID NO: 284 - DNA, 24 bp, synthetic construct
  misc_feature 1..24: note = Synthetic probe
  source 1..24: mol_type = other DNA
gttaccagacacaaatgtgttgtc

SEQ ID NO: 285 - DNA, 24 bp, synthetic construct
  misc_feature 1..24: note = Synthetic probe
  source 1..24: mol_type = other DNA
gttaccagacacaaatgtgttgtg

SEQ ID NO: 286 - DNA, 32 bp, synthetic construct
  misc_feature 1..32: note = Synthetic probe
  source 1..32: mol_type = other DNA
tgtagtaatgatttgtggttcataaaaattcc

SEQ ID NO: 287 - DNA, 24 bp, synthetic construct
  misc_feature 1..24: note = Synthetic probe
  source 1..24: mol_type = other DNA
cgatcatcagcacatctaggtttc

SEQ ID NO: 288 - DNA, 24 bp, synthetic construct
  misc_feature 1..24: note = Synthetic probe
  source 1..24: mol_type = other DNA
cgatcatcagcacatctaggtttt

SEQ ID NO: 289 - DNA, 19 bp, synthetic construct
  misc_feature 1..19: note = Synthetic probe
  source 1..19: mol_type = other DNA
gtccgggtgtgaccgaaag

SEQ ID NO: 290 - DNA, 16 bp, synthetic construct
  misc_feature 1..16: note = Synthetic probe
  source 1..16: mol_type = other DNA
cggtcacacccggacg

SEQ ID NO: 291 - DNA, 16 bp, synthetic construct
  misc_feature 1..16: note = Synthetic probe
  source 1..16: mol_type = other DNA
cggtcacacccggaca

SEQ ID NO: 292 - DNA, 23 bp, synthetic construct
  misc_feature 1..23: note = Synthetic probe
  source 1..23: mol_type = other DNA
aaacctagatgtgctgatgatcg

SEQ ID NO: 293 - DNA, 28 bp, synthetic construct
  misc_feature 1..28: note = Synthetic probe
  source 1..28: mol_type = other DNA
attggcttcacatatgtattgttctttc

SEQ ID NO: 294 - DNA, 28 bp, synthetic construct
  misc_feature 1..28: note = Synthetic probe
  source 1..28: mol_type = other DNA
attggcttcacatatgtattgttctttt

SEQ ID NO: 295 - DNA, 23 bp, synthetic construct
  misc_feature 1..23: note = Synthetic probe
  source 1..23: mol_type = other DNA
taccctccagatgaggatgaaga

SEQ ID NO: 296 - DNA, 23 bp, synthetic construct
  misc_feature 1..23: note = Synthetic probe
  source 1..23: mol_type = other DNA
cttcatcctcatctggagggtag

SEQ ID NO: 297 - DNA, 23 bp, synthetic construct
  misc_feature 1..23: note = Synthetic probe
  source 1..23: mol_type = other DNA
cttcatcctcatctggagggtaa

SEQ ID NO: 298 - DNA, 28 bp, synthetic construct
  misc_feature 1..28: note = Synthetic probe
  source 1..28: mol_type = other DNA
aaagaacaatacatatgtgaagccaatt

SEQ ID NO: 299 - DNA, 25 bp, synthetic construct
  misc_feature 1..25: note = Synthetic probe
  source 1..25: mol_type = other DNA
ttttattctctacagtgttcccacc

SEQ ID NO: 300 - DNA, 25 bp, synthetic construct
  misc_feature 1..25: note = Synthetic probe
  source 1..25: mol_type = other DNA
ttttattctctacagtgttcccact

SEQ ID NO: 301 - DNA, 25 bp, synthetic construct
  misc_feature 1..25: note = Synthetic probe
  source 1..25: mol_type = other DNA
tacaagttttggaccactagtgaga

SEQ ID NO: 302 - DNA, 25 bp, synthetic construct
  misc_feature 1..25: note = Synthetic probe
  source 1..25: mol_type = other DNA
ctcactagtggtccaaaacttgtag

SEQ ID NO: 303 - DNA, 25 bp, synthetic construct
  misc_feature 1..25: note = Synthetic probe
  source 1..25: mol_type = other DNA
ctcactagtggtccaaaacttgtaa

SEQ ID NO: 304 - DNA, 26 bp, synthetic construct
  misc_feature 1..26: note = Synthetic probe
  source 1..26: mol_type = other DNA
gtgggaacactgtagagaataaaact

SEQ ID NO: 305 - DNA, 23 bp, synthetic construct
  misc_feature 1..23: note = Synthetic probe
  source 1..23: mol_type = other DNA
gtccaaattcacacaatcgacgg

SEQ ID NO: 306 - DNA, 23 bp, synthetic construct
  misc_feature 1..23: note = Synthetic probe
  source 1..23: mol_type = other DNA
gtccaaattcacacaatcgacgt

SEQ ID NO: 307 - DNA, 23 bp, synthetic construct
  misc_feature 1..23: note = Synthetic probe
  source 1..23: mol_type = other DNA
ttcatccggagttgttaatccag

SEQ ID NO: 308 - DNA, 24 bp, synthetic construct
  misc_feature 1..24: note = Synthetic probe
  source 1..24: mol_type = other DNA
ctggattaacaactccggatgaac

SEQ ID NO: 309 - DNA, 24 bp, synthetic construct
  misc_feature 1..24: note = Synthetic probe
  source 1..24: mol_type = other DNA
ctggattaacaactccggatgaaa

SEQ ID NO: 310 - DNA, 23 bp, synthetic construct
  misc_feature 1..23: note = Synthetic probe
  source 1..23: mol_type = other DNA
cgtcgattgtgtgaatttggaca

SEQ ID NO: 311 - DNA, 26 bp, synthetic construct
  misc_feature 1..26: note = Synthetic probe
  source 1..26: mol_type = other DNA
aagcatacaatgtaacacaagctttc

SEQ ID NO: 312 - DNA, 26 bp, synthetic construct
  misc_feature 1..26: note = Synthetic probe
  source 1..26: mol_type = other DNA
aagcatacaatgtaacacaagctttt

SEQ ID NO: 313 - DNA, 18 bp, synthetic construct
  misc_feature 1..18: note = Synthetic probe
  source 1..18: mol_type = other DNA
ggcagacgtggtccagaa

SEQ ID NO: 314 - DNA, 17 bp, synthetic construct
  misc_feature 1..17: note = Synthetic probe
  source 1..17: mol_type = other DNA
ctggaccacgtctgccg

SEQ ID NO: 315 - DNA, 17 bp, synthetic construct
  misc_feature 1..17: note = Synthetic probe
  source 1..17: mol_type = other DNA
ctggaccacgtctgcca

SEQ ID NO: 316 - DNA, 27 bp, synthetic construct
  misc_feature 1..27: note = Synthetic probe
  source 1..27: mol_type = other DNA
aaagcttgtgttacattgtatgcttta

SEQ ID NO: 317 - DNA, 27 bp, synthetic construct
  misc_feature 1..27: note = Synthetic probe
  source 1..27: mol_type = other DNA
cagatgattttacaggctgcgttatag

SEQ ID NO: 318 - DNA, 27 bp, synthetic construct
  misc_feature 1..27: note = Synthetic probe
  source 1..27: mol_type = other DNA
cagatgattttacaggctgcgttatat

SEQ ID NO: 319 - DNA, 30 bp, synthetic construct
  misc_feature 1..30: note = Synthetic probe
  source 1..30: mol_type = other DNA
cttggaattctaacaatcttgattctaagg

SEQ ID NO: 320 - DNA, 30 bp, synthetic construct
  misc_feature 1..30: note = Synthetic probe
  source 1..30: mol_type = other DNA
cttagaatcaagattgttagaattccaagc

SEQ ID NO: 321 - DNA, 30 bp, synthetic construct
  misc_feature 1..30: note = Synthetic probe
  source 1..30: mol_type = other DNA
cttagaatcaagattgttagaattccaaga

SEQ ID NO: 322 - DNA, 25 bp, synthetic construct
  misc_feature 1..25: note = Synthetic probe
  source 1..25: mol_type = other DNA
tataacgcagcctgtaaaatcatct

SEQ ID NO: 323 - DNA, 75 bp, synthetic construct
  misc_feature 1..75: note = Synthetic oligonucleotide
  source 1..75: mol_type = other DNA
attaatttagtgcgtgatctccctcagggtttttcggctttagaaccattggtagatttgccaataggtattaac

SEQ ID NO: 324 - DNA, 75 bp, synthetic construct
  misc_feature 1..75: note = Synthetic oligonucleotide
  source 1..75: mol_type = other DNA
attaatttagtgcgtgatctccctcagggtttttcggttttagaaccattggtagatttgccaataggtattaac

SEQ ID NO: 325 - DNA, 75 bp, synthetic construct
  misc_feature 1..75: note = Synthetic oligonucleotide
  source 1..75: mol_type = other DNA
gttaatacctattggcaaatctaccaatggttctaaagccgaaaaaccctgagggagatcacgcactaaattaat

SEQ ID NO: 326 - DNA, 75 bp, synthetic construct
  misc_feature 1..75: note = Synthetic oligonucleotide
  source 1..75: mol_type = other DNA
gttaatacctattggcaaatctaccaatggttctaaaaccgaaaaaccctgagggagatcacgcactaaattaat

SEQ ID NO: 327 - DNA, 75 bp, synthetic construct
  misc_feature 1..75: note = Synthetic oligonucleotide
  source 1..75: mol_type = other DNA
gctagaatggctggcaatggcggtgatgctgctcttgctttgctgctgcttgacagattgaaccagcttgagagc

SEQ ID NO: 328 - DNA, 75 bp, synthetic construct
  misc_feature 1..75: note = Synthetic oligonucleotide
  source 1..75: mol_type = other DNA
gctagaatggctggcaatggcggtgatgctgctcttgttttgctgctgcttgacagattgaaccagcttgagagc

SEQ ID NO: 329 - DNA, 75 bp, synthetic construct
  misc_feature 1..75: note = Synthetic oligonucleotide
  source 1..75: mol_type = other DNA
gctctcaagctggttcaatctgtcaagcagcagcaaagcaagagcagcatcaccgccattgccagccattctagc

SEQ ID NO: 330 - DNA, 75 bp, synthetic construct
  misc_feature 1..75: note = Synthetic oligonucleotide
  source 1..75: mol_type = other DNA
gctctcaagctggttcaatctgtcaagcagcagcaaaacaagagcagcatcaccgccattgccagccattctagc

SEQ ID NO: 331 - DNA, 75 bp, synthetic construct
  misc_feature 1..75: note = Synthetic oligonucleotide
  source 1..75: mol_type = other DNA
cttgtgcagaatgaattctcgtaactacatagcacaagtagatgtagttaactttaatctcacatagcaatcttt

SEQ ID NO: 332 - DNA, 75 bp, synthetic construct
  misc_feature 1..75: note = Synthetic oligonucleotide
  source 1..75: mol_type = other DNA
cttgtgcagaatgaattctcgtaactacatagcacaattagatgtagttaactttaatctcacatagcaatcttt

SEQ ID NO: 333 - DNA, 75 bp, synthetic construct
  misc_feature 1..75: note = Synthetic oligonucleotide
  source 1..75: mol_type = other DNA
aaagattgctatgtgagattaaagttaactacatctacttgtgctatgtagttacgagaattcattctgcacaag

SEQ ID NO: 334 - DNA, 75 bp, synthetic construct
  misc_feature 1..75: note = Synthetic oligonucleotide
  source 1..75: mol_type = other DNA
aaagattgctatgtgagattaaagttaactacatctaattgtgctatgtagttacgagaattcattctgcacaag

SEQ ID NO: 335 - DNA, 75 bp, synthetic construct
  misc_feature 1..75: note = Synthetic oligonucleotide
  source 1..75: mol_type = other DNA
acaccttttgaaattaaattggcaaagaaatttgacaccttcaatggggaatgtccaaattttgtatttccctta

SEQ ID NO: 336 - DNA, 75 bp, synthetic construct
  misc_feature 1..75: note = Synthetic oligonucleotide
  source 1..75: mol_type = other DNA
acaccttttgaaattaaattggcaaagaaatttgacatcttcaatggggaatgtccaaattttgtatttccctta

SEQ ID NO: 337 - DNA, 75 bp, synthetic construct
  misc_feature 1..75: note = Synthetic oligonucleotide
  source 1..75: mol_type = other DNA
taagggaaatacaaaatttggacattccccattgaaggtgtcaaatttctttgccaatttaatttcaaaaggtgt

SEQ ID NO: 338 - DNA, 75 bp, synthetic construct
  misc_feature 1..75: note = Synthetic oligonucleotide
  source 1..75: mol_type = other DNA
taagggaaatacaaaatttggacattccccattgaagatgtcaaatttctttgccaatttaatttcaaaaggtgt

SEQ ID NO: 339 - DNA, 75 bp, synthetic construct
  misc_feature 1..75: note = Synthetic oligonucleotide
  source 1..75: mol_type = other DNA
ggcttattgttggcgttgcacttcttgctgtttttcagagcgcttccaaaatcataaccctcaaaaagagatggc

SEQ ID NO: 340 - DNA, 75 bp, synthetic construct
  misc_feature 1..75: note = Synthetic oligonucleotide
  source 1..75: mol_type = other DNA
ggcttattgttggcgttgcacttcttgctgtttttcatagcgcttccaaaatcataaccctcaaaaagagatggc

SEQ ID NO: 341 - DNA, 75 bp, synthetic construct
  misc_feature 1..75: note = Synthetic oligonucleotide
  source 1..75: mol_type = other DNA
gccatctctttttgagggttatgattttggaagcgctctgaaaaacagcaagaagtgcaacgccaacaataagcc

SEQ ID NO: 342 - DNA, 75 bp, synthetic construct
  misc_feature 1..75: note = Synthetic oligonucleotide
  source 1..75: mol_type = other DNA
gccatctctttttgagggttatgattttggaagcgctatgaaaaacagcaagaagtgcaacgccaacaataagcc

SEQ ID NO: 343 - DNA, 75 bp, unidentified
  misc_feature 1..75: note = WT template strand
  source 1..75: mol_type = other DNA
tgaatttcaattttgtaatgatccatttttgggtgtttattaccacaaaaacaacaaaagttggatggaaagtga

SEQ ID NO: 344 - DNA, 75 bp, synthetic construct
  misc_feature 1..75: note = Synthetic oligonucleotide
  source 1..75: mol_type = other DNA
gtgaatttcaattttgtaatgatccatttttgggtgtttaccacaaaaacaacaaaagttggatggaaagtgagt

SEQ ID NO: 345 - DNA, 75 bp, unidentified
  misc_feature 1..75: note = WT cDNA strand
  source 1..75: mol_type = other DNA
tcactttccatccaacttttgttgtttttgtggtaataaacacccaaaaatggatcattacaaaattgaaattca

SEQ ID NO: 346 - DNA, 75 bp, synthetic construct
  misc_feature 1..75: note = Synthetic oligonucleotide
  source 1..75: mol_type = other DNA
actcactttccatccaacttttgttgtttttgtggtaaacacccaaaaatggatcattacaaaattgaaattcac

SEQ ID NO: 347 - DNA, 75 bp, synthetic construct
  misc_feature 1..75: note = Synthetic oligonucleotide
  source 1..75: mol_type = other DNA
aagtttctgcctttccaacaatttggcagagacattgctgacactactgatgctgtccgtgatccacagacactt

SEQ ID NO: 348 - DNA, 75 bp, synthetic construct
  misc_feature 1..75: note = Synthetic oligonucleotide
  source 1..75: mol_type = other DNA
aagtttctgcctttccaacaatttggcagagacattgatgacactactgatgctgtccgtgatccacagacactt

SEQ ID NO: 349 - DNA, 75 bp, synthetic construct
  misc_feature 1..75: note = Synthetic oligonucleotide
  source 1..75: mol_type = other DNA
aagtgtctgtggatcacggacagcatcagtagtgtcagcaatgtctctgccaaattgttggaaaggcagaaactt

SEQ ID NO: 350 - DNA, 75 bp, synthetic construct
  misc_feature 1..75: note = Synthetic oligonucleotide
  source 1..75: mol_type = other DNA
aagtgtctgtggatcacggacagcatcagtagtgtcatcaatgtctctgccaaattgttggaaaggcagaaactt

SEQ ID NO: 351 - DNA, 75 bp, synthetic construct
  misc_feature 1..75: note = Synthetic oligonucleotide
  source 1..75: mol_type = other DNA
tcagttgcttactctaataactctattgccatacccacaaattttactattagtgttaccacagaaattctacca

SEQ ID NO: 352 - DNA, 75 bp, synthetic construct
  misc_feature 1..75: note = Synthetic oligonucleotide
  source 1..75: mol_type = other DNA
tcagttgcttactctaataactctattgccatacccataaattttactattagtgttaccacagaaattctacca

SEQ ID NO: 353 - DNA, 75 bp, synthetic construct
  misc_feature 1..75: note = Synthetic oligonucleotide
  source 1..75: mol_type = other DNA
tggtagaatttctgtggtaacactaatagtaaaatttgtgggtatggcaatagagttattagagtaagcaactga

SEQ ID NO: 354 - DNA, 75 bp, synthetic construct
  misc_feature 1..75: note = Synthetic oligonucleotide
  source 1..75: mol_type = other DNA
tggtagaatttctgtggtaacactaatagtaaaatttatgggtatggcaatagagttattagagtaagcaactga

SEQ ID NO: 355 - DNA, 75 bp, synthetic construct
  misc_feature 1..75: note = Synthetic oligonucleotide
  source 1..75: mol_type = other DNA
ttttggtgcaatttcaagtgttttaaatgatatcctttcacgtcttgacaaagttgaggctgaagtgcaaatatt

SEQ ID NO: 356 - DNA, 75 bp, synthetic construct
  misc_feature 1..75: note = Synthetic oligonucleotide
  source 1..75: mol_type = other DNA
ttttggtgcaatttcaagtgttttaaatgatatccttgcacgtcttgacaaagttgaggctgaagtgcaaatatt

SEQ ID NO: 357 - DNA, 75 bp, synthetic construct
  misc_feature 1..75: note = Synthetic oligonucleotide
  source 1..75: mol_type = other DNA
aatatttgcacttcagcctcaactttgtcaagacgtgaaaggatatcatttaaaacacttgaaattgcaccaaaa

SEQ ID NO: 358 - DNA, 75 bp, synthetic construct
  misc_feature 1..75: note = Synthetic oligonucleotide
  source 1..75: mol_type = other DNA
aatatttgcacttcagcctcaactttgtcaagacgtgcaaggatatcatttaaaacacttgaaattgcaccaaaa

SEQ ID NO: 359 - DNA, 75 bp, synthetic construct
  misc_feature 1..75: note = Synthetic oligonucleotide
  source 1..75: mol_type = other DNA
acaaaggaatttttatgaaccacaaatcattactacagacaacacatttgtgtctggtaactgtgatgttgtaat

SEQ ID NO: 360 - DNA, 75 bp, synthetic construct
  misc_feature 1..75: note = Synthetic oligonucleotide
  source 1..75: mol_type = other DNA
acaaaggaatttttatgaaccacaaatcattactacacacaacacatttgtgtctggtaactgtgatgttgtaat

SEQ ID NO: 361 - DNA, 75 bp, synthetic construct
  misc_feature 1..75: note = Synthetic oligonucleotide
  source 1..75: mol_type = other DNA
attacaacatcacagttaccagacacaaatgtgttgtctgtagtaatgatttgtggttcataaaaattcctttgt

SEQ ID NO: 362 - DNA, 75 bp, synthetic construct
  misc_feature 1..75: note = Synthetic oligonucleotide
  source 1..75: mol_type = other DNA
attacaacatcacagttaccagacacaaatgtgttgtgtgtagtaatgatttgtggttcataaaaattcctttgt

SEQ ID NO: 363 - DNA, 75 bp, synthetic construct
  misc_feature 1..75: note = Synthetic oligonucleotide
  source 1..75: mol_type = other DNA
gtccgtgttgcagccgatcatcagcacatctaggtttcgtccgggtgtgaccgaaaggtaagatggagagccttg

SEQ ID NO: 364 - DNA, 75 bp, synthetic construct
  misc_feature 1..75: note = Synthetic oligonucleotide
  source 1..75: mol_type = other DNA
gtccgtgttgcagccgatcatcagcacatctaggttttgtccgggtgtgaccgaaaggtaagatggagagccttg

SEQ ID NO: 365 - DNA, 75 bp, synthetic construct
  misc_feature 1..75: note = Synthetic oligonucleotide
  source 1..75: mol_type = other DNA
caaggctctccatcttacctttcggtcacacccggacgaaacctagatgtgctgatgatcggctgcaacacggac

SEQ ID NO: 366 - DNA, 75 bp, synthetic construct
  misc_feature 1..75: note = Synthetic oligonucleotide
  source 1..75: mol_type = other DNA
caaggctctccatcttacctttcggtcacacccggacaaaacctagatgtgctgatgatcggctgcaacacggac

SEQ ID NO: 367 - DNA, 75 bp, synthetic construct
  misc_feature 1..75: note = Synthetic oligonucleotide
  source 1..75: mol_type = other DNA
gtgagtttaaattggcttcacatatgtattgttctttctaccctccagatgaggatgaagaagaaggtgattgtg

SEQ ID NO: 368 - DNA, 75 bp, synthetic construct
  misc_feature 1..75: note = Synthetic oligonucleotide
  source 1..75: mol_type = other DNA
gtgagtttaaattggcttcacatatgtattgttctttttaccctccagatgaggatgaagaagaaggtgattgtg

SEQ ID NO: 369 - DNA, 75 bp, synthetic construct
  misc_feature 1..75: note = Synthetic oligonucleotide
  source 1..75: mol_type = other DNA
cacaatcaccttcttcttcatcctcatctggagggtagaaagaacaatacatatgtgaagccaatttaaactcac

SEQ ID NO: 370 - DNA, 75 bp, synthetic construct
  misc_feature 1..75: note = Synthetic oligonucleotide
  source 1..75: mol_type = other DNA
cacaatcaccttcttcttcatcctcatctggagggtaaaaagaacaatacatatgtgaagccaatttaaactcac

SEQ ID NO: 371 - DNA, 75 bp, synthetic construct
  misc_feature 1..75: note = Synthetic oligonucleotide
  source 1..75: mol_type = other DNA
gcaaactttaatgttttattctctacagtgttcccacctacaagttttggaccactagtgagaaaaatatttgtt

SEQ ID NO: 372 - DNA, 75 bp, synthetic construct
  misc_feature 1..75: note = Synthetic oligonucleotide
  source 1..75: mol_type = other DNA
gcaaactttaatgttttattctctacagtgttcccacttacaagttttggaccactagtgagaaaaatatttgtt

SEQ ID NO: 373 - DNA, 75 bp, synthetic construct
  misc_feature 1..75: note = Synthetic oligonucleotide
  source 1..75: mol_type = other DNA
aacaaatatttttctcactagtggtccaaaacttgtaggtgggaacactgtagagaataaaacattaaagtttgc

SEQ ID NO: 374 - DNA, 75 bp, synthetic construct
  misc_feature 1..75: note = Synthetic oligonucleotide
  source 1..75: mol_type = other DNA
aacaaatatttttctcactagtggtccaaaacttgtaagtgggaacactgtagagaataaaacattaaagtttgc

SEQ ID NO: 375 - DNA, 75 bp, synthetic construct
  misc_feature 1..75: note = Synthetic oligonucleotide
  source 1..75: mol_type = other DNA
gagcctgaagaacatgtccaaattcacacaatcgacggttcatccggagttgttaatccagtaatggaaccaatt

SEQ ID NO: 376 - DNA, 75 bp, synthetic construct
  misc_feature 1..75: note = Synthetic oligonucleotide
  source 1..75: mol_type = other DNA
gagcctgaagaacatgtccaaattcacacaatcgacgtttcatccggagttgttaatccagtaatggaaccaatt

SEQ ID NO: 377 - DNA, 75 bp, synthetic construct
  misc_feature 1..75: note = Synthetic oligonucleotide
  source 1..75: mol_type = other DNA
aattggttccattactggattaacaactccggatgaaccgtcgattgtgtgaatttggacatgttcttcaggctc

SEQ ID NO: 378 - DNA, 75 bp, synthetic construct
  misc_feature 1..75: note = Synthetic oligonucleotide
  source 1..75: mol_type = other DNA
aattggttccattactggattaacaactccggatgaaacgtcgattgtgtgaatttggacatgttcttcaggctc

SEQ ID NO: 379 - DNA, 75 bp, synthetic construct
  misc_feature 1..75: note = Synthetic oligonucleotide
  source 1..75: mol_type = other DNA
gtactgccactaaagcatacaatgtaacacaagctttcggcagacgtggtccagaacaaacccaaggaaattttg

SEQ ID NO: 380 - DNA, 75 bp, synthetic construct
  misc_feature 1..75: note = Synthetic oligonucleotide
  source 1..75: mol_type = other DNA
gtactgccactaaagcatacaatgtaacacaagcttttggcagacgtggtccagaacaaacccaaggaaattttg

SEQ ID NO: 381 - DNA, 75 bp, synthetic construct
  misc_feature 1..75: note = Synthetic oligonucleotide
  source 1..75: mol_type = other DNA
caaaatttccttgggtttgttctggaccacgtctgccgaaagcttgtgttacattgtatgctttagtggcagtac

SEQ ID NO: 382 - DNA, 75 bp, synthetic construct
  misc_feature 1..75: note = Synthetic oligonucleotide
  source 1..75: mol_type = other DNA
caaaatttccttgggtttgttctggaccacgtctgccaaaagcttgtgttacattgtatgctttagtggcagtac

SEQ ID NO: 383 - DNA, 75 bp, synthetic construct
  misc_feature 1..75: note = Synthetic oligonucleotide
  source 1..75: mol_type = other DNA
ttataaattaccagatgattttacaggctgcgttatagcttggaattctaacaatcttgattctaaggttggtgg

SEQ ID NO: 384 - DNA, 75 bp, synthetic construct
  misc_feature 1..75: note = Synthetic oligonucleotide
  source 1..75: mol_type = other DNA
ttataaattaccagatgattttacaggctgcgttatatcttggaattctaacaatcttgattctaaggttggtgg

SEQ ID NO: 385 - DNA, 75 bp, synthetic construct
  misc_feature 1..75: note = Synthetic oligonucleotide
  source 1..75: mol_type = other DNA
ccaccaaccttagaatcaagattgttagaattccaagctataacgcagcctgtaaaatcatctggtaatttataa

SEQ ID NO: 386 - DNA, 75 bp, synthetic construct
  misc_feature 1..75: note = Synthetic oligonucleotide
  source 1..75: mol_type = other DNA
ccaccaaccttagaatcaagattgttagaattccaagatataacgcagcctgtaaaatcatctggtaatttataa

SEQ ID NO: 387 - DNA, 19 bp, synthetic construct
  misc_feature 1..19: note = Synthetic oligonucleotide
  source 1..19: mol_type = other DNA
tccctcagggtttttcggy

SEQ ID NO: 388 - DNA, 25 bp, synthetic construct
  misc_feature 1..25: note = Synthetic oligonucleotide
  source 1..25: mol_type = other DNA
tttagaaccattggtagatttgcca

SEQ ID NO: 389 - DNA, 25 bp, synthetic construct
  misc_feature 1..25: note = Synthetic oligonucleotide
  source 1..25: mol_type = other DNA
ggcaaatctaccaatggttctaaar

SEQ ID NO: 390 - DNA, 20 bp, synthetic construct
  misc_feature 1..20: note = Synthetic oligonucleotide
  source 1..20: mol_type = other DNA
ccgaaaaaccctgagggaga

SEQ ID NO: 391 - DNA, 18 bp, synthetic construct
  misc_feature 1..18: note = Synthetic oligonucleotide
  source 1..18: mol_type = other DNA
cggtgatgctgctcttgy

SEQ ID NO: 392 - DNA, 22 bp, synthetic construct
  misc_feature 1..22: note = Synthetic oligonucleotide
  source 1..22: mol_type = other DNA
tttgctgctgcttgacagattg

SEQ ID NO: 393 - DNA, 23 bp, synthetic construct
  misc_feature 1..23: note = Synthetic oligonucleotide
  source 1..23: mol_type = other DNA
caatctgtcaagcagcagcaaar

SEQ ID NO: 394 - DNA, 18 bp, synthetic construct
  misc_feature 1..18: note = Synthetic oligonucleotide
  source 1..18: mol_type = other DNA
caagagcagcatcaccgc

SEQ ID NO: 395 - DNA, 27 bp, synthetic construct
  misc_feature 1..27: note = Synthetic oligonucleotide
  source 1..27: mol_type = other DNA
tgaattctcgtaactacatagcacaak

SEQ ID NO: 396 - DNA, 30 bp, synthetic construct; identical to SEQ ID NO: 241
  misc_feature 1..30: note = Synthetic oligonucleotide
  source 1..30: mol_type = other DNA

SEQ ID NO: 397 - DNA, 31 bp, synthetic construct
  misc_feature 1..31: note = Synthetic oligonucleotide
  source 1..31: mol_type = other DNA
gctatgtgagattaaagttaactacatctam

SEQ ID NO: 398 - DNA, 26 bp, synthetic construct
  misc_feature 1..26: note = Synthetic oligonucleotide
  source 1..26: mol_type = other DNA
ttgtgctatgtagttacgagaattca

SEQ ID NO: 399 - DNA, 31 bp, synthetic construct
  misc_feature 1..31: note = Synthetic oligonucleotide
  source 1..31: mol_type = other DNA
ttgaaattaaattggcaaagaaatttgacay

SEQ ID NO: 400 - DNA, 26 bp, synthetic construct
  misc_feature 1..26: note = Synthetic oligonucleotide
  source 1..26: mol_type = other DNA
cttcaatggggaatgtccaaattttg

SEQ ID NO: 401 - DNA, 26 bp, synthetic construct
  misc_feature 1..26: note = Synthetic oligonucleotide
  source 1..26: mol_type = other DNA
aaaatttggacattccccattgaagr

SEQ ID NO: 402 - DNA, 31 bp, synthetic construct; identical to SEQ ID NO: 250
  misc_feature 1..31: note = Synthetic oligonucleotide
  source 1..31: mol_type = other DNA

SEQ ID NO: 403 - DNA, 24 bp, synthetic construct
  misc_feature 1..24: note = Synthetic oligonucleotide
  source 1..24: mol_type = other DNA
gttgcacttcttgctgtttttcak

SEQ ID NO: 404 - DNA, 23 bp, synthetic construct
  misc_feature 1..23: note = Synthetic oligonucleotide
  source 1..23: mol_type = other DNA
agcgcttccaaaatcataaccct

SEQ ID NO: 405 - DNA, 23 bp, synthetic construct
  misc_feature 1..23: note = Synthetic oligonucleotide
  source 1..23: mol_type = other DNA
gggttatgattttggaagcgctm

SEQ ID NO: 406 - DNA, 23 bp, synthetic construct
  misc_feature 1..23: note = Synthetic oligonucleotide
  source 1..23: mol_type = other DNA
tgaaaaacagcaagaagtgcaac

SEQ ID NO: 407 - DNA, 29 bp, synthetic construct
  misc_feature 1..29: note = Synthetic oligonucleotide
  source 1..29: mol_type = other DNA
ttttgtaatgatccatttttgggtgttta

SEQ ID NO: 408 - DNA, 28 bp, synthetic construct
  misc_feature 1..28: note = Synthetic oligonucleotide
  source 1..28: mol_type = other DNA
aattttgtaatgatccatttttgggtgt

SEQ ID NO: 409 - DNA, 27 bp, synthetic construct
  misc_feature 1..27: note = Synthetic oligonucleotide
  source 1..27: mol_type = other DNA
ttaccacaaaaacaacaaaagttggat

SEQ ID NO: 410 - DNA, 28 bp, synthetic construct
  misc_feature 1..28: note = Synthetic oligonucleotide
  source 1..28: mol_type = other DNA
ccaacttttgttgtttttgtggtaataa

SEQ ID NO: 411 - DNA, 27 bp, synthetic construct
  misc_feature 1..27: note = Synthetic oligonucleotide
  source 1..27: mol_type = other DNA
atccaacttttgttgtttttgtggtaa

SEQ ID NO: 412 - DNA, 28 bp, synthetic construct
  misc_feature 1..28: note = Synthetic oligonucleotide
  source 1..28: mol_type = other DNA
acacccaaaaatggatcattacaaaatt

SEQ ID NO: 413 - DNA, 24 bp, synthetic construct
  misc_feature 1..24: note = Synthetic oligonucleotide
  source 1..24: mol_type = other DNA
ccaacaatttggcagagacattgm

SEQ ID NO: 414 - DNA, 21 bp, synthetic construct
  misc_feature 1..21: note = Synthetic oligonucleotide
  source 1..21: mol_type = other DNA
tgacactactgatgctgtccg

SEQ ID NO: 415 - DNA, 22 bp, synthetic construct
  misc_feature 1..22: note = Synthetic oligonucleotide
  source 1..22: mol_type = other DNA
cggacagcatcagtagtgtcak

SEQ ID NO: 416 - DNA, 24 bp, synthetic construct
  misc_feature 1..24: note = Synthetic oligonucleotide
  source 1..24: mol_type = other DNA
caatgtctctgccaaattgttgga

SEQ ID NO: 417 - DNA, 26 bp, synthetic construct
  misc_feature 1..26: note = Synthetic oligonucleotide
  source 1..26: mol_type = other DNA
tctaataactctattgccatacccay

SEQ ID NO: 418 - DNA, 32 bp, synthetic construct; identical to SEQ ID NO: 271
  misc_feature 1..32: note = Synthetic oligonucleotide
  source 1..32: mol_type = other DNA

SEQ ID NO: 419 - DNA, 33 bp, synthetic construct
  misc_feature 1..33: note = Synthetic oligonucleotide
  source 1..33: mol_type = other DNA
gaatttctgtggtaacactaatagtaaaatttr

SEQ ID NO: 420 - DNA, 29 bp, synthetic construct
  misc_feature 1..29: note = Synthetic oligonucleotide
  source 1..29: mol_type = other DNA
tgggtatggcaatagagttattagagtaa

SEQ ID NO: 421 - DNA, 31 bp, synthetic construct
  misc_feature 1..31: note = Synthetic oligonucleotide
  source 1..31: mol_type = other DNA
gcaatttcaagtgttttaaatgatatccttk

SEQ ID NO: 422 - DNA, 22 bp, synthetic construct
  misc_feature 1..22: note = Synthetic oligonucleotide
  source 1..22: mol_type = other DNA
cacgtcttgacaaagttgaggc

SEQ ID NO: 423 - DNA, 23 bp, synthetic construct
  misc_feature 1..23: note = Synthetic oligonucleotide
  source 1..23: mol_type = other DNA
gcctcaactttgtcaagacgtgm

SEQ ID NO: 424 - DNA, 31 bp, synthetic construct; identical to SEQ ID NO: 280
  misc_feature 1..31: note = Synthetic oligonucleotide
  source 1..31: mol_type = other DNA

SEQ ID NO: 425 - DNA, 32 bp, synthetic construct
  misc_feature 1..32: note = Synthetic oligonucleotide
  source 1..32: mol_type = other DNA
gaatttttatgaaccacaaatcattactacas

SEQ ID NO: 426 - DNA, 24 bp, synthetic construct
  misc_feature 1..24: note = Synthetic oligonucleotide
  source 1..24: mol_type = other DNA
acaacacatttgtgtctggtaact

SEQ ID NO: 427 - DNA, 24 bp, synthetic construct
  misc_feature 1..24: note = Synthetic oligonucleotide
  source 1..24: mol_type = other DNA
gttaccagacacaaatgtgttgts

SEQ ID NO: 428 - DNA, 32 bp, synthetic construct; identical to SEQ ID NO: 286
  misc_feature 1..32: note = Synthetic oligonucleotide
  source 1..32: mol_type = other DNA

SEQ ID NO: 429 - DNA, 24 bp, synthetic construct
  misc_feature 1..24: note = Synthetic oligonucleotide
  source 1..24: mol_type = other DNA
cgatcatcagcacatctaggttty

SEQ ID NO: 430 - DNA, 19 bp, synthetic construct
  misc_feature 1..19: note = Synthetic oligonucleotide
  source 1..19: mol_type = other DNA
gtccgggtgtgaccgaaag

SEQ ID NO: 431 - DNA, 16 bp, synthetic construct
  misc_feature 1..16: note = Synthetic oligonucleotide
  source 1..16: mol_type = other DNA
cggtcacacccggacr

SEQ ID NO: 432 - DNA, 23 bp, synthetic construct
  misc_feature 1..23: note = Synthetic oligonucleotide
  source 1..23: mol_type = other DNA
aaacctagatgtgctgatgatcg

SEQ ID NO: 433 - DNA, 28 bp, synthetic construct
  misc_feature 1..28: note = Synthetic oligonucleotide
  source 1..28: mol_type = other DNA
attggcttcacatatgtattgttcttty

SEQ ID NO: 434 - DNA, 23 bp, synthetic construct
  misc_feature 1..23: note = Synthetic oligonucleotide
  source 1..23: mol_type = other DNA
taccctccagatgaggatgaaga

SEQ ID NO: 435 - DNA, 23 bp, synthetic construct
  misc_feature 1..23: note = Synthetic oligonucleotide
  source 1..23: mol_type = other DNA
cttcatcctcatctggagggtar

SEQ ID NO: 436 - DNA, 28 bp, synthetic construct
  misc_feature 1..28: note = Synthetic oligonucleotide
  source 1..28: mol_type = other DNA
aaagaacaatacatatgtgaagccaatt

SEQ ID NO: 437 - DNA, 25 bp, synthetic construct
  misc_feature 1..25: note = Synthetic oligonucleotide
  source 1..25: mol_type = other DNA
ttttattctctacagtgttcccacy

SEQ ID NO: 438 - DNA, 25 bp, synthetic construct
  misc_feature 1..25: note = Synthetic oligonucleotide
  source 1..25: mol_type = other DNA
tacaagttttggaccactagtgaga

SEQ ID NO: 439 - DNA, 25 bp, synthetic construct
  misc_feature 1..25: note = Synthetic oligonucleotide
  source 1..25: mol_type = other DNA
ctcactagtggtccaaaacttgtar

SEQ ID NO: 440 - DNA, 26 bp, synthetic construct
  misc_feature 1..26: note = Synthetic oligonucleotide
  source 1..26: mol_type = other DNA
gtgggaacactgtagagaataaaact

SEQ ID NO: 441 - DNA, 23 bp, synthetic construct
  misc_feature 1..23: note = Synthetic oligonucleotide
  source 1..23: mol_type = other DNA
gtccaaattcacacaatcgacgk

SEQ ID NO: 442 - DNA, 23 bp, synthetic construct
  misc_feature 1..23: note = Synthetic oligonucleotide
  source 1..23: mol_type = other DNA
ttcatccggagttgttaatccag

SEQ ID NO: 443 - DNA, 24 bp, synthetic construct
  misc_feature 1..24: note = Synthetic oligonucleotide
  source 1..24: mol_type = other DNA
ctggattaacaactccggatgaam

SEQ ID NO: 444 - DNA, 23 bp, synthetic construct
  misc_feature 1..23: note = Synthetic oligonucleotide
  source 1..23: mol_type = other DNA
cgtcgattgtgtgaatttggaca

SEQ ID NO: 445 - DNA, 26 bp, synthetic construct
  misc_feature 1..26: note = Synthetic oligonucleotide
  source 1..26: mol_type = other DNA
aagcatacaatgtaacacaagcttty

SEQ ID NO: 446 - DNA, 18 bp, synthetic construct
  misc_feature 1..18: note = Synthetic oligonucleotide
  source 1..18: mol_type = other DNA
ggcagacgtggtccagaa

SEQ ID NO: 447 - DNA, 17 bp, synthetic construct
  misc_feature 1..17: note = Synthetic oligonucleotide
  source 1..17: mol_type = other DNA
ctggaccacgtctgccr

SEQ ID NO: 448 - DNA, 27 bp, synthetic construct
  misc_feature 1..27: note = Synthetic oligonucleotide
  source 1..27: mol_type = other DNA
aaagcttgtgttacattgtatgcttta

SEQ ID NO: 449 - DNA, 27 bp, synthetic construct
  misc_feature 1..27: note = Synthetic oligonucleotide
  source 1..27: mol_type = other DNA
cagatgattttacaggctgcgttatak

SEQ ID NO: 450 - DNA, 30 bp, synthetic construct; identical to SEQ ID NO: 319
  misc_feature 1..30: note = Synthetic oligonucleotide
  source 1..30: mol_type = other DNA

SEQ ID NO: 451 - DNA, 30 bp, synthetic construct
  misc_feature 1..30: note = Synthetic oligonucleotide
  source 1..30: mol_type = other DNA
cttagaatcaagattgttagaattccaagm

SEQ ID NO: 452 - DNA, 25 bp, synthetic construct
  misc_feature 1..25: note = Synthetic oligonucleotide
  source 1..25: mol_type = other DNA
tataacgcagcctgtaaaatcatct

SEQ ID NO: 453 - DNA, 22 bp, synthetic construct
  misc_feature 1..22: note = Synthetic primer
  source 1..22: mol_type = other DNA
cgtggtgtttattaccctgaca

SEQ ID NO: 454 - DNA, 22 bp, synthetic construct
  misc_feature 1..22: note = Synthetic primer
  source 1..22: mol_type = other DNA
ataagtagggactgggtcttcg

SEQ ID NO: 455 - DNA, 22 bp, synthetic construct
  misc_feature 1..22: note = Synthetic primer
  source 1..22: mol_type = other DNA
ggatggaaagtgagttcagagt

SEQ ID NO: 456 - DNA, 22 bp, synthetic construct
  misc_feature 1..22: note = Synthetic primer
  source 1..22: mol_type = other DNA
gctgtccaacctgaagaagaat

SEQ ID NO: 457 - DNA, 22 bp, synthetic construct
  misc_feature 1..22: note = Synthetic primer
  source 1..22: mol_type = other DNA
caggctgcgttatagcttggaa

SEQ ID NO: 458 - DNA, 22 bp, synthetic construct
  misc_feature 1..22: note = Synthetic primer
  source 1..22: mol_type = other DNA
tgctggtgcatgtagaagttca

SEQ ID NO: 459 - DNA, 22 bp, synthetic construct
  misc_feature 1..22: note = Synthetic primer
  source 1..22: mol_type = other DNA
tgagtgtgacatacccattggt

SEQ ID NO: 460 - DNA, 22 bp, synthetic construct
  misc_feature 1..22: note = Synthetic primer
  source 1..22: mol_type = other DNA
tgctgcattcagttgaatcacc

SEQ ID NO: 461 - DNA, 27 bp, synthetic construct
  misc_feature 1..27: note = Synthetic primer
  source 1..27: mol_type = other DNA
acattcaactcaggacttgttcttacc

SEQ ID NO: 462 - DNA, 30 bp, synthetic construct
  misc_feature 1..30: note = Synthetic primer
  source 1..30: mol_type = other DNA
cagcctcttattatgttagacttctcagtg

SEQ ID NO: 463 - DNA, 27 bp, synthetic construct
  misc_feature 1..27: note = Synthetic primer
  source 1..27: mol_type = other DNA
gttggatggaaagtgagttcagagttt

SEQ ID NO: 464 - DNA, 27 bp, synthetic construct
  misc_feature 1..27: note = Synthetic primer
  source 1..27: mol_type = other DNA
tggcaaatctaccaatggttctaaagc

SEQ ID NO: 465 - DNA, 25 bp, synthetic construct
  misc_feature 1..25: note = Synthetic primer
  source 1..25: mol_type = other DNA
caggaagagaatcagcaactgtgtt

SEQ ID NO: 466 - DNA, 24 bp, synthetic construct
  misc_feature 1..24: note = Synthetic primer
  source 1..24: mol_type = other DNA
tccaagctataacgcagcctgtaa

SEQ ID NO: 467 - DNA, 25 bp, synthetic construct
  misc_feature 1..25: note = Synthetic primer
  source 1..25: mol_type = other DNA
caatcttgattctaaggttggtggt

SEQ ID NO: 468 - DNA, 27 bp, synthetic construct
  misc_feature 1..27: note = Synthetic primer
  source 1..27: mol_type = other DNA
gtactactactctgtatggttggtaac

SEQ ID NO: 469 - DNA, 27 bp, synthetic construct
  misc_feature 1..27: note = Synthetic primer
  source 1..27: mol_type = other DNA
tccacagacacttgagattcttgacat

SEQ ID NO: 470 - DNA, 26 bp, synthetic construct
  misc_feature 1..26: note = Synthetic primer
  source 1..26: mol_type = other DNA
aacacgccaagtaggagtaagttgat

SEQ ID NO: 471 - DNA, 31 bp, synthetic construct
  misc_feature 1..31: note = Synthetic primer
  source 1..31: mol_type = other DNA
gctagttatcagactcagactaattctcctc

SEQ ID NO: 472 - DNA, 26 bp, synthetic construct
  misc_feature 1..26: note = Synthetic primer
  source 1..26: mol_type = other DNA
attgctgcattcagttgaatcaccac

SEQ ID NO: 473 - DNA, 30 bp, synthetic construct
  misc_feature 1..30: note = Synthetic primer
  source 1..30: mol_type = other DNA
gtcagtgtgttaatcttacaaccagaactc

SEQ ID NO: 474 - DNA, 30 bp, synthetic construct
  misc_feature 1..30: note = Synthetic primer
  source 1..30: mol_type = other DNA
gcctcttattatgttagacttctcagtgga

SEQ ID NO: 475 - DNA, 25 bp, synthetic construct
  misc_feature 1..25: note = Synthetic primer
  source 1..25: mol_type = other DNA
tggatggaaagtgagttcagagttt

SEQ ID NO: 476 - DNA, 25 bp, synthetic construct
  misc_feature 1..25: note = Synthetic primer
  source 1..25: mol_type = other DNA
ctgaagaagaatcaccaggagtcaa

SEQ ID NO: 477 - DNA, 24 bp, synthetic construct
  misc_feature 1..24: note = Synthetic primer
  source 1..24: mol_type = other DNA
tacaggctgcgttatagcttggaa

SEQ ID NO: 478 - DNA, 24 bp, synthetic construct
  misc_feature 1..24: note = Synthetic primer
  source 1..24: mol_type = other DNA
aacagttgctggtgcatgtagaag

SEQ ID NO: 479 - DNA, 28 bp, synthetic construct
  misc_feature 1..28: note = Synthetic primer
  source 1..28: mol_type = other DNA
tcatgcagatcaacttactcctacttgg

SEQ ID NO: 480 - DNA, 30 bp, synthetic construct
  misc_feature 1..30: note = Synthetic primer
  source 1..30: mol_type = other DNA
catagacactggtagaatttctgtggtaac

SEQ ID NO: 481 - DNA, 20 bp, synthetic construct
  misc_feature 1..20: note = Synthetic primer
  source 1..20: mol_type = other DNA
aacgccaccagatttgcatc

SEQ ID NO: 482 - DNA, 21 bp, synthetic construct
  misc_feature 1..21: note = Synthetic primer
  source 1..21: mol_type = other DNA
acgcagcctgtaaaatcatct

SEQ ID NO: 483 - DNA, 23 bp, synthetic construct
  misc_feature 1..23: note = Synthetic primer
  source 1..23: mol_type = other DNA
actgtgttgctgattattctgtc

SEQ ID NO: 484 - DNA, 23 bp, synthetic construct
  misc_feature 1..23: note = Synthetic primer
  source 1..23: mol_type = other DNA
ctataacgcagcctgtaaaatca

SEQ ID NO: 485 - DNA, 20 bp, synthetic construct
  misc_feature 1..20: note = Synthetic primer
  source 1..20: mol_type = other DNA
aaacttgtgcccttttggtg

SEQ ID NO: 486 - DNA, 21 bp, synthetic construct
  misc_feature 1..21: note = Synthetic primer
  source 1..21: mol_type = other DNA
aggggtactgctgttatgtct

SEQ ID NO: 487 - DNA, 20 bp, synthetic construct
  misc_feature 1..20: note = Synthetic primer
  source 1..20: mol_type = other DNA
gcagtagcgcgaacaaaatc

SEQ ID NO: 488 - DNA, 25 bp, synthetic construct
  misc_feature 1..25: note = Synthetic primer
  source 1..25: mol_type = other DNA
atttgacatgagtaaatttcccctt

SEQ ID NO: 489 - DNA, 21 bp, synthetic construct
  misc_feature 1..21: note = Synthetic primer
  source 1..21: mol_type = other DNA
gcatccttgatttcaccttgc

SEQ ID NO: 490 - DNA, 26 bp, synthetic construct
  misc_feature 1..26: note = Synthetic primer
  source 1..26: mol_type = other DNA
ttaaattaaggggtactgctgttatg

SEQ ID NO: 491 - DNA, 22 bp, synthetic construct
  misc_feature 1..22: note = Synthetic primer
  source 1..22: mol_type = other DNA
cgcgaacaaaatctgaaggagt

SEQ ID NO: 492 - DNA, 48 bp, synthetic construct
  misc_feature 1..48: note = Synthetic probe
  source 1..48: mol_type = other DNA
actggtaacccagacatgatcggtgctgattttgacacatggtttagt

SEQ ID NO: 493 - DNA, 48 bp, synthetic construct
  misc_feature 1..48: note = Synthetic probe
  source 1..48: mol_type = other DNA
actccctgtgggtgagcttaatgggctgattttgacacatggtttagc

SEQ ID NO: 494 - DNA, 21 bp, synthetic construct
  misc_feature 1..21: note = Synthetic probe
  source 1..21: mol_type = other DNA
cagcgtggtggtagttatact

SEQ ID NO: 495 - DNA, 53 bp, synthetic construct
  misc_feature 1..53: note = Synthetic probe
  source 1..53: mol_type = other DNA
catttggtcattggttcaagacgagatatcggtaattatacagtttcctgttc

SEQ ID NO: 496 - DNA, 53 bp, synthetic construct
  misc_feature 1..53: note = Synthetic probe
  source 1..53: mol_type = other DNA
ctggtccgttgtggtccttctaacgatatcggtaattatacagtttcctgttt

SEQ ID NO: 497 - DNA, 26 bp, synthetic construct
  misc_feature 1..26: note = Synthetic probe
  source 1..26: mol_type = other DNA
accttttacaattaattgccaggaac

SEQ ID NO: 498 - DNA, 47 bp, synthetic construct
  misc_feature 1..47: note = Synthetic probe
  source 1..47: mol_type = other DNA
ctaatagctcctgtgccctcgtatcaggttgctgttctttatcagga

SEQ ID NO: 499 - DNA, 47 bp, synthetic construct
  misc_feature 1..47: note = Synthetic probe
  source 1..47: mol_type = other DNA
aatccgtcgactagcctgagaattcaggttgctgttctttatcaggg

SEQ ID NO: 500 - DNA, 22 bp, synthetic construct
  misc_feature 1..22: note = Synthetic probe
  source 1..22: mol_type = other DNA
tgttaactgcacagaagtccct

SEQ ID NO: 501 - DNA, 54 bp, synthetic construct
  misc_feature 1..54: note = Synthetic probe
  source 1..54: mol_type = other DNA
gtggcaacagaaatcaggtggtgaagtactcaatggtctttgttcttttttttg

SEQ ID NO: 502 - DNA, 54 bp, synthetic construct
  misc_feature 1..54: note = Synthetic probe
  source 1..54: mol_type = other DNA
agaggactgctaaaggtttgtaggagtactcaatggtctttgttcttttttttt

SEQ ID NO: 503 - DNA, 28 bp, synthetic construct
  misc_feature 1..28: note = Synthetic probe
  source 1..28: mol_type = other DNA
tatgaaaatgcctttttaccttttgcta

SEQ ID NO: 504 - DNA, 51 bp, synthetic construct
  misc_feature 1..51: note = Synthetic probe
  source 1..51: mol_type = other DNA
gtggcaacagaaatcaggtggtgagcaaaaggtaaaaaggcattttcatac

SEQ ID NO: 505 - DNA, 51 bp, synthetic construct
  misc_feature 1..51: note = Synthetic probe
  source 1..51: mol_type = other DNA
agaggactgctaaaggtttgtagggcaaaaggtaaaaaggcattttcataa

SEQ ID NO: 506 - DNA, 30 bp, synthetic construct
  misc_feature 1..30: note = Synthetic probe
  source 1..30: mol_type = other DNA
aaaaaaaagaacaaagaccattgagtactc

SEQ ID NO: 507 - DNA, 75 bp, synthetic construct
  misc_feature 1..75: note = Synthetic oligonucleotide
  source 1..75: mol_type = other DNA
ttgctaacaaacatgctgattttgacacatggtttagtcagcgtggtggtagttatactaatgacaaagcttgcc

SEQ ID NO: 508 - DNA, 75 bp, synthetic construct
  misc_feature 1..75: note = Synthetic oligonucleotide
  source 1..75: mol_type = other DNA
ttgctaacaaacatgctgattttgacacatggtttagccagcgtggtggtagttatactaatgacaaagcttgcc

SEQ ID NO: 509 - DNA, 75 bp, synthetic construct
  misc_feature 1..75: note = Synthetic oligonucleotide
  source 1..75: mol_type = other DNA
ggcaagctttgtcattagtataactaccaccacgctgactaaaccatgtgtcaaaatcagcatgtttgttagcaa

SEQ ID NO: 510 - DNA, 75 bp, synthetic construct
  misc_feature 1..75: note = Synthetic oligonucleotide
  source 1..75: mol_type = other DNA
ggcaagctttgtcattagtataactaccaccacgctggctaaaccatgtgtcaaaatcagcatgtttgttagcaa

SEQ ID NO: 511 - DNA, 75 bp, synthetic construct
  misc_feature 1..75: note = Synthetic oligonucleotide
  source 1..75: mol_type = other DNA
cagtacatcgatatcggtaattatacagtttcctgttcaccttttacaattaattgccaggaacctaaattgggt

SEQ ID NO: 512 - DNA, 75 bp, synthetic construct
  misc_feature 1..75: note = Synthetic oligonucleotide
  source 1..75: mol_type = other DNA
cagtacatcgatatcggtaattatacagtttcctgtttaccttttacaattaattgccaggaacctaaattgggt

SEQ ID NO: 513 - DNA, 75 bp, synthetic construct
  misc_feature 1..75: note = Synthetic oligonucleotide
  source 1..75: mol_type = other DNA
acccaatttaggttcctggcaattaattgtaaaaggtgaacaggaaactgtataattaccgatatcgatgtactg

SEQ ID NO: 514 - DNA, 75 bp, synthetic construct
  misc_feature 1..75: note = Synthetic oligonucleotide
  source 1..75: mol_type = other DNA
acccaatttaggttcctggcaattaattgtaaaaggtaaacaggaaactgtataattaccgatatcgatgtactg

SEQ ID NO: 515 - DNA, 75 bp, synthetic construct
  misc_feature 1..75: note = Synthetic oligonucleotide
  source 1..75: mol_type = other DNA
acaaatacttctaaccaggttgctgttctttatcaggatgttaactgcacagaagtccctgttgctattcatgca

SEQ ID NO: 516 - DNA, 75 bp, synthetic construct
  misc_feature 1..75: note = Synthetic oligonucleotide
  source 1..75: mol_type = other DNA
acaaatacttctaaccaggttgctgttctttatcagggtgttaactgcacagaagtccctgttgctattcatgca

SEQ ID NO: 517 - DNA, 75 bp, synthetic construct
  misc_feature 1..75: note = Synthetic oligonucleotide
  source 1..75: mol_type = other DNA
tgcatgaatagcaacagggacttctgtgcagttaacatcctgataaagaacagcaacctggttagaagtatttgt

SEQ ID NO: 518 - DNA, 75 bp, synthetic construct
  misc_feature 1..75: note = Synthetic oligonucleotide
  source 1..75: mol_type = other DNA
tgcatgaatagcaacagggacttctgtgcagttaacaccctgataaagaacagcaacctggttagaagtatttgt

SEQ ID NO: 519 - DNA, 75 bp, synthetic construct
  misc_feature 1..75: note = Synthetic oligonucleotide
  source 1..75: mol_type = other DNA
tactccagagtactcaatggtctttgttcttttttttgtatgaaaatgcctttttaccttttgctatgggtatta

SEQ ID NO: 520 - DNA, 75 bp, synthetic construct
  misc_feature 1..75: note = Synthetic oligonucleotide
  source 1..75: mol_type = other DNA
tactccagagtactcaatggtctttgttcttttttttttatgaaaatgcctttttaccttttgctatgggtatta

SEQ ID NO: 521 - DNA, 75 bp, synthetic construct
  misc_feature 1..75: note = Synthetic oligonucleotide
  source 1..75: mol_type = other DNA
taatacccatagcaaaaggtaaaaaggcattttcatacaaaaaaaagaacaaagaccattgagtactctggacta

SEQ ID NO: 522 - DNA, 75 bp, synthetic construct
  misc_feature 1..75: note = Synthetic oligonucleotide
  source 1..75: mol_type = other DNA
taatacccatagcaaaaggtaaaaaggcattttcataaaaaaaaaagaacaaagaccattgagtactctggacta

SEQ ID NO: 523 - DNA, 24 bp, synthetic construct
  misc_feature 1..24: note = Synthetic oligonucleotide
  source 1..24: mol_type = other DNA
gctgattttgacacatggtttagy

SEQ ID NO: 524 - DNA, 21 bp, synthetic construct
  misc_feature 1..21: note = Synthetic oligonucleotide
  source 1..21: mol_type = other DNA
cagcgtggtggtagttatact

SEQ ID NO: 525 - DNA, 29 bp, synthetic construct
  misc_feature 1..29: note = Synthetic oligonucleotide
  source 1..29: mol_type = other DNA
gatatcggtaattatacagtttcctgtty

SEQ ID NO: 526 - DNA, 26 bp, synthetic construct
  misc_feature 1..26: note = Synthetic oligonucleotide
  source 1..26: mol_type = other DNA
accttttacaattaattgccaggaac

SEQ ID NO: 527 - DNA, 23 bp, synthetic construct
  misc_feature 1..23: note = Synthetic oligonucleotide
  source 1..23: mol_type = other DNA
caggttgctgttctttatcaggr

SEQ ID NO: 528 - DNA, 22 bp, synthetic construct
  misc_feature 1..22: note = Synthetic oligonucleotide
  source 1..22: mol_type = other DNA
tgttaactgcacagaagtccct

SEQ ID NO: 529 - DNA, 30 bp, synthetic construct
  misc_feature 1..30: note = Synthetic oligonucleotide
  source 1..30: mol_type = other DNA
agtactcaatggtctttgttcttttttttk

SEQ ID NO: 530 - DNA, 28 bp, synthetic construct
  misc_feature 1..28: note = Synthetic oligonucleotide
  source 1..28: mol_type = other DNA
tatgaaaatgcctttttaccttttgcta

SEQ ID NO: 531 - DNA, 27 bp, synthetic construct
  misc_feature 1..27: note = Synthetic oligonucleotide
  source 1..27: mol_type = other DNA
gcaaaaggtaaaaaggcattttcatam

SEQ ID NO: 532 - DNA, 30 bp, synthetic construct; identical to SEQ ID NO: 506
  misc_feature 1..30: note = Synthetic oligonucleotide
  source 1..30: mol_type = other DNA

SEQ ID NO: 533 - DNA, 51 bp, synthetic construct
  misc_feature 1..51: note = Synthetic probe
  source 1..51: mol_type = other DNA
cgtaccattgaatctggagaccttacatagaagttatttgactcctggtga

SEQ ID NO: 534 - DNA, 51 bp, synthetic construct
  misc_feature 1..51: note = Synthetic probe
  source 1..51: mol_type = other DNA
tagatgccgctgcaggtatggaaaacatagaagttatttgactcctggtgg

SEQ ID NO: 535 - DNA, 23 bp, synthetic construct
  misc_feature 1..23: note = Synthetic probe
  source 1..23: mol_type = other DNA
ttcttcttcaggttggacagctg

SEQ ID NO: 536 - DNA, 47 bp, synthetic construct
  misc_feature 1..47: note = Synthetic probe
  source 1..47: mol_type = other DNA
cgtaccattgaatctggagaccttagctgtccaacctgaagaagaat

SEQ ID NO: 537 - DNA, 47 bp, synthetic construct
  misc_feature 1..47: note = Synthetic probe
  source 1..47: mol_type = other DNA
tagatgccgctgcaggtatggaaaagctgtccaacctgaagaagaac

SEQ ID NO: 538 - DNA, 29 bp, synthetic construct
  misc_feature 1..29: note = Synthetic probe
  source 1..29: mol_type = other DNA
caccaggagtcaaataacttctatgtaaa

SEQ ID NO: 539 - DNA, 75 bp, synthetic construct
  misc_feature 1..75: note = Synthetic oligonucleotide
  source 1..75: mol_type = other DNA
ttacttgctttacatagaagttatttgactcctggtgattcttcttcaggttggacagctggtgctgcagcttat

SEQ ID NO: 540 - DNA, 75 bp, synthetic construct
  misc_feature 1..75: note = Synthetic oligonucleotide
  source 1..75: mol_type = other DNA
ttacttgctttacatagaagttatttgactcctggtggttcttcttcaggttggacagctggtgctgcagcttat

SEQ ID NO: 541 - DNA, 75 bp, synthetic construct
  misc_feature 1..75: note = Synthetic oligonucleotide
  source 1..75: mol_type = other DNA
ataagctgcagcaccagctgtccaacctgaagaagaatcaccaggagtcaaataacttctatgtaaagcaagtaa

SEQ ID NO: 542 - DNA, 75 bp, synthetic construct
  misc_feature 1..75: note = Synthetic oligonucleotide
  source 1..75: mol_type = other DNA
ataagctgcagcaccagctgtccaacctgaagaagaaccaccaggagtcaaataacttctatgtaaagcaagtaa

SEQ ID NO: 543 - DNA, 27 bp, synthetic construct
  misc_feature 1..27: note = Synthetic oligonucleotide
  source 1..27: mol_type = other DNA
acatagaagttatttgactcctggtgr

SEQ ID NO: 544 - DNA, 23 bp, synthetic construct
  misc_feature 1..23: note = Synthetic oligonucleotide
  source 1..23: mol_type = other DNA
ttcttcttcaggttggacagctg

SEQ ID NO: 545 - DNA, 23 bp, synthetic construct
  misc_feature 1..23: note = Synthetic oligonucleotide
  source 1..23: mol_type = other DNA
agctgtccaacctgaagaagaay

SEQ ID NO: 546 - DNA, 29 bp, synthetic construct
  misc_feature 1..29: note = Synthetic oligonucleotide
  source 1..29: mol_type = other DNA
caccaggagtcaaataacttctatgtaaa

SEQ ID NO: 547 - protein, 6 aa, synthetic construct
  REGION 1..6: note = Synthetic 6xHis tag
HHHHHH

SEQ ID NO: 548 - protein, 8 aa, synthetic construct
  REGION 1..8: note = Synthetic peptide
DYKDDDDK

SEQ ID NO: 549 - DNA, 46 bp, synthetic construct
  misc_feature 1..46: note = Synthetic
  source 1..46: mol_type = other DNA
catttggtcattggttcaagacgaagcacaccttgtaatggtgttc

SEQ ID NO: 550 - DNA, 57 bp, synthetic construct
  misc_feature 1..57: note = Synthetic
  source 1..57: mol_type = other DNA
catttggtcattggttcaagacgatgattgtaaaggaaagtaacaattaaaaccttg

SEQ ID NO: 551 - DNA, 54 bp, synthetic construct
  misc_feature 1..54: note = Synthetic
  source 1..54: mol_type = other DNA
ctaatagctcctgtgccctcgtatactagtctctagtcagtgtgttaatcttac

SEQ ID NO: 552 - DNA, 54 bp, synthetic construct
  misc_feature 1..54: note = Synthetic
  source 1..54: mol_type = other DNA
aatccgtcgactagcctgagaattactagtctctagtcagtgtgttaatcttag

SEQ ID NO: 553 - DNA, 22 bp, synthetic construct
  misc_feature 1..22: note = Synthetic
  source 1..22: mol_type = other DNA
aaccagaactcaattaccccct

SEQ ID NO: 554 - DNA, 47 bp, synthetic construct
  misc_feature 1..47: note = Synthetic
  source 1..47: mol_type = other DNA
ctaatagctcctgtgccctcgtatagggggtaattgagttctggttg

SEQ ID NO: 555 - DNA, 47 bp, synthetic construct
  misc_feature 1..47: note = Synthetic
  source 1..47: mol_type = other DNA
aatccgtcgactagcctgagaattagggggtaattgagttctggttc

SEQ ID NO: 556 - DNA, 29 bp, synthetic construct
  misc_feature 1..29: note = Synthetic
  source 1..29: mol_type = other DNA
taagattaacacactgactagagactagt

SEQ ID NO: 557 - DNA, 52 bp, synthetic construct
  misc_feature 1..52: note = Synthetic
  source 1..52: mol_type = other DNA
ctggtccgttgtggtccttctaacctagttatcagactcagactaattctcg

SEQ ID NO: 558 - DNA, 40 bp, synthetic construct
  misc_feature 1..40: note = Synthetic
  source 1..40: mol_type = other DNA
ctggtccgttgtggtccttctaacctacgtgcccgccgac

SEQ ID NO: 559 - DNA, 48 bp, synthetic construct
  misc_feature 1..48: note = Synthetic
  source 1..48: mol_type = other DNA
tagatgccgctgcaggtatggaaacatgtgacttatgtccctgcacaa

SEQ ID NO: 560 - DNA, 48 bp, synthetic construct
  misc_feature 1..48: note = Synthetic
  source 1..48: mol_type = other DNA
cgtaccattgaatctggagaccttcatgtgacttatgtccctgcacat

SEQ ID NO: 561 - DNA, 25 bp, synthetic construct
  misc_feature 1..25: note = Synthetic
  source 1..25: mol_type = other DNA
gaaaagaacttcacaactgctcctg

SEQ ID NO: 562 - DNA, 50 bp, synthetic construct
  misc_feature 1..50: note = Synthetic
  source 1..50: mol_type = other DNA
tagatgccgctgcaggtatggaaacaggagcagttgtgaagttcttttct

SEQ ID NO: 563 - DNA, 50 bp, synthetic construct
  misc_feature 1..50: note = Synthetic
  source 1..50: mol_type = other DNA
cgtaccattgaatctggagaccttcaggagcagttgtgaagttcttttca

SEQ ID NO: 564 - DNA, 22 bp, synthetic construct
  misc_feature 1..22: note = Synthetic
  source 1..22: mol_type = other DNA
tgtgcagggacataagtcacat

SEQ ID NO: 565 - DNA, 46 bp, synthetic construct
  misc_feature 1..46: note = Synthetic
  source 1..46: mol_type = other DNA
actggtaacccagacatgatcgggaaatctatcaggccggtarcac

SEQ ID NO: 566 - DNA, 47 bp, synthetic construct
  misc_feature 1..47: note = Synthetic
  source 1..47: mol_type = other DNA
actccctgtgggtgagcttaatgggaaatctatcaggccggtarcaa

SEQ ID NO: 567 - DNA, 27 bp, synthetic construct
  misc_feature 1..27: note = Synthetic
  source 1..27: mol_type = other DNA
accttgtaatggtgttvaaggttttaa

SEQ ID NO: 568 - DNA, 51 bp, synthetic construct
  misc_feature 1..51: note = Synthetic
  source 1..51: mol_type = other DNA
actggtaacccagacatgatcggttaaaaccttbaacaccattacaaggtg

SEQ ID NO: 569 - DNA, 52 bp, synthetic construct
  misc_feature 1..52: note = Synthetic
  source 1..52: mol_type = other DNA
actccctgtgggtgagcttaatggttaaaaccttbaacaccattacaaggtt

SEQ ID NO: 570 - DNA, 21 bp, synthetic construct
  misc_feature 1..21: note = Synthetic
  source 1..21: mol_type = other DNA
tgytaccggcctgatagattt

SEQ ID NO: 571 - DNA, 46 bp, synthetic construct
  misc_feature 1..46: note = Synthetic
  source 1..46: mol_type = other DNA
catttggtcattggttcaagacgaacaatcacttctggttggacct

SEQ ID NO: 572 - DNA, 46 bp, synthetic construct
  misc_feature 1..46: note = Synthetic
  source 1..46: mol_type = other DNA
ctggtccgttgtggtccttctaacacaatcacttctggttggaccc

SEQ ID NO: 573 - DNA, 17 bp, synthetic construct
  misc_feature 1..17: note = Synthetic
  source 1..17: mol_type = other DNA
ttggtgcaggtgctgca

SEQ ID NO: 574 - DNA, 41 bp, synthetic construct
  misc_feature 1..41: note = Synthetic
  source 1..41: mol_type = other DNA
catttggtcattggttcaagacgagcagcacctgcaccaaa

SEQ ID NO: 575 - DNA, 41 bp, synthetic construct
  misc_feature 1..41: note = Synthetic
  source 1..41: mol_type = other DNA
ctggtccgttgtggtccttctaacgcagcacctgcaccaag

SEQ ID NO: 576 - DNA, 23 bp, synthetic construct
  misc_feature 1..23: note = Synthetic
  source 1..23: mol_type = other DNA
ggtccaaccagaagtgattgtac

SEQ ID NO: 577 - DNA, 48 bp, synthetic construct
  misc_feature 1..48: note = Synthetic
  source 1..48: mol_type = other DNA
ctaatagctcctgtgccctcgtatacatctctggcattaatgcttcag

SEQ ID NO: 578 - DNA, 48 bp, synthetic construct
  misc_feature 1..48: note = Synthetic
  source 1..48: mol_type = other DNA
aatccgtcgactagcctgagaattacatctctggcattaatgcttcat

SEQ ID NO: 579 - DNA, 29 bp, synthetic construct
  misc_feature 1..29: note = Synthetic
  source 1..29: mol_type = other DNA
ttgtaaacattcaaaaagaaattgaccgc

SEQ ID NO: 580 - DNA, 54 bp, synthetic construct
  misc_feature 1..54: note = Synthetic
  source 1..54: mol_type = other DNA
ctaatagctcctgtgccctcgtatgcggtcaatttctttttgaatgtttacaac

SEQ ID NO: 581 - DNA, 54 bp, synthetic construct
  misc_feature 1..54: note = Synthetic
  source 1..54: mol_type = other DNA
aatccgtcgactagcctgagaattgcggtcaatttctttttgaatgtttacaaa

SEQ ID NO: 582 - DNA, 24 bp, synthetic construct
  misc_feature 1..24: note = Synthetic
  source 1..24: mol_type = other DNA
tgaagcattaatgccagagatgtc

SEQ ID NO: 583 - DNA, 49 bp, synthetic construct
  misc_feature 1..49: note = Synthetic
  source 1..49: mol_type = other DNA
gtggcaacagaaatcaggtggtgatatatgcgctagttatcagactcmg

SEQ ID NO: 584 - DNA, 49 bp, synthetic construct
  misc_feature 1..49: note = Synthetic
  source 1..49: mol_type = other DNA
agaggactgctaaaggtttgtaggtatatgcgctagttatcagactcmt

SEQ ID NO: 585 - DNA, 49 bp, synthetic construct
  misc_feature 1..49: note = Synthetic
  source 1..49: mol_type = other DNA
agaggactgctaaaggtttgtaggtatatgcgctagttatcagactcmc

SEQ ID NO: 586 - DNA, 19 bp, synthetic construct
  misc_feature 1..19: note = Synthetic
  source 1..19: mol_type = other DNA
actaattctcvtcggcggg

SEQ ID NO: 587 - DNA, 44 bp, synthetic construct
  misc_feature 1..44: note = Synthetic
  source 1..44: mol_type = other DNA
gtggcaacagaaatcaggtggtgacccgccgabgagaattagtc

SEQ ID NO: 588 - DNA, 44 bp, synthetic construct
  misc_feature 1..44: note = Synthetic
  source 1..44: mol_type = other DNA
agaggactgctaaaggtttgtaggcccgccgabgagaattagta

SEQ ID NO: 589 - DNA, 44 bp, synthetic construct
  misc_feature 1..44: note = Synthetic
  source 1..44: mol_type = other DNA
agaggactgctaaaggtttgtaggcccgccgabgagaattagtg

SEQ ID NO: 590 - DNA, 24 bp, synthetic construct
  misc_feature 1..24: note = Synthetic
  source 1..24: mol_type = other DNA
kgagtctgataactagcgcatata

SEQ ID NO: 591 - DNA, 53 bp, synthetic construct
  misc_feature 1..53: note = Synthetic
  source 1..53: mol_type = other DNA
tagatgccgctgcaggtatggaaattttgtaatgatccatttttgggtgttta

SEQ ID NO: 592 - DNA, 52 bp, synthetic construct
  misc_feature 1..52: note = Synthetic
  source 1..52: mol_type = other DNA
cgtaccattgaatctggagaccttaattttgtaatgatccatttttgggtgt

SEQ ID NO: 593 - DNA, 27 bp, synthetic construct
  misc_feature 1..27: note = Synthetic
  source 1..27: mol_type = other DNA
ttaccacaaaaacaacaaaagttggat

SEQ ID NO: 594 - DNA, 52 bp, synthetic construct
  misc_feature 1..52: note = Synthetic
  source 1..52: mol_type = other DNA
tagatgccgctgcaggtatggaaaccaacttttgttgtttttgtggtaataa

SEQ ID NO: 595 - DNA, 51 bp, synthetic construct
  misc_feature 1..51: note = Synthetic
  source 1..51: mol_type = other DNA
cgtaccattgaatctggagaccttatccaacttttgttgtttttgtggtaa

SEQ ID NO: 596 - DNA, 28 bp, synthetic construct
  misc_feature 1..28: note = Synthetic
  source 1..28: mol_type = other DNA
acacccaaaaatggatcattacaaaatt

SEQ ID NO: 597 - DNA, 76 bp, synthetic construct
  misc_feature 1..76: note = Synthetic
  source 1..76: mol_type = other DNA
aatctatcaggccggtagcacaccttgtaatggtgttcaaggttttaattgttactttcctttacaatcatatggt

SEQ ID NO: 598 - DNA, 75 bp, synthetic construct
  misc_feature 1..75: note = Synthetic
  source 1..75: mol_type = other DNA
ccatatgattgtaaaggaaagtaacaattaaaaccttgaacaccattacaaggtgtgctaccggcctgatagatt

SEQ ID NO: 599 - DNA, 75 bp, synthetic construct
  misc_feature 1..75: note = Synthetic
  source 1..75: mol_type = other DNA
ttattgccactagtctctagtcagtgtgttaatcttacaaccagaactcaattaccccctgcatacactaattct

SEQ ID NO: 600 - DNA, 75 bp, synthetic construct
  misc_feature 1..75: note = Synthetic
  source 1..75: mol_type = other DNA
ttattgccactagtctctagtcagtgtgttaatcttagaaccagaactcaattaccccctgcatacactaattct

SEQ ID NO: 601 - DNA, 75 bp, synthetic construct
  misc_feature 1..75: note = Synthetic
  source 1..75: mol_type = other DNA
agaattagtgtatgcagggggtaattgagttctggttgtaagattaacacactgactagagactagtggcaataa

SEQ ID NO: 602 - DNA, 75 bp, synthetic construct
  misc_feature 1..75: note = Synthetic
  source 1..75: mol_type = other DNA
agaattagtgtatgcagggggtaattgagttctggttctaagattaacacactgactagagactagtggcaataa

SEQ ID NO: 603 - DNA, 75 bp, synthetic construct
  misc_feature 1..75: note = Synthetic
  source 1..75: mol_type = other DNA
aggtatatgcgctagttatcagactcagactaattctcgtcggcgggcacgtagtgtagctagtcaatccatcat

SEQ ID NO: 604 - DNA, 75 bp, synthetic construct
  misc_feature 1..75: note = Synthetic
  source 1..75: mol_type = other DNA
atgatggattgactagctacactacgtgcccgccgacgagaattagtctgagtctgataactagcgcatatacct

SEQ ID NO: 605 - DNA, 75 bp, synthetic construct
  misc_feature 1..75: note = Synthetic
  source 1..75: mol_type = other DNA
gtgtagtcttcttgcatgtgacttatgtccctgcacaagaaaagaacttcacaactgctcctgccatttgtcatg

SEQ ID NO: 606 - DNA, 75 bp, synthetic construct
  misc_feature 1..75: note = Synthetic
  source 1..75: mol_type = other DNA
gtgtagtcttcttgcatgtgacttatgtccctgcacatgaaaagaacttcacaactgctcctgccatttgtcatg

SEQ ID NO: 607 - DNA, 75 bp, synthetic construct
  misc_feature 1..75: note = Synthetic
  source 1..75: mol_type = other DNA
catgacaaatggcaggagcagttgtgaagttcttttcttgtgcagggacataagtcacatgcaagaagactacac

SEQ ID NO: 608 - DNA, 75 bp, synthetic construct
  misc_feature 1..75: note = Synthetic
  source 1..75: mol_type = other DNA
catgacaaatggcaggagcagttgtgaagttcttttcatgtgcagggacataagtcacatgcaagaagactacac

SEQ ID NO: 609 - DNA, 75 bp, synthetic construct
  misc_feature 1..75: note = Synthetic
  source 1..75: mol_type = other DNA
agagatatttcaactgaaatctatcaggccggtarcacaccttgtaatggtgttvaaggttttaattgttacttt

SEQ ID NO: 610 - DNA, 75 bp, synthetic construct
  misc_feature 1..75: note = Synthetic
  source 1..75: mol_type = other DNA
agagatatttcaactgaaatctatcaggccggtarcaaaccttgtaatggtgttvaaggttttaattgttacttt

SEQ ID NO: 611 - DNA, 75 bp, synthetic construct
  misc_feature 1..75: note = Synthetic
  source 1..75: mol_type = other DNA
aaagtaacaattaaaaccttbaacaccattacaaggtgtgytaccggcctgatagatttcagttgaaatatctct

SEQ ID NO: 612 - DNA, 75 bp, synthetic construct
  misc_feature 1..75: note = Synthetic
  source 1..75: mol_type = other DNA
aaagtaacaattaaaaccttbaacaccattacaaggtttgytaccggcctgatagatttcagttgaaatatctct

SEQ ID NO: 613 - DNA, 75 bp, synthetic construct
  misc_feature 1..75: note = Synthetic
  source 1..75: mol_type = other DNA
tgcactgttagcgggtacaatcacttctggttggacctttggtgcaggtgctgcattacaaataccatttgctat

SEQ ID NO: 614 - DNA, 75 bp, synthetic construct
  misc_feature 1..75: note = Synthetic
  source 1..75: mol_type = other DNA
tgcactgttagcgggtacaatcacttctggttggacccttggtgcaggtgctgcattacaaataccatttgctat

SEQ ID NO: 615 - DNA, 75 bp, synthetic construct
  misc_feature 1..75: note = Synthetic
  source 1..75: mol_type = other DNA
atagcaaatggtatttgtaatgcagcacctgcaccaaaggtccaaccagaagtgattgtacccgctaacagtgca

SEQ ID NO: 616 - DNA, 75 bp, synthetic construct
  misc_feature 1..75: note = Synthetic
  source 1..75: mol_type = other DNA
atagcaaatggtatttgtaatgcagcacctgcaccaagggtccaaccagaagtgattgtacccgctaacagtgca

SEQ ID NO: 617 - DNA, 75 bp, synthetic construct
  misc_feature 1..75: note = Synthetic
  source 1..75: mol_type = other DNA
tgttgatttaggtgacatctctggcattaatgcttcagttgtaaacattcaaaaagaaattgaccgcctcaatga

SEQ ID NO: 618 - DNA, 75 bp, synthetic construct
  misc_feature 1..75: note = Synthetic
  source 1..75: mol_type = other DNA
tgttgatttaggtgacatctctggcattaatgcttcatttgtaaacattcaaaaagaaattgaccgcctcaatga

SEQ ID NO: 619 - DNA, 75 bp, synthetic construct
  misc_feature 1..75: note = Synthetic
  source 1..75: mol_type = other DNA
tcattgaggcggtcaatttctttttgaatgtttacaactgaagcattaatgccagagatgtcacctaaatcaaca

SEQ ID NO: 620 - DNA, 75 bp, synthetic construct
  misc_feature 1..75: note = Synthetic
  source 1..75: mol_type = other DNA
tcattgaggcggtcaatttctttttgaatgtttacaaatgaagcattaatgccagagatgtcacctaaatcaaca

SEQ ID NO: 621 - DNA, 75 bp, synthetic construct
  misc_feature 1..75: note = Synthetic
  source 1..75: mol_type = other DNA
ccattggtgcaggtatatgcgctagttatcagactcmgactaattctcvtcggcgggcacgtagtgtagctagtc

SEQ ID NO: 622 - DNA, 75 bp, synthetic construct
  misc_feature 1..75: note = Synthetic
  source 1..75: mol_type = other DNA
ccattggtgcaggtatatgcgctagttatcagactcmtactaattctcvtcggcgggcacgtagtgtagctagtc

SEQ ID NO: 623 - DNA, 75 bp, synthetic construct
  misc_feature 1..75: note = Synthetic
  source 1..75: mol_type = other DNA
ccattggtgcaggtatatgcgctagttatcagactcmcactaattctcvtcggcgggcacgtagtgtagctagtc

SEQ ID NO: 624 - DNA, 75 bp, synthetic construct
  misc_feature 1..75: note = Synthetic
  source 1..75: mol_type = other DNA
gactagctacactacgtgcccgccgabgagaattagtckgagtctgataactagcgcatatacctgcaccaatgg

SEQ ID NO: 625 - DNA, 75 bp, synthetic construct
  misc_feature 1..75: note = Synthetic
  source 1..75: mol_type = other DNA
gactagctacactacgtgcccgccgabgagaattagtakgagtctgataactagcgcatatacctgcaccaatgg

SEQ ID NO: 626 - DNA, 75 bp, synthetic construct
  misc_feature 1..75: note = Synthetic
  source 1..75: mol_type = other DNA
gactagctacactacgtgcccgccgabgagaattagtgkgagtctgataactagcgcatatacctgcaccaatgg

SEQ ID NO: 627 - DNA, 22 bp, synthetic construct
  misc_feature 1..22: note = Synthetic
  source 1..22: mol_type = other DNA
agcacaccttgtaatggtgtts

SEQ ID NO: 628 - DNA, 33 bp, synthetic construct
  misc_feature 1..33: note = Synthetic
  source 1..33: mol_type = other DNA
tgattgtaaaggaaagtaacaattaaaacctts

SEQ ID NO: 629 - DNA, 30 bp, synthetic construct
  misc_feature 1..30: note = Synthetic
  source 1..30: mol_type = other DNA
actagtctctagtcagtgtgttaatcttas

SEQ ID NO: 630 - DNA, 22 bp, synthetic construct
  misc_feature 1..22: note = Synthetic
  source 1..22: mol_type = other DNA
aaccagaactcaattaccccct

SEQ ID NO: 631 - DNA, 23 bp, synthetic construct
  misc_feature 1..23: note = Synthetic
  source 1..23: mol_type = other DNA
agggggtaattgagttctggtts

SEQ ID NO: 632 - DNA, 29 bp, synthetic construct
  misc_feature 1..29: note = Synthetic
  source 1..29: mol_type = other DNA
taagattaacacactgactagagactagt

SEQ ID NO: 633 - DNA, 28 bp, synthetic construct
  misc_feature 1..28: note = Synthetic
  source 1..28: mol_type = other DNA
ctagttatcagactcagactaattctcs

SEQ ID NO: 634 - DNA, 16 bp, synthetic construct
  misc_feature 1..16: note = Synthetic
  source 1..16: mol_type = other DNA
ctacgtgcccgccgas

SEQ ID NO: 635 - DNA, 24 bp, synthetic construct
  misc_feature 1..24: note = Synthetic
  source 1..24: mol_type = other DNA
catgtgacttatgtccctgcacaw

SEQ ID NO: 636 - DNA, 25 bp, synthetic construct
  misc_feature 1..25: note = Synthetic
  source 1..25: mol_type = other DNA
gaaaagaacttcacaactgctcctg

SEQ ID NO: 637 - DNA, 26 bp, synthetic construct
  misc_feature 1..26: note = Synthetic
  source 1..26: mol_type = other DNA
caggagcagttgtgaagttcttttcw

SEQ ID NO: 638 - DNA, 22 bp, synthetic construct
  misc_feature 1..22: note = Synthetic
  source 1..22: mol_type = other DNA
tgtgcagggacataagtcacat

SEQ ID NO: 639 - DNA, 23 bp, synthetic construct
  misc_feature 1..23: note = Synthetic
  source 1..23: mol_type = other DNA
gaaatctatcaggccggtarcam

SEQ ID NO: 640 - DNA, 27 bp, synthetic construct
  misc_feature 1..27: note = Synthetic
  source 1..27: mol_type = other DNA
accttgtaatggtgttvaaggttttaa

SEQ ID NO: 641 - DNA, 28 bp, synthetic construct
  misc_feature 1..28: note = Synthetic
  source 1..28: mol_type = other DNA
ttaaaaccttbaacaccattacaaggtk

SEQ ID NO: 642 - DNA, 21 bp, synthetic construct
  misc_feature 1..21: note = Synthetic
  source 1..21: mol_type = other DNA
tgytaccggcctgatagattt

SEQ ID NO: 643 - DNA, 22 bp, synthetic construct
  misc_feature 1..22: note = Synthetic
  source 1..22: mol_type = other DNA
acaatcacttctggttggaccy

SEQ ID NO: 644 - DNA, 17 bp, synthetic construct
  misc_feature 1..17: note = Synthetic
  source 1..17: mol_type = other DNA
ttggtgcaggtgctgca

SEQ ID NO: 645 - DNA, 17 bp, synthetic construct
  misc_feature 1..17: note = Synthetic
  source 1..17: mol_type = other DNA
gcagcacctgcaccaar

SEQ ID NO: 646 - DNA, 23 bp, synthetic construct
  misc_feature 1..23: note = Synthetic
  source 1..23: mol_type = other DNA
ggtccaaccagaagtgattgtac

SEQ ID NO: 647 - DNA, 24 bp, synthetic construct
  misc_feature 1..24: note = Synthetic
  source 1..24: mol_type = other DNA
acatctctggcattaatgcttcak

SEQ ID NO: 648 - DNA, 29 bp, synthetic construct
  misc_feature 1..29: note = Synthetic
  source 1..29: mol_type = other DNA
ttgtaaacattcaaaaagaaattgaccgc

SEQ ID NO: 649 - DNA, 30 bp, synthetic construct
  misc_feature 1..30: note = Synthetic
  source 1..30: mol_type = other DNA
gcggtcaatttctttttgaatgtttacaam

SEQ ID NO: 650 - DNA, 24 bp, synthetic construct
  misc_feature 1..24: note = Synthetic
  source 1..24: mol_type = other DNA
tgaagcattaatgccagagatgtc

SEQ ID NO: 651 - DNA, 25 bp, synthetic construct
  misc_feature 1..25: note = Synthetic
  source 1..25: mol_type = other DNA
tatatgcgctagttatcagactcmb

SEQ ID NO: 652 - DNA, 19 bp, synthetic construct
  misc_feature 1..19: note = Synthetic
  source 1..19: mol_type = other DNA
actaattctcvtcggcggg

SEQ ID NO: 653 - DNA, 20 bp, synthetic construct
  misc_feature 1..20: note = Synthetic
  source 1..20: mol_type = other DNA
cccgccgabgagaattagtv

SEQ ID NO: 654 - DNA, 24 bp, synthetic construct
  misc_feature 1..24: note = Synthetic
  source 1..24: mol_type = other DNA
kgagtctgataactagcgcatata

SEQ ID NO: 655 - DNA, 24 bp, synthetic construct
  misc_feature 1..24: note = Synthetic
  source 1..24: mol_type = other DNA
tarcatggaaccaagtaacattgg

SEQ ID NO: 656 - DNA, 28 bp, synthetic construct
  misc_feature 1..28: note = Synthetic
  source 1..28: mol_type = other DNA
gagaattagtvkgagtctgataactagc

SEQ ID NO: 657 - DNA, 57 bp, synthetic construct; identical to SEQ ID NO: 108
  misc_feature 1..57: note = Synthetic
  source 1..57: mol_type = other DNA

SEQ ID NO: 658 - DNA, 57 bp, synthetic construct; identical to SEQ ID NO: 109
  misc_feature 1..57: note = Synthetic
  source 1..57: mol_type = other DNA

SEQ ID NO: 659 - DNA, 22 bp, synthetic construct
  misc_feature 1..22: note = Synthetic
  source 1..22: mol_type = other DNA
aacaccattacaaggtktgyta

SEQ ID NO: 660 - DNA, 47 bp, synthetic construct
  misc_feature 1..47: note = Synthetic
  source 1..47: mol_type = other DNA
ctaatagctcctgtgccctcgtatagrgggtaattgagttctgkttg

SEQ ID NO: 661 - DNA, 47 bp, synthetic construct
  misc_feature 1..47: note = Synthetic
  source 1..47: mol_type = other DNA
aatccgtcgactagcctgagaattagrgggtaattgagttctgkttc

SEQ ID NO: 662 - DNA, 29 bp, synthetic construct
  misc_feature 1..29: note = Synthetic
  source 1..29: mol_type = other DNA
taarattaacacactgamtagagactagt

SEQ ID NO: 663 - DNA, 21 bp, synthetic construct
  misc_feature 1..21: note = Synthetic
  misc_feature 21: note = n is a, c, g, or t
  source 1..21: mol_type = other DNA
aggggtactgctgttatgtcn

SEQ ID NO: 664 - DNA, 21 bp, synthetic construct
  misc_feature 1..21: note = Synthetic
  misc_feature 21: note = n is a, c, g, or t
  source 1..21: mol_type = other DNA
gcatccttgatttcaccttgn

SEQ ID NO: 665 - DNA, 21 bp, synthetic construct
  misc_feature 1..21: note = Synthetic
  source 1..21: mol_type = other DNA
gcatccttgatttcaccttgm

SEQ ID NO: 666 - DNA, 46 bp, synthetic construct
  misc_feature 1..46: note = Synthetic
  source 1..46: mol_type = other DNA
gtggcaacagaaatcaggtggtgaacaatcacttctggttggacct

SEQ ID NO: 667 - DNA, 46 bp, synthetic construct
  misc_feature 1..46: note = Synthetic
  source 1..46: mol_type = other DNA
agaggactgctaaaggtttgtaggacaatcacttctggttggaccc

SEQ ID NO: 668 - DNA, 41 bp, synthetic construct
  misc_feature 1..41: note = Synthetic
  source 1..41: mol_type = other DNA
gtggcaacagaaatcaggtggtgagcagcacctgcaccaaa

SEQ ID NO: 669 - DNA, 41 bp, synthetic construct
  misc_feature 1..41: note = Synthetic
  source 1..41: mol_type = other DNA
agaggactgctaaaggtttgtagggcagcacctgcaccaag

SEQ ID NO: 670 - DNA, 48 bp, synthetic construct
  misc_feature 1..48: note = Synthetic
  source 1..48: mol_type = other DNA
gtggcaacagaaatcaggtggtgatgcacaaaagtttaayggccttac

SEQ ID NO: 671 - DNA, 48 bp, synthetic construct
  misc_feature 1..48: note = Synthetic
  source 1..48: mol_type = other DNA
agaggactgctaaaggtttgtaggtgcacaaaagtttaayggccttaa

SEQ ID NO: 672 - DNA, 21 bp, synthetic construct
  misc_feature 1..21: note = Synthetic
  source 1..21: mol_type = other DNA
tgttttgccacctttgctcac

SEQ ID NO: 673 - DNA, 46 bp, synthetic construct
  misc_feature 1..46: note = Synthetic
  source 1..46: mol_type = other DNA
gtggcaacagaaatcaggtggtgagtgagcaaaggtggcaaaacag

SEQ ID NO: 674 - DNA, 46 bp, synthetic construct
  misc_feature 1..46: note = Synthetic
  source 1..46: mol_type = other DNA
agaggactgctaaaggtttgtagggtgagcaaaggtggcaaaacat

SEQ ID NO: 675 - DNA, 23 bp, synthetic construct
  misc_feature 1..23: note = Synthetic
  source 1..23: mol_type = other DNA
taaggccrttaaacttttgtgca

SEQ ID NO: 676 - DNA, 75 bp, synthetic construct
  misc_feature 1..75: note = Synthetic
  source 1..75: mol_type = other DNA
agagacctcatttgtgcacaaaagtttaayggccttactgttttgccacctttgctcacagatgaaatgattgct

SEQ ID NO: 677 - DNA, 75 bp, synthetic construct
  misc_feature 1..75: note = Synthetic
  source 1..75: mol_type = other DNA
agagacctcatttgtgcacaaaagtttaayggccttaatgttttgccacctttgctcacagatgaaatgattgct

SEQ ID NO: 678 - DNA, 75 bp, synthetic construct
  misc_feature 1..75: note = Synthetic
  source 1..75: mol_type = other DNA
agcaatcatttcatctgtgagcaaaggtggcaaaacagtaaggccrttaaacttttgtgcacaaatgaggtctct

SEQ ID NO: 679 - DNA, 75 bp, synthetic construct
  misc_feature 1..75: note = Synthetic
  source 1..75: mol_type = other DNA
agcaatcatttcatctgtgagcaaaggtggcaaaacattaaggccrttaaacttttgtgcacaaatgaggtctct

SEQ ID NO: 680 - DNA, 24 bp, synthetic construct
  misc_feature 1..24: note = Synthetic
  source 1..24: mol_type = other DNA
tgcacaaaagtttaayggccttam

SEQ ID NO: 681 - DNA, 21 bp, synthetic construct
  misc_feature 1..21: note = Synthetic
  source 1..21: mol_type = other DNA
tgttttgccacctttgctcac

SEQ ID NO: 682 - DNA, 22 bp, synthetic construct
  misc_feature 1..22: note = Synthetic
  source 1..22: mol_type = other DNA
gtgagcaaaggtggcaaaacak

SEQ ID NO: 683 - DNA, 23 bp, synthetic construct
  misc_feature 1..23: note = Synthetic
  source 1..23: mol_type = other DNA
taaggccrttaaacttttgtgca

SEQ ID NO: 684 - DNA, 24 bp, synthetic construct
  misc_feature 1..24: note = Synthetic
  source 1..24: mol_type = other DNA
agtagcatccttgatttcaccttg

SEQ ID NO: 685 - DNA, 21 bp, synthetic construct
  misc_feature 1..21: note = Synthetic
  source 1..21: mol_type = other DNA
aggggtactgctgttatgtct

SEQ ID NO: 686 - DNA, 20 bp, synthetic construct
  misc_feature 1..20: note = Synthetic
  source 1..20: mol_type = other DNA
gatgcaaatctggtggcgtt

SEQ ID NO: 687 - DNA, 23 bp, synthetic construct
  misc_feature 1..23: note = Synthetic
  source 1..23: mol_type = other DNA
ggggtactgctgttatgtcttta

SEQ ID NO: 688 - DNA, 21 bp, synthetic construct
  misc_feature 1..21: note = Synthetic
  source 1..21: mol_type = other DNA
tgcaaatctggtggcgttaaa

SEQ ID NO: 689 - DNA, 20 bp, synthetic construct
  misc_feature 1..20: note = Synthetic
  source 1..20: mol_type = other DNA
ctgagagagggtcaagtgca

SEQ ID NO: 690 - DNA, 23 bp, synthetic construct
  misc_feature 1..23: note = Synthetic
  source 1..23: mol_type = other DNA
actttagagtccaaccaacagaa

SEQ ID NO: 691 - DNA, 20 bp, synthetic construct
  misc_feature 1..20: note = Synthetic
  source 1..20: mol_type = other DNA
gcccctattaaacagcctgc

SEQ ID NO: 692 - DNA, 20 bp, synthetic construct
  misc_feature 1..20: note = Synthetic
  source 1..20: mol_type = other DNA
tttaacgccaccagatttgc

SEQ ID NO: 693 - DNA, 20 bp, synthetic construct
  misc_feature 1..20: note = Synthetic
  source 1..20: mol_type = other DNA
agcctgcacgtgtttgaaaa

SEQ ID NO: 694 - DNA, 23 bp, synthetic construct
  misc_feature 1..23: note = Synthetic
  source 1..23: mol_type = other DNA
actttagagtccaaccaacagaa

SEQ ID NO: 695 - DNA, 21 bp, synthetic construct
  misc_feature 1..21: note = Synthetic
  source 1..21: mol_type = other DNA
acctgtagaataaacacgcca

SEQ ID NO: 696 - DNA, 20 bp, synthetic construct
  misc_feature 1..20: note = Synthetic
  source 1..20: mol_type = other DNA
aacgccaccagatttgcatc

SEQ ID NO: 697 - DNA, 20 bp, synthetic construct
  misc_feature 1..20: note = Synthetic
  source 1..20: mol_type = other DNA
gcaggctgtttaataggggc

SEQ ID NO: 698 - DNA, 20 bp, synthetic construct
  misc_feature 1..20: note = Synthetic
  source 1..20: mol_type = other DNA
agtctgcctgtgatcaacct

SEQ ID NO: 699 - DNA, 20 bp, synthetic construct
  misc_feature 1..20: note = Synthetic
  source 1..20: mol_type = other DNA
ttttcaaacacgtgcaggct

SEQ ID NO: 700 - DNA, 20 bp, synthetic construct
  misc_feature 1..20: note = Synthetic
  source 1..20: mol_type = other DNA
tgcacttcagcctcaacttt

SEQ ID NO: 701 - DNA, 20 bp, synthetic construct
  misc_feature 1..20: note = Synthetic
  source 1..20: mol_type = other DNA
aacacgtgcaggctgtttaa

SEQ ID NO: 702 - DNA, 21 bp, synthetic construct
  misc_feature 1..21: note = Synthetic
  source 1..21: mol_type = other DNA
tcaatttgcacttcagcctca

SEQ ID NO: 703 - DNA, 21 bp, synthetic construct
  misc_feature 1..21: note = Synthetic
  source 1..21: mol_type = other DNA
acgtgcaggctgtttaatagg

SEQ ID NO: 704 - DNA, 22 bp, synthetic construct
  misc_feature 1..22: note = Synthetic
  source 1..22: mol_type = other DNA
ctttgaagtctgcctgtgatca

SEQ ID NO: 705 - DNA, 22 bp, synthetic construct
  misc_feature 1..22: note = Synthetic
  source 1..22: mol_type = other DNA
cacttcagcctcaactttgtca

SEQ ID NO: 706 - DNA, 20 bp, synthetic construct
  misc_feature 1..20: note = Synthetic
  source 1..20: mol_type = other DNA
aggttgatcacaggcagact

SEQ ID NO: 707 - DNA, 20 bp, synthetic construct
  misc_feature 1..20: note = Synthetic
  source 1..20: mol_type = other DNA
gtgcaacgccaacaataagc

SEQ ID NO: 708 - DNA, 21 bp, synthetic construct
  misc_feature 1..21: note = Synthetic
  source 1..21: mol_type = other DNA
acaaagttgaggctgaagtgc

SEQ ID NO: 709 - DNA, 20 bp, synthetic construct
  misc_feature 1..20: note = Synthetic
  source 1..20: mol_type = other DNA
acgccaacaataagccatcc

SEQ ID NO: 710 - DNA, 22 bp, synthetic construct
  misc_feature 1..22: note = Synthetic
  source 1..22: mol_type = other DNA
cttgacaaagttgaggctgaag

SEQ ID NO: 711 - DNA, 20 bp, synthetic construct
  misc_feature 1..20: note = Synthetic
  source 1..20: mol_type = other DNA
agggagtgaggcttgtatcg

SEQ ID NO: 712 - DNA, 22 bp, synthetic construct
  misc_feature 1..22: note = Synthetic
  source 1..22: mol_type = other DNA
gctgaagtgcaaattgataggt

SEQ ID NO: 713 - DNA, 21 bp, synthetic construct
  misc_feature 1..21: note = Synthetic
  source 1..21: mol_type = other DNA
gccaacaataagccatccgaa

SEQ ID NO: 714 - DNA, 54 bp, synthetic construct
  misc_feature 1..54: note = Synthetic
  source 1..54: mol_type = other DNA
catttggtcattggttcaagacgaatttaatgatggtgtttattttgcttccac

SEQ ID NO: 715 - DNA, 54 bp, synthetic construct
  misc_feature 1..54: note = Synthetic
  source 1..54: mol_type = other DNA
ctggtccgttgtggtccttctaacatttaatgatggtgtttattttgcttccat

SEQ ID NO: 716 - DNA, 28 bp, synthetic construct
  misc_feature 1..28: note = Synthetic
  source 1..28: mol_type = other DNA
tgagaagtytaacataataagaggctgg

SEQ ID NO: 717 - DNA, 52 bp, synthetic construct
  misc_feature 1..52: note = Synthetic
  source 1..52: mol_type = other DNA
catttggtcattggttcaagacgacagcctcttattatgttaracttctcag

SEQ ID NO: 718 - DNA, 52 bp, synthetic construct
  misc_feature 1..52: note = Synthetic
  source 1..52: mol_type = other DNA
ctggtccgttgtggtccttctaaccagcctcttattatgttaracttctcaa

SEQ ID NO: 719 - DNA, 30 bp, synthetic construct
  misc_feature 1..30: note = Synthetic
  source 1..30: mol_type = other DNA
tggaagcaaaataaacaccatcattaaatg

SEQ ID NO: 720 - DNA, 52 bp, synthetic construct
  misc_feature 1..52: note = Synthetic
  source 1..52: mol_type = other DNA
ctaatagctcctgtgccctcgtatctaatattacaaacttgtgcccttttgg

SEQ ID NO: 721 - DNA, 52 bp, synthetic construct
  misc_feature 1..52: note = Synthetic
  source 1..52: mol_type = other DNA
aatccgtcgactagcctgagaattctaatattacaaacttgtgcccttttga

SEQ ID NO: 722 - DNA, 26 bp, synthetic construct
  misc_feature 1..26: note = Synthetic
  source 1..26: mol_type = other DNA
tgaagtttttaacgccaccaratttg

SEQ ID NO: 723 - DNA, 50 bp, synthetic construct
  misc_feature 1..50: note = Synthetic
  source 1..50: mol_type = other DNA
ctaatagctcctgtgccctcgtataaatytggtggcgttaaaaacttcac

SEQ ID NO: 724 - DNA, 50 bp, synthetic construct
  misc_feature 1..50: note = Synthetic
  source 1..50: mol_type = other DNA
aatccgtcgactagcctgagaattaaatytggtggcgttaaaaacttcat

SEQ ID NO: 725 - DNA, 28 bp, synthetic construct
  misc_feature 1..28: note = Synthetic
  source 1..28: mol_type = other DNA
caaaagggcacaagtttgtaatattagg

SEQ ID NO: 726 - DNA, 46 bp, synthetic construct
  misc_feature 1..46: note = Synthetic
  source 1..46: mol_type = other DNA
gtggcaacagaaatcaggtggtgaggctgtttaataggggytgaac

SEQ ID NO: 727 - DNA, 46 bp, synthetic construct
  misc_feature 1..46: note = Synthetic
  source 1..46: mol_type = other DNA
agaggactgctaaaggtttgtaggggctgtttaataggggytgaat

SEQ ID NO: 728 - DNA, 27 bp, synthetic construct
  misc_feature 1..27: note = Synthetic
  source 1..27: mol_type = other DNA
atgtyaayaactcatatgagtgtgaca

SEQ ID NO: 729 - DNA, 51 bp, synthetic construct
  misc_feature 1..51: note = Synthetic
  source 1..51: mol_type = other DNA
gtggcaacagaaatcaggtggtgagtcacactcatatgagttrttracatg

SEQ ID NO: 730 - DNA, 51 bp, synthetic construct
  misc_feature 1..51: note = Synthetic
  source 1..51: mol_type = other DNA
agaggactgctaaaggtttgtagggtcacactcatatgagttrttracata

SEQ ID NO: 731 - DNA, 22 bp, synthetic construct
  misc_feature 1..22: note = Synthetic
  source 1..22: mol_type = other DNA
ttcarcccctattaaacagcct

SEQ ID NO: 732 - DNA, 75 bp, synthetic construct
  misc_feature 1..75: note = Synthetic
  source 1..75: mol_type = other DNA
gtcctaccatttaatgatggtgtttattttgcttccactgagaagtctaacataataagaggctggatttttggt

SEQ ID NO: 733 - DNA, 75 bp, synthetic construct
  misc_feature 1..75: note = Synthetic
  source 1..75: mol_type = other DNA
gtcctaccatttaatgatggtgtttattttgcttccattgagaagtctaacataataagaggctggatttttggt

SEQ ID NO: 734 - DNA, 75 bp, synthetic construct
  misc_feature 1..75: note = Synthetic
  source 1..75: mol_type = other DNA
accaaaaatccagcctcttattatgttagacttctcagtggaagcaaaataaacaccatcattaaatggtaggac

SEQ ID NO: 735 - DNA, 75 bp, synthetic construct
  misc_feature 1..75: note = Synthetic
  source 1..75: mol_type = other DNA
accaaaaatccagcctcttattatgttagacttctcaatggaagcaaaataaacaccatcattaaatggtaggac

SEQ ID NO: 736 - DNA, 75 bp, synthetic construct
  misc_feature 1..75: note = Synthetic
  source 1..75: mol_type = other DNA
ttagatttcctaatattacaaacttgtgcccttttggtgaagtttttaacgccaccagatttgcatctgtttatg

SEQ ID NO: 737 - DNA, 75 bp, synthetic construct
  misc_feature 1..75: note = Synthetic
  source 1..75: mol_type = other DNA
ttagatttcctaatattacaaacttgtgcccttttgatgaagtttttaacgccaccagatttgcatctgtttatg

SEQ ID NO: 738 - DNA, 75 bp, synthetic construct
  misc_feature 1..75: note = Synthetic
  source 1..75: mol_type = other DNA
ataaacagatgcaaatctggtggcgttaaaaacttcaccaaaagggcacaagtttgtaatattaggaaatctaac

SEQ ID NO: 739 - DNA, 75 bp, synthetic construct
  misc_feature 1..75: note = Synthetic
  source 1..75: mol_type = other DNA
ataaacagatgcaaatctggtggcgttaaaaacttcatcaaaagggcacaagtttgtaatattaggaaatctaac

SEQ ID NO: 740 - DNA, 75 bp, synthetic construct
  misc_feature 1..75: note = Synthetic
  source 1..75: mol_type = other DNA
ttttcaaacacgtgcaggctgtttaataggggctgaacatgtcaacaactcatatgagtgtgacatacccattgg

SEQ ID NO: 741 - DNA, 75 bp, synthetic construct
  misc_feature 1..75: note = Synthetic
  source 1..75: mol_type = other DNA
ttttcaaacacgtgcaggctgtttaataggggctgaatatgtcaacaactcatatgagtgtgacatacccattgg

SEQ ID NO: 742 - DNA, 75 bp, synthetic construct
  misc_feature 1..75: note = Synthetic
  source 1..75: mol_type = other DNA
ccaatgggtatgtcacactcatatgagttgttgacatgttcagcccctattaaacagcctgcacgtgtttgaaaa

SEQ ID NO: 743 - DNA, 75 bp, synthetic construct
  misc_feature 1..75: note = Synthetic
  source 1..75: mol_type = other DNA
ccaatgggtatgtcacactcatatgagttgttgacatattcagcccctattaaacagcctgcacgtgtttgaaaa

SEQ ID NO: 744 - DNA, 30 bp, synthetic construct
  misc_feature 1..30: note = Synthetic
  source 1..30: mol_type = other DNA
atttaatgatggtgtttattttgcttccay

SEQ ID NO: 745 - DNA, 28 bp, synthetic construct
  misc_feature 1..28: note = Synthetic
  source 1..28: mol_type = other DNA
tgagaagtytaacataataagaggctgg

SEQ ID NO: 746 - DNA, 28 bp, synthetic construct
  misc_feature 1..28: note = Synthetic
  source 1..28: mol_type = other DNA
cagcctcttattatgttaracttctcar

SEQ ID NO: 747 - DNA, 30 bp, synthetic construct; identical to SEQ ID NO: 719
  misc_feature 1..30: note = Synthetic
  source 1..30: mol_type = other DNA

SEQ ID NO: 748 - DNA, 28 bp, synthetic construct
  misc_feature 1..28: note = Synthetic
  source 1..28: mol_type = other DNA
ctaatattacaaacttgtgcccttttgr

SEQ ID NO: 749 - DNA, 26 bp, synthetic construct
  misc_feature 1..26: note = Synthetic
  source 1..26: mol_type = other DNA
tgaagtttttaacgccaccaratttg

SEQ ID NO: 750 - DNA, 26 bp, synthetic construct
  misc_feature 1..26: note = Synthetic
  source 1..26: mol_type = other DNA
aaatytggtggcgttaaaaacttcay

SEQ ID NO: 751 - DNA, 28 bp, synthetic construct
  misc_feature 1..28: note = Synthetic
  source 1..28: mol_type = other DNA
caaaagggcacaagtttgtaatattagg

SEQ ID NO: 752 - DNA, 22 bp, synthetic construct
  misc_feature 1..22: note = Synthetic
  source 1..22: mol_type = other DNA
ggctgtttaataggggytgaay

SEQ ID NO: 753 - DNA, 27 bp, synthetic construct
  misc_feature 1..27: note = Synthetic
  source 1..27: mol_type = other DNA
atgtyaayaactcatatgagtgtgaca

SEQ ID NO: 754 - DNA, 27 bp, synthetic construct
  misc_feature 1..27: note = Synthetic
  source 1..27: mol_type = other DNA
gtcacactcatatgagttrttracatr

SEQ ID NO: 755 - DNA, 22 bp, synthetic construct
  misc_feature 1..22: note = Synthetic
  source 1..22: mol_type = other DNA
ttcarcccctattaaacagcct

SEQ ID NO: 756 - DNA, 21 bp, synthetic construct
  misc_feature 1..21: note = Synthetic
  source 1..21: mol_type = other DNA
aggggtactgctgttatgtct

SEQ ID NO: 757 - DNA, 21 bp, synthetic construct
  misc_feature 1..21: note = Synthetic
  source 1..21: mol_type = other DNA
gcatccttgatttcaccttgc

SEQ ID NO: 758 - DNA, 21 bp, synthetic construct
  misc_feature 1..21: note = Synthetic
  source 1..21: mol_type = other DNA
gcatccttgatttcaccttgc

(19) United States
(12) Patent Application Publication
(10) Pub. No.: US 2025/0362296 A1
(43) Pub. Date: Nov. 27, 2025

(54) VIRAL STRAIN SEROLOGY ASSAYS

(71) Applicants: MESO SCALE TECHNOLOGIES, LLC., Rockville, MD (US); The United States of America,as represented by the Secretary,Department of Health and Human Services, Rockville, MD (US)

(72) Inventors: Jacob N. WOHLSTADTER, Palm Beach Gardens, FL (US); George SIGAL, Rockville, MD (US); James WILBUR, Germantown, MD (US); Jeffery DEBAD, Ijamsville, MD (US); Hans BIEBUYCK, Potomac, MD (US); Priscilla KRAI, Germantown, MD (US); Alan KISHBAUGH, Rockville, MD (US); Leonid DZANTIEV, Ellicott City, MD (US); Christopher SHELBURNE, Rockville, MD (US); Christopher CAMPBELL, Columbia, MD (US); Anastasia AKSYUK, Rockville, MD (US); Seth B. HARKINS, Bethesda, MD (US); John FULKERSON, Silver Spring, MD (US); Jocelyn Jean JAKUBIK, Frederick, MD (US); Adrian MCDERMOTT, Bethesda, MD (US); Sarah O'CONNELL, Bethesda, MD (US); Sandeep NARPALA, Bethesda, MD (US); Daniel DOUEK, Bethesda, MD (US); Ananda CHOWDHURY, Clarksburg, MD (US); Alicen SPAULDING, Minneapolis, MN (US); Leonid SEREBRYANNYY, Gaithersburg, MD (US)

(21) Appl. No.: 18/874,674

(22) PCT Filed: Jun. 16, 2023
(86) PCT No.: PCT/US2023/068614

§ 371 Date: Dec. 13, 2024

Related U.S. Application Data
(60) Provisional application No. 63/353,225, filed on Jun. 17, 2022.
(60) Provisional application No. 63/371,309, filed on Aug. 12, 2022.
(60) Provisional application No. 63/382,391, filed on Nov. 4, 2022.
(60) Provisional application No. 63/384,673, filed on Nov. 22, 2022.
(60) Provisional application No. 63/482,083, filed on Jan. 30, 2023.

(51) Int. Cl. G01N 33/569; G01N 33/543; G01N 33/58

(52) U.S. Cl. CPC G01N 33/56983; G01N 33/5438; G01N 33/582; G01N 2333/165; G01N 2469/20

(57) ABSTRACT

The invention relates to methods and kits for determining a SARS-CoV-2 strain in a sample. The invention also provides methods and kits for detecting a single nucleotide polymorphism (SNP) in a target nucleic acid, wherein the target nucleic acid is a SARS-CoV-2 nucleic acid. The invention further provides methods and kits for detecting one or more antibody biomarkers in a sample.

### INCORPORATION BY REFERENCE

Reference is made to U.S. Publication No. 2022/0003766; U.S. Publication No. 2021/0349104; PCT Publication No. WO 2021/222827; PCT Publication No. WO 2021/222830; PCT Publication No. WO 2021/222832; U.S. Publication No. 2022/0404360; U.S. Publication No. 2022/0381780; PCT Publication No. WO 2022/246213; and PCT Publication No. WO 2022/246215, the contents of each of which is incorporated by reference in its entirety.

### SEQUENCE LISTING

The instant application contains a Sequence Listing which has been submitted electronically in XML format and is hereby incorporated by reference in its entirety. Said Sequence Listing XML, created on Aug. 5, 2022, is named 0076-0031PR33.xml and is 975,567 bytes in size.

### FIELD OF THE INVENTION

The invention relates to methods and kits for determining a SARS-CoV-2 strain in a sample. The invention also provides methods and kits for detecting a single nucleotide polymorphism (SNP) in a target nucleic acid, wherein the target nucleic acid is a SARS-CoV-2 nucleic acid. The invention further provides methods and kits for detecting one or more antibody biomarkers in a sample.

### BACKGROUND

Respiratory viruses, including coronaviruses, can cause outbreaks of severe respiratory illnesses that place great burden on communities and healthcare systems. During an outbreak, large-scale tests are needed to identify infected but asymptomatic or mildly ill individuals, which can mitigate widespread disease transmission.

The COVID-19 pandemic created an urgent need for assays for multiple reasons, for example: to detect infection, to determine the stage of infection, e.g., viral load, to determine transmissibility of the virus, to determine presence or absence of virus, e.g., on surfaces, to aid in the development of vaccines, for epidemiological studies, to follow the immune status and past viral exposure of individuals, for research into factors contributing to morbidity and mortality of viral infection. Although some assays were developed early in the pandemic, they were slow or low throughput, lacked sensitivity, were inaccurate, were expensive, or otherwise inadequate. For example, current PCR-based tests, e.g., for SARS-CoV-2, are analytically sensitive but require a lengthy, complex, and expensive sample processing procedure, and may be difficult to run at the scale needed to screen large populations. Moreover, accurate and sensitive serology tests can be useful for epidemiological studies and to identify individuals who are immune or at low risk of infection. Thus, high-quality assays are desperately needed to address the pandemic.

### SUMMARY OF THE INVENTION

In embodiments, the invention provides a method for determining a SARS-CoV-2 strain in a sample, comprising: (a) detecting at least a first antibody biomarker in the sample that binds to an antigen, e.g., an S protein, N protein, and/or S-RBD, from a first SARS-CoV-2 strain and at least a second antibody biomarker in the sample that binds to an antigen, e.g., an S protein, N protein, and/or S-RBD, from a second SARS-CoV-2 strain, wherein the detecting comprises contacting the sample with a surface comprising one or more binding domains, wherein the S protein from the first SARS-CoV-2 strain is immobilized on a first binding domain, and the S protein from the second SARS-CoV-2 strain is immobilized on a second binding domain; and (b) determining a ratio of the first antibody biomarker to the second antibody biomarker, thereby determining the SARS-CoV-2 strain. In embodiments, the detecting comprises forming a binding complex in each binding domain that comprises an antibody biomarker and the antigen, e.g., the S protein, N protein, or S-RBD; contacting the binding complex in each binding domain with a detection reagent; and measuring concentration of the antibody biomarker in each binding complex.

In embodiments, the invention provides method for detecting a single nucleotide polymorphism (SNP) in a target nucleic acid, wherein the target nucleic acid is a SARS-CoV-2 nucleic acid, comprising: (a) contacting a sample comprising the target nucleic acid with (i) a targeting probe, wherein the targeting probe comprises a first region complementary to a polymorphic site of the target nucleic acid that comprises the SNP, and wherein the targeting probe comprises an oligonucleotide tag; and (ii) a detection probe, wherein the detection probe comprises a second region complementary to an adjacent region of the target nucleic acid comprising the polymorphic site, and wherein the detection probe comprises a detectable label; (b) hybridizing the targeting and detection probes to the target nucleic acid; (c) ligating the targeting and detection probes that hybridize with perfect complementarity at the polymorphic site to form a ligated target complement comprising the oligonucleotide tag and the detectable label; (d) contacting the product of (c) with a surface comprising an immobilized binding reagent, wherein the binding reagent comprises an oligonucleotide complementary to the oligonucleotide tag; (e) forming a binding complex on the surface, wherein the binding complex comprises the binding reagent and the ligated target complement; and (f) detecting the binding complex, thereby detecting the SNP at the polymorphic site.

In embodiments, the invention provides a kit for detecting one or more antibody biomarkers of interest in a sample, the kit comprising, in one or more vials, containers, or compartments: (a) a surface comprising one or more binding domains, wherein each binding domain comprises an antigen immobilized thereon; and (b) one or more detection reagents, wherein each detection reagent comprises a detection antibody, a detection antigen, or an ACE detection reagent.

In embodiments, the invention provides a method of detecting one or more antibody biomarkers of interest in a sample, comprising: (a) contacting the sample with a surface comprising one or more binding domains, wherein each binding domain comprises an antigen immobilized thereon; (b) forming a binding complex in each binding domain, wherein the binding complex comprises the antigen and an antibody biomarker that binds to the antigen; (c) contacting the binding complex in each binding domain with a detection reagent; and (d) detecting the binding complexes on the surface, thereby detecting the one or more antibody biomarkers in the sample.

### BRIEF DESCRIPTION OF THE DRAWINGS

The following drawings form part of the present specification and are included to further demonstrate exemplary embodiments of certain aspects of the present invention.

FIG. 1 relates to Example 1. FIG. 1 shows the results of an embodiment of a bridging serology assay described herein. SARS-CoV-2 S-RBD was immobilized as binding reagent, and labeled S-RBD was used as detection reagent. The bridging serology assay was tested on serum samples from COVID-19 positive (red circles) and normal (non-COVID-19) (blue circles) patients, diluted 10-fold or 100-fold. Higher signal indicates increased number of antibodies bound to the immobilized antigen.

FIG. 2 relates to Example 2. FIG. 2 shows the results of an embodiment of a neutralization serology assay described herein. SARS-CoV-2 S protein was immobilized as binding reagent, and labeled ACE2 was added as a competitor to SARS-CoV-2 antibodies that may be present. The neutralization serology assay was tested on serum samples from COVID-19 positive (red circles) and normal (non-COVID-19) (blue circles) patients, diluted 10-fold or 100-fold. Lower signal (generated by competitor) indicates increased number of antibodies bound to the immobilized antigen.

FIGS. 3A-3D illustrate an embodiment of the methods described herein for detecting a single nucleotide polymorphism (SNP) in a viral nucleic acid. In FIGS. 9A-9C, a target nucleic acid (1) that comprises an SNP (2) is contacted with: a targeting probe (3) that comprises an oligonucleotide tag (4) and a sequence that is complementary to the SNP, and a detection probe (5) that comprises detectable label (6). The targeting and detection probes (3, 5) hybridize to the target nucleic acid, and the targeting and detection probes that hybridize with perfect complementarity at the SNP are ligated to form a ligated target complement (11) comprising the oligonucleotide tag and detectable label. The reaction mixture containing the ligated target complement is contacted with a surface comprising one or more binding reagents (7) immobilized in one or more binding domains (9). A signal (10) is detected if the ligated target complement is immobilized on the surface via hybridization of the complementary oligonucleotides in the oligonucleotide tag and the binding reagent. In FIG. 3D, the targeting probe has a mismatch with the SNP in the target nucleic acid, and thus, hybridization and ligation do not occur.

FIG. 4 illustrates an embodiment of the methods described herein for detecting a viral nucleic acid. RNA is extracted from a sample containing an RNA virus (e.g., SARS-CoV-2), and the extracted RNA is converted to cDNA. A “Master Mix” is prepared by combining a forward primer comprising a 5′ binding reagent complement sequence and a cDNA complement sequence, a reverse primer comprising a cDNA reverse complement sequence and a 3′ binding partner of a detectable label, and other PCR components such as dNTPs and DNA polymerase. The cDNA and Master Mix are combined, and PCR is performed for 30 to 40 cycles to form a plurality of PCR products, each PCR product comprising the 5′ binding reagent complement sequence and 3′ binding partner of a detectable label. Each PCR product hybridizes to a binding reagent on a surface. The surface is then contacted with a detectable label, which binds to the PCR product. The PCR product bound to the detectable label is then subjected to detection as described herein.

FIGS. 5, 6A, and 6B relate to Example 4. FIG. 5 shows the correlation between embodiments of serology assays described herein.

FIG. 6A shows the correlation results of the indirect serology assays for IgG against SARS-CoV-2 S with four other scrology assays: IgG against SARS-CoV-2 N, IgG against SARS-CoV-2 S-RBD, IgM against SARS-CoV-2 S, and ACE2 competitor assay. FIG. 6B shows the assay performance (sensitivity and specificity) for the assay pairings of FIG. 6A.

FIG. 7 relates to Example 5. FIG. 7 shows the assay performance (sensitivity at early and late infections and specificity) of IgG indirect serology assay and IgM indirect serology assay and ACE2 competitor assay.

FIGS. 8-10 relate to Example 6. FIG. 8 shows the results from an exemplary oligonucleotide ligation assay (OLA) for detection of SARS-CoV-2 single nucleotide polymorphisms (SNPs) at genome locations 8782, 11083, 23403, and 28144, with a synthetic template oligonucleotide.

FIG. 9 shows the results of an exemplary singleplex OLA assay for detecting SARS-CoV-2 SNPs at genome locations 8782, 11083, 23403, and 28144, with samples obtained from SARS-CoV-2 positive patients.

FIG. 10 shows the results of an exemplary multiplex OLA assay for detecting SARS-CoV-2 SNPs at genome locations 8782, 11083, 23403, and 28144, with samples obtained from SARS-CoV-2 positive patients.

FIGS. 11-13 relate to Example 7. FIG. 11 shows the results of an exemplary assay for measuring the concentration (fg/mL) of SARS-CoV-2 nucleocapsid (N) protein from the following samples: nasopharyngeal swabs from 12 patients who tested positive for COVID-19, nasopharyngeal swabs from 6 patients who tested negative for COVID-19, and normal (COVID-19 negative) human saliva, serum, and EDTA plasma.

FIG. 12 shows the percent recovery results of an exemplary test to assess dilution linearity of the SARS-CoV-2 N protein detection assay. The normal human serum, EDTA plasma, saliva, and COVID-19 negative human nasopharyngeal swab samples were spiked with calibrator and tested at different dilutions.

FIG. 13 shows the percent recovery results of an exemplary test to assess spike recovery of the SARS-CoV-2 N protein detection assay. The normal human serum, EDTA plasma, saliva, and COVID-19 negative human nasopharyngeal swab samples were spiked with calibrator at three levels.

FIG. 14 shows the results of an exemplary serology assay performed on samples obtained from SARS-CoV-2-infected individuals in the United States during early 2020 (known to be infected with wild-type SARS-CoV-2 (“Wuhan”)); SARS-CoV-2-infected individuals in the United Kingdom (dominating strain: SARS-CoV-2 strain B.1.1.7); or SARS-CoV-2-infected individuals in South Africa (dominating strain: SARS-CoV-2 strain 501Y.V2, also known as B.1.351). The measured ratios of antibodies against wild-type SARS-CoV-2 versus SARS-CoV-2 strain B.1.1.7 were plotted on the x-axis, and the measured ratios of antibodies against wild-type SARS-CoV-2 versus SARS-CoV-2 strain 501Y.V2 were plotted on the y-axis.

FIG. 15 shows the results of an exemplary serology assay to determine antibody concentrations for endemic coronaviruses in finger-stick blood, saliva, and serum in samples from subjects as described in Table 7.

FIG. 16 shows the results of an exemplary serology assay to determine reactivity to SARS-CoV-2 antigens in finger-stick blood and saliva samples from subjects described in Table 7. Dashed lines indicate assay sensitivity and quantitation (LLOD=lower limit of detection; LLOQ=lower limit of quantitation; ULOQ=upper limit of quantitation). The dotted line labeled “98%” is drawn at the threshold set at the 98^{th} percentile for the presumed naïve (PN) donors. Filled circles indicates donors whose IgG levels in finger-stick blood exceeded the threshold for SARS-CoV-2 spike.

FIG. 17 shows the results of an exemplary serology assay to determine total immunoglobulin concentrations in finger-stick blood samples from subjects described in Table 7.

FIG. 18 shows the results of an exemplary serology to determine total immunoglobulin concentrations in saliva samples from subjects described in Table 7.

FIG. 19 shows the results of an exemplary serology assay to determine correlation in reactivity to SARS-CoV-2 antigens measured in self-collected saliva versus finger-stick blood from subjects described in Table 7.

FIG. 20 shows the results of an exemplary serology assay to determine correlation in salivary IgG levels for CoV-2 spike, RBD, and N antigens in samples from subjects described in Table 7. Dashed lines indicate the selected classification thresholds set at the 98^{th} percentile of saliva from subjects who reported no COVID-19 diagnosis, recent symptoms, or household exposure to COVID-19.

FIG. 21 shows the relative reactivity to the SARS-CoV-2 nucleocapsid (N) and spike (S) antigens measured in finger-stick blood and saliva from subjects whose levels of anti-spike IgG exceeded the threshold as shown in FIG. 16. Spearman coefficient=0.95, p=0.001. The dashed line has slope of 1 and represents the expected correlation.

FIG. 22 shows the results of exemplary indirect IgG serology and ACE2 competition assays using a 10-spot SARS-CoV-2 S-RBD antigen panel. The graph shows the signals for each of the antigens in the S-RBD antigen panel (identified in the inset table) after normalization to the signal from the wild-type SARS-CoV-2 S-RBD antigen spot.

FIGS. 23 and 24 show heat map results of the data in FIG. 22. Each lower row shows the signal for one of the 10 S-RBD antigen spots after normalization across the column. Each column is one individual sample (˜200 samples infected with wild-type SARS-CoV-2 and 32 samples infected with strain B.1.351). FIG. 23 shows results from the ACE2 competition assay, and FIG. 24 shows results from the IgG indirect serology assay.

FIG. 25 shows a subset of the data in FIGS. 22-24, with signals from two spots in the 10-spot S-RBD antigen panel. Each dot represents one individual.

FIGS. 26A and 66B show the results of exemplary indirect IgG serology (FIG. 26A) and ACE2 competition (FIG. 26B) assays to detect anti-CoV-2 spike antibodies. Both assays were tested against a set of 214 serum samples collected from individuals at different time points after confirmed SARS-CoV-2 infection (diagnosis by PCR; 0-14 days, 15-28 days, 29-56 days, and 57+ days) and 200 control samples collected prior to the emergence of SARS-CoV-2 in 2020. Horizontal line A shows the optimal threshold for classification accuracy.

FIG. 27 shows the results of exemplary multiplexed oligonucleotide ligation assay (OLA) panel for detection of SARS-CoV-2 single nucleotide polymorphisms (SNPs) in the S protein: 69-70del, D215G, D253G, K417N, K417T, L452R, E484K, N501Y, D614G, and P681H. The top panel shows the results from a known SARS-CoV-2 wild-type or B.1.1.7 strain. The bottom panel shows the results from 23 nasal swab samples from March or August 2020.

FIG. 28 show the results of an exemplary biomarker assay to assess levels of IL-6, IL-10, IL-12p70, IL-4, TNF-α, IL-2, IL-1β, IFN-γ, and IL-17A, performed on cerebrospinal fluid (CSF) and serum samples from acute COVID-19 patients and non-COVID-19 control subjects.

FIGS. 29A and 29B illustrate exemplary assay surfaces described in embodiments herein. FIG. 29A shows a well of an exemplary 384-well assay plate, comprising four distinct binding domains (“spots”). FIG. 29B shows a well of an exemplary 96-well assay plate, comprising ten distinct binding domains (“spots”).

### DETAILED DESCRIPTION OF THE INVENTION

Certain inventions disclosed herein were made jointly under Research Collaboration Agreement 2020-0351 between the National Institute of Allergy and Infectious Diseases (NIAID), which is a component of the National Institutes of Health (NIH), which is an agency of the U.S. Department of Health and Human Services, and Meso Scale Diagnostics, LLC., which is an affiliate of Meso Scale Technologies, LLC.

The disclosed embodiments fulfill the urgent need for high-quality viral assays and methods useful for the COVID-19 pandemic. Disclosed embodiments have been widely adopted for COVID-19 research, epidemiology, and vaccine development and have had a significant impact on the COVID-19 public health response. For example, serology embodiments are widely used (e.g., Johnson M et al. J Clin Virol 2020; 130:104572; Corbett K S et al. N Engl J Med 2020; 383:1544-55; Folegatti P M et al. The Lancet 2020; 396:467-78; Ramasamy M N et al. The Lancet 2020; 396:1979-93; Goldblatt D et al. J Hosp Infect 2021; 110:60-6; Majdoubi A et al. JCI Insight 2021, doi.org/10.1172/jci.insight.146316; Amjadi M F et al. MedRxiv 2021:2021.01.05.21249240, doi.org/10.1101/2021.01.05.21249240; Grandjean L et al. MedRxiv 2020:2020.07.16.20155663, doi.org/10.1101/2020.07.16.20155663; Majdoubi A et al. MedRxiv 2020:2020.10.05.20206664, doi.org/10.1101/2020.10.05.20206664). Certain embodiments disclosed herein were chosen by the United States government initiative, Operation Warp Speed, as the basis of its standard binding assay for immunogenicity assessments in all funded Phase III clinical trials of vaccines. Serology assay embodiments (e.g., assays to detect immunoglobulin(s) conducted on non-bodily samples or bodily samples (e.g., serum, plasma, saliva)) disclosed herein aid in assessing human immune responses to COVID-19 infection and vaccination and in understanding the interplay between COVID-19 and immunity to other coronaviruses and respiratory pathogens. The disclosed nucleic acid detection embodiments have advantages over PCR methods, e.g., in their speed, simplicity, cost, and high throughput. The disclosed intact virus detection embodiments provide improved accuracy and specificity of an active infection diagnosis as compared to detection of an individual viral component. Serology assays, nucleic acid detection assays, and other embodiments related to mutations and variants of SARS-CoV-2 are proving important as new mutations and variants arise. Other biomarker detection embodiments disclosed herein, e.g., detection of inflammatory and/or tissue damage response biomarkers and/or extracellular vesicles, e.g., from virus-infected cells, have wide applicability, regardless of viral mutation status, to studies on morbidity and mortality to understand factors underlying severe illness, death, and persistent symptoms following acute infection and may lead to better interventions. Data showing the high-quality nature of the disclosed embodiments are described in the Examples and elsewhere herein.

Immunoassays described herein for the detection of respiratory viruses, including coronaviruses, provide numerous advantages compared with nucleic acid amplification (e.g., PCR) based detection methods. For example, immunoassays are conducted in a simple and streamlined format with improved sensitivity. Improved sensitivity with immunoassays occurs because these assays not only detect viral particles, but also individual viral proteins in damaged tissue being cleared by the body at the site of infections. Moreover, immunoassays for biomarkers produced by the body in response to infection (e.g., antibodies against the virus or inflammatory factors associated with the host response to infection) take advantage of the natural amplification associated with the immune response.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### Respiratory Virus Detection

In embodiments, the invention provides an immunoassay method for detecting at least one respiratory virus, including a coronavirus, in a biological sample. As used herein, a “respiratory virus” refers to a virus that can cause a respiratory tract infection, e.g., in a human. Exemplary respiratory viruses include, but are not limited to, coronavirus, influenza virus, respiratory syncytial virus (RSV), paramyxovirus, adenovirus, parainfluenza virus (PIV), bocavirus, metapneumovirus (MPV), orthopneumovirus, enterovirus, rhinovirus (RV), parechovirus (PeV), and the like. Respiratory virus infections can be difficult to diagnose because different viruses can often cause similar symptoms in a patient. For example, coughing and low-grade fever are typical symptoms of early disease progression or mild cases of a coronavirus infection (e.g., COVID-19), as well as influenza or a respiratory syncytial virus (RSV) infection. An assay that can simultaneously test for several potential causes of infection would advantageously allow a respiratory virus infection to be correctly and efficiently diagnosed in a single assay run and utilizing a single patient sample. In embodiments, the methods herein distinguish between and among different types of a given virus (e.g., distinguishing PIV-1, PIV-2, PIV-3, and PIV-4 from each other or influenza A from influenza B from each other), as well as between and among different subtypes or strains (e.g., distinguishing influenza A (H1N1) from influenza A (H3N2)).

In embodiments, the invention provides an immunoassay method for detecting at least one respiratory virus in a biological sample, comprising: (a) contacting the biological sample with a binding reagent that specifically binds a component of at least one respiratory virus in the biological sample; (b) forming a binding complex comprising the binding reagent and the respiratory virus component; and (c) detecting the binding complex, thereby detecting the at least one respiratory virus in the biological sample.

In embodiments, the at least one respiratory virus comprises a coronavirus, an influenza virus, a parainyxovirus, an adenovirus, a bocavirus, a pneumovirus, an enterovirus, a rhinovirus, or a combination thereof. Exemplary coronaviruses and methods for their detection are described herein and include, but are not limited to, SARS-CoV (also known as SARS-CoV-1), MERS-CoV, SARS-CoV-2, HCoV-OC43, HCoV-229E, HCoV-NL63, and HCoV-HKU1. In embodiments, the method detects a coronavirus by detecting a coronavirus nonstructural protein, e.g., nsp1, nsp2, nsp3, nsp4, nsp5, nsp6, nsp7, nsp8, nsp9, nsp10, nsp11, nsp12, nsp13, nsp14, nsp15, or nsp16. In embodiments, the method detects a coronavirus by detecting a coronavirus structural protein, e.g., the E, S (including S1, S2, S-NTD, S-ECD, and S-RBD), M, HE, or N proteins. Coronaviruses and their proteins are further described herein.

Exemplary influenza viruses include, but are not limited to, influenza A (Flu A), influenza B (Flu B), and influenza C (Flu C). Typically, the seasonal flu is caused by Flu A and/or Flu B. Flu A viruses can be further characterized into various subtypes based on the hemagglutinin (HA) and neuraminidase (N) proteins present on the surface of the viral particle, e.g., H1N1, H1N2, H2N2, H3N2, H5N1, H7N2, H7N3, H7N7, H9N2, and H10N7. Flu A strains include, e.g., H1 strains (such as H1/Michigan strain, H1/Wisconsin strain (also referred to as H1/Wisconsin 2019, H1/Wisconsin/588/2019 or H1N1)), H3 strains (such as H3/Hong Kong strain, H3/Darwin strain (also referred to as H3/Darwin, H3/Darwin/9/2021 or H3N2)), H7 strains (such as H7/Shanghai strain), and the like. Flu B viruses can be further characterized into genetic lineages, e.g., the Flu B/Victoria lineage (including, e.g., the strain B/Austria/1359417/2021) or Flu B/Yamagata lineage (including, e.g., the strain B/Phuket/3073/2013). In embodiments, the immunoassay detects an influenza virus component, e.g., an influenza virus-specific protein. In embodiments, the immunoassay detects an influenza structural protein. In embodiments, the immunoassay detects an influenza nonstructural protein. In embodiments, the immunoassay detects an influenza virus by detecting the influenza HA protein. In embodiments, the immunoassay detects an influenza virus by detecting the influenza N protein. In embodiments, the immunoassay detects an influenza virus by detecting an influenza nucleoprotein (NP). In embodiments, the immunoassay detects a FluA virus and is further capable of determining the subtype of the FluA virus. In embodiments, the immunoassay detects a FluB virus and is further capable of determining the lineage of the FluB virus.

In embodiments, the method detects SARS-CoV, MERS-CoV, SARS-CoV-2, HCoV-OC43, HCoV-229E, HCoV-NL63, HCoV-HKU1, influenza A, influenza B, RSV, or a combination thereof. In embodiments, the method is a multiplexed method capable of simultaneously detecting one or more of SARS-CoV, MERS-CoV, SARS-CoV-2, HCoV-OC43, HCoV-229E, HCoV-NL63, HCoV-HKU1, influenza A, influenza B, and RSV. In embodiments, the method further comprises repeating one or more of the method steps described herein to detect one or more respiratory viruses in the sample. In embodiments, the method further comprises repeating steps (a)-(c) of the method described herein, wherein each detected respiratory virus comprises a component that binds to a different binding reagent, thereby detecting the at least one respiratory virus. In embodiments, each of steps (a)-(c) is performed for each respiratory virus in parallel.

As used herein, the term “simultaneous” in reference to one or more events (e.g., detection of one or more viruses, viral components, or biomarkers as described herein) means that the events occur at exactly the same time or at substantially the same time, e.g., simultaneous events described herein can occur less than or about 30 minutes apart, less than or about 20 minutes apart, less than or about 15 minutes apart, less than or about 10 minutes apart, less than or about 5 minutes apart, less than or about 2 minutes apart, less than or about 1 minute apart, or less than or about 30 seconds apart. In the context of embodiments of multiplexed immunoassays provided herein, “simultaneous” refers to detecting a on single surface (e.g., a particle, an assay plate, an assay cartridge, or a well of a multi-well assay plate) the presence of one or more viruses, viral components or biomarkers described herein. In embodiments, a multiplexed assay is performed on a single assay plate. In embodiments, a multiplexed assay is performed in a single well of an assay plate. In embodiments, a multiplexed assay is performed in a single assay cartridge. In embodiments, a multiplexed immunoassay is performed on more than one assay plates. In embodiments, more than one multiplexed immunoassay (e.g., wherein each multiplexed immunoassay detects a combination of biomarkers and/or viral components as described herein) is performed on a single surface, e.g., a single well of an assay plate or a single assay cartridge. The number of assay wells and/or assay plates that may be required to perform a multiplexed assay can be determined, e.g., based on the number of substances of interest to be detected in one or more samples (e.g., a multiplex of about 2 to about 100, or about 2 to about 90, or about 2 to about 80, or about 2 to about 70, or about 2 to about 60, or about 2 to about 50, or about 2 to about 40, or about 2 to about 35, about 2 to about 30, or 2, 3, 4, 5, 6, 7, 8, 9, 10, 11, 12, 13, 14, 15, 16, 17, 18, 19, 20, 21, 22, 23, 24, 25, 26, 27, 28, 29, 30, or more viruses, viral components, and/or biomarkers described herein); the number of samples being assayed (e.g., from one or more subjects); the number of calibration reagents being measured to generate a calibration curve (e.g., 3, 4, 5, 6, 7, 8, 9, 10, 11, 12, or more); the number of control reagents being measured (e.g., 0, 1, 2, 3, or more); the number of replicates for each sample, calibration reagent, and/or control reagent being measured (e.g., singlicate, duplicate, triplicate, or more); and the number of wells per assay plate (e.g., 6, 12, 48, 96, 384, or 1536 wells per assay plate). When multiplexed immunoassay is conducted on multiple assay plates, the assay plates can be read simultaneously or at different times. The timing of reading the assay plates can be determined, e.g., based on the capacity of the assay reader instrument (e.g., capable of reading 1, 2, 3, 4, or more plates at once); the read-time of the assay reader instrument (e.g., about 1 s to about 600 s, about 10 s to about 500 s, about 20 s to about 300 s, about 30 s to about 180 s, about 60 s to about 120 s, about 70 s, or about 90 s per assay plate); the time required to prepare the assay components (e.g., about 10 s, 20 s, 30 s, 1 min, 2 min, 5 min, 10 min, 15 min, 30 min, 1 hr, or more per plate); and the equipment for performing the assay (e.g., a single-channel pipettor may require a longer time for pipetting the assay components as compared to a multi-channel pipettor; handling liquids from different containers, e.g., tubes, vials, or plates, may require different lengths of time). In embodiments, “simultaneous” refers to events occurring with respect to a single sample (e.g., a biological sample in a single vial or container from a single subject) or replicates or dilutions of a single sample. Factors affecting the timing of simultaneous events include the following: the number of multiplexed assays being performed at the same time on a single sample (e.g., a multiplex of or about 2 to about 100, or about 2 to about 90, or about 2 to about 80, or about 2 to about 70, or about 2 to about 60, or about 2 to about 50, or about 2 to about 40, or about 2 to about 35, about 2 to about 30, or 2, 3, 4, 5, 6, 7, 8, 9, 10, 11, 12, 13, 14, 15, 16, 17, 18, 19, 20, 21, 22, 23, 24, 25, 26, 27, 28, 29, 30, or more assays in a single well or cartridge); the number of assay modules in a panel (e.g., 1, 2, 3, or more plates or cartridges in a panel); the number of samples being assayed at the same time (e.g., a number of samples capable of being assayed in one kit or more than one kit); the number of points on a calibration curve (e.g., 5, 6, 7, 8, 9, 10, 12, or more); the presence and number of controls (e.g., 0, 1, 2, 3, or more controls); the read-time of the instrument (e.g., about 1 s to about 600 s, about 10 s to about 500 s, about 20 s to about 300 s, about 30 s to about 180 s, about 60 s to about 120 s, about 70 s, or about 90 s); the number of replicates of each calibrator, control, or sample (e.g., singlicate, duplicate, triplicate, or more); the number of wells per plate (e.g., 6, 12, 48, 96, 384, or 1536 wells per plate); and/or the type of equipment for performing the assay (e.g., a single channel or a multi channel pipettor, tubes or plates for dilution).

In embodiments, the binding reagent that specifically binds to the respiratory virus component described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof. In embodiments, the binding reagent is a receptor for the respiratory virus component. In embodiments, the binding reagent is a binding partner of the respiratory virus component. In embodiments, the binding reagent is angiotensin-converting enzyme 2 (ACE2). In embodiments, the binding reagent is a neuropilin (NRP) receptor. In embodiments, the binding reagent is NRP1. In embodiments, the binding reagent is NRP2.

### Coronavirus Detection

Coronaviruses, which belong to the Coronaviridae family of viruses, are enveloped viruses with a positive-sense single-stranded RNA genome and a nucleocapsid of helical geometry. A characteristic feature of coronaviruses is the club-shaped spikes that project from the virus surface. In general, a coronavirus particle is assembled from its structural proteins, including an envelope (E), a spike glycoprotein (S), which includes S1 and S2 subunits that form the ectodomain (S-ECD), a viral membrane protein (M), a hemagglutinin-esterase dimer (HE), nucleocapsid (N), and RNA. The S protein comprises a N-terminal domain (N-Term or NTD). The S1 subunit comprises a receptor binding domain (S-RBD), which binds a host receptor (e.g., ACE2) during infection. The S1 subunit can also bind to the cell surface neuropilin-1 (NRP1) receptor. See, e.g., Daly et al., bioRxiv 2020.06.05. 134114 (2020) doi:10.1101/2020.06.05.134114. In embodiments, coronavirus S proteins, including recombinantly expressed S proteins and variants thereof, are further described, e.g., in WO 2018/081318. For example, two variants of SARS-CoV-2 each has a single polynucleotide morphism (SNP) at genome location 23403, which is in the gene encoding the S protein, resulting in a different amino acid at position 614 of the S protein: D614 and G614 (denoted as S: 23403A>G, D614G; see, e.g., Korber et al., bioRxiv 2020.04.29. 069054 (2020) doi:10.1101/2020.04.29.069054; also published as Korber et al., Cell 182(4):P812-827 (2020)), referred to herein respectively as S-D614 and S-D614G. Further mutations of the SARS-CoV-2 S protein are described in Tables 1A and 1B. Sequence alignments between the genetic material of various coronavirus species have also revealed additional conserved open reading frames for Coronaviruses also encode a number of nonstructural proteins (NSPs), which are expressed in infected cells but are generally not incorporated into the viral particle itself. Exemplary coronavirus NSPs include, but are not limited to, nsp1, nsp2, nsp3, nsp4, nsp5, nsp6, nsp7, nsp8, nsp9 (replicase), nsp10, nsp11, nsp12 (multi-domain RNA polymerase), nsp13 (helicase, RNA 5′ triphosphatase), nsp14 (N7-methyl transferase, exonuclease), nsp15 (endoribonuclease), nsp16 (2′-O-methyl transferase), and the like. See, e.g., Snijder et al., Adv Virus Res 96:59-126 (2016); Fehr et al., Coronaviruses 1281:1-23 (2015). Sequence alignments between the genetic material of various coronavirus species have revealed conserved open reading frames for several structural and nonstructural proteins, e.g., N, M, S, nsp1, nsp3, nsp6, nsp7, and nsp8. See, e.g., Grifoni et al., bioRxiv 2020.02.12.946087 (2020) doi:10.1101/2020.02.12.

While assays for a specific coronavirus species can identify infection by that particular coronavirus, such assays may have limited usefulness when new strains of infectious coronaviruses emerge. In embodiments, the invention provides a method for detecting a coronavirus in a sample by detecting a conserved coronavirus component, e.g., a protein that is generally conserved across all coronavirus species. Such a method would enable detection of novel coronaviruses of interest.

In embodiments, the invention provides an immunoassay method for detecting a coronavirus in a biological sample, comprising: a) contacting the biological sample with a binding reagent that specifically binds a component of the coronavirus; b) forming a binding complex comprising the binding reagent and the coronavirus component; and c) detecting the binding complex, thereby detecting the coronavirus in the biological sample. In embodiments, the method detects SARS-CoV, MERS-CoV, SARS-CoV-2, HcoV-OC43, HcoV-229E, HcoV-NL63, HcoV-HKU1, or a combination thereof. In embodiments, the biological sample is saliva.

In embodiments, the coronavirus component is on the outer surface of the viral particle. In embodiments, the coronavirus component is integrated in the membrane of the viral particle. In embodiments, the coronavirus component is a protein. In embodiments, the coronavirus component comprises a sugar, e.g., a glycoprotein. In embodiments, the coronavirus component is a structural protein. In embodiments, the coronavirus component is an envelope (E) protein. In embodiments, the coronavirus component is a spike glycoprotein (S) or a variant or subunit thereof, e.g., S-D614, S-D614G, or any of the S protein variants in Tables 1A and 1B, subunit 1 (S1), subunit 2 (S2), ectodomain (S-ECD), N-terminal domain (S-NTD or S-N-Term), or receptor binding domain (S-RBD). In embodiments, the S protein subunit (e.g., S1, S2, S-ECD, S-NTD, or S-RBD) comprises a mutation as described in Tables 1A and 1B. In embodiments, the coronavirus component is a viral membrane (M1) protein. In embodiments, the coronavirus component is a hemagglutinin-esterase dimer (HE). In embodiments, the coronavirus component is a nucleocapsid (N) protein. In embodiments, the coronavirus component comprises a mutation as described in Table 1A.

In embodiments, the coronavirus component is a non-structural protein. In embodiments, the coronavirus component is nsp1, nsp2, nsp3, nsp4, nsp5, nsp6, nsp7, nsp8, nsp9, nsp10, nsp11, nsp12, nsp13, nsp14, nsp15, or nsp16. In embodiments, the coronavirus component is a protein substantially conserved across coronaviruses. It will be understood by one of ordinary skill in the art that a protein that is “substantially conserved” across a viral family, e.g., the coronavirus family, means that at least 50%, at least 60%, at least 70%, at least 80%, at least 90%, or at least 95% of species in the viral family contains a protein with at least 50%, at least 60%, at least 70%, at least 80%, at least 90%, or at least 95% sequence similarity, structural similarity, or both. Methods and tools for determining sequence and/or structural similarity are known in the field and include, e.g., algorithms such as Align, BLAST, and CLUSTAL for sequence similarity, and TM-align, DALI, STRUCTAL, and MINRMS.

In embodiments, the immunoassay method detects a coronavirus by detecting the coronavirus E protein. In embodiments, the immunoassay method detects a coronavirus by detecting the coronavirus S protein. In embodiments, the immunoassay method detects a coronavirus by detecting the coronavirus S1 protein subunit. In embodiments, the immunoassay method detects a coronavirus by detecting the coronavirus S2 protein subunit. In embodiments, the immunoassay method detects a coronavirus by detecting the coronavirus S-ECD. In embodiments, the immunoassay method detects a coronavirus by detecting the coronavirus S-RBD. In embodiments, the immunoassay method detects a coronavirus by detecting the coronavirus S-NTD. In embodiments, the immunoassay method detects a coronavirus by detecting the coronavirus M protein. In embodiments, the immunoassay method detects a coronavirus by detecting the coronavirus HE protein. In embodiments, the immunoassay method detects a coronavirus by detecting the coronavirus N protein. In embodiments, the immunoassay method detects a coronavirus by detecting one or more of the coronavirus nsp1, nsp2, nsp3, nsp4, nsp5, nsp6, nsp7, nsp8, nsp9, nsp10, nsp11, nsp12, nsp13, nsp14, nsp15, or nsp16. In embodiments, the immunoassay detects a coronavirus by detecting a combination of the coronavirus proteins described herein. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein and/or S-RBD. In embodiments, the immunoassay method detects SARS-CoV-2 by detecting any of the SARS-CoV-2 S protein and/or S-RBD variants in Tables 1A and 1B. In embodiments, the immunoassay method detects SARS-CoV-2 by detecting SARS-CoV-2 E protein. In embodiments, the immunoassay method detects SARS-CoV-2 by detecting SARS-CoV-2 M protein. In embodiments, the immunoassay detects SARS-CoV-2 by detecting SARS-CoV-2 N protein and S protein. In embodiments, the immunoassay method detects SARS-CoV-2 by detecting SARS-CoV-2 S protein, N protein, E protein, and M protein. SARS-CoV-2 nonstructural proteins include the Orf1a and Orf1ab replicase/transcriptase proteins; the Orf3a protein; the Orf6a protein; the Orf7a and Orf7b accessory proteins; the Orf8 protein monomer, which is known to form oligomers; and the Orf10 protein. SARS-CoV-2 nonstructural proteins are further described in, e.g., Khailany et al., Gene Rep 19:100682 (2020); and Flower et al., Proc Nat Acad Sci 118(2): e2021785118 (2021). In embodiments, the immunoassay detects SARS-CoV-2 by detecting any of SARS-CoV-2 Orf1a, Orf1ab, Orf3a, Orf6a, Orf7a, Orf7b, Orf8 monomer, Orf8 oligomer, Orf10, RNA-dependent RNA polymerase (RdRp), or a combination thereof. In embodiments, the immunoassay method detects SARS-CoV-2 by detecting any of the SARS-CoV-2 protein variants in Table 1A.

In embodiments, the immunoassay method for detecting SARS-CoV-2 comprises: a) contacting the biological sample with a binding reagent that specifically binds a SARS-CoV-2 S, N, E, or M protein; b) forming a binding complex comprising the binding reagent and the SARS-CoV-2 S, N, E, or M protein; and c) detecting the binding complex, thereby detecting SARS-CoV-2 in the biological sample. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises any of the mutations shown in Tables 1A and 1B. In embodiments, the SARS-CoV-2 N protein comprises any of the mutations shown in Table 1A. In embodiments, the SARS-CoV-2 E protein comprises any of the mutations shown in Table 1A. In embodiments, the binding complex further comprises a detection reagent that specifically binds to the SARS-CoV-2 S, N, E, or M protein. In embodiments, the detection reagent comprises a detectable label. In embodiments, the detection reagent comprises a nucleic acid probe. Detection reagents are further described herein. In embodiments, the biological sample is saliva.

In humans, coronaviruses can cause respiratory tract infections ranging from mild to lethal. Infection by the coronaviruses SARS-CoV, MERS-CoV, and SARS-CoV-2 can cause severe respiratory illness symptoms, i.e., severe acute respiratory syndrome (SARS), Middle East respiratory syndrome (MERS), or coronavirus disease 2019 (COVID-19), respectively. Infection by the coronaviruses HcoV-OC43, HcoV-229E, HcoV-NL63, or HcoV-HKU1 can lead to mild respiratory illness symptoms, e.g., the common cold. Coronaviruses can also cause disease in animals such as cats, birds, chickens, cows, and pigs. As used herein, “respiratory tract infection” or “respiratory infection” can refer to an upper respiratory tract infection (URI or URTI) or a lower respiratory tract infection (LRI or LRTI). URTIs include infection of the nose, sinuses, pharynx, and larynx, e.g., tonsillitis, pharyngitis, laryngitis, sinusitis, otitis media, and the common cold. LRTIs include infection of the trachea, bronchial tubes, bronchioles, and the lungs, e.g., bronchitis and pneumonia. Symptoms of illnesses caused by coronaviruses include, e.g., fever, cough, shortness of breath, fatigue, congestion, chills, muscle pain, headache, sore throat, loss of taste or smell, diarrhea, etc.

In embodiments, the coronavirus component is a fragment of any of the proteins described herein, e.g., a structural or non-structural coronavirus protein. In embodiments, the fragment comprises a domain of the full length protein. For example, the S protein includes an N-terminal domain (S-NTD) and an ectodomain (S-ECD), which includes the spike S1 and S2 subunits. The S1 subunit also includes a receptor binding domain (S-RBD), which is responsible for binding the host receptor (e.g., ACE2 and/or NRP1). In some embodiments, the immunoassay detects a coronavirus by detecting the coronavirus S1 subunit. In some embodiments, the immunoassay detects a coronavirus by detecting the coronavirus S2 subunit. In some embodiments, the immunoassay method detects a coronavirus by detecting the coronavirus S-NTD. In some embodiments, the immunoassay method detects a coronavirus by detecting the coronavirus S-ECD. In some embodiments, the immunoassay method detects a coronavirus by detecting the coronavirus S-RBD. In embodiments, the S protein subunit (e.g., S1, S2, S-ECD, S-NTD, or S-RBD) comprises a mutation as described in Tables 1A and 1B. In embodiments, the immunoassay detects a coronavirus by detecting a combination of the coronavirus proteins described herein. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein and/or S-RBD. In embodiments, the immunoassay method detects SARS-CoV-2 by detecting any of the SARS-CoV-2 S protein and/or S-RBD variants in Tables 1A and 1B. In embodiments, the immunoassay detects SARS-CoV-2 by detecting SARS-CoV-2 N protein and S protein.

In embodiments, the coronavirus component is a nucleic acid. As used herein in the context of viral components, a viral nucleic acid refers to a viral genome or portion thereof. The viral nucleic acid can encode a viral protein, or the viral nucleic acid can be a non-coding sequence. In embodiments, detection of a viral nucleic acid comprises detecting a sequence that is present in the viral genome, but not in the host genome. In embodiments, the coronavirus component is DNA or RNA. In embodiments, the coronavirus component comprises a nucleic acid secondary structure, e.g., an RNA loop. In embodiments, the coronavirus component is a lipid, e.g., that forms part of the viral envelope.

In embodiments, the invention provides methods for distinguishing between strains of a coronavirus. In embodiments, the coronavirus is SARS-CoV-2. In embodiments, the invention provides methods for assessing the transmissibility of a COVID-19 infection outbreak by determining the SARS-CoV-2 strain. In embodiments, the invention provides methods for assessing the virulence of a SARS-CoV-2 strain by determining the SNPs in the strain. In embodiments, the invention provides methods for assessing effectiveness of a vaccine against a particular strain of SARS-CoV-2. The term “strain” is used interchangeably herein with “variant,” “lineage,” and “type.” In embodiments, a mutant strain or variant of a virus described herein, e.g., SARS-CoV-2, comprises one or more mutations relative to a reference or parent or wild-type strain of the virus. As referred to throughout this application, the SARS-CoV-2 NC_045512 strain is the “reference” or “wild-type” strain, and all SNPs described herein are attributed to one or more “mutant” strains or “variants.” In embodiments, the invention provides methods to trace the lineage of a coronavirus in a population. For example, two strains of SARS-CoV-2 have been identified, referred to as the “L” strain (also known as “lineage B”) and “S” strain (also known as “lineage A”). The L strain can be differentiated from the more ancestral S strain based on two different SNPs that show nearly complete linkage: one at location 8782 (orf1ab: T8517C, synonymous) and one at location 28144 (ORF8: C251T, S84L). See, e.g., Tang et al., Natl Sci Rev, nwaa036; doi:10.1093/nsr/nwaa036 (3 Mar. 2020). Moreover, as discussed herein, two SARS-CoV-2 strains have been identified to contain an SNP at genome location 23403, which encodes the S protein, and are referred to herein as the “S-D614” and “S-D614G” strains. A further SARS-CoV-2 SNP of interest is at location 11083, where the 11083G to T mutation (denoted as “11083G>T”) is associated with asymptomatic presentation. In embodiments, the SARS-CoV-2 reference strain comprises the “L strain” SNP at genome locations 8782 and 28144, the “S-D614” SNP at genome location 23403, and a G nucleotide at genome location 11083.

Mutations in the SARS-CoV-2 S protein can affect, e.g., binding to the ACE2 receptor, overall structure and antibody recognition, and/or protein conformation. Critical residues in the SARS-CoV-2 S-RBD for binding to the ACE2 receptor include, e.g., K417, N439, Y453, L452, S477, T478, E484, Q493, and N501. See, e.g., Lan et al., Nature 581:215-220 (2020). In embodiments, mutations in the SARS-CoV-2 S protein alter binding of the S protein to its host binding partner, e.g., ACE2. In embodiments, mutations in the SARS-CoV-2 S protein affect transmissibility of the virus. In embodiments, mutations in the SARS-CoV-2 S protein affect vaccine effectiveness against the virus. In embodiments, SARS-CoV-2 strains are characterized by SNPs in the coding sequence of the S protein. Such SARS-CoV-2 strains include, e.g., A.23.1 (also referred to as the “Uganda strain”); A.VOI.V2 (also referred to as the “Tanzania strain”); B.1; B.1.1.519 (also referred to as the “Mexico/Texas BV-2 strain”); B.1.1.529 (also referred to as the “Omicron variant” or “BA.1,” which comprises sub-lineages BA.2 and BA.3); B.1.1.7 (also referred to as the “UK strain” or “Alpha variant”); B.1.351 or 501Y.V2 (referred to as the “South Africa strain” or “Beta variant”); B.1.429 or Cal.20C (referred to as the “California strain” or “Epsilon variant”); B.1.525 (also referred to as the “Nigeria strain” or “Eta variant”); B.1.526 (also referred to as the “New York strain” or “Iota variant”); B.1.617 (also referred to as the “India strain”); the B.1.617.1 strain (also referred to as the “Kappa strain”); B.1.617.2 (also referred to as the “Delta variant”), which has been further reclassified into sub-lineages designated as “AY”; B.1.617.3; Texas BV-1; B.1.621 (also referred to as the “Mu variant”); C.37 (also referred to as the “Chile/Peru strain” or “Lambda variant”); P.1 (also referred to as the “Brazil strain” or “Gamma variant”); P.2 (also referred to as the “Zeta variant”); P.3 (also referred to as the “Philippines strain”); and R.1 (also referred to as the Kentucky strain). The B.1.1.529 strain comprises the following mutations in the S protein: A67V, A69-70, T95I, G142D/Δ143-145, A211/L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, of which G339D, S371L, S373P, S375F, K417N, N440K, G446S, S477N, T478K, E484A, Q493R, G496S, Q498R, N501Y, and Y505H are in the S-RBD. The B.1.1.7 strain is characterized by the following mutations in the S protein: a deletion of amino acid residues 69-70, E484K, N501Y, D614G, and P681H. The 501Y.V2 strain is characterized by the following mutations in the S protein: D215G, K417N, E484K, N501Y, and D614G. The P.1 strain is characterized by the following mutations in the S protein: K417T, E484K, N501Y, and D614G. The Cal.20C strain is characterized by a L452R mutation in the S protein. The B.1.526 strain comprises the following mutations in the S protein: L5F, T95I, D253G, D614G, A701V, and either E484K or S477N. The B.1.526 strain comprising E484K is referred to herein as “B.1.526” or “B.1.526/E484K” and the B.1.526 strain comprising S477N is referred to herein as “B.1.526.2” “B.1.526/S477N.”

In embodiments, mutations in SARS-CoV-2 proteins, e.g., S protein, result from genetic recombination between two or more SARS-CoV-2 variants. For example, a host subject may be simultaneously infected by two variants, e.g., the B.1.617.2/AY.4 (“Delta”) and B.1.1.529/BA.1 (“Omicron”) variants, which may recombine when replicating in the host to produce a recombinant variant. The recombinant variant may be designated as the cross between its parent variants. For example, the recombinant variant resulting from Delta (AY.4) and Omicron (BA.1) variants is designated as the BA.1×AY.4 recombinant.

As used herein, all strain designations include all of its sub-strains. For example, the B.1.526 strain includes the B.1.526, B.1.526.1, and the B.1.526.2 strains, and the B.1.617 strain includes the B.1.617, B.1.617.1, B.1.617.2, and B.1.617.3 strains. The B.1.617.2 strain (“Delta variant”) includes all “AY” sub-lineage designations, including AY.1, AY.2, AY.3, AY.4, AY.5, AY.6, AY.7, AY.8, AY.9, AY.10, AY.11, AY.12, AY.13, AY.14, AY.15, AY.16, AY.17, AY.18, AY.19, AY.20, AY.21, AY.22, AY.23, AY.24, AY.25, and all sub-lineages thereof (e.g., AY.4.2). As used herein, strains “characterized” by particular mutations include at least those particular mutations and may include additional mutations. These strains and associated mutations are summarized in Table 1A. Additional variants of SARS-CoV-2 comprise mutations in the S protein as shown in Tables 11B and 1D and are further described, e.g., in Faria et al., “Genomic characterisation of an emergent SARS-CoV-2 lineage in Manaus: preliminary findings” (2020). Accessed at virological.org/t/586; Wu et al., bioRxiv doi:10.1101/2021.01.25.427948 (2021); Guruprasad, Proteins 2021:1-8 (2021); Zhou et al., bioRxiv doi:10.1101/2021.03.24.436620 (2021). Further strains and mutations of SARS-CoV-2 are provided in the PANGO lineages database (cov-lineages.org); the Nextstrain database (nextstrain.org); the Global Evaluation of SARS-CoV-2/hCoV-19 Sequences (GESS) database provided by Fang et al., Nucleic Acid Res 49(D1):D706-D714 (2021) (wan-bioinfo.shinyapps.io/GESS); and the SARS-CoV-2 Mutation Browser provided by Rakha et al., bioRxiv doi: 10.1101/2020.06.10.145292 (2020) (covid-19.dnageography.com). The mutations denoted as “del” or “A” indicate a deletion of the indicated amino acid residues present in the reference sequence. For example, a variant S protein comprising a “A69-70” mutation means that amino acid residues at positions 69 and 70 of the wild-type S protein are deleted. The mutations denoted as “ins” indicates an insertion of one or more amino acid residues at the indicated amino acid position. For example, a variant S protein comprising an “ins146N” mutation means the variant S protein comprises an asparagine residue at amino acid position 146 of the variant S protein. The mutations denoted as (X1-X2)→Y denotes that the amino acid residues X1-X2 indicated in the parentheses are mutated to a single amino acid Y. For example, a variant S protein comprising a “(L24-A27)→S” mutation means the variant S protein comprises a replacement of the amino acid residues at positions 24 to 27 with a serine residue.

Throughout this application, when referring to an S protein comprising a specific mutation, the mutation is relative to the SARS-CoV-2 reference strain NC_045512. The S protein from the SARS-CoV-2 reference strain is also known as the “wild-type” S protein. For example, the S-D614G protein from SARS-CoV-2 comprises D to G substitution at amino acid residue 614 relative to the wild-type S protein from SARS-CoV-2.

TABLE 1A
SARS-CoV-2 Strains and Associated Mutations

| | | | |
|---|---|---|---|
| Genome location (based on | | | |
| SARS-CoV-2 reference | Nucleotide | Amino Acid Change in | Exemplary Associated |
| strain NC_045512) | Change | Corresponding Protein | SARS-CoV-2 Strain(s) |

5′ UTR

| | | |
|---|---|---|
| 241 | C > T | (N/A) |

nsp3

| | | |
|---|---|---|
| 3037 | C > T | F924F |

ORF1ab

| | | | |
|---|---|---|---|
| 1059 (Orf1a) | C > T | T265I | |
| 3267 | C > T | T1001I | B.1.1.7 |
| 3828 | C > T | S1188L | P.1 |
| 5230 | G > T | K1655N | 501Y.V2 |
| 5388 | C > A | A1708D | B.1.1.7 |
| 5648 | A > C | K1795Q | P.1 |
| 6954 | T > C | I2230T | B.1.1.7 |
| 11288-11296 | deletion | SGF3675-3677 del | B.1.1.7 |
| 17259 | G > T | E5665D | P.1 |

RdRp

| | | |
|---|---|---|
| 14408 | C > T | P323L |

S Protein

| | | | |
|---|---|---|---|
| 21614 | C > T | L18F | P.1 |
| 21618 | C > G | T19R | B.1.617.2, B.1.617.3 |
| 21621 | C > A | T20N | P.1 |
| 21638 | C > T | P26S | P.1 |
| 21765-21770 | deletion | HV69-70 del | B.1.1.7, B.1.525, |
| | | | B.1.1.529 |
| 21801 | A > C | D80A | 501Y.V2 |
| 21846 | C > T | T95I | B.1.621, B.1.526, |
| | | | B.1.617.1, B.1.1.529 |
| 21974 | G > T | D138Y | P.1 |
| 21991-21993 | deletion | Y144 del | B.1.1.7 |
| 22132 | G > T | R190S | P.1 |
| 22206 | A > G | D215G | 501Y.V2 |
| 22227 | C > T | A222V | |
| 22320 | A > G | D253G | B.1.526 |
| 22578 | G > A | G339D | B.1.1.529 |
| 22812 | A > C | K417T | P.1 |
| 22813 | G > T | K417N | 507Y.V2 (B.1.351), |
| | | | B.1.617.2 (AY.1 & AY.2), |
| | | | B.1.1.529 |
| 22865 | G > T | A435S | |
| 22917 | T > G | L452R | Cal.20C, B.1.427, B.1.429, |
| | | | B.1.526.1, B.1.617 (and |
| | | | sub-lineages) |
| 22995 | C > A | T478K | B.1.1.519, B.1.617.2 |
| 23012 | G > A | E484K | 501Y.V2, P.1, P.2, |
| | | | B.1.525, B.1.526, B.1.620 |
| 23012 | G > C | E484Q | B.1.617, B.1.617.1, |
| | | | B.1.617.3 |
| 23063 | A > T | N501Y | B.1.1.7, 501Y.V2, P.1 |
| 23271 | C > A | A570D | B.1.1.7 |
| 23403 | A > G | D614G | B.1.1.7, 501Y.V2, P.1, |
| | | | B.1.429, B.1.526, |
| | | | All B.1 lineages |
| 23525 | C > T | H655Y | P.1, C.1.2, B.1.1.529 |
| 23593 | G > T | Q677H | B.1.525 |
| 23593 | G > C | Q677H | B.1.525 |
| 23604 | C > A | P681H | B.1.1.7, B.1.1.519, |
| | | | B.1.620 |
| 23604 | C > G | P681R | B.1.617.1, B.1.617.2, |
| | | | B.1.617.3 |
| 23664 | C > T | A701V | 501Y.V2 |
| 23709 | C > T | T716I | B.1.1.7 |
| 24224 | T > C | F888L | |
| 24506 | T > G | S982A | B.1.1.7 |
| 24642 | C > T | T1027I | P.1 |
| 24775 | A > T | Q1071H | B.1.617.1 |
| 24914 | G > C | D1118H | B.1.1.7 |
| 25088 | G > T | V1167F | |

Orf3a

| | | |
|---|---|---|
| 25563 | G > T | Q57H |
| 26144 | G > T | G251V |

E Protein

| | | | |
|---|---|---|---|
| 26456 | C > T | P71L | 501Y.V2 |

ORF8

| | | | |
|---|---|---|---|
| 27972 | C > T | Q27stop | B.1.1.7 |
| 28048 | G > T | R52I | B.1.1.7 |
| 28111 | A > G | Y73C | B.1.1.7 |
| 28167 | G > A | E92K | P.1 |

N Protein

| | | | |
|---|---|---|---|
| 28280 | GAT > CTA | D3L | B.1.1.7 |
| 28512 | C > G | P80R | P.1 |
| 28887 | C > T | T205I | 501Y.V2 |
| 28932 | G > T | A220V | |
| 28977 | C > T | S235F | B.1.1.7 |
| 29095 | C > T | F274F | |

Orf10

| | | |
|---|---|---|
| 29645 | G > T | V30L |

TABLE 1B
Additional Mutations of the SARS-CoV-2 S and N Proteins and Associated Strains

| | Associated |
|---|---|
| | Strain/Lineage |
| S Protein Mutation(s) (relative to S Protein | |
| of reference strain NC_045512) | |
| S Protein (e.g., S-ECD Domain) | |
| D614G | B.1 |
| A222V | |
| N439K | B.1.466.2 |
| Y453F | |
| S477N | |
| E484K | P.2; B.1.1.28.2 |
| N501T | |
| N501Y | |
| Q677H | C.36.2 |
| Q677P | |
| D936Y | |
| A222V, D614G | |
| N439K, D614G | |
| S477N, D614G | |
| Y453F, D614G | |
| E484K, D614G | |
| N501T, D614G | |
| N501Y, D614G | |
| D936Y, D614G | |
| Q677H, D614G | |
| A67V, Δ69-70, T95I, G142D/Δ143-145, Δ211/L212I, ins214EPE, G339D, | B.1.1.529 |
| S371L, S373P, S375F, K417N, N440K, G446S, S477N, T478K, E484A, | |
| Q493R, G496S, Q498R, N501Y, Y505H, T547K, D614G, H655Y, | |
| N679K, P681H, N764K, D796Y, N856K, Q954H, N969K, L981F | |
| A67V, Δ69-70, T95I, G142D/Δ143-145, Δ211/L212I, ins214EPE, G339D, | B.1.1.529 + R346K |
| R346K, S371L, S373P, S375F, K417N, N440K, G446S, S477N, T478K, | |
| E484A, Q493R, G496S, Q498R, N501Y, Y505H, T547K, D614G, | |
| H655Y, N679K, P681H, N764K, D796Y, N856K, Q954H, N969K, L981F | |
| A67V, Δ69-70, T95I, G142D/Δ143-145, Δ211/L212I, ins214EPE, G339D, | B.1.1.529 + L452R |
| S371L, S373P, S375F, K417N, N440K, L452R, G446S, S477N, T478K, | |
| E484A, Q493R, G496S, Q498R, N501Y, Y505H, T547K, D614G, | |
| H655Y, N679K, P681H, N764K, D796Y, N856K, Q954H, N969K, L981F | |
| A67V, H69-V70del, T95I, G142D/V143-Y145del, (N211-L212)→I, | B.1.1.529 + F817P, |
| ins214EPE, G339D, S371L, S373P, S375F, K417N, N440K, G446S, | A892P, A899P, A942P |
| S477N, T478K, E484A, Q493R, G496S, Q498R, N501Y, Y505H, T547K, | |
| D614G, H655Y, N679K, P681H, N764K, D796Y, F817P, N856K, A892P, | |
| A899P, A942P, Q954H, N969K, L981F | |
| T19R, A27S, T95I, G142D, E156-F157-R158→G, N211-L212→I, | BA.1 × AY.4 recombinant |
| ins214EPE, G339D, S371L, S373P, S375F, K417N, N440K, G446S, | |
| S477N, T478K, E484A, Q493R, G496S, Q498R, N501Y, Y505H, T547K, | |
| D614G, H655Y, N679K, P681H, N764K, D796Y, N856K, Q954H, N969K, L981F | |
| T19I, (L24-A27)→S, G142D, V213G, G339D, S371F, S373P, S375F, | BA.2 |
| T376A, D405N, R408S, K417N, N440K, S477N, T478K, E484A, Q493R, | |
| Q498R, N501Y, Y505H, D614G, H655Y, N679K, P681H, N764K, | |
| D796Y, Q954H, N969K | |
| T19I, (L24-A27)→S, G142D, V213G, G339D, S371F, S373P, S375F, | BA.2 + L452R |
| T376A, D405N, R408S, K417N, N440K, L452R, S477N, T478K, E484A, | |
| Q493R, Q498R, N501Y, Y505H, D614G, H655Y, N679K, P681H, | |
| N764K, D796Y, Q954H, N969K | |
| T19I, (L24-A27)→S, G142D, V213G, G339D, S371F, S373P, S375F, | BA.2 + L452M |
| T376A, D405N, R408S, K417N, N440K, L452M, S477N, T478K, E484A, | |
| Q493R, Q498R, N501Y, Y505H, D614G, H655Y, N679K, P681H, | |
| N764K, D796Y, Q954H, N969K | |
| T19I, (L24-A27)→S, G142D, V213G, G339D, S371F, S373P, S375F, | BA.2.12.1 |
| T376A, D405N, R408S, K417N, N440K, L452Q, S477N, T478K, E484A, | |
| Q493R, Q498R, N501Y, Y505H, D614G, H655Y, N679K, P681H, | |
| S704L, N764K, D796Y, Q954H, N969K | |
| T19I, (L24-A27)→S, G142D, V213G, G339D, R346T, S371F, S373P, | BA.2.74 |
| S375F, T376A, D405N, R408S, K417N, N440K, L452M, S477N, T478K, | |
| E484A, Q493R, Q498R, N501Y, Y505H, D614G, H655Y, N679K, | |
| P681H, N764K, D796Y, Q954H, N969K | |
| T19I, L24-A27→S, G142D, K147E, W152R, F157L, I210V, V213G, | BA.2.75 |
| G257S, G339H, S371F, S373P, S375F, T376A, D405N, R408S, K417N, | |
| N440K, G446S, N460K, S477N, T478K, E484A, Q498R, N501Y, Y505H, | |
| D614G, H655Y, N679K, P681H, N764K, D796Y, Q954H, N969K | |
| T19I, (L24-A27)→S, G142D, V213G, R346T, Y248N, G339D, S371F, | BA.2.76 |
| S373P, S375F, T376A, D405N, R408S, K417N, N440K, S477N, T478K, | |
| E484A, Q493R, Q498R, N501Y, Y505H, D614G, H655Y, N679K, | |
| P681H, N764K, D796Y, Q954H, N969K | |
| A67V, H69-V70del, T95I, G142D, V143-Y145del, (N211-L212)→I, | BA.3 |
| G339D, S371F, S373P, S375F, D405N, K417N, N440K, G446S, S477N, | |
| T478K, E484A, Q493R, Q498R, N501Y, Y505H, D614G, H655Y, | |
| N679K, P681H, N764K, D796Y, Q954H, N969K | |
| T19I, (L24-A27)→S, H69-V70del, G142D, V213G, G339D, S371F, | BA.4 |
| S373P, S375F, T376A, D405N, R408S, K417N, N440K, L452R, S477N, | |
| T478K, E484A, F486V, Q498R, N501Y, Y505H, D614G, H655Y, N658S, | |
| N679K, P681H, N764K, D796Y, Q954H, N969K | |
| T19I, (L24-A27)→S, H69-V70del, G142D, V213G, G339D, R346T, | BA.4.6 |
| S371F, S373P, S375F, T376A, D405N, R408S, K417N, N440K, L452R, | |
| S477N, T478K, E484A, F486V, Q498R, N501Y, Y505H, D614G, H655Y, | |
| N658S, N679K, P681H, N764K, D796Y, Q954H, N969K | |
| T19I, (L24-A27)→S, H69-V70del, G142D, V213G, G339D, S371F, | BA.5 |
| S373P, S375F, T376A, D405N, R408S, K417N, N440K, L452R, S477N, | |
| T478K, E484A, F486V, Q498R, N501Y, Y505H, D614G, H655Y, | |
| N679K, P681H, N764K, D796Y, Q954H, N969K | |
| T19I, (L24-A27)→S, H69-V70del, T76I, G142D, V213G, G339D, S371F, | BA.5.5 |
| S373P, S375F, T376A, D405N, R408S, K417N, N440K, L452R, S477N, | |
| T478K, E484A, F486V, Q498R, N501Y, Y505H, D614G, H655Y, | |
| N679K, P681H, N764K, D796Y, Q954H, N969K | |
| T19I, (L24-A27)→S, H69-V70del, G142D, V213G, G339D, S371F, | |
| S373P, S375F, T376A, D405N, R408S, K417N, N440K, L452R, S477N, | |
| T478K, E484A, F486V, Q493R, Q498R, N501Y, Y505H, D614G, | |
| H655Y, N658S, N679K, P681H, N764K, D796Y, Q954H, N969K | |
| T19I, (L24-A27)→S, H69-V70del, G142D, V213G, G339D, S371F, | |
| S373P, S375F, T376A, D405N, R408S, K417N, N440K, L452R, S477N, | |
| T478K, E484A, F486V, Q493R, Q498R, N501Y, Y505H, D614G, | |
| H655Y, N679K, P681H, N764K, D796Y, Q954H, N969K | |
| del69-70, del144, N501Y, A570D, P681H, T716I, S982A, D1118H, | B.1.1.7 |
| D614G | |
| L18F, D80A, D215G, L242H, R246I, K417N, E484K, N501Y, A701V, | B.1.351 + L18F, L242H, |
| D614G | R246I |
| L18F, D80A, D215G, del242-244, R246I, K417N, E484K, N501Y, | B.1.351 + L18F, del242- |
| A701V, D614G | 244, R246I |
| del69-70, del144, N501Y, A570D, P681H, T716I, S982A, D1118H, | B.1.1.7 + N439K, Y453F |
| N439K, Y453F, D614G | |
| L18F, T20N, P26S, D138Y, R190S, K417T, E484K, N501Y, D614G, | P.1 + V1176F |
| H655Y, T1027I, V1176F | |
| S13I, W152C, L452R, D614G | B.1.429 |
| E484K, N501Y, D614G | B.1.429 − S13I |
| del69-70, del144, N501Y, A570D, P681H, T716I, S982A, D1118H, | B.1.1.7 + E484K |
| D614G, E484K | |
| D80A, D215G, K417N, E484K, N501Y, A701V, D614G | B.1.351 |
| L18F, T20N, P26S, D138Y, R190S, K417T, E484K, N501Y, D614G, | P.1; B.1.1.28.1 |
| H655Y, T1027I | |
| L5F, T95I, D253G, E484K, D614G, A701V | B.1.526/E484K |
| L5F, T95I, D253G, S477N, D614G, A701V | B.1.526/S477N |
| Q52R, E484K, Q677H, D614G, F888L | B.1.525 |
| Q52R, A67V, del69-70, E484K, Q677H, D614G, F888L | B.1.525 + A67V + |
| | del69-70 |
| Q52R, A67V, del69-70, del144, E484K, Q677H, D614G, F888L | B.1.525 + A67V + |
| | del69-70, del144 |
| E484K, D614G, V1176F | P.2 |
| del141-143, E484K, N501Y, D614G, P681H | P.3 |
| del141-143, E484K, N501Y, D614G, P681H, E1092K, H1101Y, V1176F | P.3 + 1092K, 1101Y, |
| | 1176F |
| L452R, E484Q, D614G | B.1.617 |
| E154K, L452R, E484Q, D614G, P681R | B.1.617 + E154K, 681R |
| E154K, L452R, E484Q, D614G, P681R, Q1071H | B.1.617.1(2) |
| G142D, E154K, L452R, E484Q, D614G, P681R, Q1071H | B.1.617.1(3) |
| T95I, G142D, E154K, L452R, E484Q, D614G, P681R, Q1071H | B.1.617.1(4) |
| T95I, D253G, D614G | B.1.526 reference |
| D80G, Y144del, F157S, L452R, D614G, T859N, D950H | B.1.526.1 |
| L5F, T95I, D253G, S477N, D614G, Q957R | B.1.526.2 |
| R102I, F157L, V367F, Q613H, P681R | A.23.1 |
| F157L, V367F, Q613H, P681R | A.23.1 |
| D80Y, Y144del, I210del, D215G, RSY246-248 del, L249M, W258L, | A.VOI.V2 |
| R346K, T478R, E484K, H655Y, P681H, Q957H | |
| L5F, ins214TDR, Q414K, N450K, D614G, T716I, | B.1.214.2 |
| D614G, Q677H, L938F | C.36.2 + L938F |
| del69-70, del144, Q493R, N501Y, A570D, D614G, P681H, T716I, | BV-1 |
| S982A, D1118H | |
| T478K, D614G, P681H, T732A | BV-2; B.1.1.1; C37; |
| | B.1.1.519 |
| G75V, T76I, R246del, S247del, Y248del, L249del, T250del, P251del, | B.1.1.1; C37 |
| G252del, D253N, L452Q, F490S, D614G, T859N | |
| W152L, E484K, D614G, G769V | R.1 |
| T19R, del157-158, L452R, T478K, D614G, P681R, D950N | B.1.617.2 (1) |
| T19R, L452R, T478K, D614G, P681R, D950N | B.1.617.2 (2) |
| T19R, del156-157, R158G, L452R, T478K, D614G, P681R, D950N | B.1.617.2 (3) (“Delta”) |
| T19R, G142D, del156-157, R158G, K417N, N439K, L452R, T478K, | B.1.617.2 + K417N, |
| E484K, N501Y, D614G, P681R, D950N | N439K, E484K, N501Y |
| T19R, G142D, del156/157, R158G, K417N, L452R, T478K, E484K, | B.1.617.2 + K417N, |
| N501Y, D614G, P681R, D950N | E484K, N501Y |
| T19R, G142D, del156/157, R158G, L452R, T478K, E484K, N501Y, | B.1.617.2 + E484K, |
| D614G, P681R, D950N | N501Y |
| T19R, G142D, del156/157, R158G, L452R, T478K, E484K, D614G, | B.1.617.2 + E484K |
| P681R, D950N | |
| T19R, G142D, L452R, E484Q, D614G, P681R, D950N | B.1.617.3(1) |
| T19R, L452R, E484Q, D614G, P681R | B.1.617.3(2) |
| P26S, del69-70, V126A, del144, del242-244, H245Y, S477N, E484K, | B.1.620 |
| D614G, P681H, T1027I, D1118H | |
| del69-70, L189F, N439K, D614G, V772I | B.1.258.17 |
| W152R, N439K, D614G, P681R | B.1.466.2 |
| T19R, del144, del157-158, L452R, T478K, D614G, P681R, D950N | B.1.617.2 + del144 |
| delY145-146, E484K, D614G | B.1.618 |
| T19R, del157-158, K417N, L452R, T478K, D614G, P681R, D950N | AY.1 |
| | (B.1.617.2(1) + K417N) |
| T19R, T95I, G142D, E156G, Δ157/158, W258L, K417N, L452R, T478K, | AY.1 (Alt Seq 1) |
| D614G, P681R, D950N | |
| T19R, V70F, G142D, del157-158, A222V, K417N, L452R, T478K, | AY.2 |
| D614G, P681R, D950N | |
| T19R, V70F, G142D, E156G, Δ157/158, A222V, K417N, L452R, T478K, | AY.2 (Alt Seq 1) |
| D614G, P681R, D950N | |
| T19R, K417N, L452R, T478K, D614G, P681R, D950N | B.1.617.2(2) + K417N |
| T95I, del144, E484K, D614G, P681H, D796H | B.1.1.318 |
| T95I, del144-145, E484K, D614G, P681H, D796H | B.1.1.318 |
| S12F, del69-70, W152R, R346S, L452R, D614G, Q677H, A899S | C.36.3 |
| D80A, D215G, K417N, D614G, A701V | B.1.351.1 |
| L18F, D80A, D215G, del242-244, K417N, E484K, N501Y, D614G, | B.1.351.2 |
| A701V | |
| D80G, T95I, G142D, Y144del, N439K, E484K, D614G, P681R, I1130V, | AV-1 |
| D1139H | |
| I210T, N440K, E484K, D614G, D936F, S939N, T1027I | B.1.619 |
| T95I, Y144T, Y145S, ins146N, R346K, E484K, N501Y, D614G, P681H, | B.1.621 |
| D950N | |
| T95I, Y144S, Y145N, R346K, E484K, N501Y, D614G, P681H, D950N | B.1.621 |
| T19R, T95I, G142D, Δ156/157, R158G, L452R, T478K, D614G, P681R, | AY.3 and B.1.617.2; |
| D950N | AY.4 (Alt Seq 2) |
| T19R, Δ156/157, R158G, L452R, T478K, D614G, P681R, D950N | AY.3 (outbreak); AY.12 |
| ΔLGV141-143, L452R, D614G, | A.2.5 |
| S477N, D614G | B.1.160 |
| D614G, P681R | |
| L452R, D614G | |
| T478K, D614G | |
| T19R, T95I, Δ156/157, R158G, L452R, T478K, D614G, P681R, D950N | AY.4 and AY.12 |
| T19R, T95I, G142D, Y145H, Δ156/157, R158G, A222V L452R, T478K, | AY.4.2 |
| D614G, P681R, D950N | |
| T19R, G142D, E156-F157del, R158G, A222V | AY.9 |
| L452R, T478K, D614G, P681R, D950N | |
| T19R, S112L, G142D, E156-F157del, R158G, | AY.25 |
| L452R, T478K, D614G, P681R, D950N | |
| P9L, P25L, C136F, Y144del, R190S, D215G, A243del, L244del, Y449H, | C.1.2 |
| T478K, E484K, N501Y, L585F, D614G, H655Y, P681H, N679K, T7161, | |
| T859N | |
| P9L, E96Q, C136-Y144del, R190S, D215H, R346S, N394S, Y449N, | B.1.640.2 |
| E484K, F490S, N501Y, D614G, P681H, T859N, D1139H | |
| S-RBD Domain | |
| G339D, S371L, S373P, S375F, K417N, N440K, G446S, S477N, T478K, | B.1.1.529 |
| E484A, Q493R, G496S, Q498R, N501Y, Y505H | |
| G339D, R346K, S371L, S373P, S375F, K417N, N440K, G446S, S477N, | B.1.1.529 + R346K |
| T478K, E484A, Q493R, G496S, Q498R, N501Y, Y505H | (BA.1.1) |
| G339D, S371L, S373P, S375F, K417N, N440K, L452R, G446S, S477N, | B.1.1.529 + L452R |
| T478K, E484A, Q493R, G496S, Q498R, N501Y, Y505H | |
| G339D, S371F, S373P, S375F, T376A, D405N, R408S, K417N, N440K, | BA.2 |
| S477N, T478K, E484A, Q493R, Q498R, N501Y, Y505H | |
| G339D, S371F, S373P, S375F, T376A, D405N, R408S, K417N, N440K, | BA.2 + L452R |
| L452R, S477N, T478K, E484A, Q493R, Q498R, N501Y, Y505H | |
| G339D, S371F, S373P, S375F, T376A, D405N, R408S, K417N, N440K, | BA.2 + L452M |
| L452M, S477N, T478K, E484A, Q493R, Q498R, N501Y, Y505H | |
| G339D, S371F, S373P, S375F, T376A, D405N, R408S, K417N, N440K, | BA.2.12.1 |
| L452Q, S477N, T478K, E484A, Q493R, Q498R, N501Y, Y505H | |
| G339D, R346T, S371F, S373P, S375F, T376A, D405N, R408S, K417N, | BA.2.74 |
| N440K, L452M, S477N, T478K, E484A, Q493R, Q498R, N501Y, Y505H | |
| G339D, R346T, S371F, S373P, S375F, T376A, D405N, R408S, K417N, | BA.2.76 |
| N440K, S477N, T478K, E484A, Q493R, Q498R, N501Y, Y505H | |
| G339D, S371F, S373P, S375F, D405N, K417N, N440K, G446S, S477N, | BA.3 |
| T478K, E484A, Q493R, Q498R, N501Y, Y505H | |
| G339D, S371F, S373P, S375F, T376A, D405N, R408S, K417N, N440K, | BA.4; BA.5 |
| L452R, S477N, T478K, E484A, F486V, Q498R, N501Y, Y505H | |
| G339D, R346T, S371F, S373P, S375F, T376A, D405N, R408S, K417N, | BA.4.6 |
| N440K, L452R, S477N, T478K, E484A, F486V, Q498R, N501Y, Y505H | |
| N501Y | B.1.1.7 |
| K417N, E484K, N501Y | B.1.351 |
| K417T, E484K, N501Y | P.1 |
| E484K, N501Y | P.3 |
| L452R | B.1.427; B.1.429; |
| | B.1.526.1 |
| E484K | B.1.525; B.1.526; |
| | B.1.526.1; P.2; R.1 |
| N439K | |
| K417N | |
| I472V | |
| Y453F | |
| S477N | B.1.526.2 |
| S477N, E484K | B.1.620 |
| N501T | |
| L452R, E484Q | B.1.617; B.1.617.1; |
| | B.1.617.3 |
| K417N, N439K L452R, T478K, E484K, N501Y | B.1.617.2 (4) |
| K417N, L452R, T478K, E484K, N501Y | B.1.617.2 + K417N, |
| | E484K, N501Y |
| L452R, T478K, E484K, N501Y | B.1.617.2 + E484K, |
| | N501Y |
| L452R, T478K, E484K | B.1.617.2 + E484K |
| Q414K, N450K | B.1.214.2 |
| R346K, T478R, E484K | A.VOI.V2 |
| V367F | A.23.1 |
| K417N | |
| K417T | |
| Q493R, N501Y | BV-1 |
| T478K | BV-2; B.1.1.519 |
| L452Q, F490S | B.1.1.1; C37 |
| L452R, T478K | B.1.617.2 |
| K417N, L452R, T478K | B.1.617.2 + K417N |
| N440K, E484K | B.1.619 |
| R346K, E484K, N501Y | B.1.621 |
| S1 Subunit | |
| del69-70, del144, N501Y, A570D, D614G, P681H | B.1.1.7 |
| K417N, E484K, N501Y, D614G | |
| D614G | |
| Q677H | |
| Q677P | |
| N Protein Mutation(s) (relative to S Protein | |
| of reference strain NC_045512) | |
| P13L, E31-S33del, R203K, G204R | B.1.1.529 |

Further SARS-CoV-2 SNPs have been identified, for example, at the genome locations listed in Table 1C, e.g., locations 3036, 8782 18060, 11083, 1397, 2891, 14408, 17746, 17857, 23403, 26143, 28144, and 28881. See. e.g., Pachetti et al., J Transl Med 18:179 (2020); Banerjee et al., bioRxiv, doi.org/10.1101/2020.04.06.027854 (9 Apr. 2020); Alouane et al., bioRxiv doi.org/10.1101/2020.06.20.163188 (21 Jun. 2020); Brufsky, J Med Virol 2020:1-5 (2020); and Mishra et al., bioRxiv doi.org/10.1101/2020.05.07.082768 (12 May 2020). The ability to determine viral strain and/or trace viral lineage in a population provides valuable epidemiological insight into the spread and evolution of the virus. Determining the particular viral strain that has infected a patient also allows more comprehensive treatment. For example, the patient can be treated with a strain-specific drug. If a particular strain is more transmissible and/or more likely to cause severe illness, early interventions can be provided to the patient.

TABLE 1C
SARS-CoV-2 Single Nucleotide Polymorphisms

| Genome location (based on | | |
|---|---|---|
| SARS-CoV-2 reference | Nucleotide | Amino Acid |
| strain NC_045512) | Change | Change |
| 241 | C > T | 5′ UTR |
| 1059 | C > T | T > I |
| 1604 | AATG > A | delTGA |
| 3037 | C > T | Synonymous |
| 4402 | T > C | Synonymous |
| 5062 | G > T | L > F |
| 8782 | C > T | Synonymous |
| 11083 | G > T | L > F |
| 11916 | C > T | S > L |
| 14408 | C > T | P > L |
| 14805 | C > T | Synonymous |
| 15324 | C > T | T > I |
| 17247 | T > C | Synonymous |
| 17747 | C > T | P > L |
| 17858 | A > G | M > V |
| 18060 | C > T | S > F |
| 18877 | C > T | H > Y |
| 21618 | C > G | T > R |
| 21765-21770 | Deletion | del69-70 |
| 21846 | C > T | T > I |
| 21991-21993 | Deletion | del144 |
| 22206 | A > G | D > G |
| 22132 | G > T | R > S |
| 22227 | C > T | A > V |
| 22277 | C > A | Q > K |
| 22578 | G > A | G > D |
| 22661 | G > T | V > F |
| 22320 | A > G | D > G |
| 22812 | A > C | K > T |
| 22813 | G > T | K > N |
| 22865 | G > T | A > S |
| 22917 | T > G | L > R |
| 22995 | C > A | T > K |
| 23012 | G > A | E > K |
| 23012 | G > C | E > Q |
| 23063 | A > T | N > Y |
| 23271 | C > A | A > D |
| 23403 | A > G | D > G |
| 23525 | C > T | H > Y |
| 23593 | G > T or G > C | Q > H |
| 23604 | C > A | P > H |
| 23604 | C > G | P > R |
| 23664 | C > T | A > V |
| 23709 | C > T | T > I |
| 24138 | C > A | T > N |
| 24224 | T > C | F > L |
| 24506 | T > G | S > A |
| 24775 | A > T | Q > H |
| 24914 | G > C | D > H |
| 25088 | G > T | V > F |
| 25563 | G > T | Q > H |
| 26144 | G > T | G > V |
| 27046 | C > T | T > M |
| 27964 | C > T | S > L |
| 28144 | T > C | L > S |
| 28311 | C > T | P > L |
| 28881-28883 | GGG > AAC | R > K, G > R |
| 28932 | C > T | A > V |
| 29095 | C > T | Synonymous |
| 29540 | G > A | Upstream |
| 29553 | G > A | Intergenic |
| 29645 | G > T | V > L |
| 29711 | G > T | Downstream |

TABLE 1D
Selected SARS-CoV-2 Strains and S Protein Mutations

| S Protein Mutation(s) (relative to reference strain NC_045512) | Associated Strain |
|---|---|
| F157L, V367F, Q613H, P681R | A.23.1 |
| D80Y, ΔY144, ΔI210, D215G, Δ246-248, L249M, W258L, R346K, | A.VOI.V2 |
| T478R, E484K, H655Y, P681H, Q957H | |
| T19R, Δ157/158, K417N, L452R, T478K, D614G, P681R, D950N | AY.1 |
| T19R, T95I, G142D, E156G, Δ157/158, W258L, K417N, L452R, | AY.1 (Alt Seq 1) |
| T478K, D614G, P681R, D950N | |
| T19R, V70F, G142D, Δ157/158, A222V, K417N, L452R, T478K, | AY.2 |
| D614G, P681R, D950N | |
| T19R, V70F, G142D, E156G, Δ157/158, A222V, K417N, L452R, | AY.2 (Alt Seq 1) |
| T478K, D614G, P681R, D950N | |
| T19R, T95I, G142D, Y145H, Δ156/157, R158G, A222V L452R, | AY.4.2 |
| T478K, D614G, P681R, D950N | |
| T19R, Δ156/157, R158G, L452R, T478K, D614G, P681R, D950N | AY.12 |
| D614G | B.1 |
| T478K, D614G, P681H, T732A | B.1.1.519 |
| A67V, Δ69-70, T95I, G142D/Δ143-145, Δ211/L212I, ins214EPE, | B.1.1.529 (BA.1) |
| G339D, S371L, S373P, S375F, K417N, N440K, G446S, S477N, | |
| T478K, E484A, Q493R, G496S, Q498R, N501Y, Y505H, T547K, | |
| D614G, H655Y, N679K, P681H, N764K, D796Y, N856K, Q954H, | |
| N969K, L981F | |
| A67V, Δ69-70, T95I, G142D/Δ143-145, Δ211/L212I, ins214EPE, | B.1.1.529 + R346K |
| G339D, R346K, S371L, S373P, S375F, K417N, N440K, G446S, | |
| S477N, T478K, E484A, Q493R, G496S, Q498R, N501Y, Y505H, | |
| T547K, D614G, H655Y, N679K, P681H, N764K, D796Y, N856K, | |
| Q954H, N969K, L981F | |
| A67V, Δ69-70, T95I, G142D/Δ143-145, Δ211/L212I, ins214EPE, | B.1.1.529 + L452R |
| G339D, S371L, S373P, S375F, K417N, N440K, L452R, G446S, | |
| S477N, T478K, E484A, Q493R, G496S, Q498R, N501Y, Y505H, | |
| T547K, D614G, H655Y, N679K, P681H, N764K, D796Y, N856K, | |
| Q954H, N969K, L981F | |
| A67V, H69-V70del, T95I, G142D/V143-Y145del, (N211-L212)→I, | B.1.1.529 + F817P, A892P, |
| ins214EPE, G339D, S371L, S373P, S375F, K417N, N440K, G446S, | A899P, A942P |
| S477N, T478K, E484A, Q493R, G496S, Q498R, N501Y, Y505H, | |
| T547K, D614G, H655Y, N679K, P681H, N764K, D796Y, F817P, | |
| N856K, A892P, A899P, A942P, Q954H, N969K, L981F | |
| T19I, (L24-A27)→S, G142D, V213G, G339D, S371F, S373P, S375F, | BA.2 |
| T376A, D405N, R408S, K417N, N440K, S477N, T478K, E484A, | |
| Q493R, Q498R, N501Y, Y505H, D614G, H655Y, N679K, P681H, | |
| N764K, D796Y, Q954H, N969K | |
| T19I, (L24-A27)→S, G142D, V213G, G339D, S371F, S373P, S375F, | BA.2 + L452R |
| T376A, D405N, R408S, K417N, N440K, L452R, S477N, T478K, | |
| E484A, Q493R, Q498R, N501Y, Y505H, D614G, H655Y, N679K, | |
| P681H, N764K, D796Y, Q954H, N969K | |
| T19I, (L24-A27)→S, G142D, V213G, G339D, S371F, S373P, S375F, | BA.2 + L452M |
| T376A, D405N, R408S, K417N, N440K, L452M, S477N, T478K, | |
| E484A, Q493R, Q498R, N501Y, Y505H, D614G, H655Y, N679K, | |
| P681H, N764K, D796Y, Q954H, N969K | |
| T19I, (L24-A27)→S, G142D, V213G, G339D, S371F, S373P, S375F, | BA.2.12.1 |
| T376A, D405N, R408S, K417N, N440K, L452Q, S477N, T478K, | |
| E484A, Q493R, Q498R, N501Y, Y505H, D614G, H655Y, N679K, | |
| P681H, S704L, N764K, D796Y, Q954H, N969K | |
| T19I, L24-A27→S, G142D, K147E, W152R, F157L, I210V, V213G, | BA.2.75 |
| G257S, G339H, S371F, S373P, S375F, T376A, D405N, R408S, | |
| K417N, N440K, G446S, N460K, S477N, T478K, E484A, Q498R, | |
| N501Y, Y505H, D614G, H655Y, N679K, P681H, N764K, D796Y, | |
| Q954H, N969K | |
| T19I, L24-A27 > S, G142D, K147E, W152R, F157L, I210V, V213G, | BA.2.75.2 |
| G257S, G339H, R346T, S371F, S373P, S375F, T376A, D405N, | |
| R408S, K417N, N440K, G446S, N460K, S477N, T478K, E484A, | |
| F486S, Q498R, N501Y, Y505H, D614G, H655Y, N679K, P681H, | |
| N764K, D796Y, Q954H, N969K, D1199N | |
| A67V, H69-V70del, T95I, G142D, V143-Y145del, (N211-L212)→I, | BA.3 |
| G339D, S371F, S373P, S375F, D405N, K417N, N440K, G446S, | |
| S477N, T478K, E484A, Q493R, Q498R, N501Y, Y505H, D614G, | |
| H655Y, N679K, P681H, N764K, D796Y, Q954H, N969K | |
| T19I, (L24-A27)→S, H69-V70del, G142D, V213G, G339D, S371F, | BA.4 |
| S373P, S375F, T376A, D405N, R408S, K417N, N440K, L452R, | |
| S477N, T478K, E484A, F486V, Q498R, N501Y, Y505H, D614G, | |
| H655Y, N658S, N679K, P681H, N764K, D796Y, Q954H, N969K | |
| V3G, T19I, L24-A27 > S, H69-V70del, G142D, V213G, G339D, | BA.4.6 |
| R346T, S371F, S373P, S375F, T376A, D405N, R408S, K417N, | |
| N440K, L452R, S477N, T478K, E484A, F486V, Q498R, N501Y, | |
| Y505H, D614G, H655Y, N658S, N679K, P681H, N764K, D796Y, | |
| Q954H, N969K | |
| T19I, (L24-A27)→S, H69-V70del, G142D, V213G, G339D, S371F, | BA.5 |
| S373P, S375F, T376A, D405N, R408S, K417N, N440K, L452R, | |
| S477N, T478K, E484A, F486V, Q498R, N501Y, Y505H, D614G, | |
| H655Y, N679K, P681H, N764K, D796Y, Q954H, N969K | |
| T19I, L24-A27 > S, H69-V70del, G142D, V213G, G339D, R346T, | BF.7 |
| S371F, S373P, S375F, T376A, D405N, R408S, K417N, N440K, | |
| L452R, S477N, T478K, E484A, F486V, Q498R, N501Y, Y505H, | |
| D614G, H655Y, N679K, P681H, N764K, D796Y, Q954H, N969K | |
| T19I, L24-A27 > S, G142D, K147E, W152R, F157L, I210V, V213G, | BN.1 |
| G257S, G339H, R346T, K356T, S371F, S373P, S375F, T376A, | |
| D405N, R408S, K417N, N440K, G446S, N460K, S477N, T478K, | |
| E484A, F490S, Q498R, N501Y, Y505H, D614G, H655Y, N679K, | |
| P681H, N764K, D796Y, Q954H, N969K | |
| T19I, L24-A27 > S, H69-V70del, G142D, V213G, G339D, S371F, | BQ.1 |
| S373P, S375F, T376A, D405N, R408S, K417N, N440K, K444T, | |
| L452R, N460K, S477N, T478K, E484A, F486V, Q498R, N501Y, | |
| Y505H, D614G, H655Y, N679K, P681H, N764K, D796Y, Q954H, | |
| N969K | |
| T19I, L24-A27 > S, H69-V70del, G142D, V213G, G339D, R346T, | BQ.1.1 |
| S371F, S373P, S375F, T376A, D405N, R408S, K417N, N440K, | |
| K444T, L452R, N460K, S477N, T478K, E484A, F486V, Q498R, | |
| N501Y, Y505H, D614G, H655Y, N679K, P681H, N764K, D796Y, | |
| Q954H, N969K | |
| T19I, L24-A27 > S, V83A, G142D, Y144del, H146Q, Q183E, V213E, | XBB.1 |
| G252V, G339H, R346T, L368I, S371F, S373P, S375F, T376A, | |
| D405N, R408S, K417N, N440K, V445P, G446S, N460K, S477N, | |
| T478K, E484A, F486S, F490S, Q498R, N501Y, Y505H, D614G, | |
| H655Y, N679K, P681H, N764K, D796Y, Q954H, N969K | |
| T19I, L24-A27 > S, V83A, G142D, Y144del, H146Q, Q183E, V213E, | XBB.1.5 |
| G252V, G339H, R346T, L368I, S371F, S373P, S375F, T376A, | |
| D405N, R408S, K417N, N440K, V445P, G446S, N460K, S477N, | |
| T478K, E484A, F486P, F490S, Q498R, N501Y, Y505H, D614G, | |
| H655Y, N679K, P681H, N764K, D796Y, Q954H, N969K | |
| ΔH69-V70, ΔY144, N501Y, A570D, D614G, P681H, T716I, S982A, | B.1.1.7 |
| D1118H | |
| ΔH69-V70, ΔY144, E484K, N501Y, A570D, D614G, P681H, T716I, | B.1.1.7 + E484K |
| S982A, D1118H | |
| ΔH69-V70, L189F, N439K, D614G, V772I | B.1.258.17 |
| L18F, D80A, D215G, Δ242-244, R246I, K417N, E484K, N501Y, | B.1.351 |
| D614G, A701V | |
| D80A, D215G, K417N, E484K, N501Y, D614G, A701V | B.1.351.1 |
| S13I, W152C, L452R, D614G | B.1.429 |
| W152R, N439K, D614G, P681R | B.1.466.2 |
| Q52R, A67V, ΔH69-V70, Δ144, E484K, Q677H, D614G, F888L | B.1.525 |
| L5F, T95I, D253G, E484K, D614G, A701V | B.1.526 |
| D80G, ΔY144, F157S, L452R, D614G, T859N, D950H | B.1.526.1 |
| L452R, E484Q, D614G | B.1.617 |
| T95I, G142D, E154K, L452R, E484Q, D614G, P681R, Q1071H | B.1.617.1 |
| T19R, Δ157-158, L452R, T478K, D614G, P681R, D950N | B.1.617.2 |
| T19R, G142D, Δ156-157, R158G, L452R, T478K, D614G, P681R, | B.1.617.2 (AY.3; AY.5; AY.6; |
| D950N | AY.7; AY.14) (Alt Seq 1, |
| | previously designated as |
| | “CDC”) |
| T19R, T95I, G142D, Δ156/157, R158G, L452R, T478K, D614G, | B.1.617.2 (AY.4) (Alt Seq 2, |
| P681R, D950N | previously designated as |
| | “Alternative”) |
| T19R, ΔY144, Δ157/158, L452R, T478K, D614G, P681R, D950N | B.1.617.2 + ΔY144 |
| T19R, G142D, L452R, E484Q, D614G, P681R, D950N | B.1.617.3 |
| ΔY145/146, E484K, D614G | B.1.618 |
| P26S, ΔH69-V70, V126A, ΔY144, Δ242-244, H245Y, S477N, E484K, | B.1.620 |
| D614G, P681H, T1027I, D1118H | |
| T95I, Y144T, Y145S, ins146N, R346K, E484K, N501Y, D614G, | B.1.621 |
| P681H, D950N | |
| P9L, E96Q, C136-Y144del, R190S, D215H, R346S, N394S, Y449N, | B.1.640.2 |
| E484K, F490S, N501Y, D614G, P681H, T859N, D1139H | |
| Δ69-70, ΔY144, Q493R, N501Y, A570D, D614G, P681H, T716I, | BV-1 |
| S982A, D1118H | |
| G75V, T76I, ΔR246, ΔS247, ΔY248, ΔL249, ΔT250, ΔP251, ΔG252, | C.37 |
| D253N, L452Q, F490S, D614G, T859N | |
| L18F, T20N, P26S, D138Y, R190S, K417T, E484K, N501Y, D614G, | P.1 |
| H655Y, T1027I, V1176F | |
| E484K, D614G, V1176F | P.2 |
| Δ141-143, E484K, N501Y, D614G, P681H, E1092K, H1101Y, | P.3 |
| V1176F | |
| W152L, E484K, D614G, G769V | R.1 |

Alternative mutations for the S protein of SARS-CoV-2 strains AY.1, AY.2, and B.1.617.2 are listed as “Alt Seq #.”

TABLE 1E
Selected SARS-CoV-2 Strains and S-RBD Mutations

| S-RBD Mutation(s) (relative to reference strain NC_045512) | Associated Strain |
|---|---|
| V367F | A.23.1 |
| R346K, T478R, E484K | A.VOI.V2 |
| N501YK417N, L452R, T478K | BAY.1.1.7 |
| K417N, L452R, T478K | AY.2 |
| L452R, T478K | AY.3 |
| L452R, T478K | AY.4 |
| L452R, T478K | AY.5 |
| L452R, T478K | AY.6 |
| L452R, T478K | AY.7 |
| L452R, T478K | AY.12 |
| L452R, T478K | AY.14 |
| T478K | B.1.1.519 |
| G339D, S371L, S373P, S375F, K417N, N440K, G446S, S477N, T478K, E484A, | B.1.1.529 |
| Q493R, G496S, Q498R, N501Y, Y505H | |
| G339D, R346K, S371L, S373P, S375F, K417N, N440K, G446S, S477N, T478K, | B.1.1.529 + |
| E484A, Q493R, G496S, Q498R, N501Y, Y505H | R346K |
| | (BA.1.1) |
| G339D, S371L, S373P, S375F, K417N, N440K, L452R, G446S, S477N, T478K, | B.1.1.529 + |
| E484A, Q493R, G496S, Q498R, N501Y, Y505H | L452R |
| G339D, S371F, S373P, S375F, T376A, D405N, R408S, K417N, N440K, S477N, | BA.2 |
| T478K, E484A, Q493R, Q498R, N501Y, Y505H | |
| G339D, S371F, S373P, S375F, T376A, D405N, R408S, K417N, N440K, L452R, | BA.2 + L452R |
| S477N, T478K, E484A, Q493R, Q498R, N501Y, Y505H | |
| G339D, S371F, S373P, S375F, T376A, D405N, R408S, K417N, N440K, L452M, | BA.2 + L452M |
| S477N, T478K, E484A, Q493R, Q498R, N501Y, Y505H | |
| G339D, S371F, S373P, S375F, T376A, D405N, R408S, K417N, N440K, L452Q, | BA.2.12.1 |
| S477N, T478K, E484A, Q493R, Q498R, N501Y, Y505H | |
| G339H, S371F, S373P, S375F, T376A, D405N, R408S, K417N, N440K, G446S, | BA.2.75 |
| N460K, S477N, T478K, E484A, Q498R, N501Y, Y505H | |
| G339D, S371F, S373P, S375F, D405N, K417N, N440K, G446S, S477N, T478K, | BA.3 |
| E484A, Q493R, Q498R, N501Y, Y505H | |
| G339D, S371F, S373P, S375F, T376A, D405N, R408S, K417N, N440K, L452R, | BA.4; BA.5; BF.5 |
| S477N, T478K, E484A, F486V, Q498R, N501Y, Y505H | |
| G339D, R346T, S371F, S373P, S375F, T376A, D405N, R408S, K417N, N440K, | BA.4.6 |
| L452R, S477N, T478K, E484A, F486V, Q498R, N501Y, Y505H | |
| G339D, R346T, S371F, S373P, S375F, T376A, D405N, R408S, K417N, N440K, | BF.7 |
| L452R, S477N, T478K, E484A, F486V, Q498R, N501Y, Y505H | |
| G339H, R346T, K356T, S371F, S373P, S375F, T376A, D405N, R408S, K417N, | BN.1 |
| N440K, G446S, N460K, S477N, T478K, E484A, F490S, Q498R, N501Y, Y505H | |
| G339D, S371F, S373P, S375F, T376A, D405N, R408S, K417N, N440K, K444T, | BQ.1 |
| L452R, N460K, S477N, T478K, E484A, F486V, Q498R, N501Y, Y505H | |
| G339D, R346T, S371F, S373P, S375F, T376A, D405N, R408S, K417N, N440K, | BQ.1.1 |
| K444T, L452R, N460K, S477N, T478K, E484A, F486V, Q498R, N501Y, Y505H | |
| G339H, R346T, L368I, S371F, S373P, S375F, T376A, D405N, R408S, K417N, | XBB.1 |
| N440K, V445P, G446S, N460K, S477N, T478K, E484A, F486S, F490S, Q498R, | |
| N501Y, Y505H | |
| G339H, R346T, L368I, S371F, S373P, S375F, T376A, D405N, R408S, K417N, | XBB.1.5 |
| N440K, V445P, G446S, N460K, S477N, T478K, E484A, F486P, F490S, Q498R, | |
| N501Y, Y505H | |
| N501Y | B.1.1.7 |
| E484K, N501Y | B.1.1.7 + E484K |
| Q414K, N450K | B.1.214.2 |
| N439K | B.1.258.17 |
| K417N, E484K, N501Y | B.1.351 |
| K417N, E484K, N501Y | B.1.351.1 |
| L452R | B.1.427 |
| L452R | B.1.429 |
| N439K | B.1.466.2 |
| E484K | B.1.525 |
| E484K | B.1.526 |
| L452R | B.1.526.1 |
| S477N | B.1.526.2 |
| L452R, E484Q | B.1.617 |
| L452R, E484Q | B.1.617.1 |
| L452R, T478K | B.1.617.2 |
| L452R, T478K | B.1.617.2 + ΔY144 |
| L452R, E484Q | B.1.617.3 |
| E484K | B.1.618 |
| S477N, E484K | B.1.620 |
| Q493R, N501Y | BV-1 |
| L452Q, F490S | C.37 |
| K417T, E484K, N501Y | P.1 |
| E484K | P.2 |
| E484K, N501Y | P.3 |
| E484K | R.1 |

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus nucleic acid is RNA. In embodiments, the coronavirus is SARS-CoV-2. In embodiments, the binding reagent comprises an oligonucleotide comprising a sequence complementary to the coronavirus nucleic acid sequence. In embodiments, the binding reagent binds to a nucleic acid from a specific strain of the coronavirus, e.g., a SARS-CoV-2 strain as described herein. In embodiments, the binding reagent binds to a SARS-CoV-2 nucleic acid encoding the N protein (i.e., the N gene). The SARS-CoV-2 N gene can be detected at three different regions: N1, N2, and N3. The N1 and N2 regions are specific to SARS-CoV-2, and the N3 region is universal to the coronaviruses in the same clade as SARS-CoV-2 (e.g., clade 2 and 3 viruses within the subgenus Sarbecovirus, including SARS-CoV-2, SARS-CoV, and bat- and civet-SARS-like CoVs. See, e.g., Lu et al., Emerg Infect Dis 26(8):1654-1665 (2020)). In embodiments, the binding reagent binds to SARS-CoV-2 N1 region, N2 region, N3 region, or a combination thereof. In embodiments, the biological sample is saliva, the coronavirus is SARS-CoV-2 and the nucleic acid is RNA.

In embodiments, the coronavirus is capable of infecting a human. In embodiments, the coronavirus causes a respiratory tract infection in a human. In embodiments, the coronavirus is SARS-CoV, MERS-CoV, SARS-CoV-2, HcoV-OC43, HcoV-229E, HcoV-NL63, HcoV-HKU1, or a combination thereof. In embodiments, the method detects a coronavirus component that is substantially conserved in SARS-CoV, MERS-CoV, SARS-CoV-2, HcoV-OC43, HcoV-229E, HcoV-NL63, and HcoV-HKU1. In embodiments, the method detects a protein or peptide fragment that is substantially conserved in SARS-CoV, MERS-CoV, SARS-CoV-2, HcoV-OC43, HcoV-229E, HcoV-NL63, and HcoV-HKU1.

In embodiments, the immunoassay described herein is a multiplexed immunoassay method. A multiplexed immunoassay can simultaneously detect multiple substances of interest, e.g., coronavirus components, in a sample. A multiplexed immunoassay can also use multiple binding reagents that specifically bind a substance of interest, e.g., a coronavirus component, in a sample. Multiplexed immunoassays can provide reliable results while reducing processing time and cost. In embodiments, a multiplexed immunoassay for detecting a coronavirus comprises multiple binding reagents, each of which binds to a different coronavirus component, e.g., a conserved coronavirus protein. In embodiments, a multiplexed immunoassay comprising binding reagents that each specifically binds a different coronavirus component provides improved detection accuracy, e.g., over a singleplex method utilizing a single binding reagent. In embodiments, the immunoassay method detects a coronavirus by detecting one or more of the coronavirus E protein, S protein, including S1 and S2 subunits, S-NTD, S-ECD, and S-RBD, M protein, HE protein, N protein, nsp1, nsp2, nsp3, nsp4, nsp5, nsp6, nsp7, nsp8, nsp9, nsp10, nsp11, nsp12, nsp13, nsp14, nsp15, and nsp16. In embodiments, the coronavirus is SARS-CoV-2. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein and/or S-RBD. In embodiments, the immunoassay method detects SARS-CoV-2 by detecting any of the SARS-CoV-2 S protein and/or S-RBD variants in Tables 1A and 1B. In embodiments, the immunoassay detects SARS-CoV-2 by detecting SARS-CoV-2 N protein and S protein. In embodiments, the immunoassay detects SARS-CoV-2 by detecting any combination of the SARS-CoV-2 N protein, S protein, E protein, and M protein. In embodiments, the immunoassay detects SARS-CoV-2 by detecting SARS-CoV-2 N protein, S protein, E protein, and M protein. In embodiments, the immunoassay detects SARS-CoV-2 by detecting any of the SARS-CoV-2 protein variants in Table 1A.

In embodiments, the immunoassay method is a multiplexed method comprising: contacting the biological sample with a surface comprising a binding reagent in each binding domain on the surface, wherein the binding reagent in each binding domain independently binds to a viral protein selected from SARS-CoV-2 N protein, SARS-CoV-2 S protein, SARS-CoV-2 S-RBD, SARS-CoV-2 E protein, SARS-CoV-2 M protein, or a combination thereof; forming a binding complex in each binding domain comprising the viral protein and the binding reagent that binds to the viral protein; and measuring the concentration of the viral protein in each binding complex. In embodiments, the SARS-CoV-2 S protein comprises any of the mutations shown in Tables 1A and 1B. In embodiments, each binding complex further comprises a detection reagent that specifically binds to the viral protein of the binding complex. Detection reagents are further described herein.

In embodiments, the immunoassay method is a multiplexed method capable of simultaneously detecting multiple coronaviruses in a biological sample. In embodiments, the multiplexed method is capable of simultaneously detecting one or more of SARS-CoV, MERS-CoV, SARS-CoV-2, HcoV-OC43, HcoV-229E, HcoV-NL63, and HcoV-HKU1.

In embodiments, the binding reagent and/or the detection reagent that specifically binds to the coronavirus component described herein is an antibody, antigen, ligand, receptor, oligonucleotide, hapten, epitope, mimotope, or aptamer. In embodiments, the binding reagent and/or the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent and/or the detection reagent comprises at least one heavy or light chain complementarity determining region (CDR) of an antibody. In embodiments, the binding reagent and/or the detection reagent comprises at least two CDRs from one or more antibodies. In embodiments, the binding reagent and/or the detection reagent is an antibody or antigen-binding fragment thereof. In embodiments, the binding reagent and/or the detection reagent is a receptor for the coronavirus component. In embodiments, the binding reagent and/or the detection reagent is a receptor for the coronavirus S protein. In embodiments, the binding reagent and/or the detection reagent is angiotensin-converting enzyme 2 (ACE2). In embodiments, the binding reagent and/or the detection reagent is neuropilin-1 (NRP1). In embodiments, the binding reagent and/or the detection reagent is CD147.

In embodiments where the method comprises detecting one or more variants of an SARS-CoV-2 protein (e.g., an S protein comprising a mutation shown in Tables 1A and 1B or an Orf1ab, E, Orf8, or N protein comprising a mutation shown in Table 1A), the binding reagent comprises an antibody or antigen-binding fragment thereof that is capable of specifically binding the wild-type, protein variant(s), or both the protein variant and the wild-type, and the detection reagent comprises an antibody or antigen-binding fragment thereof that is capable of binding the wild-type, protein variant(s), or both the wild-type and variant forms of the protein. In embodiments, the SARS-CoV-2 protein is an S protein, an N protein, an E protein, an Orf1ab protein, an Orf8 protein, or a combination thereof. In embodiments, the SARS-CoV-2 protein is an S protein.

In embodiments, the method is capable of detecting about 1 fg/mL to about 1 ng/mL, about 1 fg/mL to about 0.8 ng/mL, about 1 fg/mL to about 0.5 ng/mL, about 1 fg/mL to about 0.1 ng/mL, about 1 fg/mL to about 50 pg/mL, about 1 fg/mL to about 20 pg/mL, about 1 fg/mL to about 10 pg/mL, about 1 fg/mL to about 5 pg/mL, about 1 fg/mL to about 2 pg/mL, about 1 fg/mL to about 1 pg/mL, about 5 fg/mL to about 100 fg/mL, about 7 fg/mL to about 75 fg/mL, or about 10 fg/mL to about 50 fg/mL of a virus (e.g., a coronavirus such as SARS-CoV-2). In embodiments, the method is capable of detecting less than or about 5 pg/mL, less than or about 2 pg/mL, less than or about 1 pg/mL, less than or about 500 fg/mL, less than or about 100 fg/mL, less than or about 75 fg/mL, less than or about 50 fg/mL, or less than or about 10 fg/mL of a virus (e.g., a coronavirus such as SARS-CoV-2). In embodiments, the method is capable of detecting less than or about 10⁹ viral particles per mL, less than or about 10⁸ viral particles per mL, less than or about 10⁷ viral particles per mL, less than or about 10⁶ viral particles per mL, less than or about 100000 viral particles per mL, less than or about 10000 viral particles per mL, less than or about 1000 viral particles per mL, or less than or about 100 viral particles per mL. In embodiments where the method detects a viral nucleic acid, one viral particle is one viral genome equivalent. In embodiments, the method is capable of detecting less than or about 10⁹ viral genome equivalents per mL, less than or about 10⁸ viral genome equivalents per mL, less than or about 10⁷ viral genome equivalents per mL, less than or about 10⁶ viral genome equivalents per mL, less than or about 100000 viral genome equivalents per mL, less than or about 10000 viral genome equivalents per mL, less than or about 1000 viral genome equivalents per mL, or less than or about 100 viral genome equivalents per mL.

### Biomarkers

In embodiments, the invention provides a method for detecting a biomarker that is produced by a host (e.g., a human subject) in response to a viral infection, e.g., by a respiratory virus, including coronaviruses such as SARS-CoV-2. As used herein, “host” refers to a subject who has been infected with or suspected of being infected with a virus described herein, e.g., a coronavirus such as SARS-CoV-2. Unless otherwise specified, the biomarkers described herein are produced by a host, e.g., a human subject, in response to viral exposure and/or infection as described herein. In embodiments, the biomarker is an immune response biomarker. In embodiments, the biomarker is an antibody. The terms “antibody biomarker” and “antibody” are used interchangeably throughout the present disclosure. In embodiments, the biomarker is an inflammation response biomarker. In embodiments, the biomarker is a damage response biomarker. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is a coronavirus. In embodiments, the virus is SARS-CoV-2.

As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility or responsiveness of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2): 182-188 (2004); Strimbu et al., Curr Opin HIV AIDS 5(6): 463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. Unless specified otherwise, the biomarkers referred to herein encompass human biomarkers.

As used herein, the term “level” in the context of a biomarker refers to the amount, concentration, or activity of a biomarker. The term “level” can also refer to the rate of change of the amount, concentration, or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide (e.g., an antibody), nucleic acid, or small molecule. “Level” can also refer to an absolute amount of a biomarker in a sample or to a relative amount of the biomarker, including amount or concentration determined under steady-state or non-steady-state conditions. “Level” can further refer to an assay signal that correlates with the amount, concentration, activity or rate of change of a biomarker. The level of a biomarker can be determined relative to a control marker in a sample.

Measurement of biomarker values and levels before and after a particular event, e.g., cellular or environmental event, may be used to gain information regarding an individual's response to the event. For example, samples or model organisms can be subjected to stress- or disease-inducing conditions, or a treatment or prevention regimen, and a particular biomarker can then be detected and quantitated in order to determine its changes in response to the condition or regimen. However, the opposite, i.e., measuring biomarker values and levels to determine whether an organism has been subjected to stress- or disease-inducing condition, tends to be much more complicated, as changes in the levels of a single biomarker are sometimes not definitively associated with a particular condition.

In embodiments, the measured levels of the one or more biomarkers described herein provides information regarding infection and immune response to infection, e.g., the course or maturity of infection, the etiology of severe illness, and the potential severity of illness. In embodiments, the measured levels of the one or more biomarkers described herein provides information regarding a subject's antibody response, cytokine response, neutrophil, macrophage, and/or monocyte production, complement activation, B cell and/or T cell activation, or a combination thereof.

In embodiments, detection and/or measurement of a single biomarker is sufficient to provide a prediction and/or diagnosis of a disease or condition. In embodiments, combinations of biomarkers are used to provide a strong prediction and/or diagnosis. Although a linear combination of biomarkers (i.e., the combination comprises biomarkers that individually provide a relatively strong correlation) can be utilized, linear combinations may not be available in many situations, for example, when there are not enough biomarkers available and/or with strong correlation. In alternative approaches, a biomarker combination is selected such that the combination is capable of achieving improved performance (i.e., prediction or diagnosis) compared with any of the individual biomarkers, each of which may not be a strong correlator on its own. Biomarkers for inclusion in a biomarker combination can be selected for based on their performance in different individuals, e.g., patients, wherein the same biomarker may not have the same performance in different individuals, but when combined with the remaining biomarkers, provide an unexpectedly strong correlation for prediction or diagnosis in a population. For example, Bansal et al., Statist Med 32: 1877-1892 (2013) describe methods of determining biomarkers to include in such a combination, noting in particular that optimal combinations may not be obvious to one of skill in the art, especially when subgroups are present or when individual biomarker correlations are different between cases and controls. Thus, selecting a combination of biomarkers for providing a consistent and accurate prediction and/or diagnosis can be particularly challenging and unpredictable.

Even when a suitable combination of biomarkers is determined, utilizing the combination of biomarkers in an assay poses its own set of difficulties. For example, detecting and/or quantitating each biomarker in the combination in its own separate assay may not be feasible with small samples, and using a separate assay to measure each biomarker in a sample may not provide consistent and comparable results. Furthermore, running an individual assay for each biomarker in a combination can be a cumbersome and complex process that can be inefficient and costly.

A multiplexed assay that can simultaneously measure the concentrations of multiple biomarkers can provide reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive; all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output measurement.

In embodiments, the invention provides methods of assessing an individual's immune response to a viral infection. In embodiments, the invention provides methods of assessing a group of individuals immune response to a viral infection. In embodiments, assessing an immune response comprises determining the type and/or strength of the immune response, e.g., detecting the molecular components produced in response to a viral infection (e.g., acute phase reactants, antibodies, cytokines, etc.) and measuring the amounts of each component produced. In embodiments, the invention provides methods of assessing the differences in immune responses by age, race, ethnicity, socioeconomic backgrounds, and/or underlying conditions, e.g., lung disease, diabetes, cancer, etc., which may be associated with poor clinical outcomes. In embodiments, the invention provides methods of determining the epidemiology of diseases caused by the viruses described herein, e.g., COVID-19. In embodiments, the virus is a coronavirus. In embodiments, the virus is SARS-CoV-2.

In embodiments, the invention provides methods of assessing cross-reactivity of an individual's immune response between different coronaviruses (e.g., SARS-CoV, MERS-CoV, SARS-CoV-2, HCoV-OC43, HCoV-229E, HCoV-NL63, and HCoV-HKU1). In embodiments, the invention provides methods of mapping the epitopes recognized by an individual's immune response, e.g., epitopes on a coronavirus S protein. In embodiments, the invention provides methods of assessing the individual's clinical outcome based on the mapped epitopes of immune responses. In embodiments, the invention provides methods of assessing an individual's immune response by detecting different IgG classes and/or subclasses. In embodiments, the invention provides methods of assessing the individual's clinical outcome based on the IgG classes and/or subclasses. In embodiments, the invention provides methods of assessing the affinity and/or avidity of an individual's immune response to different viral antigens. In embodiments, the invention provides methods of assessing the strength of an immune response, e.g., measuring the total antibody concentration or the concentration of different classes or subclasses of antibodies in an individual. In embodiments, the invention provides methods of determining the natural interacting partner(s) of the virus, e.g., a coronavirus such as SARS-CoV-2. As used in the context of viral infections, a “natural interacting partner” refers to a substance in the host cell (e.g., proteins or carbohydrate moieties on a host cell surface) that interacts with a viral component described herein. Natural interacting partners of viruses are further described in, e.g., Brito et al., Front Microbiol 8:1557 (2017). Natural interacting partners of SARS-CoV-2 include, e.g., ACE2, NRP1, and CD147, and are further described in Gordon et al., bioRxiv 2020.03.22.002386v1 (2020) doi:10.1101/2020.03.22.002386v1, Daly et al., bioRxiv 2020.06.05. 134114 (2020) doi:10.1101/2020.06.05.134114, and Bojkova et al., Nature Research (Pre-Print 11 Mar. 2020) doi:10.21203/rs.3.rs-17218/v1. In embodiments, the invention provides a competitive assay for SARS-CoV-2 utilizes ACE2, NRP1, CD147, or different sialic acid-containing substances to determine the interacting partner(s) of the SARS-CoV-2 S protein.

In embodiments, the invention provides methods of assessing changes in the immune response over time. In embodiments, the invention provides methods of assessing an individual's immune response at different time points after infection and/or after the first onset of a symptom. In embodiments, the invention provides methods of assessing the cytokines present in an individual at different time points after infection and/or after the first onset of a symptom. Symptoms of viral infections are described herein. In embodiments, the invention provides methods of assessing the long-term effects of an infection on an individual. For example, the coronavirus SARS-CoV-2 can cause post-acute COVID-19 syndrome (also known as post-COVID syndrome or “long COVID”), in which symptoms of the infection, including fatigue, headaches, shortness of breath, anosmia, muscle weakness, low fever, and cognitive dysfunction, persist for weeks or months after the typical convalescence period of COVID-19. In embodiments, the invention provides methods of assessing an individual's immune response at different time points after vaccination. In embodiments, the invention provides methods of determining the immune response components that provide immunity to a viral infection. In embodiments, the invention provides methods of assessing an individual's immune response at different time points after receiving a treatment for the viral infection. In embodiments, the invention provides methods of assessing the effect of convalescent serum treatment in an individual, e.g., comprising measuring the individual's immune response after administration of the convalescent serum. In embodiments, the invention provides methods of assessing the immune response components (e.g., antibodies) present in a convalescent serum sample, e.g., comprising determining its effectiveness, half life, and/or functional window of treatment in an individual. In embodiments, the invention provides methods of assessing the effectiveness, half life, and/or functional window of protection of a therapeutic antibody treatment. In embodiments, the virus is a coronavirus. In embodiments, the virus is SARS-CoV-2.

In embodiments, the invention provides methods of assessing an individual's immune response, e.g., an antibody, to a coronavirus (e.g., an endemic coronavirus such as HCoV-OC43, HCoV-229E, HCoV-NL63, and HCoV-HKU) to determine a clinical outcome of infection by a different coronavirus, e.g., SARS-CoV-2. In embodiments, the invention provides methods of assessing an individual's immune response, e.g., an antibody, to a respiratory virus (e.g., influenza or RSV) to determine a clinical outcome of infection by a different respiratory virus, e.g., SARS-CoV-2. In embodiments, the invention provides methods of assessing an individual's immune response and/or clinical outcome in a SARS-CoV-2 infection by determining a ratio of the individual's antibody level against the SARS-CoV-2 N protein to the individual's antibody level against the SARS-CoV-2 S protein. In embodiments, the antibody levels are measured in a blood sample. In embodiments, the antibody levels are measured in a saliva sample.

In embodiments, the invention provides a serology assay for determining the SARS-CoV-2 strain that has infected an individual. Currently, the only available methods for determining SARS-CoV-2 strain are nucleic acid-based methods such as PCR or sequencing, which typically require a nasopharyngeal or oropharyngeal sample from a subject. Assessment of the subject's antibody or immune response, as described herein, would require a further serology sample. Thus, a serology assay that determines SARS-CoV-2 strain reduces the amount and type of sample required from the subject, thereby reducing sample collection and processing time, and stress on the subject. It was surprisingly discovered that antibody biomarkers from an individual infected with a particular SARS-CoV-2 strain had highly specific activity against the S protein and/or S-RBD of that particular strain as compared to other strains. This unexpected result demonstrates a population-wide immune response (e.g., antibody response) bias for strain-specific epitopes on the SARS-CoV-2 S protein and/or S-RBD, which was not observed with other infectious diseases, e.g., other viruses, which typically have highly variable immune responses (e.g., antibody responses) between infected individuals that do not correlate with the viral strain. In embodiments, the invention provides methods of assessing an individual's immune response to different strains or variants of a coronavirus, e.g., SARS-CoV-2. In embodiments, the invention provides methods of mapping SARS-CoV-2 strain-specific epitopes on the SARS-CoV-2 S protein and/or S-RBD. Such methods are also useful for epidemiological studies to determine circulating variants in a population or geographical region.

In embodiments, the invention provides a method of determining the SARS-CoV-2 strain that has infected one or more individuals, comprising: performing a multiplexed serology assay on a sample obtained from the one or more individuals to detect one or more antibody biomarkers against S proteins and/or S-RBD from multiple SARS-CoV-2 strains; and differentiating the detected antibody biomarker(s) based on binding of the antibody biomarker(s) to the S protein and/or S-RBD from each SARS-CoV-2 strain. In embodiments, the differentiating comprises determining a ratio of: a first antibody biomarker that binds an S protein and/or S-RBD from a first SARS-CoV-2 strain (e.g., wild-type SARS-CoV-2), to a second antibody biomarker that binds an S protein and/or S-RBD from a second SARS-CoV-2 strain (e.g., SARS-CoV-2 strain B.1.1.7). SARS-CoV-2 strains are further described herein, e.g., in Table 1A. Multiplexed serology assays are further described herein. In embodiments, the multiplexed serology assay detects one or more antibody biomarkers that binds to one or more of an S protein from wild-type SARS-CoV-2, an S protein from SARS-CoV-2 strain P.1, an S protein from SARS-CoV-2 strain B.1.1.7, and an S protein from SARS-CoV-2 strain 501Y.V2. In embodiments, the multiplexed serology assay detects one or more antibody biomarkers that binds to one or more of: an S protein from wild-type SARS-CoV-2, an S-D614G from SARS-CoV-2, an S protein from SARS-CoV-2 strain P.1, an S protein from SARS-CoV-2 strain B.1.1.7, an S protein from SARS-CoV-2 strain 501Y.V2, and an S-RBD from wild-type SARS-CoV-2. In embodiments, the multiplexed serology assay detects one or more antibody biomarkers that binds to one or more of: an S protein from wild-type SARS-CoV-2, an S-RBD from wild-type SARS-CoV-2, an S protein from SARS-CoV-2 strain B.1.1.7, an S-RBD from SARS-CoV-2 strain B.1.1.7, an S protein from SARS-CoV-2 strain 501Y.V2, an S-RBD from SARS-CoV-2 strain 501Y.V2, an S protein from SARS-CoV-2 strain P.1, and an S-RBD from SARS-CoV-2 strain P.1. In embodiments, the multiplexed serology assay detects one or more antibody biomarkers that binds to one or more of: an S protein from wild-type SARS-CoV-2, an S-RBD from wild-type SARS-CoV-2, an S protein from SARS-CoV-2 strain B.1.429, an S-RBD from SARS-CoV-2 strain B.1.429, an S protein from SARS-CoV-2 strain B.1.526/E484K, an S-RBD from SARS-CoV-2 strain B.1.526/E484K, an S protein from SARS-CoV-2 strain B.1.526/S477N, and an S-RBD from SARS-CoV-2 strain B.1.526/S477N. In embodiments, the multiplexed serology assay detects one or more antibody biomarkers that binds to one or more S proteins or subunit or fragment thereof that comprises any of the mutations shown in Tables 1A and 1B. In embodiments, the multiplexed serology assay is a classical serology assay, bridging serology assay, or competitive serology assay as described herein.

In embodiments, the invention provides a method of determining one or more SARS-CoV-2 strains in a sample. The method described herein is useful for tracking spread of one or more SARS-CoV-2 strains. The method provided herein is further useful for tracking the spread of one or more SARS-CoV-2 strains in one or more geographical regions and/or for tracking the spread of one or more SARS-CoV-2 strains over time. In embodiments, the invention provides a method for determining a SARS-CoV-2 strain in a sample, comprising: detecting at least a first antibody biomarker in the sample that binds to an antigen from a first SARS-CoV-2 strain and at least a second antibody biomarker in the sample that binds to an antigen from a second SARS-CoV-2 strain, wherein the detecting comprises contacting the sample with a surface comprising at least two binding domains, wherein the antigen from the first SARS-CoV-2 strain is immobilized on a first binding domain, and the antigen from the second SARS-CoV-2 strain is immobilized on a second binding domain; and determining a ratio of the first antibody biomarker to the second antibody biomarker, thereby determining the SARS-CoV-2 strain.

In some embodiments, the sample is from one or more individuals, wherein the one or more individuals are currently infected with SARS-CoV-2. In some embodiments, the sample is from one or more individuals, wherein the one or more individuals were previously infected with SARS-CoV-2. In some embodiments, the sample is from at least two individuals, wherein at least one individual is currently infected with SARS-CoV-2 and at least one individual was previously infected with SARS-CoV-2. In some embodiments, the sample is from at least one individual, wherein the individual is currently infected and was previously infected with SARS-CoV-2. In embodiments, the sample is from one or more individuals, wherein the one or more individuals are located in one or more geographical regions. In embodiments, the sample is from one or more individuals obtained at different time points. In embodiments, the sample comprises a pooled sample from at least two individuals. Pooled samples are further described herein.

In embodiments, the method further comprises determining the SARS-CoV-2 from one or more samples. In embodiments, the one or more samples are from one or more individuals as described herein. In embodiments, the method further comprises comparing the SARS-CoV-2 in one or more samples from one or more individuals located in one or more geographical regions, thereby tracking spread of the SARS-CoV-2 strain in the one or more geographical regions.

In embodiments, the SARS-CoV-2 strain is determined by inputting the ratio of the first antibody biomarker to the second antibody biomarker into a classification algorithm. Classification algorithms are further described herein. In embodiments, the method further comprises training a classification algorithm. In embodiments, the training comprises: measuring the amount of antibody biomarkers in a sample from a subject infected with a known SARS-CoV-2 strain that bind to an antigen from one or more SARS-CoV-2 strains, wherein the one or more SARS-CoV-2 strains comprise the known SARS-CoV-2 strain; normalizing the amount of measured antibody biomarker that bind to an antigen from the known SARS-CoV-2 strain against the amount of measured antibody biomarker that bind to an antigen from a further SARS-CoV-2 strain; and providing the normalized antibody biomarker amount to the classification algorithm.

In embodiments, the invention provides a method for differentiating infection associated with different SARS-CoV-2 strains. In embodiments, the method comprises training a classification algorithm. In embodiments, the method comprises obtaining a sample from a subject infected with a known SARS-CoV-2 strain; and measuring the amount of antibody biomarkers in the sample that bind to the S protein and/or S-RBD from multiple SARS-CoV-2 strains. In embodiments, the measuring comprises performing a multiplexed serology assay, e.g., a classical, bridging, or competitive multiplexed serology assay as described herein. In embodiments, the measured antibody biomarker amount for a particular strain is normalized against the measured antibody biomarker amount for a different strain. In embodiments, the normalized antibody biomarker amount is used to train the classification algorithm. In embodiments, normalized antibody biomarker amounts from multiple subjects, each infected with a known SARS-CoV-2 strain, are used to train the classification algorithm. Classification algorithms are known in the field and include but are not limited to, e.g., linear regression, logistic regression, random forest, support vector machine, and neural network. In embodiments, the invention provides a method of determining the SARS-CoV-2 strain that has infected one or more individuals, comprising: performing a multiplexed serology assay on a sample obtained from the one or more individuals to detect one or more antibody biomarkers against S proteins and/or S-RBD from multiple SARS-CoV-2 strains as described herein; and applying the classification algorithm described herein to determine the SARS-CoV-2 strain.

Serology tests that assess the presence of an antibody biomarker against a SARS-CoV-2 antigen have received U.S. FDA Emergency Use Authorization (EUA) with specificity of 95%. In embodiments, the invention provides improved sensitivity and/or specificity in determining whether a subject is currently infected or has previously been infected with a virus, e.g., a coronavirus such as SARS-CoV-2. In embodiments, the invention provides improved sensitivity and/or specificity in determining whether a subject has immunity to a virus, e.g., a coronavirus such as SARS-CoV-2. In embodiments, the methods herein have a sensitivity of greater than 90%, greater than 95%, greater than 96%, greater than 97%, greater than 98%, greater than 99%, greater than 99.5%, or greater than 99.9%. In embodiments, the methods herein have a specificity of greater than 90%, greater than 95%, greater than 96%, greater than 97%, greater than 98%, greater than 99%, greater than 99.5%, or greater than 99.9%. Assays with high sensitivity and specificity are important to correctly diagnose active infections and to correctly determine whether an individual has been previously exposed and/or immune to a virus, e.g., a coronavirus such as SARS-CoV-2. In particular, assays with high specificity are useful for conducting epidemiological studies in populations with low disease prevalence. Moreover, assays with high specificity are important for individual assessment due to the high risk of a false positive to the individual and the individual's community; individuals who received a false positive serology test result for SARS-CoV-2 may believe themselves to be immune and therefore erroneously engage in activity that can increase the likelihood of infection and spread of the virus.

### Antibody Biomarkers

In embodiments, the invention provides a method for detecting a respiratory virus, e.g., a coronavirus such as SARS-CoV-2, in a biological sample, by detecting a biomarker produced in response to an infection by the virus. In embodiments, the biomarker produced in response to a viral infection is an antibody.

In embodiments, the invention provides a method for detecting a biomarker that is capable of binding to a viral antigen in a biological sample. As used herein, a virus or viral antigen is any component or secretion of a virus that prompts an immune response in a host (e.g., a human). In embodiments, the viral antigen is a viral protein or fragment thereof. In embodiments, the viral antigen is a virus structural protein. In embodiments, the viral antigen is a virus nonstructural protein. Structural and nonstructural proteins of viruses, e.g., respiratory viruses such as coronaviruses, are described herein. In embodiments, the method is capable of determining whether a subject has been exposed to a particular virus, e.g., a coronavirus such as SARS-CoV-2. In embodiments, the method is capable of determining whether a subject is at risk of being infected by a particular virus, e.g., a coronavirus such as SARS-CoV-2. In embodiments, the method is capable of determining whether a subject has immunity to a particular virus, e.g., a coronavirus such as SARS-CoV-2.

In embodiments, the invention provides an immunoassay method comprising: quantifying the amounts of one or more biomarkers capable of binding to a respiratory virus antigen in a biological sample, wherein the respiratory virus is a coronavirus, an influenza virus, a paramyxovirus, an adenovirus, a bocavirus, a pneumovirus, an enterovirus, a rhinovirus, a parainfluenza virus (PIV), a metapneumovirus (MPV), a parechovirus (PeV), RSV, or a combination thereof, wherein the quantifying comprises measuring the concentrations of each of the one or more biomarkers in an immunoassay. In embodiments, the immunoassay comprises detecting one or more biomarkers that bind to a panel of respiratory virus antigens in a biological sample, wherein the respiratory viruses comprise a coronavirus, an influenza virus, and RSV. In embodiments, the immunoassay comprises detecting one or more biomarkers that bind to a panel of respiratory virus antigens in a biological sample, wherein the respiratory viruses comprise an enterovirus, MPV, RSV, an influenza virus, a rhinovirus, a coronavirus, a PIV, and a parechovirus. In embodiments, the immunoassay comprises detecting one or more biomarkers that bind to a panel of respiratory virus antigens in a biological sample, wherein the respiratory viruses comprise an influenza virus and a PIV.

In embodiments, the coronavirus antigen comprises a Spike (S) protein or fragment thereof, e.g., S-RBD, or an N protein. In embodiments, the RSV antigen comprises a pre-fusion F protein. In embodiments, the MPV antigen comprises an F protein (e.g., a pre-fusion F protein). In embodiments, the PIV antigen comprises an F protein (e.g., a pre-fusion F protein). In embodiments, the influenza virus antigen comprises a hemagglutinin (HA) protein. In embodiments, the enterovirus antigen comprises a virus-like particle (VLP). In embodiments, the VLP comprises one or more enterovirus proteins (e.g., a capsid protein, e.g., VP1, VP2, VP3, VP4, or combination thereof). In embodiments, the rhinovirus antigen comprises a capsid protein, e.g., capsid protein VP0. In embodiments, the parechovirus antigen comprises a VP0 protein.

In embodiments, the immunoassay method comprises: contacting the biological sample with a surface comprising a viral antigen in a binding domain on the surface; forming a binding complex in the binding domain comprising the viral antigen and a biomarker that binds to the viral antigen; and measuring the concentration of the biomarker in the binding complex. In embodiments, the biomarker is IgG, IgA, IgM, or combination thereof. In embodiments, the concentration of the biomarker is measured by contacting the binding complex with a detection reagent that specifically binds IgG, IgA, or IgM. In embodiments, the biomarker is a human biomarker, a mouse biomarker, a rat biomarker, a ferret biomarker, a minx biomarker, a bat biomarker, or a combination thereof. In embodiments, the biomarker is human IgG, IgA, or IgM. In embodiments, the biomarker is mouse IgG, IgA, or IgM. In embodiments, the biomarker is rat IgG, IgA, or IgM. In embodiments, the biomarker is ferret IgG, IgA, or IgM. In embodiments, the biomarker is minx IgG, IgA, or IgM. In embodiments, the biomarker is bat IgG, IgA, or IgM. Detection reagents are further described herein.

In embodiments, a binding domain comprises a mixture of antigens from more than one strain or type of virus, e.g., more than one strain of influenza, PIV, or SARS-CoV-2. In embodiments, the method comprises forming one or more binding complexes in the binding domain, wherein the binding complex comprises one of the antigens and a biomarker, wherein the biomarker binds specifically to the one antigen, or wherein the biomarker is capable of binding to more than one of the antigens. In embodiments, the binding domain comprises HA proteins from multiple influenza strains, e.g., at least one HA from an influenza A strain and at least one HA from an influenza B strain. In embodiments, the binding domain comprises F proteins from multiple parainfluenza virus (PIV) strains, e.g., at least one F protein from each of PIV1, PIV2, PIV3, and PIV4. In embodiments, the binding domain comprises S proteins and/or S-RBDs from multiple SARS-CoV-2 strains, e.g., as described in Tables 1A-1D.

In embodiments, the immunoassay method detects a biomarker that binds to an N protein from SARS-CoV-2. In embodiments, the immunoassay method detects a biomarker that binds to a S protein from SARS-CoV-2. In embodiments, the immunoassay method detects a biomarker that binds to S1, S2, S-ECD, S-NTD, or S-RBD from SARS-CoV-2. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A. In embodiments, the immunoassay method comprises: contacting the biological sample with a surface comprising a viral antigen in a binding domain on the surface; forming a binding complex in the binding domain comprising the viral antigen and a biomarker that binds to the viral antigen; and measuring the concentration of the biomarker in the binding complex. In embodiments, the biomarker is IgG, IgA, IgM, or combination thereof. In embodiments, the biomarker is an IgG, IgA, and/or IgM from a human, mouse, rat, ferret, minx, bat, or combination thereof. In embodiments, the concentration of the biomarker is measured by contacting the binding complex with a detection reagent that specifically binds IgG, IgA, or IgM as described herein. In embodiments, the immunoassay method is a classical serology assay. In embodiments, the immunoassay method is a bridging serology assay. In embodiments, the immunoassay method is a competitive serology assay. In embodiments, the detection reagent comprises a labeled competitor of the biomarker. In embodiments, the competitor is ACE2. Classical, bridging, and competitive serology assays are described herein.

In embodiments, the method is a multiplexed method capable of simultaneously detecting and/or quantifying the amounts of the one or more biomarkers that bind to a respiratory virus antigen. As discussed herein, a method that is capable of simultaneously testing for several potential causes of infection (e.g., multiple different viruses) can advantageously allow a respiratory virus infection to be correctly and efficiently diagnosed in a single assay run and utilizing a single patient sample. Such a method can also be useful for assessing a patient's immune response to different respiratory virus infections.

In embodiments, the multiplexed method simultaneously detects and/or quantifies one or more biomarkers that binds to S proteins from different strains SARS-CoV-2. In embodiments, the multiplexed method is capable of determining the SARS-CoV-2 strain that has infected an individual and/or the SARS-CoV-2 strain that is circulating in a population or geographical region, as described herein. As discussed herein, when referring to an S protein comprising a specific mutation, the mutation is relative to the SARS-CoV-2 reference strain NC_045512, and the S protein from the SARS-CoV-2 reference strain is also known as the “wild-type” S protein. Moreover, an S protein (or subunit thereof) referred to herein as being from a specific SARS-CoV-2 strain includes all of the S protein mutations of that strain as described herein.

In embodiments, the invention provides a method for determining a SARS-CoV-2 strain in a sample, comprising detecting at least a first antibody biomarker in the sample that binds to an antigen, e.g., an S protein, N protein, and/or S-RBD, from a first SARS-CoV-2 strain and at least a second antibody biomarker in the sample that binds to an antigen, e.g., an S protein, N protein, and/or S-RBD, from a second SARS-CoV-2 strain, wherein the detecting comprises contacting the sample with a surface comprising one or more binding domains, wherein the antigen, e.g., the S protein, N protein, or S-RBD from the first SARS-CoV-2 strain is immobilized on a first binding domain, and the antigen, e.g., the S protein, N protein, or S-RBD from the second SARS-CoV-2 strain is immobilized on a second binding domain; and determining a ratio of the first antibody biomarker to the second antibody biomarker, thereby determining the SARS-CoV-2 strain. In embodiments, the detecting comprises performing a multiplexed method described herein. In embodiments, the multiplexed method simultaneously detects and/or quantifies one or more biomarkers that bind to an antigen, e.g., an S-protein, N protein, and/or an S-RBD from two or more SARS-CoV-2 strains as shown in Table 1A, Table 1D, and/or Table 1E. In embodiments, the sample is a biological sample. In embodiments, the sample is from one or more individuals as described herein. In embodiments, the sample is a saliva sample.

In embodiments, each antigen is immobilized on a distinct binding domain on the surface, wherein the antigens comprise an S protein, an N protein, and/or an S-RBD from a SARS-CoV-2 strain described herein. In embodiments, the antigens comprise an S protein, an N protein, and/or an S-RBD from a SARS-CoV-2 strain selected from: an S protein, an S-RBD, and/or an N protein from a SARS-CoV-2 strain selected from: wild-type; P.1; P.2; P.3; B.1.1.519; B.1.1.529; B.1.1.529 (+R346K); B.1.1.529 (+L452R); BA.1; BA.1.1; BA.2; BA.3; B.1.1.7; B.1.1.7 (+E484K); B.1.258.17; B.1.351; B.1.351.1; B.1.429; B.1.466.2; B.1.525; B.1.526/E484K; B.1.526/S477N; B.1.526.1; B.1.617; B.1.617.1; B.1.617.2; B.1.617.2 (+ΔY144); B.1.617.2 (+E484K); B.1.617.2 (+E484K/N501Y); B.1.617.2 (+K417N/N439K/E484K/N501Y); B.1.617.2 (+K417N/E484K/N501Y); AY.1; AY.2; AY.3, AY.4; AY.5, AY.6, AY.7, AY.4.2; AY.12; AY.14; B.1.617.3; B.1.618; B.1.620; B.1.621; B.1.640.2; BV-1; A.23.1; A.VOI.V2; C.37; and R.1; and/or an S protein and/or an S-RBD from SARS-CoV-2 comprising one or more mutations selected from: R346K, V367F; Q414K, K417N, K417T, N439K, N450K, L452R, L452Q, S477N, T478K, T478R, E484K, E484Q, F490S, Q493R, N501Y.

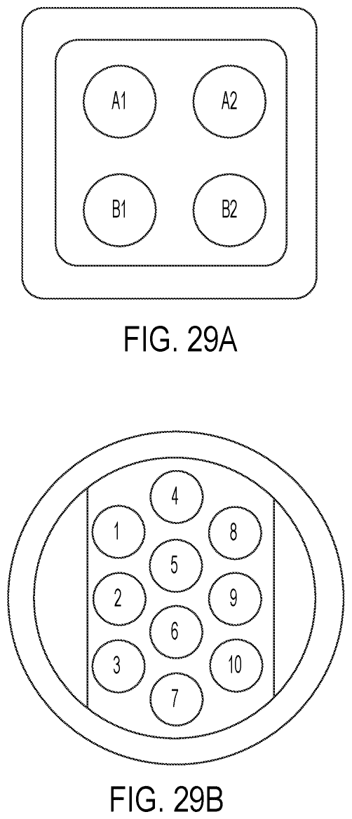

In embodiments, the multiplexed method simultaneously detects and/or quantifies one or more biomarkers that binds to a panel of antigens, wherein the panel of antigens comprises antigens from one or more respiratory viruses described herein. In embodiments, the multiplexed method simultaneously detects and/or quantifies one or more biomarkers that binds to a panel of SARS-CoV-2 antigens as shown in Tables 2A-2G. In embodiments, the S protein mutations from the SARS-CoV-2 strains of Tables 2A-2G are described in Table 1D. In embodiments, the S-RBD mutations from the SARS-CoV-2 strains of Tables 2A-2G are described in Table 1E. In embodiments, the SARS-CoV-2 antigens are immobilized on a surface. In embodiments, the surface comprises a well of a multi-well assay plate as described herein. In embodiments, the surface, e.g., well of a multi-well plate, comprises ten distinct binding domains (“Spots”), e.g., as shown in FIG. 29B. In embodiments, the SARS-CoV-2 antigens are immobilized on a surface comprising Spots 1-10 as shown in FIG. 29B, wherein the antigens are arranged as shown in Tables 2A-2G. In embodiments, the one or more biomarkers is IgG, IgA, IgM, or combination thereof. In embodiments, the IgG, IgA, and/or IgM is from a human, mouse, rat, ferret, minx, bat, or combination thereof. The Spots indicated with “BSA” in Tables 2A-2G below indicate an immobilized bovine serum albumin.

In embodiments, the multiplexed method simultaneously detects and/or quantifies one or more biomarkers that binds to a panel comprising one or more SARS-CoV-2 antigens and one or more antigens from a different respiratory virus, e.g., a different coronavirus than SARS-CoV-2 (e.g., SARS-CoV, MERS-CoV, HCoV-OC43, HCoV-229E, HCoV-NL63, or HCoV-HKU1), an influenza virus, an enterovirus, MPV, RSV, a rhinovirus, PIV, or a parechovirus.

In embodiments, the multiplexed method simultaneously detects and/or quantifies one or more biomarkers that binds to a panel of respiratory virus antigens as shown in Table 2H. In embodiments, the S protein mutations from the SARS-CoV-2 strains of Table 2H are described in Table 1D. In embodiments, the respiratory virus antigens are immobilized on a surface. In embodiments, the surface comprises a well of a multi-well assay plate as described herein. In embodiments, the surface, e.g., well of a multi-well plate, comprises ten distinct binding domains (“Spots”), e.g., as shown in FIG. 29B. In embodiments, the respiratory virus antigens are immobilized on a surface comprising Spots 1-10 as shown in FIG. 29B, wherein the antigens are arranged as shown in Table 2H. In embodiments, the one or more biomarkers is IgG, IgA, IgM, or combination thereof. In embodiments, the IgG, IgA, and/or IgM is from a human, mouse, rat, ferret, minx, bat, or combination thereof. The Spots indicated with “BSA” in Table 2H below indicate an immobilized bovine serum albumin.

TABLE 2A
SARS-CoV-2 Antigen Panels

| | ID | | | | |
|---|---|---|---|---|---|
| Spot | Panel 5 | Panel 6 | Panel 6A | Panel 7 | Panel 8 |
| 1 | wild-type Spike | wild-type Spike | wild-type Spike | wild-type Spike | wild-type Spike |
| 2 | BSA | S-D614G | S-D614G | 501Y.V2 S- | B.1.429 S-RBD |
| | | | | RBD | |
| 3 | wild-type N | wild-type N | wild-type N | wild-type N | wild-type N |
| 4 | BSA | BSA | BSA | P.1 S-RBD | B.1.526/E484K |
| | | | | | S-RBD |
| 5 | BSA | BSA | BSA | BSA | BSA |
| 6 | BSA | BSA | BSA | B.1.1.7 S-RBD | B.1.526/S477N |
| | | | | | S-RBD |
| 7 | P.1 Spike | P.1 Spike | P.1 Spike | P.1 Spike | B.1.526/E484K |
| | | | | | Spike |
| 8 | B.1.1.7 Spike | B.1.1.7 Spike | B.1.1.7 Spike | B.1.1.7 Spike | B.1.526/S477N |
| | | | | | Spike |
| 9 | 501Y.V2 Spike | 501Y.V2 Spike | 501Y.V2 Spike | 501Y.V2 Spike | B.1.429 Spike |
| 10 | BSA | wild-type S- | BSA | wild-type S- | wild-type S- |
| | | RBD | | RBD | RBD |

TABLE 2B
SARS-CoV-2 Antigen Panels

| | ID | | | | |
|---|---|---|---|---|---|
| | | Panel 9 | Panel 11 | Panel 9A | Panel 12 |
| | | S-RBDs with | S-RBDs with | S-RBDs with | S-RBDs with |
| Spot | Panel 8A | mutations: | mutations: | mutations: | mutations: |
| 1 | wild-type Spike | L452R | L452R | L452R | V367F |
| 2 | B.1.429 S-RBD | K417N, E484K, | K417N, E484K, | K417N, E484K | L452Q, F490S |
| | | N501Y | N501Y | N501Y | |
| 3 | wild-type N | E484K | E484K | E484K | E484K |
| 4 | B.1.526 S-RBD | K417T, E484K, | K417T, E484K, | K417T, E484K, | Q493R, N501Y |
| | | N501Y | N501Y | N501Y | |
| 5 | BSA | S477N | S477N | S477N | T478K |
| 6 | B.1.526.2 S- | N501Y | N501Y, A570D | N501Y | R346K, T478R, |
| | RBD | | | | E484K |
| 7 | BSA | E484K, N501Y | E484K, N501Y | E484K, N501Y | E484K, N501Y |
| 8 | B.1.526 S-RBD | L452R, E484Q | L452R, E484Q | L452R, E484Q | L452R, E484Q |
| 9 | B.1.429 Spikc | Q414K, N450K | Q414K, N450K | L452R, T478K | L452R, T478K |
| 10 | wild-type S- | wild-type S- | wild-type S- | wild-type S- | wild-type S- |
| | RBD | RBD | RBD | RBD | RBD |

TABLE 2C
SARS-CoV-2 Antigen Panels

| | ID | | | | |
|---|---|---|---|---|---|
| | Panel 13 | Panel 14 | Panel 15 | Panel 16 | |
| | Spike from | Spike from | Spike from | S-RBDs with | |
| Spot | strains: | strains: | strains: | mutations: | Panel 17 |
| 1 | wild-type | wild-type | wild-type | K417N, L452R, | wild-type Spike |
| | | | | T478K | |
| 2 | P.2 | A.23.1 | AY.1 | K417N, E484K, | S-D614G |
| | | | | N501Y | |
| 3 | B.1.617.1 | A.VOI.V2 | AY.2 | E484K | wild-type N |
| 4 | B.1.617.2 | B.1.617.2 | B.1.617.2 + | S477N and | B.1.617.2 Spike |
| | | (Alt Seq 1) | ΔY144 | E484K | (Alt Seq 1) |
| 5 | B.1.617.3 | C.37 | B.1.620 | BSA | BSA |
| 6 | B.1.617 | R.1 | B.1.258.17 | BSA | BSA |
| 7 | P.1 | P.3 | B.1.466.2 | E484K, N501Y | P.1 Spike |
| 8 | B.1.1.7 | B.1.525 | B.1.1.7 + E484K | N439K | B.1.1.7 Spike |
| 9 | B.1.351 | B.1.1.519 | B.1.351.1 | L452R, T478K | B.1.351 Spike |
| 10 | B.1.526.1 | BV-1 | B.1.618 | wild-type S- | wild-type S- |
| | | | | RBD | RBD |

TABLE 2D
SARS-CoV-2 Antigen Panels

| | ID | | | | |
|---|---|---|---|---|---|
| | Panel 18 | Panel 18A | Panel 19 | Panel 20 | Panel 21 |
| | Spike from | Spike from | Spike from | Spike from | Spike from |
| Spot | strains: | strains: | strains: | strains: | strains: |
| 1 | wild-type | wild-type | wild-type | wild-type | wild-type |
| 2 | P.2 | P.2 | B.1.621 | B.1.617.2 + | B.1.1.529 |
| | | | | K417N, N439K, | |
| | | | | E484K, N501Y | |
| 3 | B.1.617.1 | BSA | AY.2 | B.1.617.2 + | AY.4.2 |
| | | | (Alt Seq 1) | K417N, E484K, | |
| | | | | N501Y | |
| 4 | B.1.617.2 | BSA | B.1.617.2 | AY.4 | AY.4 |
| | (Alt Seq 2) | | (AY.4) | (Alt Seq 2) | |
| | | | (Alt Seq 2) | | |
| 5 | B.1.617.3 | B.1.617.3 | C.37 | B.1.617.2 + | BSA |
| | | | | E484K, N501Y | |
| 6 | B.1.617 | B.1.617 | AY.12 | B.1.617.2 + | BSA |
| | | | | E484K | |
| 7 | P.1 | P.1 | P.1 | P.1 | P.1 |
| 8 | B.1.1.7 | B.1.1.7 | AY.1 | B.1.1.7 | B.1.1.7 |
| | | | (Alt Seq 1) | | |
| 9 | B.1.351 | BSA | B.1.351 | B.1.351 | B.1.351 |
| 10 | B.1.526.1 | BSA | B.1.617.2 | B.1.617.2 | B.1.617.2 |
| | | | (Alt Seq 1) | (Alt Seq 1) | (Alt Seq 1) |

TABLE 2E
SARS-CoV-2 Antigen Panels

| | ID | | | | |
|---|---|---|---|---|---|
| | Panel 22 | | Panel 25 | Panel 26 | Panel 27 |
| | S-RBD from | | Spike from | S-RBD from | Spike from |
| Spot | strains: | Panel 24 | strains: | strains: | strains: |
| 1 | B.1.1.529 | wild-type Spike | wild-type | B.1.1.529 (BA.1) | wild-type |
| 2 | B.1.351 | B.1.1.529 Spike | B.1.1.529 | B.1.351 | BA.2.12.1 |
| 3 | BSA | wild-type N | BA.2 | BA.2 | BA.2 |
| 4 | P.1 | AY.4 Spike | AY.4 | P.1 | AY.4 |
| | | (Alt Seq 2) | (Alt Seq 2) | | (Alt Seq 2) |
| 5 | BSA | BSA | BA.3 | BSA | BA.3 |
| 6 | B.1.1.7 | BSA | B.1.1.529 + | B.1.1.7 | BA.2 + L452M |
| | | | R346K | | |
| 7 | BSA | P.1 Spike | B.1.1.529 + | BSA | BA.2 + L452R |
| | | | L452R | | |
| 8 | BSA | B.1.1.7 Spike | B.1.1.7 | BA.1.1 | BA.4 |
| 9 | B.1.617.2 | B.1.351 Spike | B.1.351 | B.1.617.2 | B.1.351 |
| 10 | wild-type S- | wild-type S- | B.1.640.2 | wild-type | BA.5 |
| | RBD | RBD | | | |

TABLE 2F
SARS-CoV-2 Antigen Panels

| | ID | | | | |
|---|---|---|---|---|---|
| | Panel 28 | Panel 29 | Panel 30 | | |
| | S-RBD from | Spike from | S-RBD from | | |
| Spot | strains: | strains: | strains: | Panel VS-1 | Panel VR-1 |
| 1 | BA.2.12.1 | wild-type | BA.2.12.1 | wild-type Spike | BA.2.12.1 S-RBD |
| 2 | B.1.351 | BA.2.12.1 | B.1.351 | BA.2.12.1 Spike | B.1.351 S-RBD |
| 3 | BA.2 | BA.2 | BA.2 | wild-type N | wild-type N |
| 4 | BA.2 + L452M | AY.4 | BSA | BA.2.75 Spike | BA.1 S-RBD |
| | | (Alt Seq 2) | | | |
| 5 | BA.2 + L452R | BSA | BSA | BA.2 Spike | BA.2 S-RBD |
| 6 | B.1.1.7 | BSA | B.1.1.7 | BA.1 Spike | B.1.1.7 S-RBD |
| 7 | BA.4/BA.5 | BA.2.75 | BA.4/BA.5 | B.1.617.2 Spike | B.1.617.2 S-RBD |
| | | | | (Alt Seq 2) | |
| 8 | BA.3 | BA.4 | BA.2.75 | B.1.1.7 Spike | BA.2.75 S-RBD |
| 9 | AY.4 | B.1.351 | AY.3 | B.1.351 Spike | BA.4/BA.5 S-RBD |
| 10 | wild-type | BA.5 | wild-type | BA.5 Spike | wild-type S-RBD |

TABLE 2G
SARS-CoV-2 Antigen Panels

| | ID | | | |
|---|---|---|---|---|
| | Panel 32 | Panel 33 | Panel 34 | Panel 35 |
| | Spike from | S-RBD from | Spike from | S-RBD from |
| Spot | strains: | strains: | strains: | strains: |
| 1 | wild-type | BA.1 | wild-type | BA.1 |
| 2 | BA.1 | BQ.1.1 | BA.1 | BQ.1.1 |
| 3 | XBB.1 | BA.2.75.2 | XBB.1 | XBB.1.5 |
| 4 | BF.7 | BA.4.6/BF.7 | BF.7 | BN.1 |
| 5 | BA.2.75.2 | BSA | XBB.1.5 | BSA |
| 6 | BQ.1.1 | XBB.1 | BQ.1.1 | XBB.1 |
| 7 | BA.2.75 | BA.4/BA.5/BF.5 | BA.2.75 | BA.5 |
| 8 | BA.4.6 | BA.2.75 | BN.1 | BA.2.75 |
| 9 | BQ.1 | BQ.1 | BQ.1 | BQ.1 |
| 10 | BA.5 | wild-type | BA.5 | wild-type |

TABLE 2H
Respiratory Virus Antigen Panels

| | ID | |
|---|---|---|
| | Panel R5 | Panel R6 |
| | Virus | Virus |
| Spot | Antigen | Antigen |
| 1 | Enterovirus (EV)-D68 | Flu A/H1 (e.g., |
| | VLP | H1/Wisconsin 2019) |
| | | HA |
| 2 | Enterovirus (EV)-71 | Flu A/H3 (e.g., |
| | VLP | H3/Darwin 2021) |
| | | HA |
| 3 | Metapneumovirus (MPV) | Flu B/Victoria (e.g., |
| | F | Austria 2021) |
| | | HA |
| 4 | RSV | BSA |
| | Pre-Fusion F | |
| 5 | Flu A/H1 (e.g., H1/Wisconsin 2019), | BSA |
| | Flu A/H3 (e.g., H3/Darwin 2021), | |
| | and Flu B/Victoria (e.g., | |
| | Austria 2021) | |
| | Mixture of HAS | |
| 6 | Rhinovirus C (RV-C) | PIV1 |
| | VP0 | F |
| 7 | SARS-CoV-2 BA.5 | PIV2 |
| | Spike | F |
| 8 | PIV1, PIV2, PIV3, and PIV4 | PIV3 |
| | Mixture of Fs | F |
| 9 | Parechovirus (PeV)-A3 | PIV4 |
| | VP0 | F |
| 10 | BSA | BSA |

In embodiments, the multiplexed method comprises: contacting the biological sample with a surface as described herein that comprises an immobilized viral (e.g., SARS-CoV-2) antigen in each binding domain on the surface; forming a binding complex in each binding domain comprising the viral antigen and a biomarker that binds to the viral antigen; and measuring the concentration of the biomarker in each binding complex. In embodiments, biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises four distinct binding domains. In embodiments, the surface comprises a single assay plate. In embodiments, the surface comprises a multi-well assay plate, wherein each well comprises ten distinct binding domains.

In embodiments, the immunoassay method comprises detecting one or more viral antigens that are specific to SARS-CoV-2. As discussed herein, SARS-CoV-2 causes the respiratory illness COVID-19, which can cause mild to severe symptoms in patients. Sensitive and specific detection of SARS-CoV-2 is important for providing an accurate diagnosis, identifying asymptomatic infected individuals, and tracking spread of the disease. A method that detects biomarkers produced by an individual in response to a SARS-CoV-2 infection (e.g., antibodies) is also useful for identifying those who may be immune to the virus and therefore may be at lower risk when interacting with the general public or infected patients, and also may be potential candidates for plasma transfusions. In embodiments, the one or more biomarkers is capable of binding to a SARS-CoV-2 S-D614 protein, S-D614G, S1 subunit, S2 subunit, S-NTD, S-RBD, M protein, E protein, N protein, or a combination thereof. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the multiplexed method is capable of simultaneously quantifying the one or more biomarkers that bind to a SARS-CoV-2 antigen. In embodiments, the immunoassay comprises: (a) contacting the biological sample with the viral antigen that specifically binds to a first biomarker of the one or more biomarkers; (b) forming a binding complex comprising the viral antigen and the first biomarker; and (c) measuring the concentration of the first biomarker in the binding complex. In embodiments, the method further comprises repeating one or more of the method steps described herein to quantify the amounts of one or more biomarkers in the sample. In embodiments, the method further comprises repeating steps (a)-(c), wherein each biomarker specifically binds to a different viral antigen, thereby quantifying one or more biomarkers. In embodiments, each of steps (a)-(c) is performed for each biomarker in parallel.

In embodiments, the multiplexed method is capable of simultaneously quantifying at least two biomarkers in the biological sample, wherein each of the at least two biomarkers is independently capable of binding to a viral antigen, e.g., any of HA, F, S, S1, S2, S-NTD, S-ECD, S-RBD, M, E, or N as described herein. In embodiments, the multiplexed method is capable of simultaneously quantifying two, three, four, five, or more than five biomarkers in the biological sample, wherein each biomarker is independently capable of binding to a viral antigen, e.g., any of HA, F, S, S1, S2, S-NTD, S-ECD, S-RBD, M, E, or N as described herein. In embodiments, the multiplexed method comprising quantifying a combination of the biomarkers provided herein has improved sensitivity and/or dynamic range, compared to a method in which only a single biomarker is quantified. For example, a multiplexed method can provide earlier and more sensitive detection compared to a method that detects a single biomarker, since responses to each viral antigen may vary between individuals. Moreover, the ability to simultaneously measure antibody responses against multiple similar viruses, e.g., a newly-emerged coronavirus such as SARS-CoV-2 and similar coronaviruses viruses such as hCoV-OC43, hCoV-HKU1, and hCoV-NL63, which have been circulating in the general population, improves understanding of how an individual's prior exposure to similar circulating viruses affects the individual's response to the newly-emerged virus of interest.

In embodiments, the method is used to diagnose whether a subject is infected with a virus, e.g. SARS-CoV-2. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is a coronavirus. In embodiments, the virus is SARS-CoV-2.

In embodiments, the method is used to identify individuals with previous virus exposure for epidemiological studies (e.g., to understand true disease prevalence and evaluate the efficacy of infection control measures). In embodiments, the method is used to identify individuals at lower risk of future infection. Moreover, the method can be an important tool in the research, development, and validation of a vaccine for the virus. In embodiments, the method is used to assess differences in immune responses (e.g., antibody response) between individuals whose immunity is achieved by natural infection or vaccination. For example, a multiplexed method differentiates an individual's response to vaccination with different constructs of a viral antigen (e.g., different fragments of the S protein), compared with the individual's response to natural infection by the virus. Such a method can advantageously distinguish between individuals with biomarkers produced in response an active infection and are potentially contagious and individuals with biomarkers produced in response to the vaccine. In embodiments, the virus is a coronavirus. In embodiments, the virus is SARS-CoV-2.

In embodiments, the biomarker capable of binding to a viral antigen is an immune biomarker. In embodiments, the biomarker is an antibody or antigen-binding fragment thereof. In embodiments, the biomarker is an immunoglobulin A (IgA), immunoglobulin G (IgG; including IgG subclasses IgG1, IgG2, IgG3, and IgG4), immunoglobulin M (IgM), immunoglobulin E (IgE), or immunoglobulin D (IgD), or antigen-binding fragments thereof capable of binding to S, S1, S2, S-NTD, S-ECD, S-RBD, M, E, and/or N. In embodiments, the IgG, IgA, IgM, IgD, and/or IgE is from a human, mouse, rat, ferret, minx, bat, or combination thereof. In embodiments, the biomarker is an IgA or antigen-binding fragment thereof capable of binding to S, S1, S2, S-NTD, S-ECD, S-RBD, M, E, and/or N. In embodiments, the biomarker is an IgG or antigen-binding fragment thereof capable of binding to S, S1, S2, S-NTD, S-ECD, S-RBD, M, E, and/or N. In embodiments, the biomarker is an IgG1 or antigen-binding fragment thereof capable of binding to S, S1, S2, S-NTD, S-ECD, S-RBD, M, E, and/or N. In embodiments, the biomarker is an IgG2 or antigen-binding fragment thereof capable of binding to S, S1, S2, S-NTD, S-ECD, S-RBD, M, E, and/or N. In embodiments, the biomarker is an IgG3 or antigen-binding fragment thereof capable of binding to S, S1, S2, S-NTD, S-ECD, S-RBD, M, E, and/or N. In embodiments, the biomarker is an IgG4 or antigen-binding fragment thereof capable of binding to S, S1. S2, S-NTD, S-ECD, S-RBD, M, E, and/or N. In embodiments, the biomarker is an IgM or antigen-binding fragment thereof capable of binding to S, S1, S2, S-NTD, S-ECD, S-RBD, M, E, and/or N. In embodiments, the biomarker is an IgE or antigen-binding fragment thereof capable of binding to S, S1, S2, S-NTD, S-ECD, S-RBD, M, E, and/or N. In embodiments, the biomarker is an IgD or antigen-binding fragment thereof capable of binding to S, S1, S2, S-NTD, S-ECD, S-RBD, M, E, and/or N. In embodiments, the viral antigen is a coronavirus antigen. In embodiments, the coronavirus is SARS-CoV-2. In embodiments, the biomarker binds to SARS-CoV-2 S-D614. In embodiments, the biomarker binds to SARS-CoV-2 S-D614G. In embodiments, the biomarker binds to a SARS-CoV-2 S protein or subunit or fragment thereof that comprises a mutation as shown in Tables 1A and 1B. In embodiments, the biomarker binds to a SARS-CoV-2 N protein that comprises a mutation as shown in Table 1A.

In embodiments, the biomarker to be detected is an antibody biomarker, and the binding reagent is a viral antigen that is bound by the antibody biomarker. In embodiments, the binding reagent is a viral protein described herein, e.g., HA, F, S, S1, S2, S-NTD, S-ECD, S-RBD, M, E, N.

In embodiments, the binding reagent is a peptide antigen. Peptide antigens are short peptides of a native, full-length protein that include the antibody binding epitope. Peptide antigens can be easier to produce and provide greater flexibility in performing an immunoassay to detect an antibody biomarker. Peptide antigens can also have higher specificity to the antibody biomarker compared with a full-length viral protein or domain described herein. In embodiments, an immunoassay utilizing a peptide antigen as the binding reagent has reduced cross-reactivity with antibody biomarkers for a different virus that are present in the biological sample. For example, an immunoassay utilizing a SARS-CoV-2 peptide antigen can have reduced cross-reactivity for antibodies that may be present in a subject for a circulating coronavirus.

In embodiments, the peptide antigen is a fragment of a viral protein, e.g., a coronavirus protein. In embodiments, the peptide antigen comprises about 10 to about 100 amino acids. In embodiments, the peptide antigen comprises about 20 to about 80 amino acids. In embodiments, the peptide antigen comprises about 30 to about 60 amino acids. In embodiments, the peptide antigen comprises about 40 to about 50 amino acids. In embodiments, the peptide antigen is a fragment of S, S1, S2, S-NTD, S-ECD, S-RBD, M, E, or N. In embodiments, the peptide antigen comprises an immunodominant region (IDR) of a viral protein. In embodiments, the peptide antigen comprises amino acids 1-49 of the N protein IDR. In embodiments, the peptide antigen comprises amino acids 340-390 of the N protein TDR. In embodiments, the peptide antigen comprises amino acids 192-220 of the of the N protein IDR. In embodiments, the peptide antigen comprises amino acids 182-216 of the M protein IDR.

IgA, IgG (and subclasses thereof), IgM, IgE, and IgD are different isotypes of antibodies that have different immunological properties and functional locations. For example, IgA is typically found in the mucosal areas, such as the respiratory and gastrointestinal tracts, saliva, and tears and can prevent colonization by pathogens. IgG, the most abundant antibody isotype, has four subclasses as described herein and is found in all bodily fluids and provides the majority of antibody-based immunity against pathogens. IgM is mainly found in the blood and lymph fluid and is typically the first antibody made by the body to fight a new infection. IgE is mainly associated with allergic reactions (e.g., as part of aberrant immune response) and is found in the lungs, skin, and mucous membranes. IgD mainly functions as an antigen receptor on B cells and may activate basophils and mast cells to produce antimicrobial factors. Based on the timing and/or type of infection, different amounts of each isotype are produced.

In embodiments, the method is a multiplexed immunoassay method capable of quantifying the amount of each isotype of antibodies, e.g., IgG, IgA, IgE, and IgM, present in the biological sample. In embodiments, the amounts of the different isotypes of antibodies measured in a biological sample, e.g., the amounts of each of IgG, IgA, IgE, and IgM, can be used to determine whether a subject has been previously exposed to a virus. In embodiments, the amounts of the different isotypes of antibodies measured in a biological sample, e.g., the amounts of each of IgG, IgA, IgE, and IgM, can be used to estimate the time of virus exposure and/or infection. In embodiments, the amounts of the different isotypes of antibodies measured in a biological sample, e.g., the amounts of each of IgG, IgA, IgE, and IgM, can be used to determine whether a subject has immunity to a virus, e.g., a coronavirus such as SARS-CoV-2.

In embodiments, the method comprises: (a) contacting the biological sample with: at least a first, second, third, and fourth viral antigens, wherein each viral antigen specifically binds to IgG, IgA, IgE, and IgM, respectively; (b) forming at least a first, second, third, and fourth binding complex comprising the viral antigens and IgG, IgA, IgE, or IgM; and (c) measuring the concentration of IgG, IgA, IgE, or IgM in each of the binding complexes. In embodiments, each viral antigen is independently S, S1, S2, S-NTD, S-ECD, S-RBD, M, E, N, or a peptide antigen described herein. In embodiments, the IgG, IgA, IgE, and/or IgM is from a human, mouse, rat, ferret, minx, bat, or combination thereof.

IgG is further divided into four subclasses, IgG1, IgG2, IgG3, and IgG4, based on properties such as ability to activate complement, bind to macrophages, and/or pass through the placenta. Each subclass also has a distinct biological function. For example, the response to protein antigens is primarily mediated by IgG1 and IgG3, while IgG2 primarily mediates the response to polysaccharide antigens. IgG4 plays a role in protection against certain hypersensitivity reactions and pathogenesis of some autoimmune diseases. IgG subclass screening is performed to monitor a subject's infection response and/or determine whether a subject has antibody deficiency, and/or assess a subject's risk of an adverse response to infection. In embodiments, the method comprises determining the amount of IgG1, IgG2, IgG3, and IgG4 in the biological sample. In embodiments, the IgG is from a human, mouse, rat, ferret, minx, bat, or combination thereof.

In embodiments, the method comprises: (a) contacting the biological sample with: at least a first, second, third, and fourth viral antigens, wherein each viral antigen specifically binds to IgG1, IgG2, IgG3, and IgG4 respectively; (b) forming at least a first, second, third, and fourth binding complex comprising the viral antigens and IgG1, IgG2, IgG3, or IgG4; and (c) measuring the concentration of IgG1, IgG2, IgG3, or IgG4 in each of the binding complexes. In embodiments, each viral antigen is independently S, S1, S2, S-NTD, S-ECD, S-RBD, M, E, N, or a peptide antigen described herein. In embodiments, the IgG is from a human, mouse, rat, ferret, minx, bat, or combination thereof.

In embodiments, the method comprises: (a) contacting the biological sample with: a plurality of viral antigens, wherein each viral antigen specifically binds to an immunoglobulin selected from IgG1, IgG2, IgG3, IgG4, IgA, IgE, and IgM; (b) forming a plurality of binding complexes comprising the viral antigens and immunoglobulins; and (c) measuring the concentration of the immunoglobulin in each of the binding complexes. In embodiments, each viral antigen is independently S, S1, S2, S-NTD, S-ECD, S-RBD, M, E, N, or a peptide antigen described herein. In embodiments, the IgG, IgA, IgE, and/or IgM is from a human, mouse, rat, ferret, minx, bat, or combination thereof.

### Inflammatory/Tissue Damage Response Biomarkers

In embodiments, the invention provides a method for detecting a biomarker in a subject to detect a viral infection, e.g., by a respiratory virus, including coronaviruses such as SARS-CoV-2. In embodiments, the invention provides a method for detecting a biomarker in a subject to assess the severity and/or prognosis of a viral infection. e.g., by a respiratory virus, including coronaviruses such as SARS-CoV-2. In embodiments, the biomarker is produced in response to the viral infection. In embodiments, the biomarker is a stress response protein. In embodiments, the biomarker is an inflammatory response biomarker. In embodiments, the biomarker is a tissue damage response biomarker. In embodiments, the biomarker is a T cell activation biomarker. In embodiments, the biomarker is an extracellular vesicle.

In embodiments, the binding reagent that specifically binds the biomarker described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof.

### Extracellular Vesicles

In embodiments, the biomarker is an extracellular vesicle. Extracellular vesicles, also known as EVs or exosomes, are small membrane vesicles released by most cell types. For example, virus-infected cells release EVs that can mediate further in vivo viral spread in a variety of ways and produce other pathogenic effects. For example, EVs have been shown to transfer membrane-associated viral proteins, viral cargo proteins or RNAs, indirectly assist pathogens in escaping the immune system, or inhibit an immune response. EVs can also transfer viral genes from SARS-CoV-2 infected to non-infected cells and can induce inflammation in the absence of direct viral infection.

In embodiments, detecting EVs from infected cells is used to identify reservoirs of infection. In embodiments, EV populations in a biological sample are analyzed to determine the mechanism of infection, disease prognosis, and adaptive immunity. In embodiments, an EV released from a particular cell, e.g., an immune cell, comprises one or more of the same surface marker as that cell. In embodiments, the biomarker is an EV comprising an inflammatory damage and/or a tissue damage protein as described herein, on the surface of the EV.

### Viral Component and Biomarker Detection

In embodiments, the invention provides a method comprising simultaneously detecting a host biomarker (e.g., an antibody biomarker or inflammatory and/or tissue damage response biomarker) described herein and a viral component described herein. A method that simultaneously determines, from a single sample, whether a subject is infected by a virus (e.g., a coronavirus such as SARS-CoV-2) and assesses the subject's immune response is capable of determining the subject's disease prognosis, for example, determining whether the subject will likely have poor disease progression and increased likelihood of intensive care treatment. Thus, the method enables preparation of an early response to a potentially serious illness.

In embodiments, the method is a multiplexed immunoassay method. In embodiments, the multiplexed immunoassay method detects a viral nucleic acid, a host antibody biomarker, a host inflammatory and/or tissue damage response biomarker, or a combination thereof.

In embodiments, a subject's infection status, disease progression, prognosis, or combination thereof is assessed by simultaneously detecting (1) a viral component, (2) a host antibody biomarker, and (3) a host inflammatory and/or tissue damage response biomarker as described herein. For example, Table 3 provides exemplary outcomes and assessments based on the combined detection for diagnosis and prognosis of COVID-19, the disease caused by SARS-CoV-2 infection.

TABLE 3
Exemplary Scenarios and Predicted Outcomes

| | SARS-CoV-2 | Host | Inflammatory/Tissue | COVID-19 |
|---|---|---|---|---|
| Scenario | Viral | Antibody | Damage Response (e.g., | Diagnosis, |
| # | Component | (Serology) | cytokine/chemokine) | Disease Stage |
| 1 | + | − | Normal | Early stage; |
| | | | | asymptomatic |
| 2 | − | + | Normal | Convalescent; |
| | | | | recovered |
| 3 | + | + | Elevated | Late stage, active |
| | | | | infection |
| 4 | − | − | Elevated | Non-COVID-19 |
| | | | | inflammation |
| 5 | + | +/− | Highly Elevated | Active acute stage |
| | | | | infection. May require |
| | | | | hospital admission |
| 6 | − | − | Normal | No exposure to |
| | | | | COVID-19 |

### Samples and Assay Devices

In embodiments, the viruses, viral components, and/or biomarkers described herein are measured in a biological sample. In embodiments, the biological sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, a respiratory sample, bronchial/bronchoalveolar lavage, saliva, mucus, oropharyngeal swab, sputum, endotracheal aspirate, pharyngeal/nasal swab, throat swab, amniotic fluid, nasal secretions, nasopharyngeal wash or aspirate, nasal mid-turbinate swab, vaginal secretions, a surface biopsy, sperm, semen/seminal fluid, wound secretions and excretions, ear secretions or discharge, or an extraction, purification therefrom, or dilution thereof. In embodiments, the biological sample is diluted such that the assay signal is within the upper and lower detection limits of the assay. In embodiments, the biological sample is diluted to achieve a desired assay sensitivity. Further exemplary biological samples include but are not limited to physiological samples, samples containing suspensions of cells such as mucosal swabs, tissue aspirates, endotracheal aspirates, tissue homogenates, cell cultures, and cell culture supernatants. In embodiments, the biological sample is a respiratory sample obtained from the respiratory tract of a subject. Examples of respiratory samples include, but are not limited to, bronchial/bronchoalveolar lavage, saliva, mucus, endotracheal aspirate, sputum, nasopharyngeal/nasal swab, throat swab, oropharyngeal swab and the like. In embodiments, the biological sample is whole blood, serum, plasma, cerebrospinal fluid (CSF), urine, saliva, sputum, endotracheal aspirate, nasopharyngeal/nasal swab, bronchoalveolar lavage, or an extraction or purification therefrom, or dilution thereof. In embodiments, the biological sample is blood that has been dried and reconstituted. In embodiments, the biological sample is serum or plasma. In embodiments, the plasma is in EDTA, heparin, or citrate. In embodiments, the biological sample is saliva. In embodiments, the biological sample is endotracheal aspirate. In embodiments, the biological sample is a nasal swab. In embodiments, the virus, viral component, and/or biomarkers described herein have substantial levels in the saliva or endotracheal aspirate of a subject. In embodiments, the virus, viral components, and/or biomarkers described herein are present in higher amounts in certain bodily fluids (e.g., saliva) compared to others (e.g., throat swab). In embodiments, certain antibody biomarker levels, e.g., IgG (including subclasses thereof) and IgA, are substantially similar in blood and saliva of a subject. In embodiments, the ratio of antibody levels to different components from a virus (e.g., SARS-CoV-2 S and N proteins) are highly correlated in blood and saliva of a subject. In embodiments, the ratio of antibody levels to different components from a virus, e.g., the ratio of the antibody levels against the SARS-CoV-2 S protein and the SARS-CoV-2 N protein is used to assess the immune response and/or clinical outcome of a subject infected with SARS-CoV-2.

In embodiments, the biological sample is from an animal. In embodiments, the biological sample from an animal is useful for animal model studies, e.g., for vaccine and/or drug research and development, and/or to better understand disease progression and infection lethality. Exemplary animals that are useful for animal model studies include, but are not limited to, mouse, rat, rabbit, pig, primate such as monkey, and the like.

In embodiments, the biological sample is from a human or an animal subject. In embodiments, the subject is susceptible or suspected to be susceptible to infection by the viruses described herein. In embodiments, the subject is known or suspected to transmit the viruses described herein. Virus transmission may occur among the same species (e.g., human-to-human) or inter-species (e.g., bat-to-human). Non-limiting examples of animal subjects include domestic animals, such as dog, cat, horse, goat, sheep, donkey, pig, cow, chicken, duck, rabbit, gerbil, hamster, guinea pig, and the like; non-human primates (NHP) such as macaque, baboon, marmoset, gorilla, orangutan, chimpanzee, monkey, and the like; big cats such as tiger, lion, puma, leopard, snow leopard, and the like; and other mammals such as bats and pangolins. In embodiments, the biological sample is from a human, a mouse, a rat, a ferret, a minx, or a bat. In embodiments, the subject is a host that has been exposed to and/or infected by a virus as described herein. In embodiments, the biological ample comprises a plasma (e.g., in EDTA, heparin, or citrate) sample from a subject. In embodiments, the biological sample comprises a serum sample from a subject. In embodiments, the biological sample is from a healthy subject. In embodiments, the biological sample is from a subject known to never have been exposed to a virus described herein. In embodiments, the biological sample is from a subject known to be immune to a virus described herein. In embodiments, the biological sample is from a subject known to be infected with a virus described herein. In embodiments, the biological sample is from a subject suspected of having been exposed to a virus described herein. In embodiments, the biological sample is from a subject at risk of being exposed to a virus described herein. In embodiments, the virus is a coronavirus. In embodiments, the virus is SARS-CoV-2.

In embodiments, the sample is an environmental sample. In embodiments, the environmental sample is aqueous, including but not limited to, fresh water, drinking water, marine water, reclaimed water, treated water, desalinated water, sewage, wastewater, surface water, ground water, runoff, aquifers, lakes, rivers, streams, oceans, and other natural or non-natural bodies of water. In embodiments, the aqueous sample contains bodily solids or fluids (e.g., feces or urine) from subjects who have been exposed to or infected with a virus herein (e.g., a coronavirus such as SARS-CoV-2). In embodiments, the environmental sample is from a air filtration device, e.g., air filters in a healthcare or long-term care facility or other communal places of gathering. Detection of a virus described herein (e.g., a coronavirus such as SARS-CoV-2) in an environmental sample can provide early identification and/or tracing of an outbreak or potential outbreak, thereby allowing a more prompt and robust response. Moreover, detection of a biomarker, e.g., one or more antibody biomarkers that specifically binds a viral antigen (e.g., from a coronavirus such as SARS-CoV-2) in an environmental sample can provide an estimation of the percentage of a population with detectable antibodies against the virus (i.e., seroconversion), which is useful for epidemiology studies. In some embodiments, the sample comprises wastewater. Detection of SARS-CoV-2 in wastewater is described, e.g., in U.S. Publication No. 2022/0003766 and U.S. Publication No. 2021/0349104.

Wastewater samples are also useful for determining the viral strain, i.e., the genotype, of SARS-CoV-2 in a population. SARS-CoV-2 strains are further described herein and include, e.g., the L strain and the S strain, which differ at genome locations 8782 and 28144; and the S-D614 strain and the S-D614G strain, which differ by a single polynucleotide at genome location 23403, and the strains described in Table 1A, e.g., strains B.1.1.7, 501Y.V2, P.1, and Cal.20C. In embodiments, the invention provides a method for detecting SARS-CoV-2 nucleic acid in a wastewater sample, comprising: a) contacting the wastewater sample with a binding reagent that specifically binds a SARS-CoV-2 nucleic acid; b) forming a binding complex comprising the binding reagent and the SARS-CoV-2 nucleic acid; and c) detecting the binding complex, thereby detecting the SARS-CoV-2 nucleic acid in the wastewater sample. In embodiments, the SARS-CoV-2 nucleic acid comprises a SARS-CoV-2 single nucleotide polymorphism (SNPs) or mutation as described herein, e.g., in Tables 1A and 1C. In embodiments, the method is a multiplexed method that simultaneously detects one or more, two or more, three or more, four or more, five or more, six or more, seven or more, eight or more, nine or more, or ten or more SARS-CoV-2 SNPs. Methods of detecting SNPs in viral nucleic acids, e.g., SARS-CoV-2 RNA, are provided herein. In embodiments, levels of IgA, IgG, and/or IgM in wastewater samples are used as controls for normalizing the detected amount of viral protein and/or genetic material (e.g., RNA) in the wastewater sample.

In embodiments where the sample comprises a liquid (e.g., endotracheal aspirate, saliva, blood, serum, plasma and the like), the sample is about 0.05 mL to about 50 mL, about 0.1 mL to about 10 mL, about 0.2 mL to about 5 mL, or about 0.3 mL to about 3 mL. In embodiments where the sample is solid or semi-solid (e.g., a swab such as a nasopharyngeal swab or oropharyngeal swab, mucus, sputum and the like), the sample is provided into a storage liquid of about 0.05 mL to about 50 mL, about 0.1 mL to about 10 mL, about 0.2 mL to about 5 mL, or about 0.3 mL to about 3 mL. In embodiments, the storage liquid is Viral Transport Medium (VTM), Amies transport medium, or sterile saline. In embodiments, the storage liquid comprises a substance for stabilizing nucleic acids, e.g., EDTA. In embodiments, the storage liquid comprises a reagent for inactivating live virus as described herein.

In embodiments, the sample comprises saliva. In embodiments, the invention provides a method of identifying a saliva sample in which the viral component and/or biomarker of interest has degraded, i.e., a low quality saliva sample. In embodiments, a low quality saliva sample is not suitable for the assays described herein. In embodiments, a low quality saliva sample comprises low levels of total antibodies as compared to a freshly obtained sample and/or as compared to a threshold total antibody level. In embodiments, a low quality saliva sample comprises low levels of IgA as compared to a freshly obtained sample and/or as compared to a threshold antibody level. In embodiments, the threshold antibody level is determined based on the average of an aggregate of samples. In embodiments, a low quality saliva sample comprises low levels of antibodies against circulating coronaviruses (e.g., hCoV-NL63, hCoV-HKU1, hCoV-229E, and/or hCoV-OC43) as compared to a freshly obtained sample and/or a threshold antibody level. In embodiments, identifying the low quality saliva sample comprises determining the total antibody level in a sample and, if the sample has low antibody levels as compared to a freshly isolated control sample and/or as compared to a threshold total antibody level, identifying the sample as a low quality saliva sample. In embodiments, identifying the low quality saliva sample comprises determining the IgA level in a sample and, if the sample has low IgA levels as compared to a freshly isolated control sample and/or as compared to a threshold antibody level, identifying the sample as a low quality saliva sample. In embodiments, identifying the low quality saliva sample comprises determining the levels of antibodies against one or more circulating coronaviruses in a sample and, if the sample has low antibody levels against the one or more circulating coronaviruses as compared to a freshly isolated control sample and/or a threshold antibody level, identifying the sample as a low quality saliva sample.

In embodiments, the sample comprises an extracellular vesicle. As described herein, extracellular vesicles (also known as EVs or exosomes) are small membrane vesicles released by most cell types, including immune cells and infected cells (e.g., by a respiratory virus described herein such as SARS-CoV-2). Detection and analysis of EVs are further described, e.g., in US 2022/0003766; US 2021/0349104; WO 2019/222708; and WO 2020/086751.

In embodiments, the sample is pretreated prior to being subjected to the methods provided herein. In embodiments, the sample is pretreated prior to being handled by, processed by, or in contact with laboratory and/or clinical personnel. In embodiments, pretreating the sample comprises subjecting the sample to conditions sufficient to inactivate live virus in the sample. Inactivation of live virus that may be present in the sample reduces the risk of infection of the laboratory and/or clinical personnel handling and/or processing the sample, e.g., by performing the methods described herein on the sample. In embodiments, pretreating the sample comprises heating the sample to at least 55° C., at least 56° C., at least 57° C., at least 58° C., at least 59° C., at least 60° C., at least 65° C., at least 70° C., at least 75° C., at least 80° C., at least 85° C., at least 90° C., at least 95° C., or at least 100° C. In embodiments, the sample is heated for about 10 minutes to about 4 hours, about 20 minutes to about 2 hours, or about 30 minutes to about 1 hour. In embodiments, the sample is heated to about 65° C. for at least 10 minutes. In embodiments, the sample is heated to about 65° C. for at least 30 minutes. In embodiments, the sample is heated to about 58° C. for at least 1 hour.

In embodiments, pretreating the sample comprises contacting the sample with an inactivation reagent. In embodiments, the inactivation reagent comprises a detergent, a chaotropic agent, a fixative, or a combination thereof. Non-limiting examples of detergents include sodium dodecyl sulfate and TRITON™ X-100. Non-limiting examples of chaotropic agents include guanidium thiocyanate, guanidium isothiocyanate, and guanidium hydrochloride. Non-limiting examples of fixatives include formaldehyde, formalin, paraformaldehyde, and glutaraldehyde. In embodiments, pretreating the sample comprises subjecting the sample to UV or gamma irradiation. In embodiments, pretreating the sample comprises subjecting the sample to a highly alkaline (e.g., above pH 10, above pH 11, or above pH 12) condition. In embodiments, pretreating the sample comprises subjecting the sample to a highly acidic (e.g., below pH 4, below pH 3, below pH 2) condition. Additional methods of pretreating samples, e.g., containing the viruses described herein, is further discussed in Bain et al., Curr Protoc Cytometry 93:e77 (2020).

In embodiments, the sample comprises a viral nucleic acid. In embodiments, the sample comprising the viral nucleic acid is pretreated with a reagent that stabilizes and/or prevents degradation of the viral nucleic acid. In embodiments, the pretreating comprises removing and/or inhibiting activity of a nuclease, e.g., an RNase, in the sample. In embodiments, the viral nucleic acid is SARS-CoV-2 RNA.

In embodiments, the sample comprises an RT-PCR product. In embodiments, the RT-PCR product comprises a cDNA that is generated from a viral RNA. In embodiments, the sample comprising the RT-PCR product is pretreated to remove the viral RNA and/or a reagent used in the RT-PCR. In embodiments, the pretreating comprises contacting the sample with RNase. In embodiments, the pretreating comprises heating the sample, e.g., as described herein. In embodiments, the viral RNA is SARS-CoV-2 RNA.

In embodiments, the sample is pretreated immediately after being collected, e.g., from a subject described herein. Sample collection methods are provided herein. In embodiments, the sample is pretreated while being transported to a facility, e.g., a laboratory, for processing and analyzing the sample, e.g. using the methods described herein. In embodiments, the sample is pretreated after arrival at a facility, e.g., a laboratory, for processing and analyzing the sample, e.g. using the methods described herein. In embodiments, the sample is pretreated prior to being stored. In embodiments, the sample is stored prior to processing and analysis, e.g. using the methods described herein. In embodiments, the sample is stored at about −80° C. to about 30° C., about −70° C. to about 25° C., about −60° C. to about 20° C., about −20° C. to about 15° C., about 0° C. to about 10° C., about 2° C. to about 8° C., or about 4° C. to about 12° C. Methods and conditions for storing the samples described herein are known to one of ordinary skill in the art.

As used herein, the term “exposure,” in the context of a subject being exposed to a virus, refers to the introduction of a virus into the subject's body. “Exposure” does not imply any particular amount of virus; introduction of a single viral particle into the subject's body can be referred to herein as an “exposure” to the virus. As used herein, the term “infection,” in the context of a subject being infected with a virus, means that the virus has penetrated a host cell and has begun to replicate, assemble, and release new viruses from the host cell. The term “infection” can also be used to refer to an illness or condition caused by a virus, e.g., respiratory tract infection as described herein.

In embodiments, the virus, viral component, and/or biomarker are detectable in a subject immediately (e.g., within seconds) after the subject is exposed to the virus and/or infected with the virus. In embodiments, the virus, viral component, and/or biomarker are detectable in a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is exposed to the virus and/or infected with the virus. In embodiments, the virus, viral component, and/or biomarker are detectable in a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is exposed to the virus and/or infected with the virus. Different biomarkers, e.g., antibody biomarkers or inflammatory or tissue damage response biomarkers, in the same subject may have a varying magnitude of change in response to virus exposure and/or infection, for example, depending on whether the biomarker is an acute response biomarker or a biomarker related to a long-term effect. For some viral infections, the antibody biomarker IgG typically plateaus after 10 days of disease onset and persist (e.g., potentially signifying longer-term immunity); the antibody biomarkers IgA and IgM are detectable within 6 days of disease onset, peak around 10 days, and diminish after approximately 14 days (e.g., as part of the initial infection response). Different viruses can trigger biomarker responses at different times. For example, antibodies to SARS-CoV-2 may not be consistently detected in a subject until about three weeks after infection, which is longer than the typical timing for other types of viral infections. The timing of producing the same biomarker type, e.g., IgM or IgG antibody, can also vary widely among different subjects. Thus, in embodiments, the methods for multiplexed assays for a combination of biomarkers disclosed herein includes a determination or consideration of the response timing of each of the biomarkers.

In embodiments, the biological sample is obtained from a subject who has not been exposed to the virus. In embodiments, the biological sample is obtained from a subject immediately (e.g., within seconds) after the subject is known or suspected to be exposed to the virus. In embodiments, the biological sample is obtained from a subject within about 5 minutes to about 1 year, about 1 hour to about 9 months, about 6 hours to about 6 months, about 12 hours to about 90 days, 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is known or suspected to be exposed to the virus. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is known or suspected to be exposed to the virus.

In embodiments, the biological sample is obtained from a subject prior to the subject showing any symptoms of a viral infection. In embodiments, the biological sample is obtained from a subject immediately (e.g., within seconds) after the subject begins to show symptoms of a viral infection. In embodiments, the biological sample is obtained from a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject begins to show symptoms of a viral infection. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject begins to show symptoms of a viral infection. Symptoms of a viral infection are described herein and include, e.g., cough, shortness of breath, fever, and fatigue.

In embodiments, the biological sample is obtained from a subject after the subject is diagnosed with a viral infection. As described herein, the SARS-CoV-2 virus can cause post-acute COVID-19 syndrome, with certain symptoms persisting weeks or months after the initial illness period. In embodiments, the biological sample is obtained from a subject after about 1 day, about 2 days, about 3 days, about 4 days, about 5 days, about 6 day s, about 1 week, about 2 weeks, about 3 weeks, about 1 month, about 2 months, about 3 months, about 4 months, about 5 months, about 6 months, about 9 months, about 1 year, about 2 years, about 3 years, about 4 years, about 5 years, about 6 years, about 7 years, about 8 years, about 9 years, about 10 years, or more than 10 years after the subject is diagnosed with the viral infection.

In embodiments, the biological sample is obtained from a subject prior to the subject being administered with a vaccine or a treatment for the virus described herein. In embodiments, the biological sample is obtained from a subject immediately (e.g., within seconds) after a vaccine or a treatment is administered to the subject. In embodiments, the biological sample is obtained from a subject within about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after a vaccine or a treatment is administered to the subject. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after a vaccine or a treatment is administered to the subject.

### Sample Pooling

Samples may be obtained from a single source described herein, or may contain a mixture from two or more sources, e.g., pooled from one or more individuals who may have been exposed to or infected by a particular virus in a similar manner. Sample pooling strategies are further described, e.g., in U.S. Publication No. 2022/0003766 and U.S. Publication No. 2021/0349104. For example, the individuals may live or have lived in the same household, visited the same location(s), and/or associated with the same people. In embodiments, samples are pooled from two or more, three or more, four or more, five or more, six or more, seven or more, eight or more, nine or more, ten or more, 15 or more, 20 or more, 25 or more, 30 or more, 40 or more, 50 or more, 100 or more, 150 or more, 200 or more, 300 or more, 400 or more, 500 or more, 1000 or more, 5000 or more, or 10000 or more individuals. For example, a “negative” result for an active viral infection from a pooled sample indicates that none of the individuals from the pooled sample have an active infection, which can significantly reduce the number of tests needed to test every individual in a population. In embodiments, the sample comprises a respiratory sample, e.g., bronchial/bronchoalveolar lavage, saliva, mucus, oropharyngeal swab, sputum, endotracheal aspirate, pharyngeal/nasal swab, throat swab, nasal secretion, or combination thereof. In embodiments, the sample comprises saliva. In embodiments, the sample comprises blood. In embodiments, the sample comprises serum or plasma. In embodiments, the virus is a coronavirus. In embodiments, the virus is SARS-CoV-2. In embodiments, a “positive” result for an active viral infection in the pooled sample prompts or indicates a need for further testing using the methods and/or kits provided by the invention of individual samples comprised in the pool of samples.

In embodiments, the pooled sample is subjected to a single layer pooling strategy. A “single layer pooling strategy,” as used herein, refers to testing a pooled sample, and if the result of the pooled sample is “positive” for an active viral infection, each individual sample comprised in the pooled sample is then individually tested, e.g., using the methods and/or kits provided in the invention. In embodiments, the pooled sample is subjected to a multi-layer pooling strategy, e.g., a two-layer pooling strategy. In a “multi-layer pooling strategy,” a pooled sample containing n number of individual sample is tested in a first round, and if the result of the first round is “positive” for an active viral infection, then the pooled sample is divided into smaller pools, e.g., wherein each smaller pool comprises a number of individual samples equal to the square root of n, and re-tested in a second round. The smaller pool(s) with the “positive” results can be further divided into even smaller pools for one or more additional rounds of testing until the positive individual samples are identified. In an exemplary two-layer pooling strategy, a pooled sample containing 100 individual samples is tested in a first round, and if the pooled sample is tested to be “positive” for an active viral infection, then the pooled sample is divided into pools containing 10 individual samples. Each individual sample comprised in any 10-sample pools that tested “positive” are then tested.

In embodiments, the invention provides a method for determining the number of individual samples to be included in a pooled sample. In embodiments, the number of individual samples included in a pooled sample is based on disease prevalence in a population. For example, if disease prevalence is high, the likelihood of a pooled sample, containing a large number of individual samples, testing “positive” is also high, which reduces the benefits of testing pooled samples because additional tests are required to determine the positive individual samples.

In embodiments, each individual sample is about 0.1 mL to about 10 mL, about 0.2 mL to about 5 mL, or about 0.3 mL to about 3 mL. In embodiments, about 1%, about 2%, about 3%, about 4%, about 5%, about 10%, or about 20% of the total volume of each individual sample is added to the pooled sample. In embodiments, about 1 μL to about 100 μL, about 5 μL to about 50 μL, or about 10 μL to about 20 μL of each individual sample is added to the pooled sample. In embodiments, the amount of each individual sample not added to the pooled sample is sufficient for one or more additional rounds of testing (e.g., in a multi-layered pooling strategy as described herein).

### Collection and Assay Devices

In embodiments, the biological sample is a liquid sample. In embodiments, the biological sample is in contact with a sample collection device. In embodiments, the sample collection device is an applicator stick. In embodiments, the sample collection device comprises an elongated handle (e.g., a rod or a rectangular prism) and a sample collection head configured to collect sample from a biological tissue (e.g., from a subject's nasal or oral cavity) or a surface. In embodiments, the sample collection head comprises an absorbent material (e.g., cotton) or a scraping blade. In embodiments, the sample collection device is a swab. In embodiments, the sample collection device is a tissue scraper. In embodiments, the sample collection device is capable of collecting a sample described herein that may contain analytes at a concentration too low to support an accurate or reliable analysis result.

In embodiments, the sample collection device or the liquid sample is contacted with an assay cartridge. Assay cartridges are further described in, e.g., U.S. Publication No. 2022/0003766 and U.S. Publication No. 2021/0349104. Assay cartridges may be used with assay cartridge readers known in the art. An exemplary assay cartridge reader is the MSD® Cartridge Reader instrument. Further exemplary assay cartridges and assay cartridge readers are described, e.g., in U.S. Pat. Nos. 9,921,166; 10,184,884; 9,731,297; 8,343,526; 10,281,678; 10,272,436; US 2018/0074082; and US 2019/0391170.

In embodiments, the method is performed in an assay plate. Assay plates are known in the art and described, e.g., in U.S. Publication No. 2022/0003766 and U.S. Publication No. 2021/0349104. Further exemplary assay plates are disclosed in, e.g., U.S. Pat. Nos. 7,842,246; 8,790,578; and 8,808,627. In embodiments, the assay plate result is read in a plate reader, e.g., the MESO® QUICKPLEX® or MESO® SECTOR® instruments.

In embodiments, the method is performed on a particle. Particles known in the art, e.g., as described in U.S. Publication No. 2022/0003766 and U.S. Publication No. 2021/0349104, can be used in conjunction with the methods and kits described herein. In embodiments, the particle comprises a microsphere.

Further exemplary devices for performing the methods herein include, but are not limited to, cassettes, measurement cells, dipsticks, reaction vessels, and assay modules described in, e.g., U.S. Pat. Nos. 8,298,934 and 9,878,323.

### Assay Methods and Components

The viruses, viral components, and/or biomarkers described herein can be measured using a number of techniques available to a person of ordinary skill in the art, e.g., direct physical measurements (e.g., mass spectrometry) or binding assays (e.g., immunoassays, agglutination assays and immunochromatographic assays). Exemplary methods are described in, e.g., U.S. Publication No. 2022/0003766 and U.S. Publication No. 2021/0349104.

Exemplary binding assay methods include sandwich or competitive binding assays. Examples of sandwich immunoassays are described in U.S. Pat. Nos. 4,168,146 and 4,366,241. Examples of competitive immunoassays include those described in U.S. Pat. Nos. 4,235,601; 4,442,204; and 5,208,535.

Multiple viruses, viral components, and/or biomarkers can be measured using a multiplexed assay format, e.g., as described in US 2022/0003766; US 2021/0349104; US 2003/0113713; US 2003/0207290; US 2004/0022677; US 2004/0189311; US 2005/0052646; US 2005/0142033; US 2006/0069872; U.S. Pat. Nos. 5,807,522; 6,110,426; 6,977,722; 7,842,246; 10,189,023; and 10,201,812.

The methods herein can be conducted in a single assay chamber, such as a single well of an assay plate. The methods herein can also be conducted in an assay chamber of an assay cartridge as described herein. The assay modules, e.g., assay plates or assay cartridges, methods and apparatuses for conducting assay measurements suitable for the present invention, are described, e.g., in U.S. Pat. Nos. 8,343,526; 9,731,297; 9,921,166; 10,184,884; 10,281,678; 10,272,436; US 2004/0022677; US 2004/0189311; US 2005/0052646; US 2005/0142033; US 2018/0074082; and US 2019/0391170.

### Binding

Binding reagents that specifically bind to viruses, viral components, and/or biomarkers are described herein and, e.g., in U.S. Publication No. 2022/0003766 and U.S. Publication No. 2021/0349104. In embodiments where the method comprises quantifying the amounts of one or more biomarkers capable of binding to a viral antigen (e.g., an antibody biomarker), the binding complex comprises the binding reagent and the antibody biomarker. In embodiments, the binding reagent is immobilized on a binding domain. In embodiments, the binding complex is formed on the binding domain.

In embodiments where the method is a multiplexed immunoassay method, more than one binding complex is formed, and each binding complex comprises a different binding reagent and its binding partner (e.g., a biomarker described herein). Multiplexed immunoassay methods are described herein and, e.g., in U.S. Publication No. 2022/0003766 and U.S. Publication No. 2021/0349104. In embodiments, each of the binding reagents are immobilized on separate binding domains. In embodiments, each binding domain comprises a targeting agent capable of binding to a targeting agent complement, wherein the targeting agent complement is connected to a linking agent, and each binding reagent comprises a supplemental linking agent capable of binding to the linking agent.

In embodiments, an optional bridging agent, which is a binding partner of both the linking agent and the supplemental linking agent, bridges the linking agent and supplemental linking agent, such that the binding reagents, each bound to its respective targeting agent complement, are contacted with the binding domains and bind to their respective targeting agents via the bridging agent, the targeting agent complement on each of the binding reagents, and the targeting agent on each of the binding domains.

In embodiments, the targeting agent and targeting agent complement, and the linking agent and supplemental linking agent, are each two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments, each binding domain is an element of an array of binding elements. In embodiments, the binding domains are on a surface. In embodiments, the surface is a plate. In embodiments, the surface is a well in a multi-well plate. In embodiments, the array of binding elements is located within a well of a multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In embodiments, the particle comprises a microsphere. In embodiments, the particle comprises a paramagnetic bead. In embodiments, each binding domain is positioned on one or more particles. In embodiments, the particles are in a particle array. In embodiments, the particles are coded to allow for identification of specific particles and distinguish between each binding domain. In embodiments, the surface is an assay cartridge surface. In embodiments, each binding domain is positioned in a distinct location on the assay cartridge surface.

### Detection

In embodiments, the method further comprises detecting the binding complex described herein. In embodiments, the binding complex comprising a binding reagent and its binding partner (e.g., a biomarker described herein) further comprises a detection reagent. In embodiments, the detection reagent specifically binds to the biomarker described herein. Detection methods are known in the art and further described, e.g., in U.S. Publication No. 2022/0003766 and U.S. Publication No. 2021/0349104.

In embodiments, the method comprises contacting the binding reagent with its binding partner and the detection reagent simultaneously or substantially simultaneously to form a binding complex. In embodiments, the method comprises contacting the binding reagent with its binding partner and the detection reagent sequentially to form a binding complex. In embodiments, the method comprises contacting the detection reagent with its binding partner and the binding reagent sequentially. In embodiments, the binding partner comprises a biomarker, e.g., antibody biomarker described herein.

In embodiments, the detection reagent is an antibody, antigen, ligand, receptor, oligonucleotide, hapten, epitope, mimotope, or aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof. In embodiments where the method comprises detecting and/or quantifying the amounts of one or more biomarkers capable of binding to a viral antigen (e.g., an antibody biomarker), the detection reagent comprises an antigen (e.g., a viral protein described herein).

In embodiments, the detection reagent comprises a detectable label. In embodiments, measuring the concentration of the biomarkers in each of the binding complexes comprises measuring the presence and/or amount of the detectable label. In embodiments, the detectable label is measured by light scattering, optical absorbance, fluorescence, luminescence, chemiluminescence, electrochemiluminescence (ECL), bioluminescence, phosphorescence, radioactivity, magnetic field, or combination thereof. In embodiments, the detectable label comprises an electrochemiluminescence label. In embodiments, the detectable label comprises ruthenium. In embodiments, measuring the concentration of the biomarkers comprises measuring the presence and/or amount of the detectable label by electrochemiluminescence. In embodiments, the measuring of the detectable label comprises measuring an electrochemiluminescence signal.

In embodiments, detection reagent comprises a nucleic acid probe. In embodiments, the immunoassay further comprises binding the nucleic acid probe to a template oligonucleotide and extending the nucleic acid probe to form an extended sequence. In embodiments, the extended sequence binds to an anchoring reagent immobilized on the surface comprising the binding reagent. In embodiments, the virus, viral component, and/or biomarker is detected and/or quantified by detecting or quantifying the amount of extended sequence bound to the surface. In embodiments, the surface is contacted with a labeled probe that binds to the extended sequence, wherein the labeled probe comprises a detectable label.

In embodiments, the binding complex comprising the binding reagent and its binding partner (e.g., a biomarker described herein) further comprises a first detection reagent and a second detection reagent. In embodiments, the first detection reagent comprises a first nucleic acid probe, and the second detection reagent comprises a second nucleic acid probe. In embodiments, the immunoassay method further comprises binding the first and second nucleic acid probes to a template oligonucleotide and extending the second nucleic acid probe to form an extended sequence. In embodiments, the extended sequence binds to an anchoring reagent immobilized on the surface comprising the binding reagent. In embodiments, the biomarker is detected and/or quantified by detecting or quantifying the amount of extended sequence bound to the surface. In embodiments, the surface is contacted with a labeled probe that binds to the extended sequence, wherein the labeled probe comprises a detectable label. Detection methods are further described, e.g., in WO2014/165061; WO2014/160192; WO2015/175856; WO2020/180645; U.S. Pat. No. 9,618,510; U.S. Ser. No. 10/908,157; and U.S. Ser. No. 10/114,015.

In embodiments, the immunoassay is described in U.S. Publication No. 2022/0003766 and U.S. Publication No. 2021/0349104 and comprises:
(a) contacting a biotinylated binding reagent and a biotinylated anchoring reagent with a surface comprising streptavidin or avidin, e.g., for about 30 minutes to about 2 hours at room temperature, or about 6 hours to about 12 hours at 4° C.; and optionally washing the surface to remove unbound binding reagent and/or anchoring reagent;
(b) contacting a sample comprising the analyte of interest (e.g., biomarker described herein) with the surface, e.g., for about 1 hour to about 2 hours at about 20° C. to about 35° C. (e.g., about 27° C.); and optionally washing the surface to remove unbound analyte;
(c) contacting a detection reagent comprising a nucleic acid probe with the surface, e.g., for about 30 minutes to about 1 hour at about 20° C. to about 35° C. (e.g., about 27° C.), thereby forming a binding complex comprising the binding reagent, the analyte, and the detection reagent; and optionally washing the surface to remove unbound detection reagent;
(d) contacting a template oligonucleotide with the surface and ligating the template oligonucleotide to form a circular template, e.g., for about 10 minutes to about 30 minutes at about 20° C. to about 35° C. (e.g., about 27° C.), thereby hybridizing the nucleic acid probe to the circular template; and optionally washing the surface to remove excess template oligonucleotide;
(e) incubating the surface under conditions sufficient to perform rolling circle amplification, e.g., for about 5 minutes to about 30 minutes, thereby forming an extended sequence that binds to the anchoring reagent;
(f) contacting a labeled probe comprising a detectable label with the surface, e.g., for about 1 hour to about 2 hours at about 20° C. to about 35° C. (e.g., about 27° C.), thereby binding the labeled probe to the extended sequence; and optionally washing the surface to remove excess labeled probe; and
(g) measuring the amount of extended sequence by quantifying the amount of detectable label, thereby detecting and/or measuring the amount of analyte (e.g., biomarker described herein) in the sample.

In embodiments, the surface comprising the binding domains described herein comprises an electrode. In embodiments, the electrode is a carbon ink electrode. In embodiments, the measuring of the detectable label comprises applying a potential to the electrode and measuring electrochemiluminescence. In embodiments, applying a potential to the electrode generates an electrochemiluminescence signal. In embodiments, the strength of the electrochemiluminescence signal is based on the amount of detected analyte, e.g., biomarker described herein, in the binding complex.

In embodiments, the immunoassay described herein further comprises measuring the concentration of one or more calibration reagents. In embodiments, a calibration reagent comprises a known concentration of a biomarker described herein. In embodiments, the calibration reagent comprises a mixture of known concentrations of multiple biomarkers. Measurement of calibration reagents is known in the art and further described, e.g., in U.S. Publication No. 2022/0003766 and U.S. Publication No. 2021/0349104.

### Competitive Assays

In embodiments, the methods provided herein are in a competitive assay format. In general terms, a competitive assay, e.g., a competitive immunoassay or a competitive inhibition assay, an analyte (e.g., a biomarker described herein) and a competitor compete for binding to a binding reagent (e.g., a viral antigen described herein). In such assays, the analyte is typically indirectly measured by directly measuring the competitor. As used herein, “competitor” refers to a compound capable of binding to the same binding reagent as an analyte, such that the binding reagent can only bind either the analyte or the competitor, but not both. In embodiments, competitive assays are used to detect and measure analytes that are not capable of binding more than one binding reagents, e.g., small molecule analytes or analytes that do not have more than one distinct binding sites. Examples of competitive immunoassays include those described in U.S. Pat. Nos. 4,235,601; 4,442,204; and 5,028,535.

### Assays for Antibody Biomarkers

In embodiments where the biomarker detected is an antibody, e.g., an antibody capable of binding to a viral antigen such as S, S1, S2, S-NTD, S-ECD, S-RBD, M, E, or N, the binding reagent is an antigen that is bound by the antibody biomarker. In embodiments, antibody biomarkers are detected using a bridging serology assay. In a bridging serology assay, the binding complex further comprises a detection reagent described herein, and both the binding reagent and the detection reagent are an antigen that that is bound by the antibody biomarker. Since antibodies are typically bivalent, the antibody biomarker can bind both the binding reagent antigen and the detection reagent antigen.

In embodiments, antibody biomarkers are detected using a regular bridging serology assay. In a regular bridging serology assay, the antibody biomarker, binding reagent antigen, and detection reagent antigen are incubated together to form a complex where the antibody biomarker bivalently binds both the binding reagent antigen and the detection reagent antigen, e.g., a bridged complex. The incubation can be performed in any appropriate container, for example, in the well of a polypropylene plate, or in a chamber of an assay cartridge. In embodiments, the binding reagent antigen is conjugated to a biotin, and the bridged complex solution can be transferred to contact a surface comprising streptavidin, e.g., a streptavidin plate. In this embodiment, the biotin conjugated to the binding reagent antigen binds to the streptavidin plate, causing the entire bridged complex to be immobilized on the streptavidin plate.

In embodiments, antibody biomarkers are detected using a stepwise bridging serology assay. In a first step of a stepwise bridging serology assay, the binding reagent antigen is first immobilized on a surface. In embodiments where the binding reagent antigen is conjugated to biotin, the binding reagent antigen can be immobilized on a streptavidin plate. In a second step, after the binding reagent antigen is immobilized on the surface, a solution containing the antibody biomarker is contacted with the surface, allowing the first bivalent position on the antibody biomarker to bind the binding reagent antibody. In a third step, the detection reagent antigen is then contacted with the surface, allowing the second bivalent position on the antibody to bind the detection reagent antibody. In this stepwise method, the bridging complex is formed stepwise on the surface, rather than forming the entire bridging complex before immobilization, as is done in the regular bridging assay described above. In the stepwise bridging assay, the surface may optionally be rinsed or washed between any of the steps.

In either of the regular bridging serology assay or stepwise bridging serology assay, a method may be used where the detectable label is not directly conjugated to the detection reagent antigen but is instead attached to the detection antigen reagent using a binding complex such as streptavidin/biotin or other binding pair. The advantage of using this method is that it is not necessary to prepare separately conjugated binding reagent antigen and detection reagent antigen. In a non-limiting example of this method, a biotin conjugated antigen is prepared. Some of this biotin conjugated antigen is then incubated with a detectable label conjugated with streptavidin. The binding of biotin to streptavidin causes the detectable label to become attached to the biotin conjugated antigen, creating a detection reagent antigen comprising a detectable label as follows:
Antigen-biotin-streptavidin-detectable label
   In embodiments, additional free biotin is added to the antigen-detectable label reagent to fully occupy the streptavidin binding sites and prevent other biotin conjugates from binding to the antigen-detectable label reagent. An additional amount of the biotin conjugated antigen, which is not attached to a detectable label, is then used as the binding reagent antigen. Binding reagent antigen and detection reagent antigen prepared in this way may be used in any of the assay methods described herein.

In embodiments, the antibody biomarker is detected using a classical serology assay. In embodiments of a classical serology assay, the binding reagent is an antigen that is bound by the antibody biomarker. After the antibody biomarker is bound by the binding reagent antigen, the binding complex is detected using a detection reagent antibody that binds the antibody biomarker. In embodiments, the detection reagent antibody is an anti-human antibody that binds human antibody biomarkers. In embodiments, the detection reagent antibody is an anti-human IgG, an anti-human IgM or an anti-human IgA isotype antibody. In embodiments, the detection reagent antibody is an anti-mouse antibody that binds mouse antibody biomarkers, or an anti-rat antibody that binds rat antibody biomarkers, or an anti-ferret antibody that binds ferret antibody biomarkers, or an anti-minx antibody that binds minx antibody biomarkers, or an anti-bat antibody that binds bat antibody biomarkers. In embodiments, the detection reagent antibody is an anti-mouse IgG, IgM, or IgA antibody, an anti-rat IgG, IgM, or IgA antibody, an anti-ferret IgG, IgM, or IgA antibody, an anti-minx IgG, IgM, or IgA antibody, or an anti-bat IgG, IgM, or IgA antibody.

In embodiments, the antibody biomarker is detected using a competitive serology assay (also termed a neutralization serology assay). Competitive immunoassays are described herein. In embodiments of a competitive serology assay, the binding reagent is an antigen that is bound by the antibody biomarker and by a competitor. In embodiments, the competitor is a substance that binds a specific region of the viral antigen. In embodiments, the competitor is a recombinant antibody or antigen-binding fragment thereof that binds specifically to an epitope of the viral antigen, e.g., a neutralizing epitope. In embodiments, the competitor is a monoclonal antibody against an epitope of the viral antigen, e.g., a neutralizing epitope. In embodiments, the competitor comprises a detectable label described herein. For example, the biomarker can be an antibody that binds specifically to a coronavirus spike protein, and the competitor can be the ACE2 receptor, NRP1 receptor, or CD147, i.e., natural interaction partners of the spike protein. In embodiments, the competitor is the ACE2 receptor. In embodiments, the receptor is the NRP1 receptor. In embodiments, the competitor is CD147. In embodiments, the competitor comprises a sialic acid. In embodiments, the binding reagent is a substance that binds a viral antigen (e.g., ACE2, NRP1, or CD147), and the competitor is the viral antigen (e.g., spike protein or a variant thereof described herein, such as, e.g., S1, S2, S-NTD, S-ECD, or S-RBD). In embodiments, the coronavirus is SARS-CoV-2. In embodiments, a competitive serology assay as described herein is used to assess a potential protective serological response, e.g., the ability of the immune response to block binding of a viral antigen to its host cell receptor such as ACE2, NRP1, or CD147.

In embodiments, the antibody biomarker serology assay (either bridging, classical, or competitive) described herein comprises measuring the concentration of one or more calibration reagents. In embodiments, the calibration reagent is a positive control. In embodiments, the positive control comprises an antigen for which an antibody is known or expected to be present in the biological sample. In embodiments, the positive control comprises an antigen from a prevalent influenza strain, to which most subjects are expected to have antibodies. In embodiments, the positive control is an antigen from the H1 Michigan influenza virus. In embodiments, the positive control is immobilized in a binding domain of a surface that further comprises one or more viral antigens immobilized thereon in one or more additional binding domains, as described herein. In embodiments, antibody biomarker serology assay further comprises measuring the total levels of a particular antibody, e.g., total IgG, IgA, or IgM.

In embodiments, the calibration reagent is a negative control. In embodiments, the negative control comprises an antigen for which no antibodies are expected to be present in the biological sample. In embodiments, the negative control comprises a substance obtained from a non-human subject, and the biological sample is obtained from a human subject. In embodiments, the negative control comprises bovine serum albumin (BSA). In embodiments, the negative control, e.g., BSA, is immobilized in a binding domain of a surface that further comprises one or more viral antigens immobilized thereon in one or more additional binding domains, as described herein.

In embodiments, the calibration reagent comprises a combination of biological samples from subjects known to be infected or exposed to a virus described herein. In embodiments, the calibration reagent comprises a pooled sample of serum and/or plasma from subjects known to be infected or exposed to a virus described herein. In embodiments, the calibration reagent is the same biological material as the sample to be assayed. For example, if the biological sample for the antibody biomarker serology assay is a serum sample, then the calibration reagent is a pooled serum sample. Similarly, if the biological sample for the antibody biomarker serology assay is a plasma sample, then the calibration reagent is a pooled plasma sample. In embodiments, the pooled sample comprises a known amount of IgG, IgA, and/or IgM that specifically bind to one or more viral antigens of interest. Methods of measuring IgG, IgA, and/or IgM concentration in a serum or plasma sample is known in the art, e.g., as described in Quataert et al., Clinical and Diagnostic Laboratory Immunology 2(5):590-597 (1995). In embodiments, the antibody biomarker serology assay comprises measuring the concentration of viral antigen-specific IgG, IgA, and/or IgM in multiple pooled samples to provide a calibration curve. In embodiments, the antibody biomarker serology assay comprises measuring the concentration of viral antigen-specific IgG, IgA, and/or IgM in multiple pooled samples, wherein the multiple pooled samples correspond to high, medium, and low levels of viral antigen-specific IgG, IgA, and/or IgM (referred to herein as “high pooled sample,” “medium pooled sample,” and “low pooled sample,” respectively). In embodiments, the pooled sample comprises serum and/or plasma from subjects known to never have been exposed to a virus described herein, i.e., a negative pooled sample. Pooled samples as calibration reagents allow baseline immune response thresholds to be defined and provides a better understanding of the levels of antibody response to a viral infection. In embodiments, the virus is a coronavirus. In embodiments, the virus is SARS-CoV-2.

In embodiments, the biological sample for the antibody biomarker serology assay is a saliva sample, and the calibration reagent comprises a calibration saliva sample. In embodiments, the calibration saliva sample contains a known amount of viral antigen-specific IgG, IgA, and/or IgM. In embodiments, the calibration saliva sample comprises serum from a subject known to be infected or exposed to a virus described herein. In embodiments, the calibration saliva sample comprises about 0.1% to about 1% of high pooled serum sample described herein. In embodiments, the calibration saliva sample comprises about 0.1%, about 0.2%, about 0.3%, about 0.4%, or about 0.5% of high pooled serum sample described herein. In embodiments, the calibration saliva sample comprises levels of viral antigen-specific IgG, IgA, and/or IgM equivalent to a 1:500 dilution of the high pooled serum sample as described herein. In embodiments, the calibration saliva sample is obtained from a subject known to never have been exposed to a virus described herein, i.e., a negative saliva sample. In embodiments, the calibration saliva sample provides a consistent threshold for comparing viral antigen-specific IgG, IgA, and/or IgM levels in saliva samples. In embodiments, the virus is a coronavirus. In embodiments, the virus is SARS-CoV-2.

In embodiments, the calibration reagents, e.g., the pooled sample and/or the calibration saliva sample described herein, is subjected to an antibody biomarker serology assay, e.g., the classical, bridging, and/or competitive serology assays described herein. In embodiments, the assay comprises measuring the total amount of IgG, IgA, and/or IgM in a dilution series of the calibration reagent. In embodiments, the assay further comprises generating a standard curve based on the measured amounts of IgG, IgA, and/or IgM in the calibration reagent dilution series. In embodiments, the assay comprises determining the amount of IgG, IgA, and/or IgM in a biological sample based on the standard curve. In embodiments, the IgG, IgA, and/or IgM is from a human, a mouse, a rat, a ferret, a minx, a bat, or a combination thereof.

An exemplary multiplexed classical or bridging serology assay detecting human IgG and/or IgM against SARS-CoV-2 antigens, and/or an exemplary multiplexed competitive serology assay detecting human neutralizing antibodies (also known as blocking antibodies) against SARS-CoV-2 antigens, as described in embodiments herein, comprises:

1. Preparation of assay plate. In embodiments, the assay plate is a 384-well assay plate. In embodiments, the assay plate is a 96-well assay plate. In embodiments, each well comprises four distinct binding domains, e.g., as shown in FIG. 29A. In embodiments, each well comprises ten distinct binding domains, e.g., as shown in FIG. 29B.

In embodiments, each well of the assay plate comprises ten distinct binding domains, wherein each binding domain comprises an immobilized viral (e.g., a respiratory virus such as SARS-CoV-2) antigen as described herein. In embodiments, the viral antigens, e.g., SARS-CoV-2 antigens, are immobilized on a surface comprising Spots 1-10 as shown in FIG. 29B, wherein the antigens are arranged as shown in Tables 2A-2H. In embodiments, the S protein mutations from the SARS-CoV-2 strains of Tables 2A-2H are described in Table 1D. In embodiments, the S-RBD mutations from the SARS-CoV-2 strains of Tables 2A-2H are described in Table 1E.

In embodiments, about 1 μL to about 200 μL, about 3 μL to about 150 μL, about 5 μL to about 100 μL, about 10 μL to about 90 μL, about 15 μL to about 80 μL, about 20 μL to about 70 μL, about 30 μL to about 60 μL, about 50 μL, or about 150 μL of a blocking solution to each well of the plate. In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

2. Preparation of reagents. In embodiments, the assay comprises measuring the amount of one or more calibration reagents. In embodiments, the calibration reagent comprises a known quantity of IgG and/or IgM. In embodiments, the calibration reagent comprises a blank solution containing no IgG or IgM. In embodiments, the assay comprises measuring the amount of multiple calibration reagents, e.g., at least 3, at least 4, at least 5, at least 6, at least 7, at least 8, at least 9, or at least 10 calibration reagents. In embodiments, the assay comprises generating a standard curve from the multiple calibration reagents. In embodiments, the multiple calibration reagents comprise a range of concentrations of IgG and/or IgM. In embodiments, the assay comprises diluting a concentration reagent to provide multiple calibration reagents comprising a range of concentrations. In embodiments, the calibration reagent is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. Calibration reagents are further described herein.

In embodiments, the assay comprises measuring the amount of one or more control reagents. In embodiments, the control reagent comprises a known quantity of IgG and/or IgM against the specific viral antigens in the assay, e.g., SARS-CoV-2 S, SARS-CoV-2 N, and/or SARS-CoV-2 S-RBD. In embodiments, the one or more control reagents comprises a first control reagent obtained from a subject known to never have been exposed to SARS-CoV-2, a second control reagent obtained from a subject during an early stage of infection by SARS-CoV-2, a third control reagent obtained from a subject during a late stage infection by SARS-CoV-2, a fourth control reagent obtained from a subject who has recovered from an infection by SARS-CoV-2, or a combination thereof. Control reagents are further described herein.

Examples of samples, e.g., biological samples, are provided herein. In embodiments, the sample is diluted about 2-fold, about 5-fold, about 10-fold, about 20-fold, about 30-fold, about 40-fold, about 50-fold, about 60-fold, about 70-fold, about 80-fold, about 90-fold, about 100-fold, about 250-fold, about 500-fold, about 750-fold, about 1000-fold, about 1500-fold, about 2000-fold, about 2500-fold, about 3000-fold, about 3500-fold, about 4000-fold, about 4500-fold, or about 5000-fold for use in the assay.

3. Addition of samples and reagents. In embodiments, the assay plate is washed at least once, at least twice, at least three times, at least four times, or at least five times with a wash buffer after incubation with the blocking solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

3A. Classical or Bridging Serology Assay: In embodiments, after the washing, the sample, one or more calibration reagents, and one or more control reagents are added to their respectively designated wells of the plate. In embodiments, about 5 μL to about 50 μL, about 10 L to about 40 μL, about 20 μL to about 30 μL, about 15 μL, about 25 μL, or about 50 μL of the sample, calibration reagent, or control reagent is added to each well.

3B. Competitive Serology Assay: In embodiments, after the washing, the sample and one or more calibration reagents are added to their respectively designated wells of the plate. In embodiments, about 5 μL to about 50 μL, about 10 μL to about 40 μL, about 20 μL to about 30 μL, about 15 μL, about 25 μL, or about 50 μL of the sample or calibration reagent is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 12 hours, or about 30 minutes to about 8 hours, or about 45 minutes to about 6 hours, or about 1 hour, or about 4 hours. In embodiments, the plate is incubated at about room temperature (e.g., about 22° C. to about 28° C.) while shaken at about 1500 rpm for about 4 hours. In embodiments, the plate is incubated at about room temperature (e.g., about 22° C. to about 28° C.) while shaken at about 700 rpm for about 1 hour.

4A. Classical or Bridging Serology Assay—Addition of detection reagent. In embodiments, the detection reagent is diluted from a stock solution of detection reagent to obtain a solution comprising a working concentration of detection reagent. Detection reagents are further described herein.

4B. Competitive Serology Assay—Addition of ACE2 detection reagent. In embodiments, the ACE2 detection reagent is diluted from a stock solution of detection reagent to obtain a solution comprising a working concentration of ACE2 detection reagent. ACE2 is further described herein.

In embodiments, the assay plate is washed at least once, at least twice, at least three times, at least four times, or at least five times with a wash buffer after incubation with the sample, calibration reagent, or control reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the detection reagent solution for the classical or bridging serology assay, or the ACE2 detection reagent solution for the competitive serology assay, is added to each well of the plate. In embodiments, about 5 μL to about 50 μL, about 10 μL to about 40 μL, about 10 μL to about 20 μL, about 20 μL to about 30 μL, about 15 μL, about 25 μL, or about 50 μL of the detection reagent solution or the ACE2 detection reagent solution is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) while shaken at about 1500 rpm for about 1 hour. In embodiments, the plate is incubated at about room temperature (e.g., about 22° C. to about 28° C.) while shaken at about 700 rpm for about 1 hour.

5. Addition of read buffer. In embodiments, the assay plate is washed at least once, at least twice, at least three times, at least four times, or at least five times with a wash buffer after incubation with the detection reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, the read buffer is added to each well of the plate. Read buffers are further described herein.

5A. Classical or Bridging Serology Assay: In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well.

5B. Competitive Scrology Assay: In embodiments, the read buffer is added to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, or about 40 μL of the read buffer is added to each well.

In embodiments, the assay comprises reading the plate, e.g., on a plate reader as described herein. In embodiments, the assay comprises reading the plate immediately following addition of the read buffer.

A further exemplary serology assay for detecting an antibody biomarker that binds to a SARS-CoV-2 antigen comprises:
(a) mixing (i) a coating solution comprising a binding reagent bound to a linking agent and (ii) a detection reagent, wherein each of the binding reagent and the detection reagent comprises a SARS-CoV-2 antigen, and wherein the detection reagent comprises a detectable label;
(b) contacting a surface with: (i) a sample comprising the antibody biomarker, (ii) a calibration reagent, or (iii) a control reagent, wherein the surface comprising one or more binding domains, wherein each binding domain comprises a targeting agent;
(c) contacting the surface with the mixture of (a);
(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary serology assay for detecting an antibody biomarker that binds to a SARS-CoV-2 antigen comprises:
(a) mixing (i) a biotinylated binding reagent and (ii) a detection reagent, wherein each of the binding reagent and the detection reagent comprises a SARS-CoV-2 antigen, and wherein the detection reagent comprises a detectable label;
(b) contacting a surface with: (i) a sample comprising the antibody biomarker, (ii) a calibration reagent, or (iii) a control reagent, wherein the surface comprising one or more binding domains, wherein each binding domain comprises avidin or streptavidin;
(c) contacting the surface with the mixture of (a);
(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary competitive scrology assay for detecting an antibody biomarker that binds to a SARS-CoV-2 antigen comprises:
(a) contacting a coating solution comprising a binding reagent bound to a linking reagent with a surface comprising one or more binding domains, wherein each binding domain comprises a targeting agent, and wherein the binding reagent is a SARS-CoV-2 antigen;
(b) contacting each binding domain with: (i) a sample comprising the antibody biomarker, (ii) a calibration reagent, or (iii) a control reagent;
(c) contacting each binding domain with an ACE2 detection reagent comprising a detectable label;
(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary competitive serology assay for detecting an antibody biomarker that binds to a SARS-CoV-2 antigen comprises:
(a) contacting a biotinylated binding reagent with a surface comprising one or more binding domains, wherein each binding domain comprises avidin or streptavidin, and wherein the binding reagent is a SARS-CoV-2 antigen;
(b) contacting each binding domain with: (i) a sample comprising the antibody biomarker, (ii) a calibration reagent, or (iii) a control reagent;
(c) contacting each binding domain with an ACE2 detection reagent comprising a detectable label;
(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

In embodiments, the surface is a multi-well plate. In embodiments, the assay further comprises a wash step prior to one or more of the assay steps. In embodiments, the wash step comprises washing the assay plate at least once, at least twice, at least three times, at least four times, or at least five times with a wash buffer. In embodiments, the assay plate is washed with at least about 10 μL, at least about 15 μL, at least about 20 μL, at least about 25 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer. In embodiments, the classical or bridging serology assay does not comprise a wash step prior to any of steps (a), (b), or (c). In embodiments, the competitive serology assay does not comprise a wash step prior to any of steps (a), (b), or (c).

In embodiments, prior to step (a), a blocking solution is added to the plate to reduce non-specific binding of the coating solution or the biotinylated binding reagent to the surface. In embodiments, about 50 μL to about 250 μL, about 100 μL to about 200 μL, or about 150 μL of blocking solution is added per well of the plate. In embodiments, the plate is incubated at about 15° C. to about 30° C., about 18° C. to about 28° C., about 20° C. to about 26° C., or about 22° C. to about 24° C. In embodiments, the plate is incubated while shaken at about at about 500 rpm to about 2000 rpm, about 600 rpm to about 1500 rpm, or about 700 rpm to about 1000 rpm. In embodiments, the method comprises incubating the blocking solution on the plate for about 10 minutes to about 4 hours, about 20 minutes to about 3 hours, or about 30 minutes to about 2 minutes. In embodiments, the plate is incubated at about room temperature (e.g., about 22° C. to about 28° C.) while shaken at about 700 rpm for about 30 minutes to about 2 hours.

In embodiments comprising a coating solution, the assay further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL, or about 400 μL to about 600 μL of the coating solution. In embodiments, step (a) comprises incubating the linking agent and the binding reagent at about 15° C. to about 30° C., about 18° C. to about 28° C., about 20° C. to about 26° C., or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the assay further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C., about 18° C. to about 28° C., about 20° C. to about 26° C., or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, about 10 μL to about 200 μL, about 5 μL to about 100 μL, about 10 μL to about 90 μL, about 15 μL to about 80 μL, about 20 μL to about 70 μL, about 30 μL to about 60 μL, or about 50 μL of the coating solution or a solution containing the biotinylated binding reagent are added to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, about 5 μL to about 50 μL, about 10 μL to about 40 μL, about 20 μL to about 30 μL, about 15 μL, about 25 μL, about 35 μL, or about 50 μL of the sample, calibration reagent, or control reagent are added to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments where the assay is a classical or bridging serology assay, about 5 μL to about 50 μL, about 10 μL to about 40 μL, about 10 μL to about 20 μL, about 20 μL to about 30 μL, about 15 μL, about 25 μL, about 35 μL, or about 50 μL of the mixture comprising the binding reagent and the detection reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments where the assay is a competitive serology assay, about 5 μL to about 50 μL, about 10 μL to about 40 μL, about 10 μL to about 20 μL, about 20 μL to about 30 μL, about 15 μL, about 25 μL, about 35 μL, or about 50 μL of a solution comprising the ACE2 detection reagent are added to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 L to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

### Quantitation Embodiments

In embodiments, the invention further provides a method of determining viral exposure in a subject, (a) comprising conducting an immunoassay method described herein on a biological sample of the subject; (b) detecting the virus, viral component, and/or biomarker (e.g., antibody biomarker or inflammatory or tissue damage biomarker) as described herein; (c) determining if the amount of detected virus, viral component, and/or biomarker is higher or lower relative to a control; and (d) determining the viral exposure of the subject based on the determination of (c). In embodiments, the method comprises normalizing the detected amount of biomarker (e.g., antibody biomarker) to a control and determining whether the subject is exposed to, infected by, and/or immune to the virus. In embodiments, the control is a biological sample containing a known amount of biomarkers (e.g., antibody biomarkers or inflammatory or tissue damage biomarkers). In embodiments, the control is a biological sample obtained from a subject known to have never been exposed to the virus. In embodiments, the control is a biological sample obtained from a subject known to have recovered from an infection by the virus. In embodiments, the virus is a coronavirus. In embodiments, the virus is SARS-CoV-2.

In embodiments comprising a multiplexed immunoassay for quantifying the amount of a biomarker (e.g., an antibody biomarker or an inflammatory or tissue damage biomarker), the method further comprises determining a threshold value of the biomarker in a healthy subject. In embodiments, the threshold value is determined from the aggregate results of measured biomarker amounts in multiple healthy subjects. For example, the aggregate results from a certain number of samples can determine the percentile, e.g., 99^{th} percentile or greater, of biomarker levels in a healthy subject. Various statistical models and algorithms can be utilized to calculate the extent of viral infection and/or degree of immunity in a subject by comparing and/or normalizing the subject's measured biomarker amount to the threshold value of that biomarker.

In embodiments, the multiplexed immunoassay for quantifying the amount of an antibody biomarker (e.g., a serology assay described herein) that specifically binds to a viral antigen is performed substantially with an assay that measures inhibition of binding between a viral protein and its associated host receptor, e.g., the binding of the coronavirus spike protein to the ACE2 receptor or the NRP1 receptor. In embodiments, the antibody biomarker inhibits binding between the viral protein and its associated host receptor. In embodiments, the inhibition assay indirectly detects the antibody biomarker. In embodiments, simultaneous direct detection (e.g., utilizing a viral antigen as a binding reagent) and indirect detection (e.g., measuring inhibition between a viral protein and its receptor) of the antibody biomarker improves specificity of the antibody biomarker detection.

In embodiments, the invention provides methods of assessing the affinity of an antibody biomarker to a viral antigen described herein, e.g., a SARS-CoV-2 antigen. As used in the context of antibodies, “affinity” refers to the strength of interaction between an epitope (e.g., on a viral antigen) and an antibody's antigen-binding site. In embodiments, the invention provides methods of assessing the binding kinetics between an antibody biomarker and viral antigen described herein. Methods of measuring antibody affinity and/or binding kinetics include, e.g., surface plasmon resonance (SPR) and bio-layer interference (BLI). Antibody affinity measurement is further described in, e.g., Underwood, Advances in Virus Research 34:283-309 (1988); Azimzadeh et al., J Mol Recognition 3(3):108-116 (1990); Hearty et al., Methods Mol Biol 907:411-442 (2012); and Singhal et al., Anal Chem 82(20):8671-8679 (2010).

In embodiments, the invention provides methods of assessing the affinity of a neutralizing antibody to a viral antigen described herein, e.g., a SARS-CoV-2 antigen. In embodiments, the affinity determination of a neutralizing antibody in a serum or plasma sample for SARS-CoV-2 comprises: a) titrating a labeled competitor to a surface comprising a known amount of SARS-CoV-2 S protein to determine the K_{dL} between the labeled ACE2 competitor and S protein; b) titrating: (i) a plasma sample known to contain neutralizing antibody for SARS-CoV-2 S while maintaining a constant ACE2 concentration, as described by equation 1(a); and (ii) ACE2 while maintaining a constant sample concentration, as described by equation 1(b); and c) solving the system of equations 1(a) and 1(b) to determine the average antibody concentration in sample [A] and average affinity K_{dA}. As described in equation 1(a), if the binding of fixed labeled ACE2, [L], is plotted against the log concentration of the unlabeled competitor antibody, [A], the resulting inhibition curve will be shifted by a factor of log(1+[L]/K_{dL}).

$${IC}_{50} = {K}_{dLapp} = {K}_{dL} ( 1 + [ A ] / {K}_{dA} ) \qquad 1 ( a )$$

$${EC}_{50} = {K}_{dAapp} = {K}_{dA} ( 1 + [ L ] / {K}_{dL} ) \qquad 1 ( b )$$

Affinity measurements are further described, e.g., in Hulme et al., British Journal of Pharmacology 161: 1219-1237 (2010).

### Assays for Viral Nucleic Acids

### Amplification and Detection of Viral Nucleic Acid

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus is SARS-CoV-2. In embodiments, the coronavirus nucleic acid is RNA. In embodiments, the binding reagent comprises a single stranded oligonucleotide.

In embodiments, the sample comprises a coronavirus nucleic acid. In embodiments, the method further comprises amplifying the coronavirus nucleic acid to form one or more additional copies of the coronavirus nucleic acid sequence, forming a plurality of binding complexes, each binding complex comprising a copy of the coronavirus nucleic acid sequence, and detecting the plurality of binding complexes, thereby detecting the coronavirus in the biological sample. In embodiments, the coronavirus nucleic acid is RNA, and the amplifying comprises reverse transcribing the RNA to form a cDNA, and amplifying the cDNA using polymerase chain reaction (PCR) to form a PCR product comprising a copy of the coronavirus nucleic acid sequence. In embodiments, the reverse transcription to form a cDNA and the PCR to amplify the cDNA are performed in a single reaction mixture. In embodiments, the reaction mixture further comprises a glycosylase enzyme. In embodiments, the glycosylase removes non-specific products from the reaction mixture. In embodiments, the glycosylase is uracil-N-glycosylase. In embodiments, the sample comprises an RT-PCR product, e.g., cDNA. In embodiments, the cDNA is generated from a coronavirus nucleic acid. In embodiments, the method comprises amplifying the cDNA using PCR to form a PCR product comprising a copy of the coronavirus nucleic acid sequence. In embodiments, the PCR is performed for about 10 to about 60 cycles, about 20 to about 50 cycles, or about 30 to about 40 cycles. In embodiments, the cDNA is amplified with a first primer that comprises a binding partner of the binding reagent and a second primer that comprises a detectable label or binding partner thereof, to form the PCR product. In embodiments, the first primer is a PCR forward primer and comprises the binding partner of the binding reagent at a 5′ end. In embodiments, the second primer is a PCR reverse primer and comprises the detectable label or binding partner thereof at a 3′ end. In embodiments, the PCR product comprises, in 5′ to 3′ order: the binding partner of the binding reagent, a copy of the coronavirus nucleic acid sequence, and the detectable label or binding partner thereof.

In embodiments, the first and second primers amplify a coronavirus nucleic acid sequence that encodes a protein, e.g., any of the coronavirus proteins described herein such as S, E, M, N, or a nonstructural protein. In embodiments, the first and second primers amplify a non-coding coronavirus nucleic acid sequence, i.e., that does not encode a gene. In embodiments, the first and second primers amplify a coronavirus nucleic acid sequence capable of identifying a coronavirus species. In embodiments, the coronavirus nucleic acid is SARS-CoV-2 RNA.

In embodiments, the method is a multiplexed method. In embodiments, the cDNA is amplified using multiple sets of primers, wherein each set of primers comprises a PCR forward primer and a PCR reverse primer as described herein. In embodiments, the PCR forward primer in each set of primers comprises a binding partner of the same binding reagent. In embodiments, the PCR forward primer in each set of primers comprises a binding partner of different binding reagents. In embodiments, each set of primers amplifies a different region of the cDNA to generate a plurality of PCR products, each having a different coronavirus nucleic acid sequence. For example, a first set of primers amplifies a region that encodes for the S protein, a second set of primers amplifies a region that encodes for the N protein, a third set of primers amplifies a region for a noncoding region, etc. In embodiments, each coronavirus nucleic acid sequence corresponds to a different binding reagent. In embodiments, the coronavirus nucleic acid sequence of the PCR product is identified by determining the binding reagent that binds the PCR product. In embodiments, the coronavirus nucleic acid is SARS-CoV-2 RNA. In embodiments, the primers for amplifying a region that encodes for the S protein are described in Table 14. In embodiments, the primer comprises a modified nucleotide, e.g., a locked nucleic acid (LNA).

In embodiments, the binding reagent comprises a single-stranded oligonucleotide, and the binding partner of the binding reagent comprises a complementary oligonucleotide of the binding reagent. In embodiments, the binding reagent further comprises a targeting agent complement. In embodiments, the targeting agent complement comprises an oligonucleotide that is complementary to a targeting agent on a surface, as described herein. In embodiments, the binding reagent is immobilized to the surface via the targeting agent—targeting agent complement interaction. In embodiments, each PCR product binds to a binding reagent to form one or more binding complexes on the surface. In embodiments comprising different binding reagents corresponding to different coronavirus nucleic acid sequences, each binding reagent is located at a distinct binding domain on the surface, and the detected coronavirus nucleic acid sequence is identified by the location of the binding complex on the surface.

In embodiments, the method comprises detecting the binding complex(es). In embodiments, the PCR product comprises a detectable label. In embodiments, the PCR product comprises a binding partner of a detectable label. Detectable labels are described herein. In embodiments, the detectable label is an electrochemiluminescence (ECL) label. In embodiments, the PCR product comprises biotin, and the detectable label comprises an ECL label linked to avidin or streptavidin. In embodiments, the PCR product comprises avidin or streptavidin, and the detectable label comprises an ECL label linked to biotin. Additional non-limiting examples of binding partners that can be on the detection probe and detectable label are provided herein.

In embodiments, RNA is extracted from a sample containing an RNA virus (e.g., SARS-CoV-2), and the extracted RNA is converted to cDNA. A “Master Mix” is prepared by combining a forward primer comprising a 5′ binding reagent complement sequence and a cDNA complement sequence, a reverse primer comprising a cDNA reverse complement sequence and a 3′ binding partner of a detectable label, and other PCR components such as dNTPs and DNA polymerase (e.g., Taq polymerase). The cDNA and Master Mix are combined, and PCR is performed for 30 to 40 cycles to form a plurality of PCR products, each PCR product comprising the 5′ binding reagent complement sequence and 3′ binding partner of a detectable label. Each PCR product hybridizes to a binding reagent on a surface. The surface is then contacted with a detectable label, which binds to the PCR product. The PCR product bound to the detectable label is then subjected to detection as described herein.

In an alternative embodiment, the Master Mix comprises the components for performing the reverse transcription reaction and the PCR reaction, e.g., reverse transcriptase, DNA polymerase, forward and reverse primers, nucleotides, magnesium, ribonuclease inhibitor, and glycosylase, and the RNA extracted from the sample is added to the Master Mix, such that the reverse transcription reaction and the PCR reaction are performed with a single reaction mixture to form the PCR product. In embodiments, the single reaction mixture is: (1) incubated at a first temperature sufficient to activate the glycosylase; (2) incubated at a second temperature sufficient to perform the reverse transcription; and (3) incubated at temperature sufficient to perform PCR. In embodiments, the PCR product is bound to the surface and detected as described herein.

### Detection of Viral Nucleic Acids and Single Nucleotide Polymorphisms (SNPs)

In embodiments, the invention provides a method for detecting a coronavirus nucleic acid in a biological sample. In embodiments, the invention provides a method of identifying the circulating strains of SARS-CoV-2 without sequencing a large number of SARS-CoV-2 isolates. Certain strains of SARS-CoV-2 are associated with increased transmissibility (e.g., the B.1.1.7, 501Y.V2, and P.1 strains) and diminished efficacy against currently available vaccines. In embodiments, the invention provides a method of real-time monitoring and assessing transmission patterns of SARS-CoV-2. In embodiments, the invention provides a method for determining a SARS-CoV-2 strain (e.g., the L strain or S strain, or the S-D614 or S-D614G strain, or the variants in Table 1A as described herein) in a biological sample.

In embodiments, the invention provides method for detecting a single nucleotide polymorphism (SNP) in a target nucleic acid, wherein the target nucleic acid is a SARS-CoV-2 nucleic acid, comprising: (a) contacting a sample comprising the target nucleic acid with (i) a targeting probe, wherein the targeting probe comprises a first region complementary to a polymorphic site of the target nucleic acid that comprises the SNP, and wherein the targeting probe comprises an oligonucleotide tag; and (ii) a detection probe, wherein the detection probe comprises a second region complementary to an adjacent region of the target nucleic acid comprising the polymorphic site, and wherein the detection probe comprises a detectable label; (b) hybridizing the targeting and detection probes to the target nucleic acid; (c) ligating the targeting and detection probes that hybridize with perfect complementarity at the polymorphic site to form a ligated target complement comprising the oligonucleotide tag and the detectable label; (d) contacting the product of (c) with a surface comprising an immobilized binding reagent, wherein the binding reagent comprises an oligonucleotide complementary to the oligonucleotide tag; (e) forming a binding complex on the surface, wherein the binding complex comprises the binding reagent and the ligated target complement; and (f) detecting the binding complex, thereby detecting the SNP at the polymorphic site.

In embodiments, the method comprises an oligonucleotide ligation assay (OLA). OLA and other nucleic acid detection methods are described, e.g., in WO 2020/227016. In embodiments, the OLA method is used to detect, identify, and/or quantify a coronavirus nucleic acid (e.g., RNA). In embodiments, the coronavirus nucleic acid encodes the N gene. In embodiments, the coronavirus nucleic acid is the N1 region, N2 region, or N3 region of the N gene, as described herein. In embodiments, the OLA method is used to detect, identify, and/or quantify a single nucleotide polymorphism (SNP) at a polymorphic site in a coronavirus nucleic acid (e.g., RNA). In embodiments, the coronavirus is SARS-CoV-2. In embodiments, the method detects any of the SNPs as shown in Table 1A and Table 1C.

In embodiments, the OLA method for detecting a coronavirus nucleic acid or for detecting an SNP comprises: (a) contacting the biological sample with: (i) a targeting probe, wherein, when the method is for detecting the coronavirus nucleic acid, the targeting probe is complementary to a first region of a target nucleic acid; or wherein, when the method is for detecting the SNP, the targeting probe is complementary to a polymorphic site of a target nucleic acid, wherein the target nucleic acid is, e.g., the coronavirus nucleic acid or an RT-PCR product described herein, and wherein the targeting probe comprises an oligonucleotide tag; and (ii) a detection probe, wherein, when the method is for detecting the coronavirus nucleic acid, the detection probe is complementary to a second region that is adjacent to the first region of the target nucleic acid; or wherein, when the method is for detecting the coronavirus nucleic acid, the detection probe is complementary to an adjacent region of the target nucleic acid containing the distinct SNP; (b) hybridizing the targeting and detection probes to the target nucleic acid; (c) ligating the targeting and detection probes that hybridize with perfect complementarity to the first and second regions of the target nucleic acid when the method is for detecting the coronavirus nucleic acid; or ligating the targeting and detection probes that hybridize with perfect complementarity at the polymorphic site when the method is for detecting the SNP, to form a ligated target complement comprising the oligonucleotide tag and the detectable label; (d) contacting the product of (c) with a surface comprising a binding reagent immobilized in one or more binding domains, wherein the binding reagent comprises an oligonucleotide complementary to the oligonucleotide tag; (e) forming a binding complex on the surface between the binding reagent and the ligated target complement; and (f) detecting the binding complex, thereby detecting, identifying, and/or quantifying the coronavirus nucleic acid or the SNP at the polymorphic site of the coronavirus nucleic acid. In embodiments, the coronavirus is SARS-CoV-2. In embodiments, the coronavirus nucleic acid is RNA. In embodiments, the sample comprises the coronavirus nucleic acid. In embodiments, the sample comprises an RT-PCR product, e.g., cDNA that is generated from the coronavirus nucleic acid.

In embodiments, the ligating of the oligonucleotide probes is dependent on three events: (1) the targeting and detection probes must hybridize to complementary sequences within the target nucleic acid; (2) the targeting and detection probes must be adjacent to one another in a 5′- to 3′-orientation with no intervening nucleotides; and (3) the targeting and detection probes must have perfect base-pair complementarity with the target nucleic acid at the ligation site. A single nucleotide mismatch between the primers and target may inhibit ligation. In embodiments, the melting temperature (T_{M}) of the oligonucleotide probes is about 55° C. to about 70° C., about 58° C. to about 68° C., about 60° C. to about 67° C., or about 62° C. to about 66° C. In embodiments, the ligation is performed at about 60° C. to about 70° C., about 61° C. to about 69° C., or about 62° C. to about 68° C. In embodiments, the ligation is performed at about 60° C., about 61° C., about 62° C., about 63° C., about 64° C., about 65° C. about 66° C., about 67° C., about 68° C., about 69° C., or about 70° C.

In embodiments, the targeting probe comprises, in 5′- to 3′-order: the oligonucleotide tag, and a sequence that is complementary to a first region of the target nucleic acid. In embodiments where the method detects a polymorphic site (SNP), the first region of the target nucleic acid comprises the polymorphic site. In embodiments, the oligonucleotide tag comprises a single-stranded oligonucleotide. In embodiments, the oligonucleotide tag does not hybridize with the target nucleic acid. In embodiments, the detection probe comprises, in 5′- to 3′-order: a sequence that is complementary to a second region of the target nucleic acid that is adjacent to the first region, and a detectable label or binding partner thereof. In embodiments, the 5′ end of the targeting probe is phosphorylated and is adjacent to the 3′ hydroxyl of the detection probe when the targeting and detection probes are hybridized to the target nucleic acid, such that the ends of the targeting and detection probes are ligated by formation of a phosphodiester bond. In embodiments, the 5′ end of the detection probe is phosphorylated and is adjacent to the 3′ hydroxyl of the targeting probe when the targeting and detection probes are hybridized to the target nucleic acid, such that the ends of the targeting and detection probes are ligated by formation of a phosphodiester bond. In embodiments, the targeting probe and/or the detection probe comprises a modified nucleotide, e.g., a locked nucleic acid (LNA).

In embodiments, the targeting and detection probes are ligated using a template-dependent ligase, for example, a DNA ligase such as E. coli DNA ligase, T4 DNA ligase, T. aquaticus (Taq) ligase, T. Thermophilus DNA ligase, or Pyrococcus DNA ligase. In embodiments, the ligase is a thermostable ligase. In embodiments, the targeting and detection probes are ligated by chemical ligation. In embodiments, the hybridization and ligation are performed in a combined step, for example, using multiple thermocycles and a thermostable ligase.

In embodiments where the method detects a coronavirus nucleic acid (e.g., the SARS-CoV-2 N gene or the N1, N2, and/or N3 regions thereof), the targeting probe hybridizes to the target nucleic acid such that the terminal 5′ nucleotide of the targeting probe hybridizes with the first region in the target nucleic acid, and the detection probe hybridizes to the second region in the target nucleic acid that is adjacent to the first region and provides a 3′ end for the ligation of the targeting and detection probes. In embodiments, the detection probe hybridizes to the target nucleic acid such that the terminal 5′ nucleotide of the detection probe hybridizes with the first region in the target nucleic acid, and the targeting probe hybridizes to the second region in the target nucleic acid that is adjacent to the first region and provides a 3′ end for the ligation of the targeting and detection probes. In embodiments, the detection probe hybridizes to the target nucleic acid such that the terminal 3′ nucleotide of the detection probe hybridizes with the first region in the target nucleic acid, and the targeting probe hybridizes to the second region of the target nucleic acid that is adjacent to the first region and provides a 5′ end for the ligation of the targeting and detection probes.

In embodiments where the method detects an SNP, the targeting probe hybridizes to the target nucleic acid such that the terminal 5′ nucleotide of the targeting probe hybridizes with the SNP in the target nucleic acid, and the detection probe hybridizes to the target nucleic acid adjacent to the SNP and provides a 3′ end for the ligation of the targeting and detection probes. In embodiments, the detection probe hybridizes to the target nucleic acid such that the terminal 5′ nucleotide of the detection probe hybridizes with the SNP in the target nucleic acid, and the targeting probe hybridizes to the target nucleic acid adjacent to the SNP and provides a 3′ end for the ligation of the targeting and detection probes. In embodiments, the detection probe hybridizes to the target nucleic acid such that the terminal 3′ nucleotide of the detection probe hybridizes with the SNP in the target nucleic acid, and the targeting probe hybridizes to the target nucleic acid adjacent to the SNP and provides a 5′ end for the ligation of the targeting and detection probes.

In embodiments, the method further comprises providing a blocking probe during the ligating of the targeting and detection probes. In embodiments, a blocking probe reduces non-specific bridging background during the ligation reaction. In embodiments, the blocking probe comprises a single stranded oligonucleotide that is complementary to the target nucleic acid and straddles the ligation site but does not comprise an oligonucleotide tag or a detectable label or binding partner thereof. In embodiments, the blocking probe comprises a single stranded oligonucleotide that is complementary to a probe designed to hybridize to the target nucleic acid. Without being bound by theory, it is believed that the presence of a blocking probe can reduce formation of complexes in which the target nucleic acid functions as a “bridge” for probes that are annealed to the target nucleic acid, but not ligated to one another, such that the complex can generate a false signal. In embodiments, a pair of blocking probes is provided during the ligating. In embodiments, one or more blocking probes is provided during the ligating in excess over the corresponding targeting and/or detection probes.

In embodiments, the detection probe comprises a detectable label. In embodiments, the detection probe comprises a binding partner of a detectable label. Detectable labels are described herein. In embodiments, the detectable label is an electrochemiluminescence (ECL) label. In embodiments, the detection probe comprises biotin, and the detectable label comprises an ECL label linked to avidin or streptavidin. In embodiments, the detection probe comprises avidin or streptavidin, and the detectable label comprises an ECL label linked to biotin. Additional non-limiting examples of binding partners that can be on the detection probe and detectable label are provided herein.

In embodiments, the target nucleic acid in the sample comprises a coronavirus nucleic acid. In embodiments, the target nucleic acid in the sample comprises an RT-PCR product, e.g., cDNA generated from the coronavirus nucleic acid. In embodiments, the method further comprises amplifying the target nucleic acid prior to contacting with the oligonucleotide probes. In embodiments, the method does not comprise amplifying the target nucleic acid. In embodiments, the nucleic acid is coronavirus RNA, and the method comprises reverse transcribing the coronavirus RNA into cDNA prior to step (a). In embodiments, the targeting probe and/or detection probe hybridize to the cDNA strand comprising the SNP of interest. In embodiments where the SNP of interest is in a protein coding sequence, the targeting probe and/or detection probe hybridize to the cDNA strand comprising the protein coding sequence. In embodiments, the targeting probe and/or detection probe hybridize to the cDNA strand comprising a complement of the SNP of interest. In embodiments wherein the SNP of interest is in a protein coding sequence, the targeting probe and/or detection probe hybridize to the cDNA strand comprising the complementary strand of the protein coding sequence. In embodiments, the coronavirus is SARS-CoV-2.

In embodiments, a region of the SARS-CoV-2 genome surrounding the SNP of interest is reverse transcribed prior to step (a). In embodiments, the cDNA formed by the reverse transcription is amplified by PCR. Exemplary PCR primers for amplification are shown in Tables 9 and 14, and described in Lu et al., Emerg Infect Dis 26(8):1654-1665 (2020). In embodiments, the method comprises detecting an SNP in a synthetic oligonucleotide template. In embodiments, the region surrounding a SARS-CoV-2 genome location described in Table 1A and/or Table 1C is reverse transcribed prior to step (a).

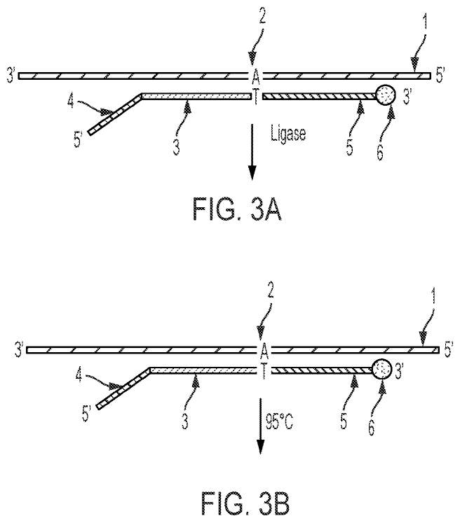

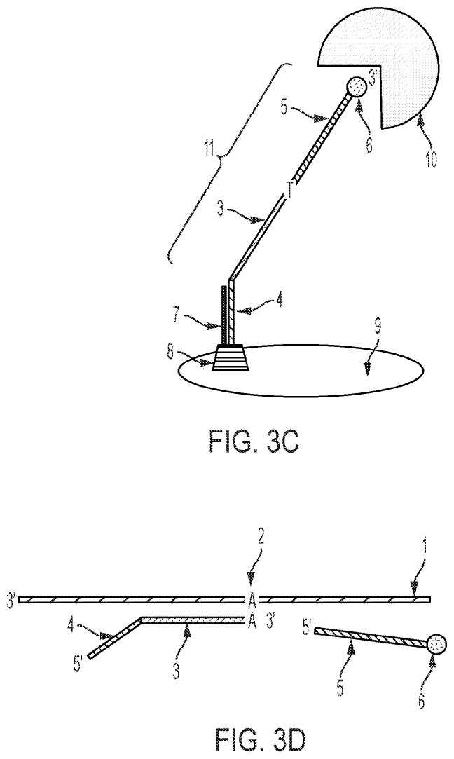

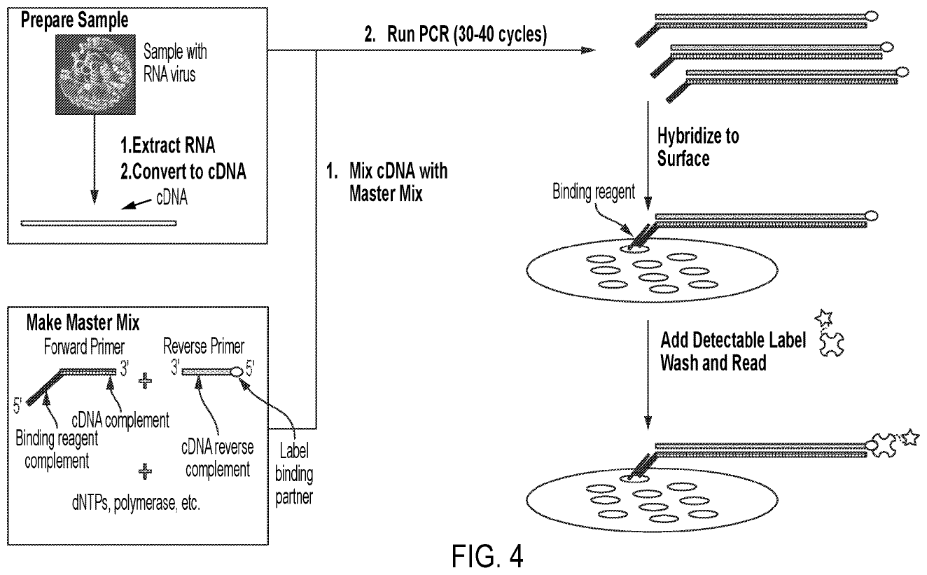

An embodiment of the OLA method for detecting an SNP described herein is represented schematically in FIG. 3. In FIGS. 3A-3C, a target nucleic acid (1) that comprises an SNP (2) is contacted with: a targeting probe (3) that comprises an oligonucleotide tag (4) and a sequence that is complementary to the SNP, and a detection probe (5) that comprises detectable label (6). The targeting and detection probes (3, 5) hybridize to the target nucleic acid, and the targeting and detection probes that hybridize with perfect complementarity at the SNP are ligated to form a ligated target complement (11) comprising the oligonucleotide tag and detectable label. The reaction mixture containing the ligated target complement is contacted with a surface comprising one or more binding reagents (7) immobilized in one or more binding domains (9). A signal (10) is detected if the ligated target complement is immobilized on the surface via hybridization of the complementary oligonucleotides in the oligonucleotide tag and the binding reagent. In FIG. 3D, the targeting probe has a mismatch with the SNP in the target nucleic acid, and thus, hybridization and ligation do not occur.

In embodiments, the method is a multiplexed OLA method. In embodiments where the method detects a coronavirus nucleic acid, the biological sample is contacted with one or more targeting probes and one or more detection probes to different regions of the coronavirus nucleic acid to form a plurality of ligated target complements. In embodiments, targeting probes for individual coronavirus nucleic acid regions comprise oligonucleotide tags corresponding to the individual coronavirus nucleic acid regions. In embodiments where the method detects an SNP, the biological sample is contacted with one or more SNP-specific targeting probes and one or more detection probes to form a plurality of ligated target complements. In embodiments, the detection probes comprise identical sequences. In embodiments, each of the one or more SNP-specific targeting probes hybridizes to a different SNP at the target nucleic acid (e.g., any of the SARS-CoV-2 genome locations and SNPs in Tables 1A and 1C herein). In embodiments, targeting probes for different SNPs comprise different oligonucleotide tags. In embodiments, the targeting probes for different coronavirus nucleic acid regions or for different SNPs have substantially the same melting temperatures (T_{M}), e.g., within about 5° C., within about 4C, within about 3° C., within about 2° C., or within about 1° C. In embodiments, the surface comprises a plurality of binding reagents capable of hybridizing to the different oligonucleotide tags. In embodiments, a plurality of binding complexes, each comprising a ligated target complement and its corresponding binding reagent, are formed on the surface, and the binding complexes are detected, thereby detecting, identifying, and/or quantifying each of the different coronavirus nucleic acid regions or each of the SNPs at the polymorphic site of the coronavirus nucleic acid. In embodiments, the coronavirus is SARS-CoV-2. In embodiments, the coronavirus nucleic acid is RNA. In embodiments, the different coronavirus regions comprise the N1, N2, and N3 regions of SARS-CoV-2.

In embodiments, the multiplexed OLA method simultaneously detects at least 2, at least 3, at least 4, at least 5, at least 6, at least 7, at least 8, at least 9, or at least 10 coronavirus nucleic acids as described herein. In embodiments, the multiplexed OLA method simultaneously detects at least 2, at least 3, at least 4, at least 5, at least 6, at least 7, at least 8, at least 9, or at least 10 SNPs. In embodiments, the multiplexed OLA method detects any combination of the SNPs in Table 1A. In embodiments, the multiplexed OLA method simultaneously detects the reference SARS-CoV-2 strain and one or more variants, e.g., by detecting both the wild-type nucleotide and the variant SNP at a genome location. As used herein, “variant” refers to a strain that has one or more mutations relative to the SARS-CoV-2 reference strain NC_045512. In embodiments, the multiplexed OLA method comprises contacting the biological sample with a surface comprising at least 2, at least 3, at least 4, at least 5, at least 6, at least 7, at least 8, at least 9, or at least 10 distinct binding domains, wherein each binding domain comprises a unique binding reagent, each unique binding reagent capable of recognizing a different oligonucleotide tag as described herein.

In embodiments, the method further comprises detecting a control gene. In embodiments, the control gene comprises an endogenous gene of the subject from which the biological sample was obtained. In embodiments, the control gene comprises the human RPP30 gene. In embodiments, the targeting probe for the human RPP30 gene comprises SEQ ID NO:35 or 37. In embodiments, the detection probe for the human RPP30 gene comprises SEQ ID NO:36 or 38. In embodiments, the blocking probe for the human RPP30 gene comprises any one of SEQ ID NOs:51-54.

In embodiments, the invention provides a method for detecting a coronavirus nucleic acid in a biological sample. In embodiments, the method comprises: (a) contacting the biological sample with (i) a polymerase; (ii) a forward primer, wherein the forward primer binds to a first region of a target nucleic acid (e.g., the coronavirus nucleic acid or an RT-PCR product described herein), and wherein the forward primer comprises an oligonucleotide tag; and (iii) a reverse primer, wherein the reverse primer binds to a second region of the target nucleic acid; (b) amplifying the target nucleic acid using the polymerase to form an amplified target nucleic acid comprising the oligonucleotide tag; (c) hybridizing the amplified target nucleic acid with an internal detection probe that is complementary to at least a portion of the amplified target nucleic acid, thereby forming a hybridized target; (d) contacting the hybridized target with a surface comprising a binding reagent immobilized in one or more binding domains, wherein the binding reagent comprises an oligonucleotide complementary to the oligonucleotide tag; (e) forming a binding complex on the surface between the binding reagent and the hybridized target; and (f) detecting the binding complex, thereby detecting, identifying, and/or quantifying the coronavirus nucleic acid. In embodiments, the coronavirus is SARS-CoV-2. In embodiments, the coronavirus nucleic acid is RNA. In embodiments, the target nucleic acid is the N1, N2, and/or N3 regions of SARS-CoV-2. In embodiments, the sample comprises the coronavirus nucleic acid. In embodiments, the sample comprises an RT-PCR product, e.g., cDNA that is generated from the coronavirus nucleic acid.

In embodiments, the method further comprises amplifying the target nucleic acid prior to contacting with the oligonucleotide probes. In embodiments, the nucleic acid is coronavirus RNA, and the method comprises reverse transcribing the coronavirus RNA into cDNA prior to step (a). In embodiments, the coronavirus is SARS-CoV-2

In embodiments, the internal detection probe comprises a detectable label. In embodiments, the internal detection probe comprises a binding partner of a detectable label. Detectable labels are described herein. In embodiments, the detectable label is an electrochemiluminescence (ECL) label. In embodiments, the internal detection probe comprises biotin, and the detectable label comprises an ECL label linked to avidin or streptavidin. In embodiments, the internal detection probe comprises avidin or streptavidin, and the detectable label comprises an ECL label linked to biotin. Additional non-limiting examples of binding partners that can be on the internal detection probe and detectable label are provided herein.

In embodiments, the method further comprises detecting a control gene. In embodiments, the control gene comprises an endogenous gene of the subject from which the biological sample was obtained. Suitable control genes are known to those of skill in the art. In embodiments, the control gene comprises the human RPP30 gene. In embodiments, the forward primer for the human RPP30 gene comprises SEQ ID NO:64. In embodiments, the reverse primer for the human RPP30 gene comprises SEQ ID NO:65. In embodiments, the internal detection probe for the human RPP30 gene comprises SEQ ID NO:66.

### Manual and Automated Embodiments

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477. In embodiments, the methods herein are performed in an automated cartridge reader as described herein. Manual and automated systems for use with the methods and kits described herein are known in the art and described, e.g., in U.S. Publication No. 2022/0003766 and U.S. Publication No. 2021/0349104.

### Antibody and Composition

In embodiments, the invention provides an antibody or antigen-binding fragment thereof that specifically binds a viral antigen described herein, e.g., a SARS-CoV-2 protein. In embodiments, the invention provides an antibody or antigen-binding fragment thereof that specifically binds a SARS-CoV-2 N protein or a SARS-CoV-2 S protein. In embodiments, the invention provides an antibody or antigen-binding fragment thereof that specifically binds SARS-CoV-2 S1, S2, S-ECD, S-NTD, or S-RBD. In embodiments, the invention provides an antibody or antigen-binding fragment thereof that specifically binds a SARS-CoV-2 S protein or subunit or fragment thereof that comprises any of the mutations in Tables 1A and 1B. In embodiments, the invention provides an antibody or antigen-binding fragment thereof that specifically binds an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, an F protein from RSV, or a combination thereof.

In embodiments, the antibody or antigen-binding fragment thereof is a binding reagent, e.g., as disclosed herein, for an assay described herein, e.g., for detecting a viral component in a sample. In embodiments, the antibody or antigen-binding fragment thereof is capable of being immobilized onto a surface, e.g., as disclosed herein. In embodiments, the invention provides a composition comprising: (i) the antibody or antigen-binding fragment thereof; and (ii) a surface. In embodiments, the antibody or antigen-binding fragment thereof is immobilized onto the surface.

In embodiments, the antibody or antigen-binding fragment thereof is a detection reagent, e.g., as disclosed herein, for an assay described herein, e.g., for detecting a viral component in a sample. In embodiments, the antibody or antigen-binding fragment thereof comprises a detectable label. In embodiments, the antibody or antigen-binding fragment is capable of being conjugated with a detectable label. In embodiments, the invention provides a composition comprising: (i) the antibody or antigen-binding fragment thereof; (ii) a detectable label, e.g., as disclosed herein; and (iii) a reagent for conjugating the detectable label to the antibody or antigen-binding fragment thereof. In embodiments, the detectable label is an ECL label. In embodiments, the antibody or antigen-binding fragment thereof comprises a nucleic acid probe. In embodiments, the antibody or antigen-binding fragment is capable of being conjugated with a nucleic acid probe. In embodiments, the invention provides a composition comprising: (i) the antibody or antigen-binding fragment thereof; (ii) a nucleic acid probe, e.g., as disclosed herein; and (iii) a reagent for conjugating the nucleic acid probe to the antibody or antigen-binding fragment thereof.

In embodiments, the antibody or antigen-binding fragment thereof is a calibration reagent, e.g., as disclosed herein, for a serology assay, e.g., a classical, bridging, or competitive serology assay, e.g., as disclosed herein. In embodiments, the antibody or antigen-binding fragment thereof is a competitor for a competitive serology assay.

In embodiments, the invention provides a therapeutic composition comprising the antibody or antigen-binding fragment thereof. In embodiments, the therapeutic composition is capable of treating or preventing infection by a virus described herein, e.g., SARS-CoV-2 and/or a variant thereof.

In embodiments, the invention provides a composition comprising (i) the antibody or antigen-binding fragment thereof and (ii) a viral antigen that specifically binds the antibody or antigen-binding fragment.

In general, an antibody (used interchangeably with the term “immunoglobulin”) comprises at least the variable domain of a heavy chain; typically, an antibody comprises the variable domains of a heavy chain and a light chain. Both the heavy and light chains are divided into regions of structural and functional homology. Generally, the variable domain of a heavy chain (V_{H}) or light chain (V_{L}) determines antigen recognition and specificity, and the constant domain of a heavy chain (C_{H1}, C_{H2}, or C_{H3}) or light chain (C_{L}) confers biological properties such as secretion, receptor binding, complement binding, and the like. Generally, the N-terminal portion of an antibody chain is a variable portion, and the C-terminal portion is a constant region; the C_{H3} and C_{L} domains typically comprise the C-terminus of the heavy chain and light chain, respectively.

In general, antibodies are encoded by immunoglobulin genes or fragments of immunoglobulin genes. The recognized immunoglobulin genes include the kappa, lambda, alpha, gamma, delta, epsilon, and mu constant region genes, as well as immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

In general, the variable region allows the antibody to selectively recognize and specifically bind epitopes on antigens. Thus, the V_{L} domain and V_{H} domain, or a subset of the complementarity determining regions (CDR) within these variable domains, of an antibody combine to form the variable region that forms an antigen binding domain. The antigen binding domain is typically defined by three CDRs on each of the V_{L} and V_{H} domains. The six “complementarity determining regions” or “CDRs” typically present in each antigen binding domain are short, non-contiguous sequences of amino acids that are specifically positioned to form the antigen binding domain. The antigen binding domain formed by the positioned CDRs defines a surface complementary to the epitope on the antigen. This complementary surface promotes the non-covalent binding of the antibody to its cognate epitope.

In embodiments, the antibody or antigen-binding fragment thereof comprises a constant region comprising an IgA, IgD, IgE, IgG, or IgM domain. In embodiments, the antibody or antigen-binding fragment thereof comprises an IgG domain. In embodiments, the antibody or antigen-binding fragment thereof is an IgG1, IgG2, IgG3, or IgG4 isotype antibody or antigen-binding fragment thereof. In embodiments, the antibody or antigen-binding fragment thereof is lgG2a, lgG2b, or lgG2c subclass antibody or antigen-binding fragment thereof.

In embodiments, the antibody or antigen-binding fragment thereof is derived from a mouse, rat, goat, rabbit, chicken, guinea pig, hamster, horse, sheep, ferret, minx, or bat. In embodiments, the antibody or antigen-binding fragment thereof is humanized. In embodiments, the antibody or antigen-binding fragment thereof is capable of being administered to a human or an animal subject described herein, e.g., mouse, rat, ferret, minx, bat, a domestic animal, or an NHP. In embodiments, the antibody or antigen-binding fragment thereof is non-immunogenic to a human or an animal subject described herein, e.g., mouse, rat, ferret, minx, bat, a domestic animal, or an NHP.

### Kits

In embodiments, the invention provides a kit comprising, in one or more vials, containers, or compartments: (a) a viral antigen that specifically binds a biomarker, e.g., an antibody biomarker; and (b) a detection reagent that specifically binds the biomarker, e.g., the antibody biomarker. In embodiments, the kit further comprises a surface. Antibody biomarkers and their binding partners, e.g., viral antigens, are described herein. In embodiments, the detection reagent is an antibody or antigen-binding fragment thereof. In embodiments, the detection reagent is a second copy of the viral antigen.

In embodiments, the viral antigen is a respiratory virus antigen. In embodiments, the respiratory virus is a coronavirus, an influenza virus, a paramyxovirus, an adenovirus, a bocavirus, a pneumovirus, an enterovirus, a rhinovirus, or a combination thereof. In embodiments, the viral antigen is a coronavirus S protein or fragment thereof. In embodiments, the coronavirus is SARS-CoV, MERs-CoV, SARS-CoV-2, HCov-OC43, HCoV-229E, HCoV-NL63, HCoV-HKU1, or a combination thereof. In embodiments, the viral antigen is SARS-CoV-2 S protein, S1 subunit, S2 subunit, S-RBD, M protein, E protein, N protein, or a combination thereof.

In embodiments, the invention provides a kit comprising, in one or more vials, containers, or compartments: (a) a binding reagent that specifically binds a biomarker, e.g., an inflammatory or tissue damage response biomarker; and (b) a detection reagent that specifically binds the biomarker, e.g., the inflammatory or tissue damage response biomarker. In embodiments, the kit further comprises a surface. Inflammatory and tissue damage response biomarkers and binding and detection reagents therefor are described herein. In embodiments, the binding reagent is an antibody or antigen-binding fragment. In embodiments, the detection reagent is an antibody or antigen-binding fragment thereof.

In embodiments, the invention provides a kit comprising: (a) one or more binding reagents that specifically bind one or more antibody biomarkers, wherein each antibody biomarker specifically binds to a SARS-CoV-2 antigen; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises the SARS-CoV-2 antigen(s) to which the antibody biomarkers specifically bind. In embodiments, the one or more detection reagents specifically binds to the antibody biomarker. In embodiments, the one or more detection reagents specifically binds IgA, IgG, or IgM. In embodiments, the one or more detection reagents comprises the SARS-CoV-2 antigen(s) to which the antibody biomarkers specifically bind. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises four distinct binding domains, e.g., as shown in FIG. 29A. In embodiments, the surface comprises a multi-well assay plate, wherein each well comprises ten distinct binding domains, e.g., as shown in FIG. 29B. In embodiments, the assay plate is a 96-well assay plate. In embodiments, the assay plate is a 384-well assay plate.

In embodiments, the kit described herein comprises a surface comprising one or more binding domains, wherein each binding domain comprises a binding reagent, e.g., a viral antigen immobilized thereon or capable of being immobilized thereon. In embodiments, each binding domain comprises an immobilized antigen of a panel of antigens. In embodiments, the surface comprises a well of an assay plate. In embodiments, each well of the assay plate comprises ten distinct binding domains, wherein each binding domain comprises an immobilized viral (e.g., a respiratory virus such as SARS-CoV-2) antigen of a panel of viral antigens as described herein. In embodiments, the panel of viral antigens, e.g., SARS-CoV-2 antigens, are immobilized or capable of being immobilized on a surface comprising Spots 1-10 as shown in FIG. 29B, wherein the antigens are arranged as shown in Tables 2A-2H. In embodiments, the S protein mutations from the SARS-CoV-2 strains of Tables 2A-2H are described in Table 1D. In embodiments, the S-RBD mutations from the SARS-CoV-2 strains of Tables 2A-2H are described in Table 1E. In embodiments, the detection reagents of the kit comprise the same antigens as those immobilized on the surface. In embodiments, the detection reagents of the kit comprise one or more antibodies that specifically bind IgA, IgG, or IgM. In embodiments, the detection reagents of the kit comprise ACE2.

In embodiments, the surface of the kits described herein comprises a multi-well assay plate. In embodiments, the surface comprises avidin or streptavidin. In embodiments, each binding reagent comprises biotin. In embodiments, the surface comprises a targeting agent. In embodiments, the kit further comprises a linking agent connected to a targeting agent complement. In embodiments, each binding reagent comprises a supplemental linking agent. In embodiments, the targeting agent and targeting agent complement comprise complementary oligonucleotides. In embodiments, the linking agent comprises avidin or streptavidin, and the supplemental linking agent comprises biotin. Targeting agents, targeting agent complements, linking agents, and supplemental linking agents are further described herein.

In embodiments, the invention provides a kit comprising: (a) one or more binding reagents, each binding reagent binding specifically to: (1) a viral component; (2) a host antibody biomarker; or (3) a host inflammatory and/or tissue damage response biomarker; and (b) one or more detection reagents, each detection reagent binding specifically to the viral component, host antibody biomarker, or the host inflammatory and/or tissue damage response biomarker. In embodiments, the detection reagent that binds to the host antibody biomarker binds IgA, IgG, or IgM. In embodiments, the detection reagent is ACE2. In embodiments, the viral component is a viral protein. In embodiments, the viral component is a viral nucleic acid. In embodiments, the virus is a coronavirus. In embodiments, the virus is SARS-CoV-2. In embodiments, the kit further comprises a surface.

In embodiments, the invention provides a combination of any of the kits described herein. In embodiments, the combination of kits is provided as a single kit, comprising the components of each of the individual kits.

In embodiments, the binding reagent is an antibody or antigen-binding fragment thereof. In embodiments, the detection reagent is an antibody or antigen-binding fragment thereof. In embodiments, any of the detection reagents described herein comprises a detectable label as described herein. In embodiments, the detection reagent comprises a nucleic acid probe as described herein. In embodiments, the kit comprises first and second detection reagents, and the first and second detection reagents respectively comprise first and second nucleic acid probes as described herein. In embodiments, the kit further comprises a reagent for conjugating the detection reagent to a detectable label or a nucleic acid probe.

In embodiments, the detection reagent is lyophilized. In embodiments, the detection reagent is provided in solution. In embodiments, the binding reagent is immobilized on the binding domain. In embodiments, the binding reagent is provided in solution. In embodiments, the reagents and other components of the kit are provided separately. In embodiments, they are provided separately according to their optimal shipping or storage temperatures.

Reagents and methods for immobilizing binding reagents to surfaces, e.g., via targeting agents/targeting agent complements, linking agents/supplemental linking agents, and bridging agents are described herein. In embodiments, the surface is a plate. In embodiments, the surface is a multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In some embodiments, the particle comprises a microsphere. In embodiments, the particle comprises a paramagnetic bead. In embodiments, the surface is a cartridge. In embodiments, the surface comprises an electrode. In embodiments, the electrode is a carbon ink electrode.

In embodiments, the kit further comprises a calibration reagent. In embodiments, the calibration reagent comprises a known quantity of the virus, viral component, or biomarker as described herein. In embodiments, multiple calibration reagents comprise a range of concentrations of the virus, viral component, or biomarker. In embodiments, the multiple calibration reagents comprise concentrations of the virus, viral component, or biomarker near the upper and lower limits of quantitation for the immunoassay. In embodiments, the multiple concentrations of the calibration reagent span the entire dynamic range of the immunoassay. In embodiments, the calibration reagent comprises an antibody biomarker. In embodiments, the antibody biomarker is a neutralizing antibody as described herein. In embodiments, the neutralizing antibody is a monoclonal antibody. In embodiments, the calibration reagent comprises a neutralizing antibody that specifically binds the SARS-CoV S protein, the SARS-CoV-2 S protein, or both. In embodiments, the calibration reagent is derived from human serum known to contain one or more antibodies that specifically bind to one or more viral antigens described herein. In embodiments, the one or more antibodies is human IgG, human IgM, or a combination thereof. In embodiments, the calibration reagent comprises an antibody that specifically binds the SARS-CoV S protein, an antibody that specifically binds SARS-CoV-2 S-NTD, an antibody that specifically binds SARS-CoV-2 S protein, an antibody that specifically binds SARS-CoV-2 S-RBD, an antibody that specifically binds SARS-CoV-2 N protein, an antibody that specifically binds HCoV-OC43 S protein, an antibody that specifically binds HCoV-HKU1 S protein, an antibody that specifically binds MERS-CoV S protein, an antibody that specifically binds HCoV-NL63 S protein, an antibody that specifically binds HCoV-229E S protein, an antibody that specifically binds influenza A/Hong Kong H3 HA protein, an antibody that specifically binds influenza B/Brisbane HA protein, an antibody that specifically binds influenza A/Shanghai H7 HA protein, an antibody that specifically binds influenza A/Michigan H1 HA protein, an antibody that specifically binds influenza B/Phuket HA protein, and an antibody that specifically binds RSV pre-fusion F protein. In embodiments, the calibration reagent comprises an IgG that specifically binds to SARS-CoV-2 S protein, an IgG that specifically binds to SARS-CoV-2 N protein, an IgG that specifically binds to SARS-CoV-2 S-RBD, an IgM that specifically binds to SARS-CoV-2 S protein, an IgM that specifically binds to SARS-CoV-2 N protein, an IgM that specifically binds to SARS-CoV-2 S-RBD, an IgA that specifically binds to SARS-CoV-2 S protein, an IgA that specifically binds to SARS-CoV-2 N protein, and an IgA that specifically binds to SARS-CoV-2 S-RBD.

In embodiments, the calibration reagents are provided in the kit at the following concentrations: about 1 to about 10 BAU/mL of an IgG that specifically binds to SARS-CoV-2 S protein, about 0.1 to about 5 BAU/mL of an IgG that specifically binds to SARS-CoV-2 N protein, about 5 to about 20 BAU/mL of an IgG that specifically binds to SARS-CoV-2 S-RBD, about 0.1 to about 2 BAU/mL of an IgM that specifically binds to SARS-CoV-2 S protein, about 1 to about 5 BAU/mL of an IgM that specifically binds to SARS-CoV-2 N protein, about 0.1 to about 2 BAU/mL of an IgM that specifically binds to SARS-CoV-2 S-RBD, about 1 to about 5 BAU/mL of an IgA that specifically binds to SARS-CoV-2 S protein, about 1 to about 10 BAU/mL of an IgA that specifically binds to SARS-CoV-2 N protein, and about 0.1 to about 5 BAU/mL of an IgA that specifically binds to SARS-CoV-2 S-RBD. Concentrations of the calibration reagents provided herein are defined according to the “First WHO International Standard for anti-SARS-CoV-2 immunoglobulin” (NIBSC code: 20/136).

In embodiments, the calibration reagents are provided in the kit at the following concentrations: about 6.31 BAU/mL of an IgG that specifically binds to SARS-CoV-2 S protein, about 1.89 BAU/mL of an IgG that specifically binds to SARS-CoV-2 N protein, about 8.16 BAU/mL of an IgG that specifically binds to SARS-CoV-2 S-RBD, about 0.867 BAU/mL of an IgM that specifically binds to SARS-CoV-2 S protein, about 2.64 BAU/mL of an IgM that specifically binds to SARS-CoV-2 N protein, about 0.466 BAU/mL of an IgM that specifically binds to SARS-CoV-2 S-RBD, about 3.09 BAU/mL of an IgA that specifically binds to SARS-CoV-2 S protein, about 5.57 BAU/mL of an IgA that specifically binds to SARS-CoV-2 N protein, and about 1.56 BAU/mL of an IgA that specifically binds to SARS-CoV-2 S-RBD.

In embodiments, the calibration reagent is a positive control reagent. In embodiments, the calibration reagent is a negative control reagent. In embodiments, the positive or negative control reagent is used to provide a basis of comparison for the biological sample to be tested with the methods of the present invention. In embodiments, the positive control reagent comprises multiple concentrations of the virus, viral component, or biomarker. In embodiments, the positive control reagent comprises an antibody. In embodiments, the positive control reagent comprises human IgG, IgM, IgA, or a combination thereof. In embodiments, the positive control reagent comprises an antibody that specifically binds the SARS-CoV-2 S protein, SARS-CoV-2 N protein, SARS-CoV-2 S-RBD, or a combination thereof. In embodiments, the positive control reagent comprises an IgG that specifically binds to SARS-CoV-2 S protein, an IgG that specifically binds to SARS-CoV-2 N protein, an IgG that specifically binds to SARS-CoV-2 S-RBD, an IgM that specifically binds to SARS-CoV-2 S protein, an IgM that specifically binds to SARS-CoV-2 N protein, an IgM that specifically binds to SARS-CoV-2 S-RBD, an IgA that specifically binds to SARS-CoV-2 S protein, an IgA that specifically binds to SARS-CoV-2 N protein, and an IgA that specifically binds to SARS-CoV-2 S-RBD.

In embodiments, the positive control reagent is provided in the kit at the following concentrations: about 0.005 to about 1 BAU/mL of an IgG that specifically binds to SARS-CoV-2 S protein; about 0.001 to about 0.1 BAU/mL of an IgG that specifically binds to SARS-CoV-2 N protein; about 0.005 to about 1 BAU/mL of an IgG that specifically binds to SARS-CoV-2 S-RBD; about 0.001 to about 0.1 BAU/mL of an IgM that specifically binds to SARS-CoV-2 S protein; about 0.01 to about 0.1 BAU/mL of an IgM that specifically binds to SARS-CoV-2 N protein; about 0.001 to about 0.1 BAU/mL of an IgM that specifically binds to SARS-CoV-2 S-RBD; about 0.005 to about 0.5 BAU/mL of an IgA that specifically binds to SARS-CoV-2 S protein; about 0.005 to about 0.5 BAU/mL of an IgA that specifically binds to SARS-CoV-2 N protein; and about 0.001 to about 0.1 BAU/mL of an IgA that specifically binds to SARS-CoV-2 S-RBD. In embodiments, the positive control reagents are provided in the kit at the following concentrations: about 0.1504, about 0.0372, and about 0.0133 BAU/mL of an IgG that specifically binds to SARS-CoV-2 S protein; about 0.0457, about 0.0078, and about 0.0025 BAU/mL of an IgG that specifically binds to SARS-CoV-2 N protein; about 0.1952, about 0.0576, and about 0.0148 BAU/mL of an IgG that specifically binds to SARS-CoV-2 S-RBD; about 0.0187, about 0.0054, and about 0.0077 BAU/mL of an IgM that specifically binds to SARS-CoV-2 S protein; about 0.061, about 0.030, and about 0.0285 BAU/mL of an IgM that specifically binds to SARS-CoV-2 N protein; about 0.011, about 0.0047, and about 0.0068 BAU/mL of an IgM that specifically binds to SARS-CoV-2 S-RBD; about 0.0768, about 0.023, and about 0.0103 BAU/mL of an IgA that specifically binds to SARS-CoV-2 S protein; about 0.1414, about 0.0237, and about 0.0379 BAU/mL of an IgA that specifically binds to SARS-CoV-2 N protein; and about 0.0394, about 0.0131, and about 0.0085 BAU/mL of an IgA that specifically binds to SARS-CoV-2 S-RBD.

In embodiments, the calibration reagent is lyophilized. In embodiments, the calibration reagent is provided in solution. In embodiments, the calibration reagent is provided as a stock concentration that is 5×, 10×, 20×, 30×, 40×, 50×, 60×, 70×, 80×, 90×, 100×, 125×, 150× or higher fold concentrations of the highest working concentration of the calibration reagent. In embodiments, the kit further comprises a diluent for preparing multiple concentrations of the calibration reagent. In embodiments, the calibration reagent provided in the kit is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. In embodiments, the kit comprises multiple calibration reagents at multiple concentrations, e.g., two or more, three or more, four or more, or five or more concentrations. In embodiments, the multiple concentrations of calibration reagents are used to calculate a standard curve. In embodiments, the multiple concentrations of calibration reagents provide thresholds indicating low, medium, or high levels of the virus, viral component, or biomarker being measured.

In embodiments, the kit further comprises a sample collection device. In embodiments, the sample collection device is an applicator stick. In embodiments, the sample collection device is a swab. In embodiments, the sample collection device is a tissue scraper. In embodiments, the sample collection device is a vial or container for collecting a liquid sample.

In embodiments, the kit further comprises one or more of a buffer, e.g., assay buffer, reconstitution buffer, storage buffer, read buffer, wash buffer and the like; a diluent; a blocking solution; an assay consumable, e.g., assay modules, vials, tubes, liquid handling and transfer devices such as pipette tips, covers and seals, racks, labels, and the like; an assay instrument; and/or instructions for carrying out the assay.

In embodiments, the kit comprises lyophilized reagents, e.g., detection reagent and/or calibration reagent. In embodiments, the kit comprises one or more solutions to reconstitute the lyophilized reagents.

In embodiments, a kit comprising the components above include stock concentrations of the components that are 5×, 10×, 20×, 30×, 40×, 50×, 60×, 70×, 80×, 90×, 100×, 125×, 150× or higher fold concentrations of the working concentrations of the immunoassays herein.

In embodiments, the invention provides a kit for collecting a biological sample. In embodiments, the kit for collecting a biological sample can be provided to a subject for collecting the subject's own sample, e.g., saliva sample. The collected sample can then be provided by the subject, e.g., delivered in person or via postal service, to a laboratory for analysis. In embodiments, the kit further comprises an assay instrument, e.g., an assay cartridge and/or a cartridge reader, for the subject to analyze the collected sample. In embodiments, the kit comprises a sample collection device, e.g., an applicator stick, a swab, a tissue scraper, or a vial or container for collecting a liquid sample. In embodiments, the sample collection device comprises a straw for collecting a saliva sample. In embodiments, the sample collection device comprises a storage solution that stabilizes the sample. In embodiments, the sample collection device comprises a unique sample identifier, e.g., a barcode. In embodiments, the kit further comprises instructions for collecting the sample and/or for analyzing the sample in an assay instrument. In embodiments, the kit further comprises an absorbent material, e.g., a tissue. In embodiments, the kit further comprises a secondary container (e.g., a bag) to secure the sample collection device. In embodiments, the kit further comprises a pre-paid postage label or a pre-paid envelope or box for mailing the collected sample.

All references cited herein, including patents, patent applications, papers, textbooks and the like, and the references cited therein, to the extent that they are not already, are hereby incorporated herein by reference in their entirety.

### Exemplary Embodiments

The following are exemplary embodiments of the inventions disclosed herein.

Embodiment (E) 1 is a kit for detecting one or more antibody biomarkers of interest in a sample, the kit comprising, in one or more vials, containers, or compartments:
(a) a surface comprising one or more binding domains, wherein each binding domain comprises an immobilized antigen of a panel of antigens, and wherein the panel of antigens comprises:
(i) an S protein from the following SARS-CoV-2 strains: wild-type; BA.2.12.1; BA.2; AY.4; BA.3; BA.2+L452M; BA.2+L452R; BA.4; B.1.351; and BA.5; or
(ii) an S-RBD from the following SARS-CoV-2 strains: BA.2.12.1; B.1.351; BA.2; BA.2+L452M; BA.2+L452R; B.1.1.7; BA.4/BA.5; BA.3; AY.4; and wild-type; or
(iii) an S protein from the following SARS-CoV-2 strains: wild-type; BA.2.12.1; BA.2; AY.4; BA.2.75; BA.4; B.1.351; and BA.5; or
(iv) an S-RBD from the following SARS-CoV-2 strains: an S-RBD from the following SARS-CoV-2 strains: BA.2.12.1; B.1.351; BA.2; B.1.1.7; BA.4/BA.5; BA.2.75; AY.3; and wild-type; or
(v) an S protein from the following SARS-CoV-2 strains: wild-type, BA.2.12.1, BA.2.75, BA.2, BA.1 (B.1.1.529), BA.1.617.2, B.1.1.7, B.1.351, and BA.5; and an N protein from wild-type SARS-CoV-2; or
(vi) an S-RBD from the following SARS-CoV-2 strains: BA.2.12.1, B.1.351, BA.1 (B.1.1.529), BA.2, B.1.1.7, B.1.617.2, BA.2.75, BA.4/BA.5, and wild-type; and an N protein from wild-type SARS-CoV-2; or
(vii) an S protein from the following SARS-CoV-2 strains: wild-type; BA.1; XBB.1; BF.7; BA.2.75.2; BQ.1.1; BA.2.75; BA.4.6; BQ.1; and BA.5; or
(viii) an S-RBD from the following SARS-CoV-2 strains: BA.1; BQ.1.1; BA.2.75.2; BA.4.6/BF.7; XBB.1; BA.4/BA.5/BF.5; BA.2.75; BQ.1; and wild-type; or
(ix) an S protein from the following SARS-CoV-2 strains: wild-type; BA.1; XBB.1; BF.7; XBB.1.5; BQ.1.1; BA.2.75; BN.1; BQ.1; and BA.5;
(x) an S-RBD from the following SARS-CoV-2 strains: BA.1 BQ.1.1; XBB.1.5, BN.1; XBB.1; BA.5; BA.2.75; BQ.1; and wild-type;
(xi) virus-like particle (VLP) from enterovirus (EV)-D68; VLP from EV-71; F protein from metapneumovirus (MPV); pre-fusion F from RSV; HA proteins from Flu A/H1 (e.g., H1/Wisconsin 2019), Flu A/H3 (e.g., H3/Darwin 2021), and Flu B/Victoria (e.g., B/Austria 2021); capsid protein VP0 from rhinovirus C (RV-C); S protein from SARS-CoV-2 strain BA.5; F proteins from PIV1, PIV2, PIV3, and PIV4; and VP0 from parechovirus (PeV) A3,
   optionally wherein:
   the HA proteins from Flu A H1/Wisconsin, Flu A H3/Darwin, and Flu B Austria are in a same binding domain; and
   the F proteins from PIV1, PIV2, PIV3, and PIV4 are in a same binding domain; or
(xii) HA from Flu A/H1 (e.g., H1/Wisconsin 2019); HA from Flu A/H3 (e.g., H3 Darwin 2021); HA from Flu B/Victoria (e.g., B/Austria 2021); F protein from PIV1; F protein from PIV2, F protein from PIV3; and F protein from PIV4, and
(b) one or more detection reagents, wherein each detection reagent comprises a detection antibody, a detection antigen, or an ACE detection reagent.

E2 includes the kit of E1, wherein the detection reagent comprises an electrochemiluminescent (ECL) label.

E3 includes the kit of E1 or E2, wherein the surface comprises an electrode.

E4 includes the kit of any one of E1 to E3, wherein the surface comprises a well of a multi-well plate, and wherein each well comprises 1 to 10 binding domains.

E5 is a method of detecting one or more antibody biomarkers of interest in a sample, comprising:
(a) contacting the sample with a surface comprising one or more binding domains, wherein each binding domain comprises an immobilized antigen of a panel of antigens, and wherein the panel of antigens comprises:
(i) an S protein from the following SARS-CoV-2 strains: wild-type; BA.2.12.1; BA.2; AY.4; BA.3; BA.2+L452M; BA.2+L452R; BA.4; B.1.351; and BA.5; or
(ii) an S-RBD from the following SARS-CoV-2 strains: BA.2.12.1; B.1.351; BA.2; BA.2+L452M; BA.2+L452R; B.1.1.7; BA.4/BA.5; BA.3; AY.4; and wild-type; or
(iii) an S protein from the following SARS-CoV-2 strains: wild-type; BA.2.12.1; BA.2; AY.4; BA.2.75; BA.4; B.1.351; and BA.5; or
(iv) an S-RBD from the following SARS-CoV-2 strains: an S-RBD from the following SARS-CoV-2 strains: BA.2.12.1; B.1.351; BA.2; B.1.1.7; BA.4/BA.5; BA.2.75; AY.3; and wild-type; or
(v) an S protein from the following SARS-CoV-2 strains: wild-type, BA.2.12.1, BA.2.75, BA.2, BA.1 (B.1.1.529), BA.1.617.2, B.1.1.7, B.1.351, and BA.5; and an N protein from wild-type SARS-CoV-2; or
(vi) an S-RBD from the following SARS-CoV-2 strains: BA.2.12.1, B.1.351, BA.1 (B.1.1.529), BA.2, B.1.1.7, B.1.617.2, BA.2.75, BA.4/BA.5, and wild-type; and an N protein from wild-type SARS-CoV-2; or
(vii) an S protein from the following SARS-CoV-2 strains: wild-type; BA.1; XBB.1; BF.7; BA.2.75.2; BQ.1.1; BA.2.75; BA.4.6; BQ.1; and BA.5; or
(viii) an S-RBD from the following SARS-CoV-2 strains: BA.1; BQ.1.1; BA.2.75.2; BA.4.6/BF.7; XBB.1; BA.4/BA.5/BF.5; BA.2.75; BQ.1; and wild-type; or
(ix) an S protein from the following SARS-CoV-2 strains: wild-type; BA.1; XBB.1; BF.7; XBB.1.5; BQ.1.1; BA.2.75; BN.1; BQ.1; and BA.5;
(x) an S-RBD from the following SARS-CoV-2 strains: BA.1; BQ.1.1; XBB.1.5; BN.1; XBB.1; BA.5; BA.2.75; BQ.1; and wild-type;
(xi) virus-like particle (VLP) from enterovirus (EV)-D68; VLP from EV-71; F protein from metapneumovirus (MPV); pre-fusion F from RSV; HA proteins from Flu A/H1 (e.g., H1/Wisconsin 2019), Flu A/H3 (e.g., H3/Darwin 2021), and Flu B/Victoria (e.g., B/Austria 2021); capsid protein VP0 from rhinovirus C (RV-C); S protein from SARS-CoV-2 strain BA.5; F proteins from PIV1, PIV2, PIV3, and PIV4; and VP0 from parechovirus (PeV) A3,
   optionally wherein:
   the HA proteins from Flu A H1/Wisconsin, Flu A H3/Darwin, and Flu B Austria are in a same binding domain; and
   the F proteins from PIV1, PIV2, PIV3, and PIV4 are in a same binding domain; or
(xii) HA from Flu A/H1 (e.g., H1/Wisconsin 2019); HA from Flu A/H3 (e.g., H3 Darwin 2021); HA from Flu B/Victoria (e.g., B/Austria 2021); F protein from PIV1; F protein from PIV2, F protein from PIV3; and F protein from PIV4,
(b) forming a binding complex in each binding domain, wherein the binding complex comprises the immobilized antigen and an antibody biomarker that binds to the immobilized antigen;
(c) contacting the binding complex in each binding domain with a detection reagent; and;
(d) detecting the binding complex in each binding domain, thereby detecting the one or more antibody biomarkers in the sample.

E6 includes the method of E5, wherein the detection reagent comprises a detection antibody, a detection antigen, or an ACE detection reagent

E7 includes the method of E5 or E6, wherein the detection reagent comprises an ECL label.

E8 includes the method of any one of E5 to E7, wherein the surface comprises an electrode.

E9 includes the method of any one of E5 to E8, wherein the surface comprises a well of a multi-well plate, and wherein each well comprises 1 to 10 binding domains.

E10 includes the method of any one of E5 to E9, wherein the detection reagent comprises an ECL label, the surface comprises an electrode, and the detecting comprises applying a voltage to the surface and measuring an ECL signal generated from the ECL label on the detection reagent.

### EXAMPLES

### Example 1. Bridging Serology Assay for SARS-CoV-2 on Patient Samples

A bridging serology assay to detect SARS-CoV-2 antibodies was performed using the SARS-CoV-2 S-RBD antigen. The bridging serology assay used the simultaneous binding of antibodies to immobilized viral antigen and detection tag-labeled viral antigen, leading to a highly specific isotype-independent measurement of immune reactivity. The tested samples were from the same COVID-19 positive and normal patients as described in Example 4, except the samples were diluted 10-fold or 100-fold.

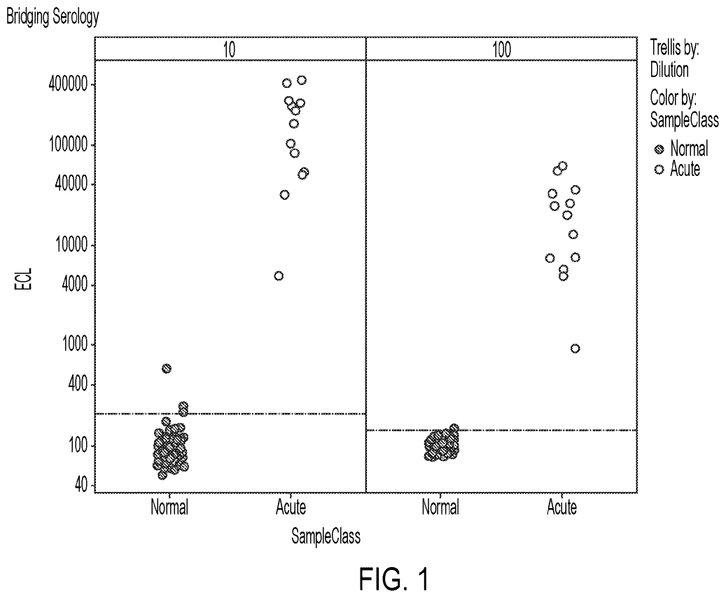

Results are shown in FIG. 1. For both 10-fold and 100-fold sample dilutions, clear separation of signal was observed between the COVID-19 patient and normal samples. High signals detected in the COVID-19 sera indicates the presence of anti-S-RBD antibodies.

### Example 2. Neutralization Serology Assay for SARS-CoV-2 on Patient Samples

As described herein, neutralization serology assays (also termed “competitive serology assay”) can allow assessment of the potential protective serological response present in the patient. A neutralization serology assay was performed to test SARS-CoV-2 antibody binding to immobilized SARS-CoV-2 S protein in the presence of the host cell protein receptor, ACE2, as competitor for the antigen. The ACE2 is labeled with a detection label. The tested samples were from the same COVID-19 positive and normal patients as described in Example 4, except the samples were diluted 10-fold or 100-fold.

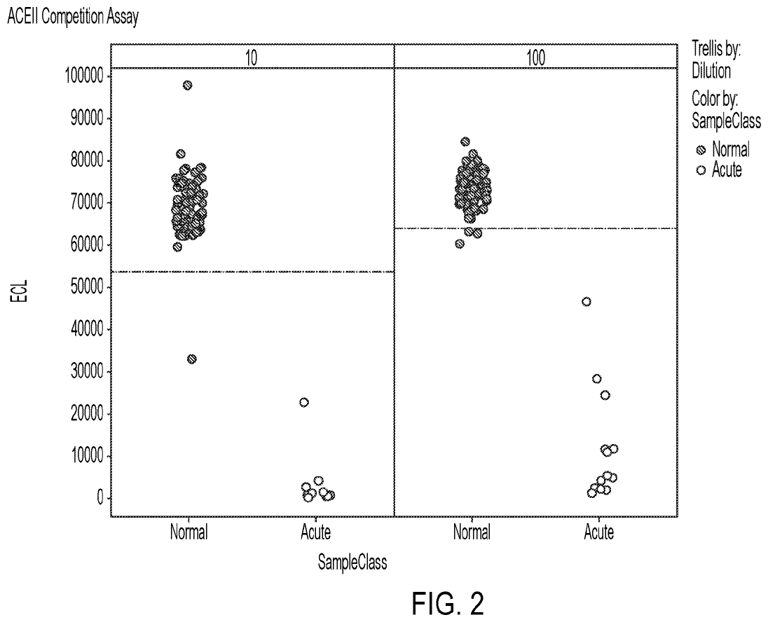

Results are shown in FIG. 2. For both the 10-fold and 100-fold sample dilutions, clear separation of signal was observed between the COVID-19 patients and normal samples. Lower signal detected in the COVID-19 sera indicates inhibition of the interaction between the SARS-CoV-2 S protein and its cognate receptor, ACE2, in COVID-19 patient samples.

### Example 3. One-Step RT-PCR for Amplifying Viral Nucleic Acid

An exemplary protocol for reverse transcribing and amplifying a viral RNA (e.g., SARS-CoV-2 RNA) is described in this Example.
1) Extract RNA from a sample containing the virus of interest, e.g., SARS-CoV-2;
2) Prepare a Master Mix containing a single primer pair or multiplexed primer pairs and a one-step RT-PCR mix (e.g., from a commercially available source);
3) Add the sample RNA to the Master Mix;
4) Program a thermocycler to run the following steps (Table 4):

TABLE 4
Thermocycler Conditions

| Step | Cycles | Temp | | Time | |
|---|---|---|---|---|---|
| UNG incubation | 1 | 25° | C. | 2 | min |
| RT incubation | 1 | 50° | C. | 15 | min |
| Polymerase activation | 1 | 95° | C. | 2 | min |
| Amplification | 45 | 95° | C. | 3 | sec |
| | | 55° | C. | 30 | min |

| | | | | |
|---|---|---|---|---|
| Hold | 1 | 4° | C. | Hold |

The number of amplification cycles and annealing temperature can be adjusted based on the experiment (e.g., length of nucleic acid to be amplified).

The PCR product is ready to be tested with the nucleic acid detection methods described herein.

### Example 4. Correlation of Different Serology Assay Formats

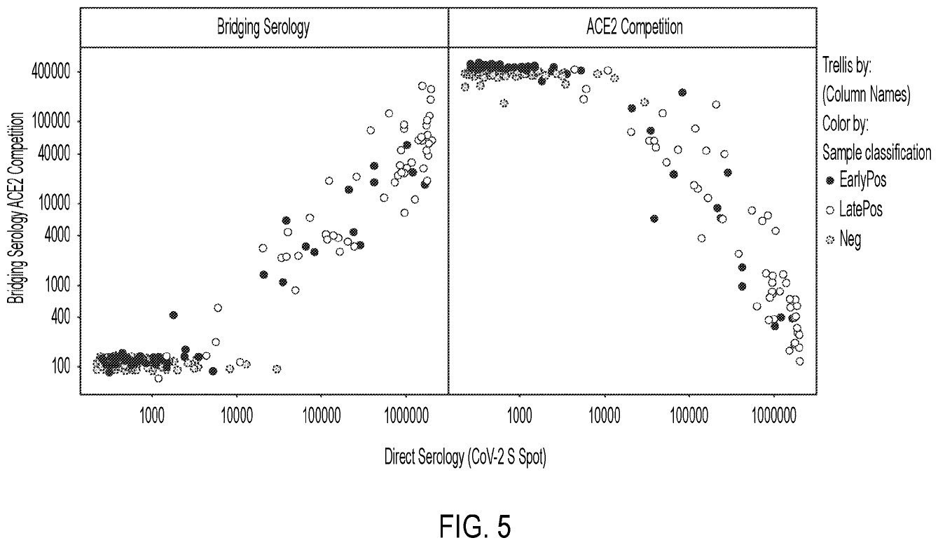

Results of a bridging serology assay and a neutralization serology assay were plotted against results of a classical serology assay to determine the correlation between the different serology assay formats. The plot is shown in FIG. 5, indicating the results between different assay formats are well-correlated.

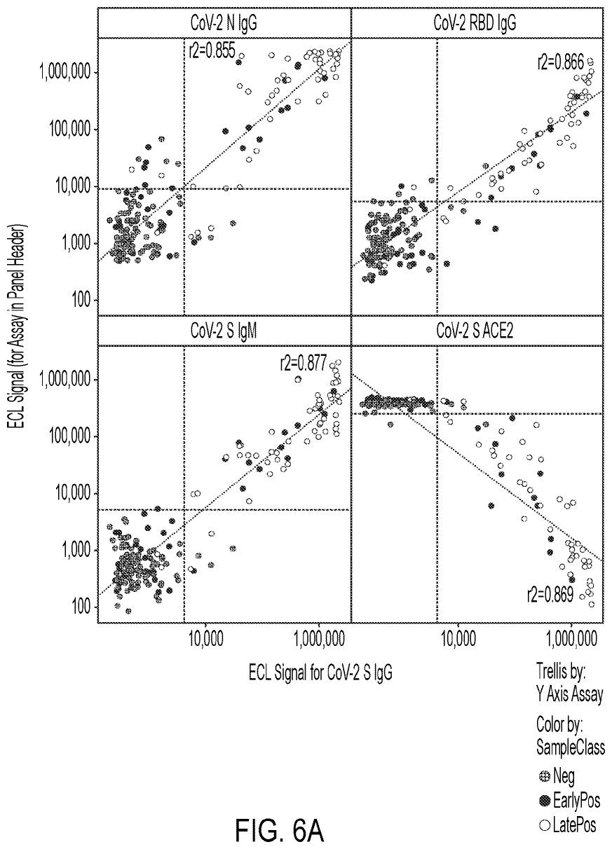

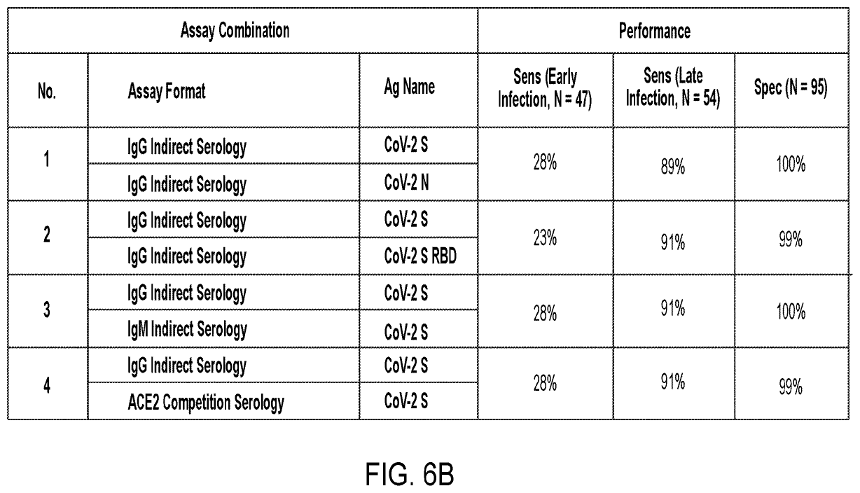

FIG. 6A shows the correlation of the indirect serology assays for IgG against SARS-CoV-2 S with four other serology assays (IgG against SARS-CoV-2 N, IgG against SARS-CoV-2 S-RBD, TgM against SARS-CoV-2 S, and ACE2 competitor assay). Elevated levels of IgG against SARS-CoV-2 S was associated with elevated levels of IgG against SARS-CoV-2 N (r²=0.86) and RBD (r²=0.87). Correlation was only observed if samples from infected patients were included in the analysis. Association between the assay signals was weak if the analysis was limited to just the naïve samples, in which case the r² values dropped to 0.07 and 0.17, respectively. A similar result was obtained by comparing the measured IgG and IgM response to SARS-CoV-2 S, which provided an r² value of 0.88 for the full sample set and 0.00 when limited to the naïve samples. Finally, generation of IgG antibodies that could bind SARS-CoV-2 S as measured by the indirect format was generally associated with the production of antibodies targeting the ACE2 binding site as measured by the ACE2 competition format (r²=0.87).

The weak correlation of signals for naïve samples provides a potential approach to improving assay performance by combining the results from multiple assays. For example, in the correlation plot for IgG against N vs. IgG against S (top left panel of FIG. 6A), there were some naïve samples that provided signals above the selected threshold for N but not S (top left area of panel) or S but not N (bottom right are of panel). The classification accuracy was further evaluated by requiring two assays to call a sample positive, as a method for reducing the impact of false positive signals for individual assays. FIG. 6B shows the classification performance for the possible pairings of the assays shown in FIG. 6A. In all four cases shown in the table in FIG. 6B, the combined assay result improved the specificity for classification of the naïve samples to 99% or 100% (0 or 1 false positive out of 95 samples). The improvement in specificity was accompanied by a small decrease in sensitivity: the sensitivities for classifying the late infection samples ranged from 89% to 91% for the two-assay combination compared to a range of 91% to 94% for the individual assays. In particular, combining the measurements of the IgG response to SARS-CoV-2 N and S proteins provided sensitivities of 28% and 89% for classifying the early and late infection samples, respectively, and a specificity of 100% for classifying the naïve samples.

### Example 5. Evaluation of Assay Performance

The sensitivity and specificity of serology assays were determined for IgG and IgM using SARS-CoV-2 S, S-RBD, and N antigens, and ACE2 competitor (neutralization) serology assays using SARS-CoV-2 S and S-RBD antigens. ROC analysis was used to identify the threshold for each combination of format and antigen that maximized the sum of the specificity and sensitivity for separating the late infections from the normal controls. This threshold was then applied to both the early infection and late infection data sets.

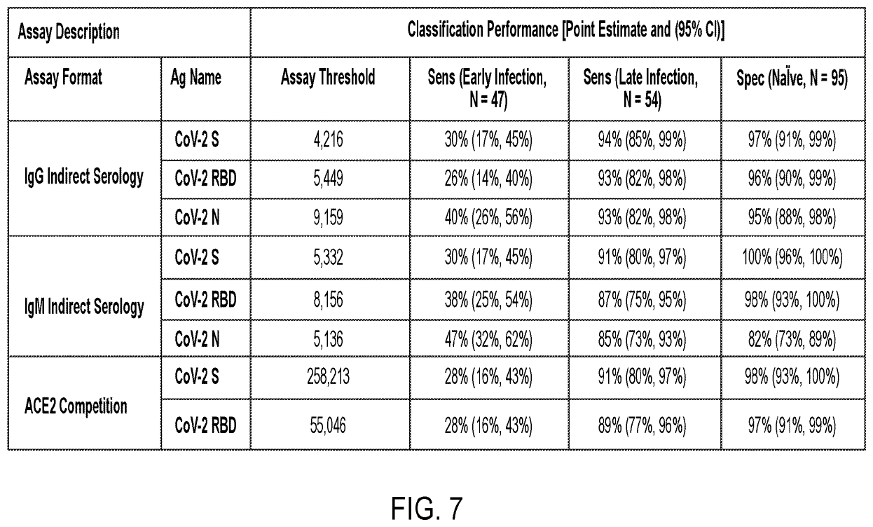

Using the optimal thresholds, the different assays shown in FIG. 7 provided similar sensitivity and specificity values, with largely overlapping 95% confidence limits. All the assays provided point estimates for specificity that were greater than or equal to 95%, except for the measurement of IgM against SARS-CoV-2 S protein. Measurements of IgG using the indirect serology format provided the highest point estimates for sensitivity in classifying late infections (point estimates of sensitivity for S, RBD and N ranged from 93% to 94%). Point estimates for measurement of IgM using the indirect serology format or inhibitory antibodies using the ACE2 competition format were slightly lower ranging from 85% to 91%, with the best sensitivity in both formats provided by the SARS-CoV-2 S antigen (91% sensitivity in both the IgM and ACE2 formats). All the assays provided lower sensitivity for measuring early infection with point estimates for sensitivity ranging from 26% to 47%. There was no evidence that measurement of IgM provided statistically higher sensitivity for classification of the early infection samples, relative to measurement of IgG.

### Example 6. SARS-CoV-2 Single Nucleotide Polymorphism (SNP) Assay

Singleplex and multiplex oligonucleotide ligation assays (OLA) were performed to detect single nucleotide polymorphisms (SNPs) at SARS-CoV-2 genome locations 8782 (C>T mutation), 11083 (G>T mutation), 23403 (A>G mutation), and 28144 (T>C mutation). A pair of targeting probes and detection probes for each polymorphic site were designed to allow for single-base discrimination at the SNP site, as described in embodiments herein. The targeting probes have unique 5′ oligonucleotide tag sequences that are complementary to binding reagents on specific binding domains on a multi-well plate. The detection probes have a 5′ phosphate group for ligation and a 3′ biotin. Taq DNA ligase was used to join the targeting probe and detection probe that aligned correctly on the sample. Fragments of unmodified template complements were added to prevent bridging of unligated probes. The OLA cycling conditions were: 2 minutes at 95° C., then 30 cycles of 30 seconds at 95° C. and 2 minutes at 65° C. The plates were blocked with a blocking solution for 30 minutes at 37° C. during the OLA cycling.

The plate was washed, and the ligated probes were hybridized to a binding reagent on a plate. In the singleplex format, each well only contained the binding reagents specific for one SNP. In the multiplex format, each well of contained ten binding domains (“spots”), wherein a unique binding reagents was immobilized in each spot, allowing for detection of up to five SNPs per well. The hybridization was performed in hybridization buffer (50 μL per well), with a one-hour incubation at 37° C.

The hybridized ligated probes were then detected by streptavidin-SULFO-TAG™. Briefly, following the hybridization, the plate was washed, and a detection solution was added (50 μL per well) and incubated for 30 minutes at 37° C. The plate was washed, and read buffer was added (150 μL), and the plate was then read using a plate reader.

Ten (10) nasal swabs from COVID-19-positive patients obtained from BOCA Biolistics (Deerfield Beach, FL) were tested using both the singleplex and multiplex OLA assay formats. RNA was extracted using the MAGMAX™ Viral/Pathogen Ultra Nucleic Acid Isolation Kit (Applied Biosystems). RT-PCR was conducted using site-specific primers using TAQPATH™ 1-step RT-qPCR Master Mix (Applied Biosystems). Reference strain RNAs were obtained from BEI Resources, NIAID, NIH: Genomic RNA from SARS-Related Coronavirus 2, Isolate USA-WA1/2020, NR-52285, GenBankMN985325, deposited by the Centers for Disease Control and Prevention; Genomic RNA from SARS-Related Coronavirus 2, Isolate Hong Kong/VM20001061/2020, NR-52388, GenBankMT547814, deposited by the University of Hong Kong.

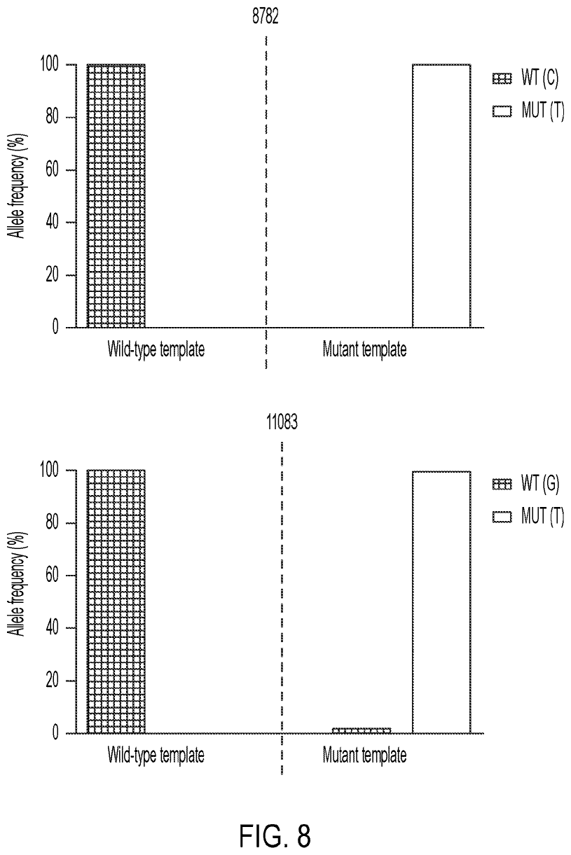

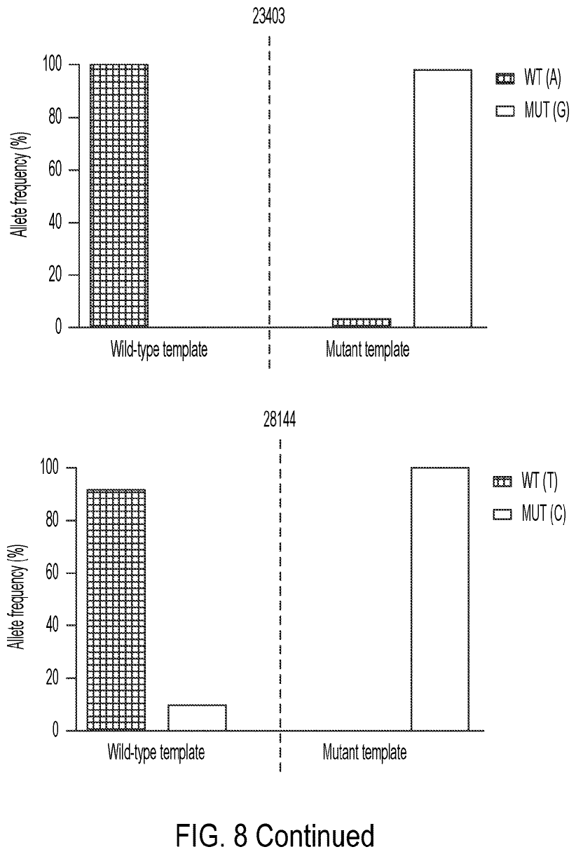

Results from a test assay using synthetic oligonucleotide template containing the SNPs of interest are shown FIG. 8. The synthetic oligonucleotide templates showed high specificity for the appropriate allele using ligation temperature of 65° C.

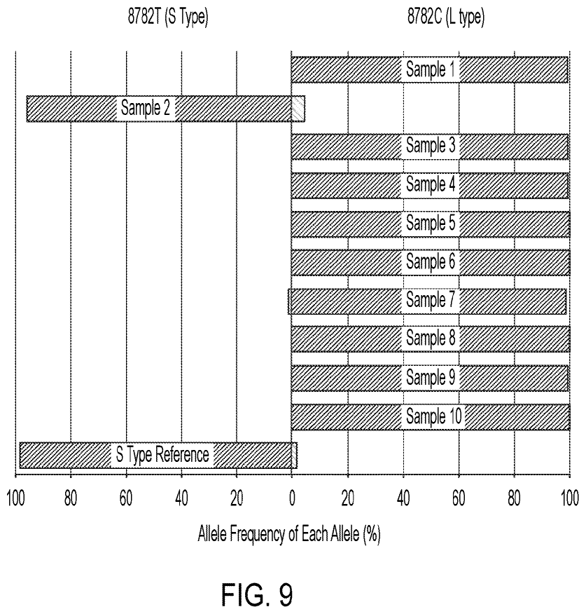

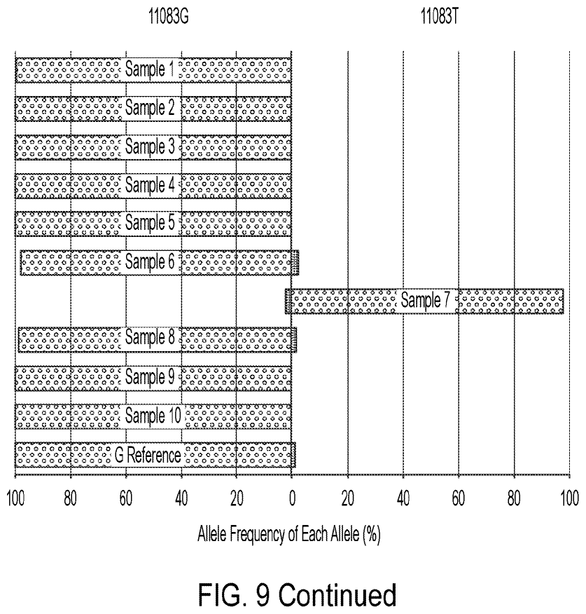

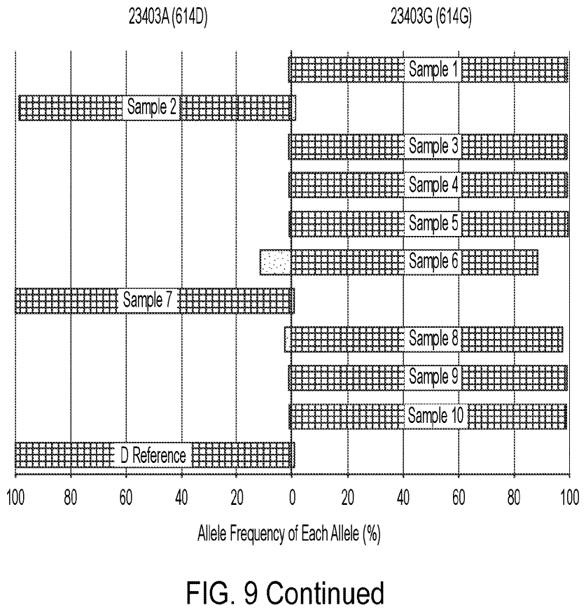

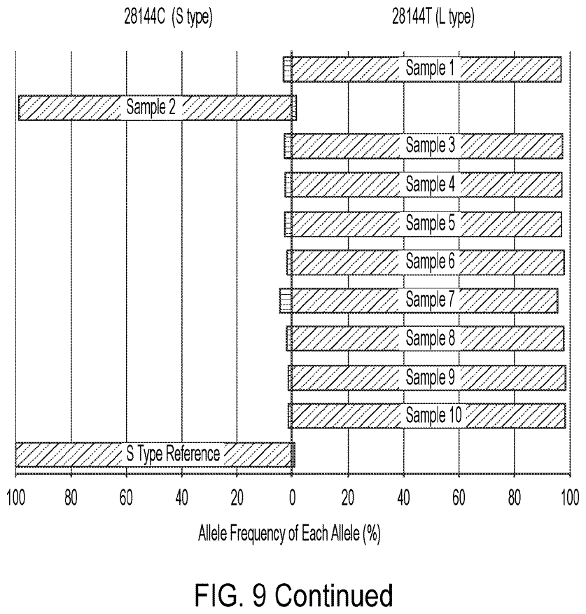

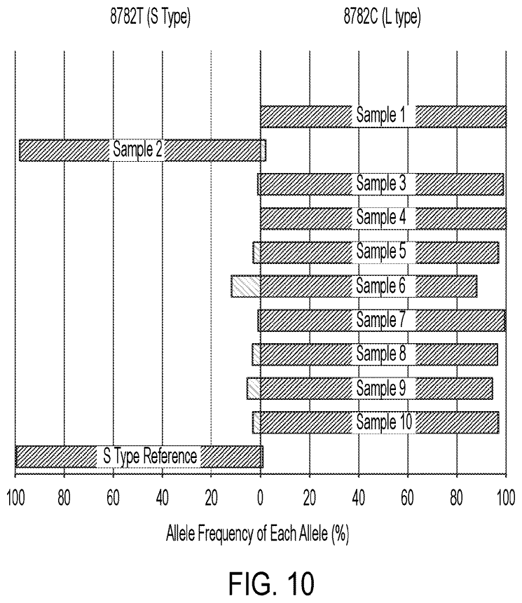

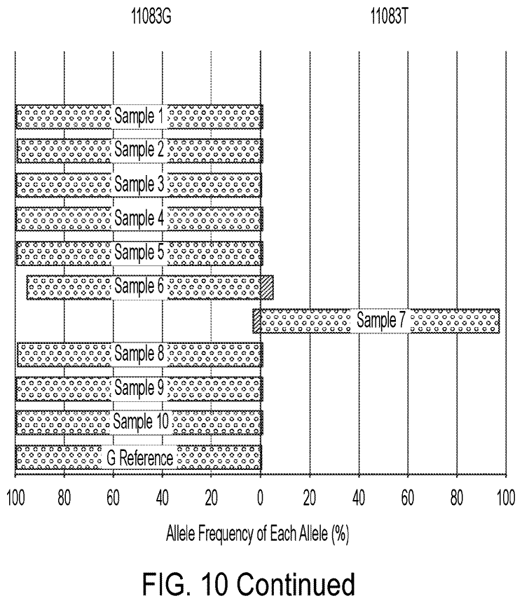

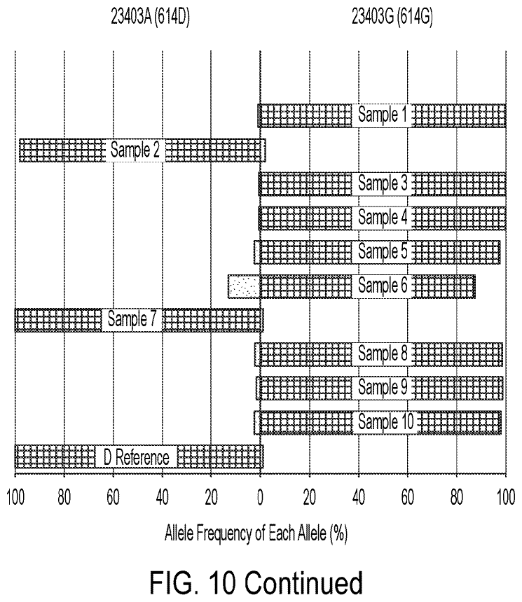

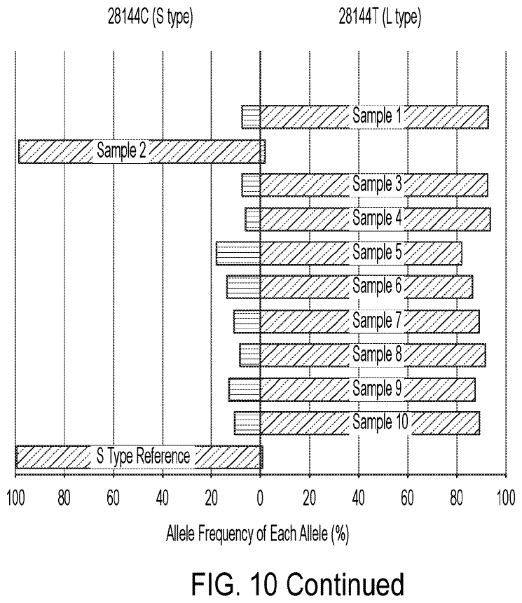

Results from the singleplex assay format with patient samples are shown in FIG. 9. There was a high prevalence of the L strain type (8782C and 28144T) that also have the D614G mutation. Only one strain had the “asymptomatic” allele (11083T). Results were also compared to a fully sequenced reference strain, with all results matching the published sequences for these strains. Results from the multiplex assay format with patient samples are shown in FIG. 10 and were consistent with the results from the singleplex assays shown in FIG. 9.

Reproducibility of the multiplex assay was also assessed. The multiplex RT-PCR products from the samples shown in FIG. 10 were subjected to three separate multiplexed OLA reactions on three different days to determine the allele frequency reproducibility from the SNP assays. The mean, standard deviation (STD), coefficient of variation (CV), and total number (N) of readings are shown in Table 5 for the samples that either had the WT or mutant (MUT) allele for the given SNP. As shown in Table 5, all assays had good allele frequency reproducibility, and the SNP determinations for all ten samples and the reference strain were consistent between the runs for all three experiments.

TABLE 5
OLA Reproducibility

| | WT samples (WT allele) | | | | MUT samples (MUT allele) | | | |
|---|---|---|---|---|---|---|---|---|
| Site | Mean | STD | CV | Total N | Mean | STD | CV | Total N |
| 8782 | 97.8 | 3.1 | 0.03 | 54 | 98.6 | 0.3 | 0.003 | 6 |
| 11083 | 99.3 | 2.8 | 0.03 | 54 | 98.1 | 1.0 | 0.01 | 6 |
| 23403 | 99.2 | 0.4 | 0.004 | 12 | 100.3 | 5.4 | 0.05 | 48 |
| 28144 | 96.3 | 10.0 | 0.1 | 54 | 98.7 | 0.8 | 0.01 | 6 |

### Example 7. Detection of SARS-CoV-2 Nucleocapsid Protein in Human Samples

A detection assay was used to test for SARS-CoV-2 N protein in the following samples: nasopharyngeal swabs from 12 patients who tested positive for COVID-19, nasopharyngeal swabs from 6 patients who tested negative for COVID-19, and normal (COVID-19 negative) human saliva, serum, and EDTA plasma. The detection assay was performed as follows: To each well of a 96-well plate containing immobilized anti-Nucleocapsid capture antibody, add 25 μL labeled anti-Nucleocapsid detection antibody labeled with nucleic acid probe, and 25 μL sample. Incubate for 1 hour at room temperature with shaking. Wash plate and add extension solution. Incubate for 15 minutes at room temperature with shaking. Wash plate and add detection solution. Incubate for 45 minutes at room temperature with shaking. Wash plate, add 150 μL ECL Read Buffer, and read plate. Total protocol time is approximately 2 hours. The samples were all tested without dilution. Assay results of the SARS-CoV-2 N protein concentration are shown in FIG. 11. The results for the negative nasopharyngeal swab and normal saliva, serum, and EDTA plasma were comparable, while the positive nasopharyngeal swab samples had significantly higher concentration of SARS-CoV-2 N protein.

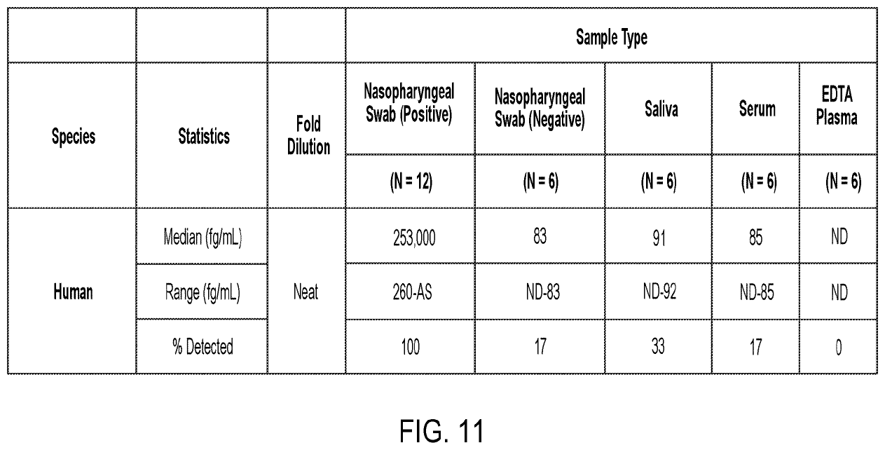

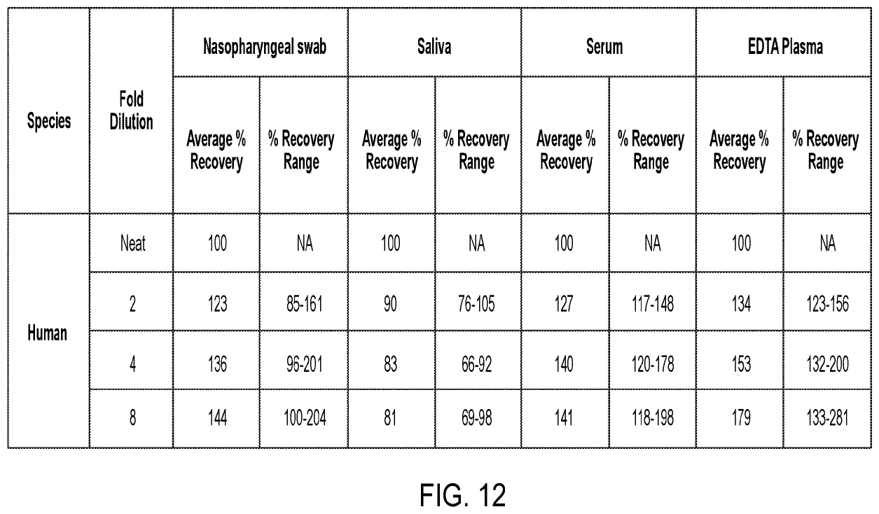

The dilution linearity and spike recovery of the assay were also tested to determine whether the assay is affected by component(s) that may be present in the biological sample matrix (also known as the “sample matrix effect”). For dilution linearity, the normal human serum, EDTA plasma, saliva, and COVID-19 negative human nasopharyngeal swab samples were spiked with calibrator and tested at different dilutions. Percent recovery at each dilution level was normalized to the dilution-adjusted, neat concentration, and shown in FIG. 12.

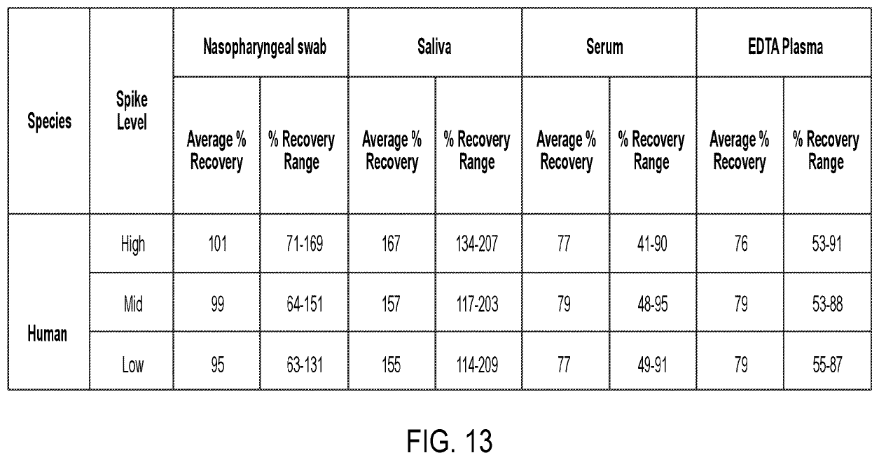

For spike recovery, normal human serum, EDTA plasma, saliva, and COVID-19 negative human nasopharyngeal swab samples were spiked with calibrator at three levels. Spiked samples were tested neat. Percent recovery is shown in FIG. 13. Based on the results in FIGS. 12 and 13, samples may be diluted to reduce sample matrix effects.

### Example 8. SARS-CoV-2 Nucleic Acid Detection Assay—OLA

The oligonucleotide ligation assay (OLA) described in Example 6 was also used to detect SARS-CoV-2 nucleic acid. Targeting and detection probes were designed for the SARS-CoV-2 N1, N2, and N3 regions (described in Lu et al., Emerg Infect Dis 26(8):1654-1665 (2020)) and the human RPP30 gene as control. The targeting probes have unique 5′ oligonucleotide tag sequences that are complementary to binding reagents on specific binding domains on a multi-well plate. The detection probes have a 5′ phosphate group for ligation and a 3′ biotin. OLA was used to ligate targeting and detection probes that aligned perfectly on the SARS-CoV-2 target nucleic acid. The ligated probes were then hybridized to the multi-well plate and detected by adding streptavidin-labeled SULFO-TAG™ and reading the signal with a plate reader. The plates were 10-spot, 96-well plates with the spot layout as shown in FIG. 29B. The binding reagents were immobilized in the spots as follows: Spot 4: binding reagent for N1; Spot 5: binding reagent for N2, Spot 9: binding reagent for N3; Spot 10: binding reagent for RPP30. The remaining spots were blank.

Probes were designed against both the viral RNA strand and the cDNA strand that is synthesized upon reverse transcription. The assay was first performed with a synthetic DNA template that included the target region (SARS-CoV-2 N1, N2, N3, and human RPP30). For viral RNA, one-step RT-PCR was performed to reverse transcribe and amplify the region surrounding each target. The OLA conditions are provided in Table 6.

TABLE 6
OLA Cycling Conditions

| | | | | |
|---|---|---|---|---|
| 95° | C. | 2 | min | |
| 95° | C. | 30 | sec | ×30 cycles |
| 65° | C. | 2 | min | |

| | | |
|---|---|---|
| 4° | C. | Hold |

Two sets of targeting and detection probes for each site were tested, designated as “OLA1” or “OLA2.” The probes sequences correspond to SEQ ID NOs: 22-54.

Primers for amplification of the SARS-CoV-2 N1, N2, N3 regions and human RPP30 are described in Lu et al., Emerg Infect Dis 26(8):1654-1665 (2020) and correspond to SEQ ID NOs: 67-74.

The OLA assays were performed on RNA extracted from swab samples of SARS-CoV-2 positive (n=15) or negative (n=6) patients. Detectable signals for all of the same targets were observed as from the results of a qRT-PCR assay. The negative control samples did not have appreciable signal, indicating good specificity to SARS-CoV-2. N1 and N3 showed the best results. RPP30 signals were high in all samples (SARS-CoV-2 positive and negative), which was expected and confirmed consistency in RNA extraction.

### Example 9. SARS-CoV-2 Nucleic Acid Detection Assay—Internal Detection Probe

A SARS-CoV-2 nucleic acid detection assay that amplifies regions of interest (N1, N2, and N3) and contacts the amplified regions with a single internal detection probe was developed. The forward primer was tagged with a 5′ oligonucleotide tag. The internal detection probes have a 3′ biotin. The SARS-CoV-2 N1, N2, and N3 regions and the human RPP30 gene (control) were amplified using multiplexed PCR, followed by hybridization of the PCR products with the internal detection probes to form hybridized. The hybridized products were then immobilized to the multi-well plate and detected by adding streptavidin-labeled SULFO-TAG™ and reading the signal with a plate reader. The plates were 10-spot, 96-well plates with the spot layout as shown in FIG. 29B. The binding reagents were immobilized in the spots as follows: Spot 8: binding reagent for N1; Spot 9: binding reagent for N2, Spot 10: binding reagent for N3; Spot 1: binding reagent for RPP30. The remaining spots were blank.

Sequences of the forward primers, reverse primers, and the internal detection probes correspond to SEQ ID NOs: 55-66.

### Example 10. SARS-CoV-2 Strain Typing and SNP Detection

The oligonucleotide ligation assay (OLA) as described in Examples 9, 13, and 23 is performed to detect mutations of the SARS-CoV-2 S protein. SARS-CoV-2 strains and the associated SNPs are shown in Table 1A and include genome locations 21765-21770, 23063, 23604, 22132, 22206, 22917, 23012, 23664, 22813, 22812, 22227, 28932, 29645, 1059, 25563, 21991-21993, 23271, 23709, 24506, 24914, 241, 3037, 14408, 26144, 29095, 22865, 22320, 21618, 23604, 24775, 22995, 24224, 25088, 23593, 24138, 21846, 22578, and 23525. Two sets of targeting and detection probes (“OLA1” or “OLA2”) for each genome location were designed. Further, two sets of targeting (“US”) probes for each genome location, one for the reference strain (“WT”) and one for the variant, were designed. The same detection (“DS”) probe for each genome location was designed for both the reference strain and the variant. The sequences for the targeting and detection probes correspond to SEQ ID NOs: 75-134, 227-322, 533-538, 549-596, 655-662, 666-675, and 714-731. The sequences of synthetic templates containing the SARS-CoV-2 genome regions of interest correspond to SEQ ID NOs: 135-174, 323-386, 539-542, 597-626, 676-679, and 732-743. The sequences for blocking oligonucleotides correspond to SEQ ID NOs: 175-216, 387-452, 543-546, 627-654, 680-683, and 744-755. The primers for amplifying the target regions correspond to SEQ ID NOs: 217-226, 453-491, 663-665, 684-713, and 756-758.

The OLA as described above is also performed to detect single polynucleotide polymorphisms (SNPs) at SARS-CoV-2 genome locations 8782, 28144, 23403, and 11083. As described herein, SNPs at genome locations 8782 and 28144 differentiate the L and S strains of the SARS-CoV-2. The A>G SNP at genome location 23403 encodes the D614G mutation in the SARS-CoV-2 S protein. The G>T SNP at genome location 11083 is associated with asymptomatic infection by SARS-CoV-2. The sequences for the targeting and detection probes correspond to SEQ ID NOs: 492-506. The sequences of synthetic templates containing the SARS-CoV-2 genome regions of interest correspond to SEQ ID NOs: 507-522. The sequences for blocking oligonucleotides correspond to SEQ ID NOs: 523-532.

### Example 11. Clinical Characterization of Serology Assay Panels and Formats

Clinical characterization of assay panels and formats as shown below was carried out using 200 pre-2019 COVID-19-negative serum samples and 200 PCR-confirmed COVID-19-positive serum samples. The samples were grouped from time of serum collection relative to positive PCR test (0 to 14, 15 to 28, 29 to 56, or 57+ days post-diagnosis (Dx).

### Assay Panels and Formats:

### (1) Indirect (Classical) IgG Serology and ACE2 Competition:

Antigens in Panel A: SARS-CoV-2 N protein, S protein, and S-RBD;
Antigens in Panel B: Wild type (WT) SARS-CoV-2 N protein; S protein and S-RBD from WT SARS-CoV-2 and SARS-CoV-2 variants B.1.1.7, B.1.351, and P.1;

### (2) Bridging Serology:

Antigens in Panel C: SARS-CoV-2 N protein and S-RBD.
Summary of results for WT antigens:

The WT antigens provided excellent clinical performance, even for early positive samples. The measured specificities for the different antigens/formats ranged from 98.5% to 100%, measured sensitivities for late positives (15+ days from Dx) ranged from 93.8% to 98.3%, measured sensitivities for early positives (≤14 days from Dx) ranged from 65.8% to 92.1%. Assay signals for the Bridging Serology assay format increased with time across the time groups, indicating the Bridging Serology assay format may measure affinity maturation of antibody responses over time.

All antigens and assay formats provided excellent separation of the negative and positive samples. While sensitivity for detecting early (≤14 day) infection was good, for most assay panels and formats there was a significant increase in concentration and sensitivity for samples collected >14 days after diagnosis. Interestingly, while antibody concentrations measured by the Indirect Serology and ACE2 Competition assay formats generally plateaued for time points longer than 14 days, concentrations measured by the Bridging Serology assay format showed significant increases with time across the measured time range. Since the Bridging Serology assay format requires two antibody-antigen binding interactions, it is generally biased towards measuring only high affinity antibodies and may be more sensitive in measuring changes in average antibody affinities as antibody responses mature over time.

The ROC curves showed near ideal separation of negative and late positive samples, as demonstrated by AUC values ranging from 0.983 to 0.992 (an AUC of 1 indicates perfect separation).

All assay formats and antigens showed excellent clinical performance. Point estimates for sensitivity for convalescent samples (15+ days post diagnosis) ranged from 93.8% to 98.3%, with the highest sensitivities obtained by the Indirect IgG Serology and ACE2 Competition assay formats using SARS-CoV-2 S protein as the antigen. Sensitivity during acute infection (0 to 14 days post diagnosis) was lower, which was expected since the antibody response is still developing during this period, but still quite good with point estimates ranging from 65.8% to 92.1%. The Indirect IgG Serology and ACE2 Competition assay formats with SARS-CoV-2 S protein as the antigen were also the most sensitive for detecting acute infection. The assays were very specific with point estimates for specificity ranging from 98.5% to 100%. The performance of all the assays was well within the requirements stated in the United States Food and Drug Administration (FDA) guidance or use of COVID-19 serology tests under an Emergency Use Authorization (EUA).

### Summary of Results for SARS-CoV-2 Variants/Mutant Antigens:

Panels that include antigens from SARS-CoV-2 variant lineages can provide important tools for assessing the immunity of individuals to the different variants. Assays with the S-RBD antigen from the B.1.351 and P.1 variants, which contain the K417N/T or E484K mutations were performed, and the antibody response from the COVID-19-positive samples against the mutant S-RBD were lower as compared to the antibody response against S-RBD without the K417N/T or E484K mutations (i.e., wild type or B.1.1.7 variant). The difference in antibody responses to the wild type vs. mutant S-RBD was much larger when measured by the ACE2 Competition assay relative to the Indirect IgG Serology assay format. These results suggest that individuals infected with a wild type or wild type-like variant may have reduced ability to avoid infection with the B.1.351 or P.1 variants.

Antibody binding to antigens from the four variants was elevated in COVID-positive vs. COVID-negative subjects, although levels were generally lower for the variant antigens, in particular the S-RBD from the B.1.351 and P.1 variants. The difference may stem from the fact that the COVID-19 positive samples were collected in late 2020 and early 2021 in the US when the wild-type lineage was predominant and there was little evidence in the US of the mutations found in the variants. While the number of mutations in the variant antigens is small, at least some of the mutations are likely present in epitopes that are sufficiently immune-dominant to account for a measurable proportion of the antibody response.

### Example 12. SARS-CoV-2 Serology Assays with Variant Panels

Samples from SARS-CoV-2-infected individuals in the United States in early 2020 (known to be infected with wild-type SARS-CoV-2 (“Wuhan”)); SARS-CoV-2-infected individuals in the United Kingdom (dominating strain: SARS-CoV-2 strain B.1.1.7); or SARS-CoV-2-infected individuals in South Africa (dominating strain: SARS-CoV-2 strain 501Y.V2, also known as B.1.351) were tested using a viral antigen panel that included the wild-type S protein from SARS-CoV-2, S-RBD from SARS-CoV-2 strain 501Y.V2, N protein from SARS-CoV-2, S-RBD from SARS-CoV-2 strain P.1, S-RBD from SARS-CoV-2 strain B.1.1.7, S protein from SARS-CoV-2 strain P.1, S protein from SARS-CoV-2 strain B.1.1.7, S protein from SARS-CoV-2 strain 501Y.V2, and wild-type S-RBD from SARS-CoV-2. The viral antigens were immobilized in a 96-well plate or within an assay cartridge. Detection was performed using an anti-IgG antibody labeled with an electrochemiluminescence (ECL) label.

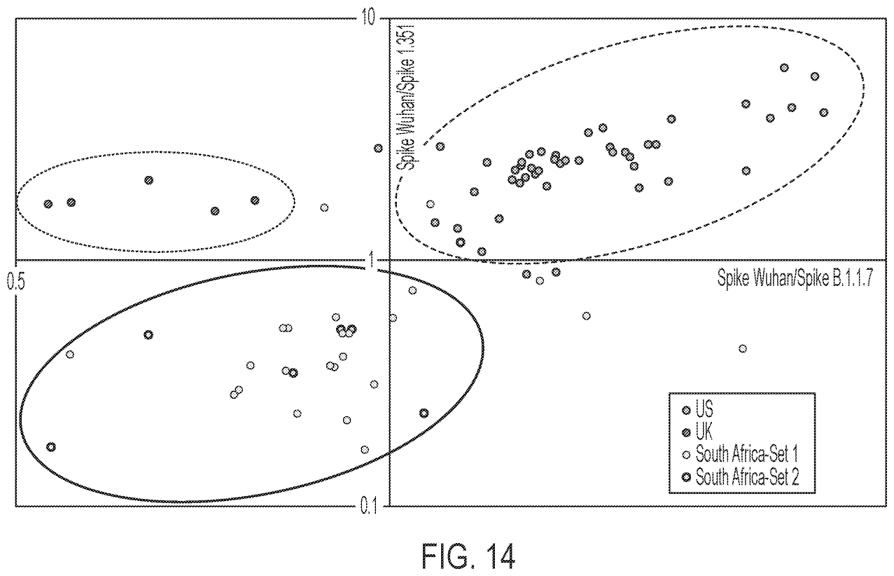

The results are shown in FIG. 14. The measured ratios of antibodies against wild-type SARS-CoV-2 versus SARS-CoV-2 strain B.1.1.7 were plotted on the x-axis, and the measured ratios of antibodies against wild-type SARS-CoV-2 versus SARS-CoV-2 strain 501Y.V2 were plotted on the y-axis. As shown in FIG. 14, samples from the wild-type SARS-CoV-2-infected patients clustered in the top right quadrant; samples from the UK clustered in the top quadrant; and samples from South Africa clustered in the lower left quadrant. Thus, the samples can be differentiated by geographical region based on binding to the S protein or S-RBD in the serology panel. This approach can be used in epidemiology studies to determine the circulating strain in a population or geographical region.

### Example 13. Matched Fingerstick Blood and Saliva Sample Testing

Specimen from 132 individuals who self-collected saliva and/or finger-stick samples were obtained. Matched saliva and finger-stick blood was provided by 125 of these donors. Six donors only provided saliva samples. The saliva sample from one donor with a PCR confirmed diagnosis of COVID-19 did not have sufficient quantity for analysis and was therefore not included. The individuals also completed a survey on COVID-19 diagnosis, exposure, and symptoms, with results summarized in Table 7.

TABLE 7
COVID-19 Survey Responses

| SARS-CoV-2 | Positive PCR | Household | COVID-19 | Number of |
|---|---|---|---|---|
| Exposure Category | Test | Exposure | Symptoms | Donors |
| Presumed Naïve (PN) | No | No | No | 107 |
| Positive COVID-19 PCR (PCR+) | Yes | Yes or No | Yes or No | 6 |
| Possibly Non-Naïve (PNN) | | | | |
| Household exposure with | No | Yes | Yes | 3 |
| symptoms (HEWS) | | | | |
| Household exposure with no | No | Yes | No | 4 |
| symptoms (HENS) | | | | |
| COVID-19 Symptomatic (CS) | No | No | Yes | 7 |
| No Survey (NS) | No response | No response | No response | 5 |

### Methods

The saliva samples were self-collected by donors in a 2 mL tube and frozen at ≤−70° C. without additional processing. The finger-stick blood samples were self-collected by donors using a Mitra collection kit, which contained a swab on which the blood dried shortly after collection. For reconstitution of the dried blood, swabs were placed into 2 mL microcentrifuge tubes containing 200 μL of diluent and extracted for 1 hour at room temperature with gentle shaking at 700 RPM. After 1 hour, the swab was removed and discarded. The microcentrifuge tube containing extracted whole blood was capped and frozen at ≤−70° C.

The samples were subjected to the multiplexed indirect serology panel shown as Coronavirus Panel 2 in Example 3 to measure IgG, IgM, and IgA antibody responses. On the day of testing, saliva and extracted finger-stick blood were thawed at room temperature. Saliva was centrifuged briefly to pull down any food particles or mucus. Prior to analysis, saliva samples were diluted five-fold by combining 20 μL of sample with 80 μL of a sample diluent. Extracted finger-stick blood was diluted 100-fold by combining 10 μL of sample with 990 μL of a different diluent.

Total levels of IgG, IgM, and IgA immunoglobulin were measured using the Isotyping Panel 1 Human/NHP Kit (Meso Scale Diagnostics, Rockville, Maryland). Extracted finger-stick blood was run at a dilution of 5,000-fold. Saliva was run at a dilution of 1,000-fold. Calibration and quantitation were carried out as described above for the indirect serology measurements.

### Sample Verification

The samples were tested for quality, e.g., whether there was deterioration of antibodies and/or high levels of food particles or phlegm. Quality of saliva samples was assessed by visual inspection and by measuring salivary antibody content. Saliva samples differed widely in appearance and volume.

The samples were verified to contain expected levels of immunoglobulins as a basic indicator of sample integrity. Median concentrations of total salivary immunoglobulin were 1.5 μg/mL, 2.9 μg/mL, and 83 μg/mL for IgG, IgM, and IgA, respectively. Concentrations of total salivary immunoglobulins were similar to published ranges measured using different assays and collection methods (IgG range=0.4-93 μg/mL; IgM=0.5-13.0 μg/mL; IgA=50.2±19.1 μg/mL). Median concentration of salivary IgG was 100-fold lower than measured in our diluted finger-stick blood samples and 7,300-fold lower than reported for undiluted serum. The variation in total immunoglobulin concentrations across donors was higher in saliva than in finger-stick blood. The ratio of the 75^{th} percentile to the 25^{th} percentile for IgG levels was 4.7 for saliva compared to a ratio of 1.6 for finger-stick blood.

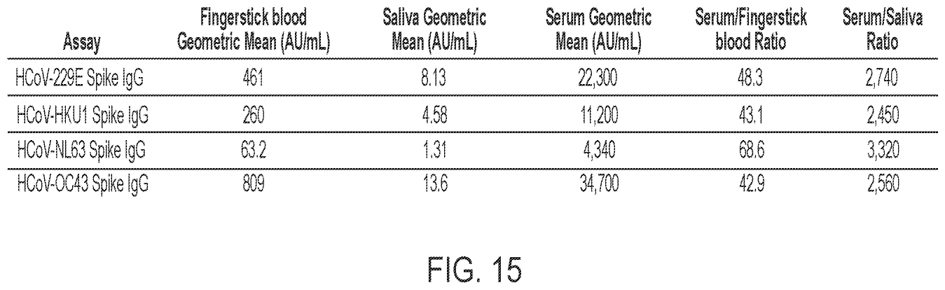

As an additional assessment of sample quality, the levels of antibodies to spike proteins for circulating coronaviruses were measured. Prior infection with these endemic viruses is common, and thus all donors were expected to have high levels of antibodies to at least one of the four circulating coronaviruses on the panel. See, e.g., Gaunt et al., J Clin Microbiol 48:2940-2947 (2010); Killerby et al., J Clin Virol 101:52-56 (2018); and Westerhuis et al., medRxiv 2020.08.21.20177857 (2020). Consistent with the levels of total immunoglobulin in finger-stick blood and saliva relative to serum discussed above, serum levels of antibodies to circulating coronaviruses were on average 51-fold and 2,800-fold higher than in finger-stick blood and saliva, respectively, as shown in FIG. 15.

Of the 125 donors providing matched saliva and finger-stick blood samples, two PN donors had normal levels of total immunoglobulin, and antibodies against the circulating coronaviruses in their blood sample, but not in saliva. One of these donors showed strong IgG reactivity to 229E Spike in finger-stick blood (850 AU/mL; above the 75^{th} percentile), but showed background IgG reactivity to 229E Spike in saliva. The other donor showed strong IgG reactivity to OC43 Spike in finger-stick blood (1,500 AU/mL; above the 75^{th} percentile), but showed background IgG reactivity to OC43 Spike in saliva. This result indicates a likely issue in the collection and/or handling of these samples, but also suggests that measurements of total immunoglobulin levels, or measurements of antibodies against high prevalence endemic viruses such as the circulating coronaviruses, could be used to identify problematic samples. Saliva from these two donors was excluded from analysis of SARS-CoV-2 antibody responses.

### Establishment of Normal Ranges in Non-Infected Individuals

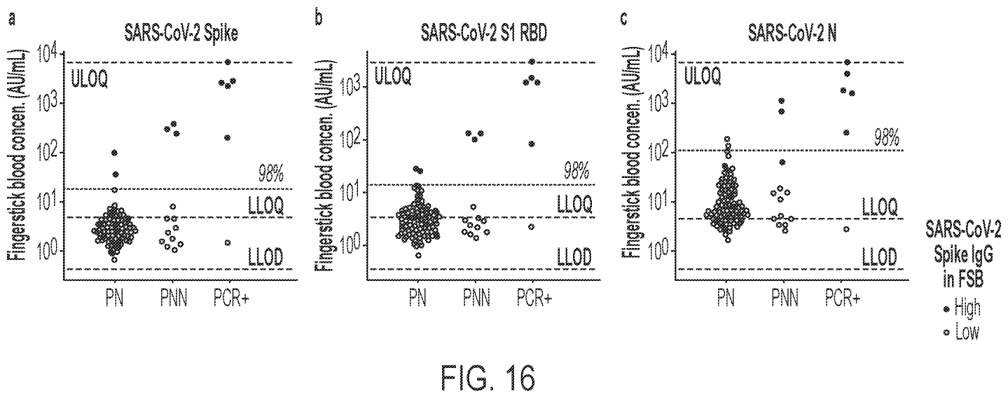

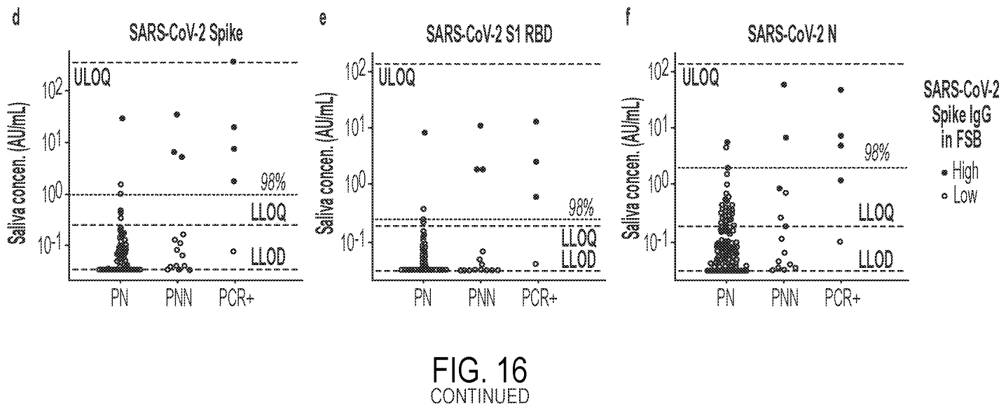

FIG. 16 shows the measured concentrations of antibodies to the SARS-CoV-2 antigens in finger-stick blood and saliva from all donors. The normal ranges for the SARS-CoV-2 serology assays were established using the samples from the 107 study donors who were unlikely to have had prior infection with COVID-19 (PN group). Preliminary threshold values for classifying individuals with prior SARS-CoV-2 infections were determined based on the 98^{th} percentile for the normal range (see Table 8). This approach provides a tolerance for a 1% to 2% rate of undetected asymptomatic infection in this PN group. Overall seropositivity at the time of this study is estimated at 4.4%, based on a study of health care personnel without patient contact within the same metropolitan area performed at approximately the same time. Since half of SARS-CoV-2 infections are thought to be asymptomatic, the expected prevalence of seropositivity resulting from asymptomatic infection is approximately 2%.

TABLE 8
Thresholds for Reactivity to SARS-CoV-2 Antigens

| | FSB Threshold | Saliva Threshold |
|---|---|---|
| Assay | (AU/mL) | (AU/mL) |
| SARS-CoV-2 N IgG | 119 | 3.18 |
| SARS-CoV-2 S1 RBD IgG | 14.3 | 0.244 |
| SARS-CoV-2 Spike IgG | 17.9 | 0.963 |

At the selected dilution, most of the saliva samples from the PN group were below the LOD for reactivity to the SARS-CoV-2 spike and RBD antigens. A higher percentage of these saliva samples had detectable reactivity against SARS-CoV-2 N antigen, which may result from the presence of cross-reactive antibodies originally induced by other coronaviruses. The selected classification thresholds for extracted finger-stick blood were 119 AU/mL, 14 AU/mL, and 18 AU/mL for IgG against SARS-CoV-2 N, RBD, and Spike, respectively. The selected classification thresholds for saliva were 3.2 AU/mL, 0.24 AU/mL, and 0.96 AU/mL for IgG against SARS-CoV-2 N, RBD, and spike, respectively.

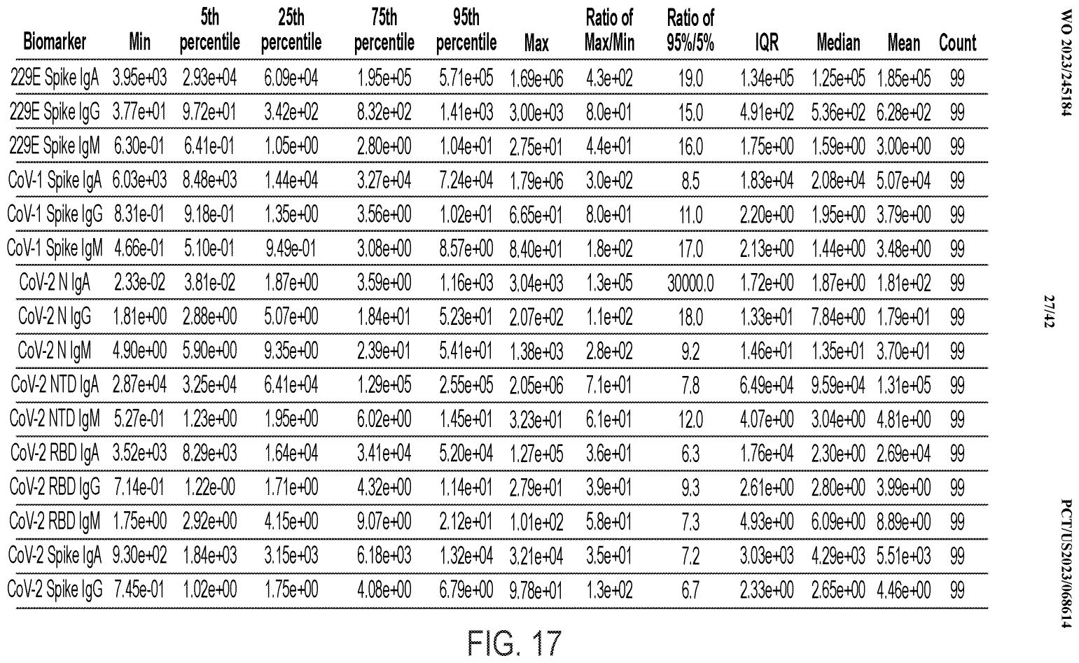

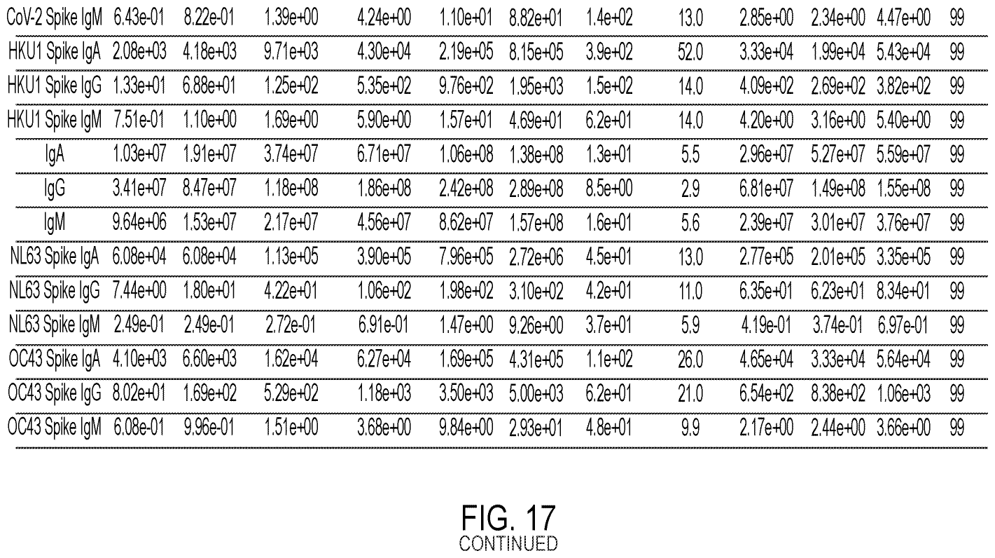

FIG. 17 shows the immunoglobulin concentrations in finger-stick blood self-collected by donors without confirmed COVID-19 diagnosis, household exposure, or recent symptoms, which were used to establish the upper limit of non-reactivity. FIG. 18 shows the immunoglobulin concentrations in saliva self-collected by donors without confirmed COVID-19 diagnosis, household exposure, or recent symptoms.

### Reactivity to SARS-CoV-2 Antigens in Finger-Stick Blood Samples

FIG. 16 shows the measured levels of IgG antibodies against the three SARS-CoV-2 antigens (spike, RBD and N) in finger-stick samples, relative to the selected thresholds. By definition, as the thresholds were defined as the 98^{th} percentiles for the PN group, 2% (2 of 107) of the PN samples were classified as positive by each assay. Each of the three assays identified 5 of 6 of the PCR+ (confirmed positive) donors. The PCR+ donor that was classified as negative reported an asymptomatic COVID-19 diagnosis more than 30 days previously, but had no significant reactivity to any SARS-CoV-2 antigen for any isotype. This individual had total immunoglobulin levels within the normal range as well as normal reactivity to circulating coronaviruses. A humoral response in this individual may have waned or not developed. For the PNN participants that were considered potentially non-naïve to SARS-CoV-2 based on symptoms and/or household exposure (the CS, HEWS and HENS groups), the spike and RBD assays classified 3 of 14 as positive (2 symptomatic and 1 asymptomatic donor with household contacts). The N assay also classified 2 of these 3 as positive, the third falling just under the threshold. Interestingly, none of the donors who reported possible COVID-19 symptoms, but no confirmed diagnosis or household exposure, had elevated antibody levels to SARS-CoV-2 antigens. This suggests that non-specific symptoms may be unreliable indicators of past infection. Alternatively, antibody levels may have waned faster in mild cases for which donors did not seek testing.

In FIG. 16, closed circles indicate samples provided by donors whose IgG levels in finger-stick blood exceeded the threshold for spike protein. Among the confirmed or possibly infected individuals (PCR+ and PNN groups), the same 8 finger-stick samples showed elevated reactivity to all three finger-stick SARS-CoV-2 antigens, although one of the samples was just under the threshold for the N assay.

### Reactivity to SARS-CoV-2 Antigens in Saliva

FIG. 16 also shows the measured levels of IgG antibodies against the three SARS-CoV-2 antigens (spike, RBD and N) in saliva samples. For samples from donors that were confirmed or possibly infected (PCR+ and PNN groups), measurements of IgG against spike and RBD proteins in finger-stick blood and saliva samples provided complete agreement in classification. Measurement of IgG against the N protein performed similarly except for one PCR+ donor who obtained a positive result for N in blood but not saliva. The two PN samples that were classified as positive varied for the different assays and sample types, although there was one PN donor that was classified as positive based on IgG against spike and RBD in blood, and spike, RBD and N in saliva, suggesting this individual may have had an asymptomatic infection.

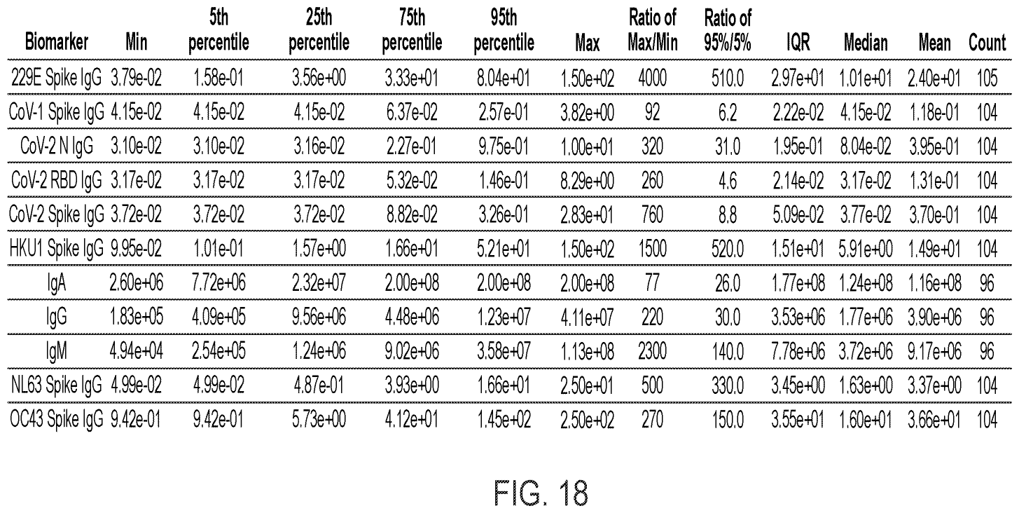

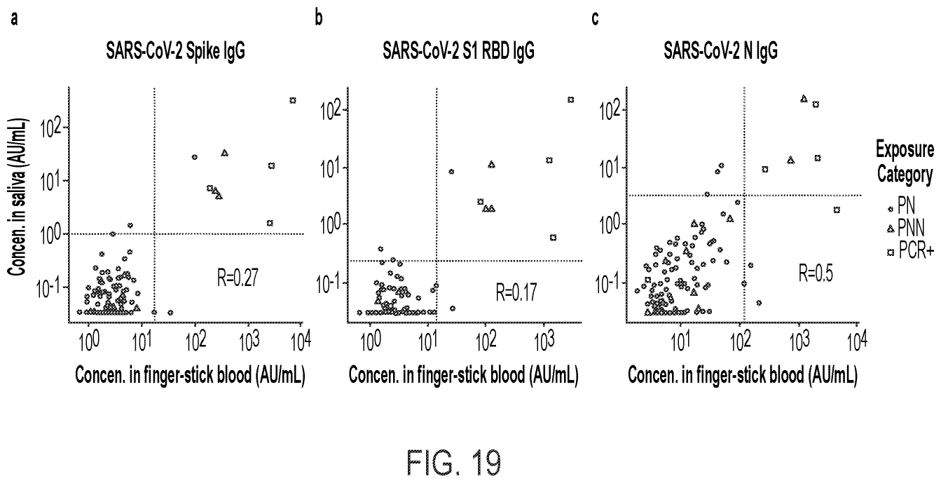

Correlations between the levels of antigen-specific IgG in saliva and blood samples are shown in FIG. 19. While the agreement between the two matrices for classification was strong, their correlation in levels was only moderate (R=0.25; p=0.005), which was consistent with previous studies (see, e.g., MacMullan et al., Scientific Reports 10:20808 (2020)). In 97.5% of donors with matched saliva and finger-stick blood, saliva and finger-stick blood measurements were concordant for classification of SARS-CoV-2 spike IgG and SARS-CoV-2 S1 RBD IgG levels as high or low relative to their matrix-specific thresholds (Cohen's κ=0.83; p=8.4e-18). 112 donors had low levels of SARS-CoV-2 Spike IgG for both saliva and finger-stick blood, and 8 donors had high levels of SARS-CoV-2 Spike IgG in both saliva and finger-stick blood. For the three discordant cases, two donors were slightly above the saliva threshold, and one donor was slightly above the finger-stick blood threshold. The concordance for the SARS-CoV-2 N IgG assays was 95.1% (Cohen's κ=0.64; p=2.5e-6).

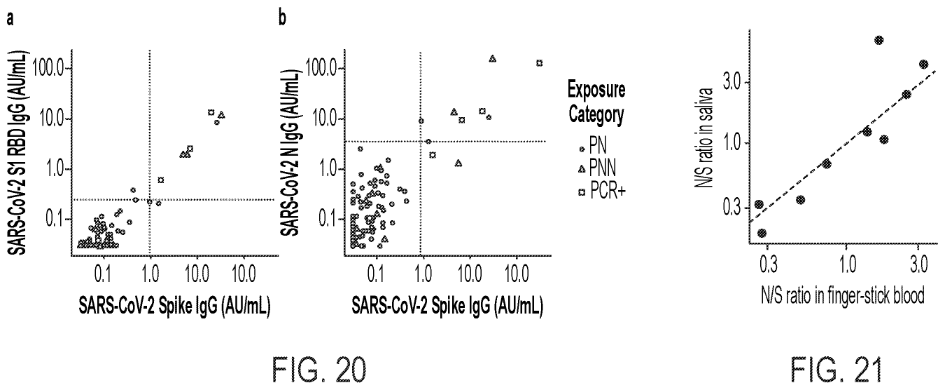

The salivary levels of antibodies to the full-length spike and RBD antigens were highly correlated (FIG. 20). Absolute signals for the full-length spike were higher than for the RBD antigen, which is expected since antibodies to the RBD are a subset of those binding to the full-length spike. The salivary levels of antibodies reactive with the N antigen were moderately correlated with antibodies for spike and RBD.

Because the relative immune responses to N versus S may be a clinically significant indicator of immune response, correlated the ratio of anti-N to anti-S levels measured in finger-stick blood versus saliva were correlated, as shown in FIG. 21. A strong correlation was found in the N to S ratio (R=0.95; p=0.001), which indicates that quantitative salivary measurements can be used to compute this ratio equivalently to finger-stick measurements.

### Transport of Samples by Mail

Due to the high stability of salivary antibodies at room temperature and without preservatives, a pilot study was performed in which donors mailed saliva specimens from Oklahoma to Maryland. Specimens (n=19) showed expected levels of antibodies to circulating coronaviruses. Although mailed-in samples spent up to two weeks in transit, the range of antibody concentrations for the circulating coronaviruses overlapped with that of locally collected samples. Anti-SARS-CoV-2 antibodies were detected only in the two samples from individuals who responded that they had previously been infected by SARS-CoV-2. Overall, this pilot study demonstrates the feasibility of a mailed test for salivary antibodies.

### Discussion

This study demonstrated that self-collected saliva provides information similar to finger-stick blood even after the saliva is at ambient temperature for hours to days without preservatives. Consistent with prior studies that report a correlation between antibody levels in serum and plasma, very high concordance was observed between self-collected saliva and finger-stick blood in the identification of individuals with IgG reactivity to the RBD and full-length forms of CoV-2 spike protein.

The degree of correlation between finger-stick blood and saliva measurements (FIG. 19) depends on variations among individuals in the rate of antibody transit into the mouth, the rate of saliva flow diluting the antibody, and possibly other factors associated with the degree of compliance with instructions for sample collection such as delaying collection after eating or drinking. The almost perfect agreement we observed in the classification of serostatus using saliva and finger-stick blood suggests that the difference in the observed antibody activity in positive subjects vs. negative controls is large enough to compensate for the increased variability in saliva samples. The ratio of anti-N antibodies to anti-S antibodies in saliva and finger-stick blood (FIG. 21) correlates more strongly than the ratio of the absolute concentrations (FIG. 19), showing that the effect of variations among donors in salivary flow rates and rates of antibody transit can be reduced through normalization approaches.

### Example 14. SARS-CoV-2 Strain Identification

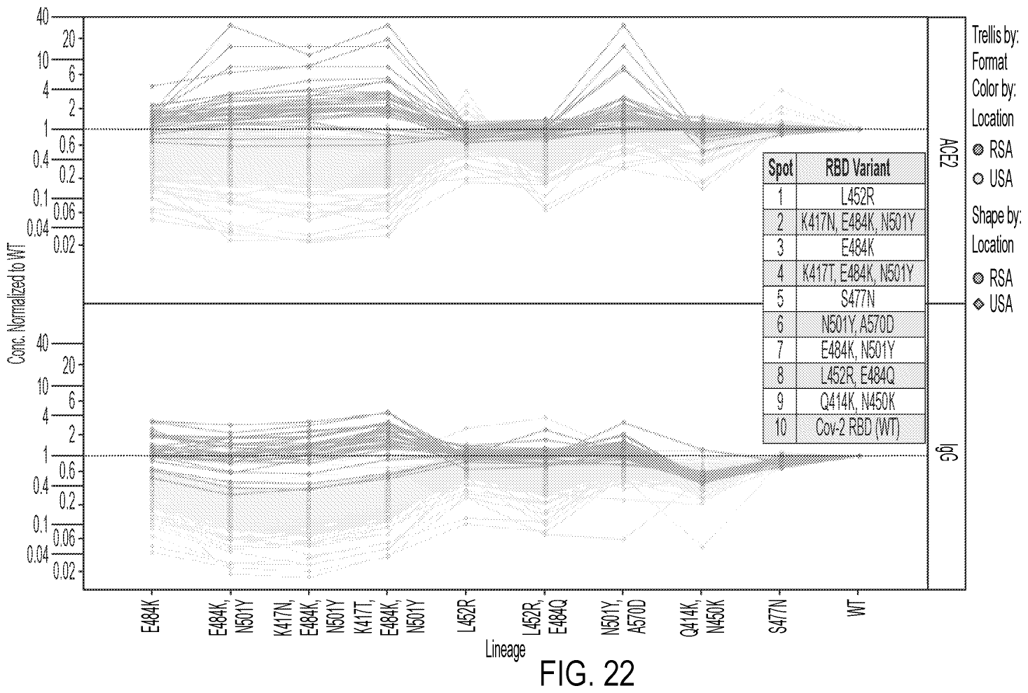

Samples from ˜200 individuals in the United States infected with wild-type SARS-CoV-2 (“Wuhan”) and 32 individuals in South Africa infected with SARS-CoV-2 strain 501Y.V2, also known as B.1.351, were tested using a 10-spot variant SARS-CoV-2 S-RBD panel as shown in FIG. 22, including the following mutations: (1) L452R; (2) K417N, E484K, and N501Y; (3) E484K; (4) K417T, E484K, and N501Y; (5) S477N; (6) N501Y and A570D; (7) E484K and N501Y; (8) L452R and E484Q; (9) Q414K and N450K mutations; and (10) wild-type SARS-CoV-2 S-RBD, wherein all mutations are relative to wild-type S-RBD from SARS-CoV-2. The panels were used in indirect IgG Serology and ACE2 competition assay formats as described in the previous Examples.

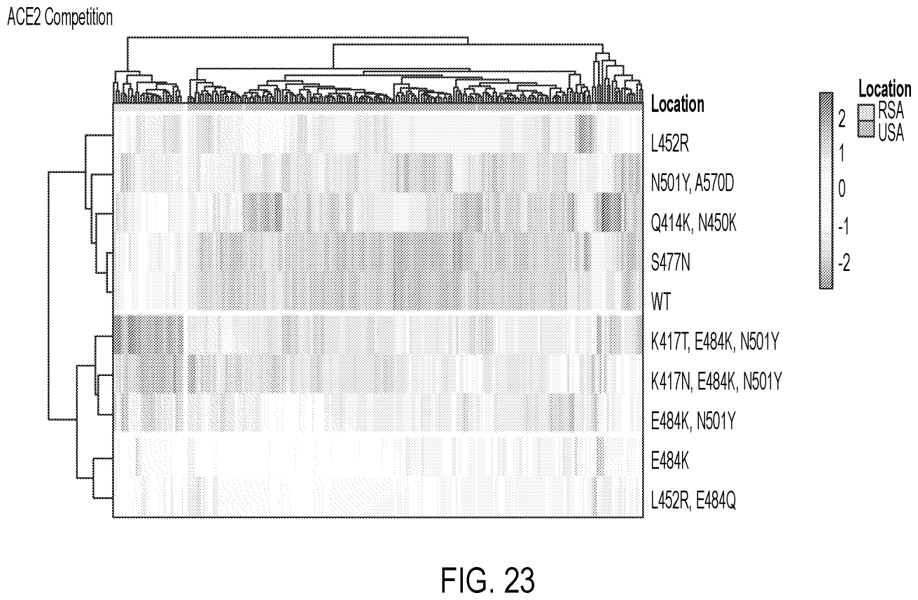

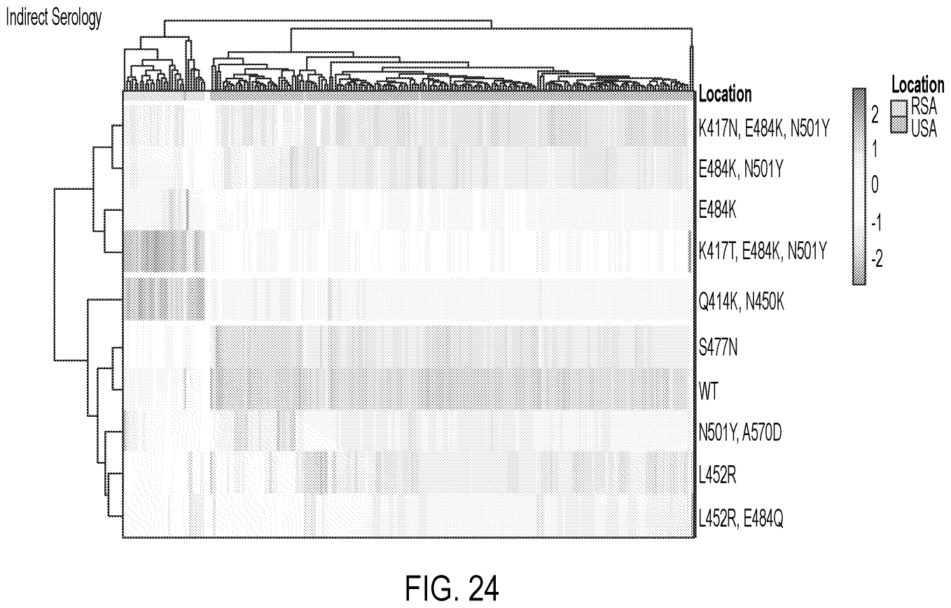

The results in FIG. 22 show the signals for each of the antigens after normalization to the signal from the wild-type SARS-CoV-2 S-RBD. Each set of connected dots shows the normalized signals from each antigen for one study subject. The upper figure shows results of the ACE-2 competition assay; the lower figure shows the serology results. The results show that the sample reactivity towards the wild-type Wuhan strain circulating in the United States at the time of sample collection can be clearly separated from the circulating B.1.351 strain in South Africa. This was further demonstrated by the heat maps from the same data in FIG. 23 (ACE2 competition assay format) and FIG. 24 (indirect IgG serology assay format), which shows a clear separation between the wild-type and B.1.351 strains.

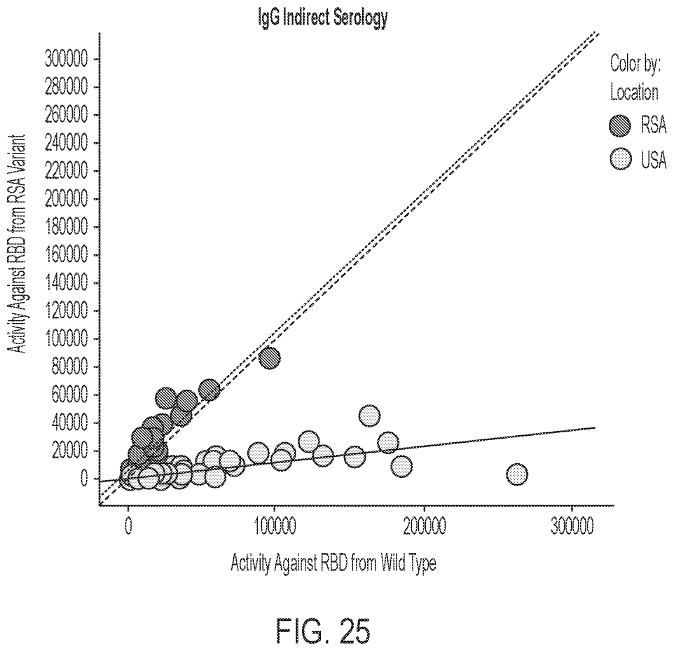

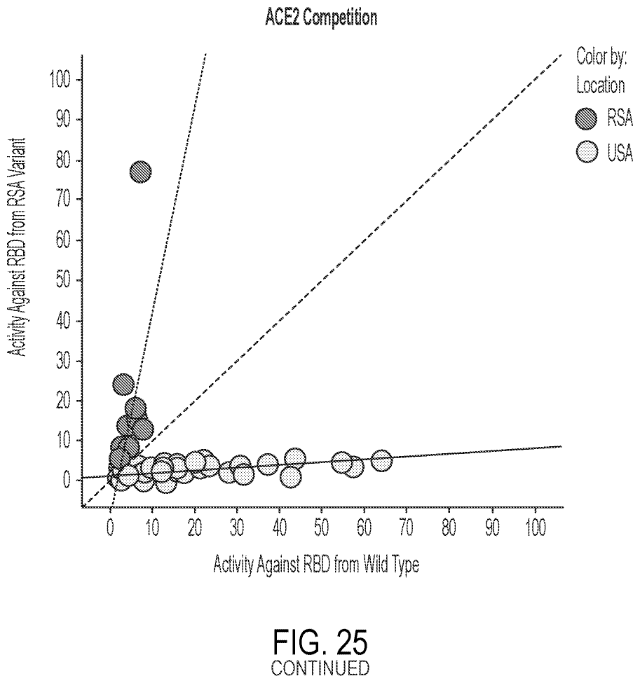

FIG. 25 shows a subset of the data in a different format: for each sample, assay signals for the wild-type SARS-CoV-2 S-RBD (x-axis) were plotted against the signals for the SARS-CoV-2 S-RBD from the B.1.351 strain. While absolute serology signals may vary from subject to subject, the ratio of the signals on the two spots remained remarkably consistent between subjects in one region (and presumably exposed to the same strain), as shown by the data points from each region falling on two different lines with two different slopes. The results in FIG. 25 show that while the signal from one spot may not be sufficient to distinguish between the wild type and B.1.351 SARS-CoV-2 infections, combining the results from two spots provided almost complete separation.

### Example 15. SARS-CoV-2 Serology Assay

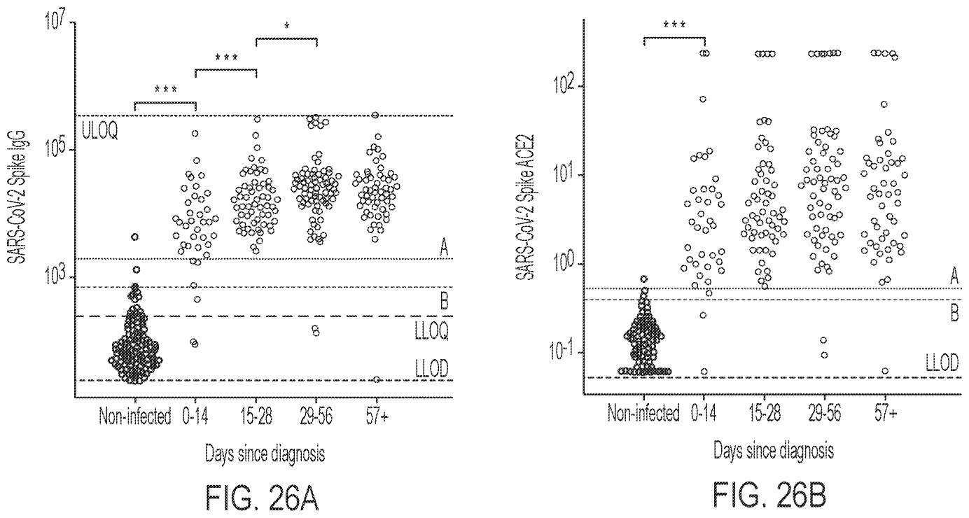

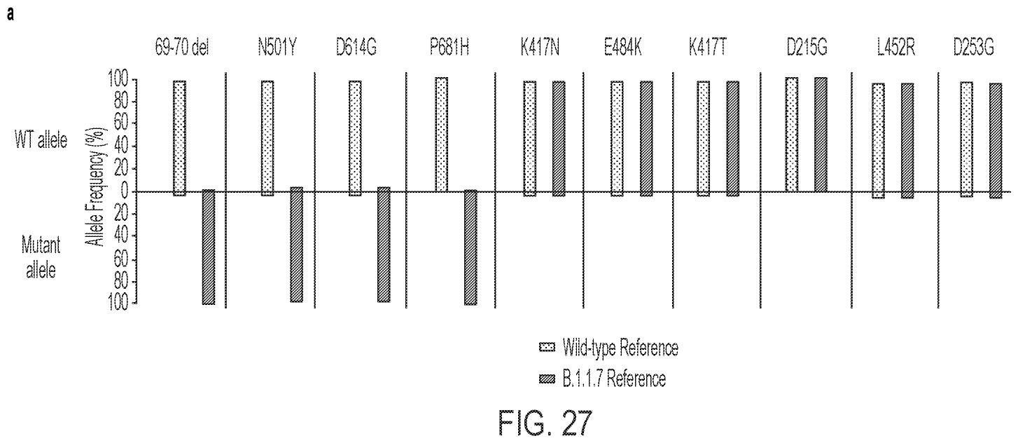

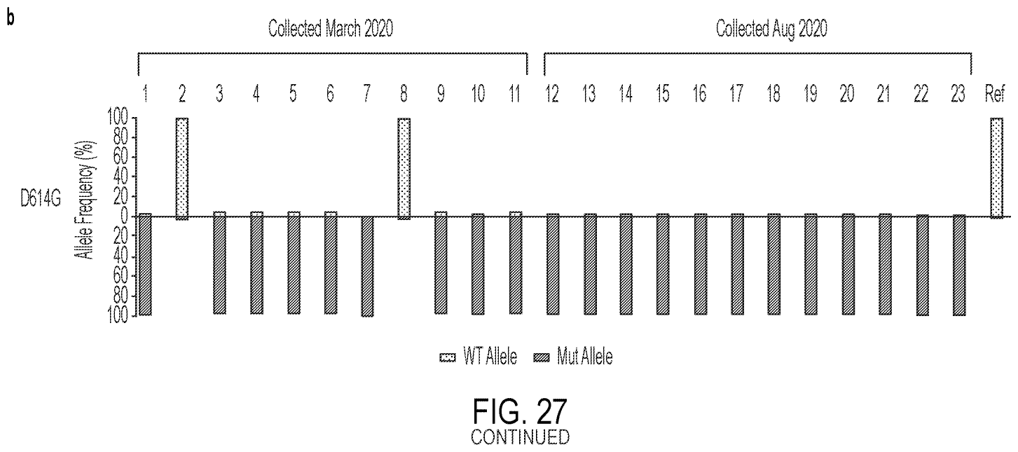

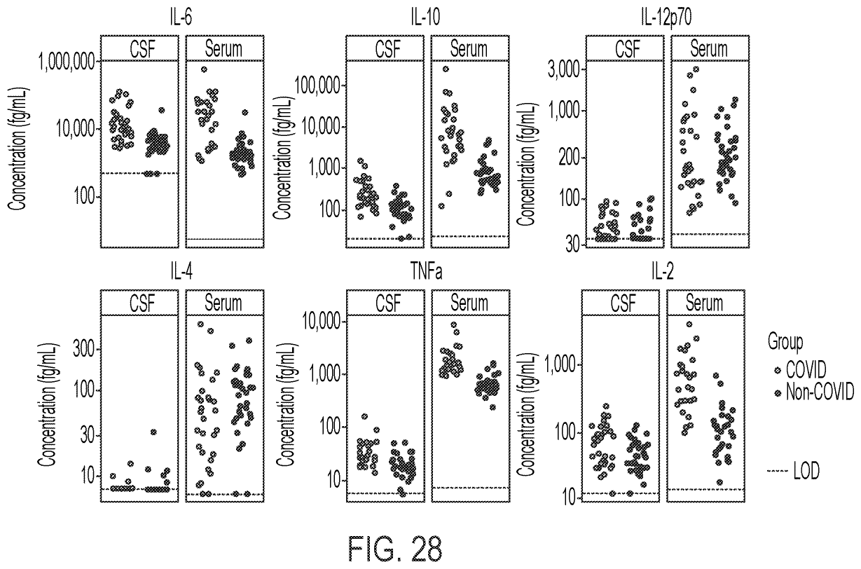

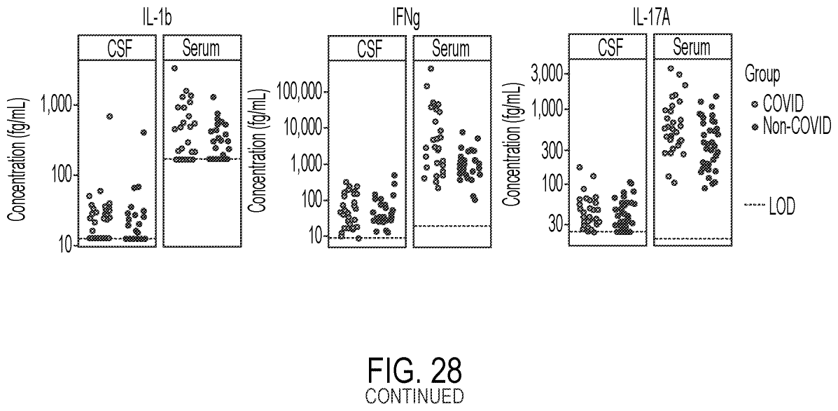

Indirect IgG serology and ACE2 competition assays were performed to measure antibodies against SARS-CoV-2 S protein in 214 serum samples collected from individuals at different time points after confirmed SARS-CoV-2 infection (diagnosis by PCR; 0-14 days, 15-28 days, 29-56 days, and 57+ days) and 200 control samples collected prior to the emergence of SARS-CoV-2 in 2020. The indirect IgG serology assay results are shown in FIG. 26A. The ACE2 competition assay results are shown in FIG. 26B. Both assays had point estimates for sensitivity of 98.3% for detecting infection 15+ days after onset, and point estimates for specificity of 99.5%. The assays also demonstrated good sensitivity for samples collected within the first two weeks after onset (84.2% for indirect serology and 92.1% for ACE2 competition).

### Example 16. SARS-CoV-2 SNP Detection

A multiplexed oligonucleotide ligation assay (OLA) was used to detect SNPs in 23 nasal swab samples from subjects who had previously tested positive for SARS-CoV-2. The mutations in the assay panels included the following: 69-70del, D215G, D253G, K417N, K417T, L452R, E484K, N501Y, D614G, and P681H. A wild-type reference SARS-CoV-2 genomic RNA sample was used for the lineage A control. A known lineage B.1.1.7 reference was also tested, which matched the known mutations as shown in FIG. 37, top panel.

The assay panels were used to assess a set of 23 samples that were collected in March or August of 2020. These samples were found not to contain the mutations in the panel, except for D614G, which was consistent with the fact that the mutations being assessed (other than D614G) were not commonly in circulation at those times. The one exception was the D614G mutation, which became prevalent very early on in the COVID-19 pandemic and is present in almost all samples. For D614G, 21/23 samples contained the D614G mutation, while the wild-type reference did not (FIG. 37, bottom panel).

### Example 17. Host Biomarker Detection in Serum and Cerebrospinal Fluid of COVID-19 Patients

Cerebrospinal fluid (CSF) and serum from acute COVID-19 patients were obtained and measured for the following panel of cytokines: IL-6, IL-10, IL-12p70, IL-4, TNF-α, IL-2, IL-1P, IFN-γ, and IL-17A. The cytokines were measured using the Format 2 immunoassay described in Example 7, i.e., each cytokine was detected using a detection reagent linked to a nucleic acid probe, wherein upon binding of the detection reagent to the cytokine, the nucleic acid probe is extended to form an extended sequence, and the extended sequence is contacted with a probe comprising a detectable label for detection.

The measured cytokine levels from COVID-19 patients were compared against cytokine levels from non-COVID-19 control subjects. Results are shown in FIG. 38, with COVID-19 patients in light grey on the left side of each CSF or serum result panel, and non-COVID-19 control subjects in dark grey on the right side of each CSF or serum result panel. The results showed that IL-2, IL-6, IL-10, IFN-γ, and TNF-α levels in serum and CSF were generally higher in the acute COVID-19 patient group than the control group. In general, the cytokine concentrations in CSF were approximately an order of magnitude lower than in serum, except for IL-6, for which the concentrations in CSF and serum were comparable.

1. A kit for detecting one or more antibody biomarkers of interest in a sample, the kit comprising, in one or more vials, containers, or compartments:
(a) a surface comprising one or more binding domains, wherein each binding domain comprises an immobilized antigen of a panel of antigens, and wherein the panel of antigens comprises:
(i) an S protein from the following SARS-CoV-2 strains: wild-type; BA.2.12.1; BA.2; AY.4; BA.3; BA.2+L452M; BA.2+L452R; BA.4; B.1.351; and BA.5; or
(ii) an S-RBD from the following SARS-CoV-2 strains: BA.2.12.1; B.1.351; BA.2; BA.2+L452M; BA.2+L452R; B.1.1.7; BA.4/BA.5; BA.3; AY.4; and wild-type; or
(iii) an S protein from the following SARS-CoV-2 strains: wild-type; BA.2.12.1; BA.2; AY.4; BA.2.75; BA.4; B.1.351; and BA.5; or
(iv) an S-RBD from the following SARS-CoV-2 strains: an S-RBD from the following SARS-CoV-2 strains: BA.2.12.1; B.1.351; BA.2; B.1.1.7; BA.4/BA.5; BA.2.75; AY.3; and wild-type; or
(v) an S protein from the following SARS-CoV-2 strains: wild-type, BA.2.12.1, BA.2.75, BA.2, BA.1 (B.1.1.529), BA.1.617.2, B.1.1.7, B.1.351, and BA.5; and an N protein from wild-type SARS-CoV-2; or
(vi) an S-RBD from the following SARS-CoV-2 strains: BA.2.12.1, B.1.351, BA.1 (B.1.1.529), BA.2, B.1.1.7, B.1.617.2, BA.2.75, BA.4/BA.5, and wild-type; and an N protein from wild-type SARS-CoV-2; or
(vii) an S protein from the following SARS-CoV-2 strains: wild-type; BA.1; XBB.1; BF.7; BA.2.75.2; BQ.1.1; BA.2.75; BA.4.6; BQ.1; and BA.5; or
(viii) an S-RBD from the following SARS-CoV-2 strains: BA.1; BQ.1.1; BA.2.75.2; BA.4.6/BF.7; XBB.1; BA.4/BA.5/BF.5; BA.2.75; BQ.1; and wild-type; or
(ix) an S protein from the following SARS-CoV-2 strains: wild-type; BA.1; XBB.1; BF.7; XBB.1.5; BQ.1.1; BA.2.75; BN.1; BQ.1; and BA.5; or
(x) an S-RBD from the following SARS-CoV-2 strains: BA.1; BQ.1.1; XBB.1.5; BN.1; XBB.1; BA.5; BA.2.75; BQ.1; and wild-type; or
(xi) virus-like particle (VLP) from enterovirus (EV)-D68; VLP from EV-71; F protein from metapneumovirus (MPV); pre-fusion F from RSV; HA proteins from Flu A/H1 (e.g., H1/Wisconsin 2019), Flu A/H3 (e.g., H3/Darwin 2021), and Flu B/Victoria (e.g., B/Austria 2021); capsid protein VP0 from rhinovirus C (RV-C); S protein from SARS-CoV-2 strain BA.5; F proteins from PIV1, PIV2, PIV3, and PIV4; and VP0 from parechovirus (PeV) A3,
optionally wherein:
the HA proteins from Flu A H1/Wisconsin, Flu A H3/Darwin, and Flu B Austria are in a same binding domain; and/or
the F proteins from PIV1, PIV2, PIV3, and PIV4 are in a same binding domain; or
(xii) HA from Flu A/H1 (e.g., H1/Wisconsin 2019); HA from Flu A/H3 (e.g., H3 Darwin 2021); HA from Flu B/Victoria (e.g., B/Austria 2021); F protein from PIV1; F protein from PIV2, F protein from PIV3; and F protein from PIV4, and
(b) one or more detection reagents, wherein each detection reagent comprises a detection antibody, a detection antigen, or an ACE detection reagent.

2. The kit of claim 1, wherein the detection reagent comprises an electrochemiluminescent (ECL) label.

3. The kit of claim 1 or 2, wherein the surface comprises an electrode.

4. The kit of any one of claims 1 to 3, wherein the surface comprises a well of a multi-well plate, and wherein each well comprises 1 to 10 binding domains.

5. A method of detecting one or more antibody biomarkers of interest in a sample, comprising:
(a) contacting the sample with a surface comprising one or more binding domains, wherein each binding domain comprises an immobilized antigen of a panel of antigens, and wherein the panel of antigens comprises:
(i) an S protein from the following SARS-CoV-2 strains: wild-type; BA.2.12.1; BA.2; AY.4; BA.3; BA.2+L452M; BA.2+L452R; BA.4; B.1.351; and BA.5; or
(ii) an S-RBD from the following SARS-CoV-2 strains: BA.2.12.1; B.1.351; BA.2; BA.2+L452M; BA.2+L452R; B.1.1.7; BA.4/BA.5; BA.3; AY.4; and wild-type; or
(iii) an S protein from the following SARS-CoV-2 strains: wild-type; BA.2.12.1; BA.2; AY.4; BA.2.75; BA.4; B.1.351; and BA.5; or
(iv) an S-RBD from the following SARS-CoV-2 strains: BA.2.12.1; B.1.351; BA.2; B.1.1.7; BA.4/BA.5; BA.2.75; AY.3; and wild-type; or
(v) an S protein from the following SARS-CoV-2 strains: wild-type, BA.2.12.1, BA.2.75, BA.2, BA.1 (B.1.1.529), BA.1.617.2, B.1.1.7, B.1.351, and BA.5; and an N protein from wild-type SARS-CoV-2; or
(vi) an S-RBD from the following SARS-CoV-2 strains: BA.2.12.1, B.1.351, BA.1 (B.1.1.529), BA.2, B.1.1.7, B.1.617.2, BA.2.75, BA.4/BA.5, and wild-type; and an N protein from wild-type SARS-CoV-2; or
(vii) an S protein from the following SARS-CoV-2 strains: wild-type; BA.1; XBB.1; BF.7; BA.2.75.2; BQ.1.1; BA.2.75; BA.4.6; BQ.1; and BA.5; or
(viii) an S-RBD from the following SARS-CoV-2 strains: BA.1; BQ.1.1; BA.2.75.2; BA.4.6/BF.7; XBB.1; BA.4/BA.5/BF.5; BA.2.75; BQ.1; and wild-type; or
(ix) an S protein from the following SARS-CoV-2 strains: wild-type; BA.1; XBB.1; BF.7; XBB.1.5; BQ.1.1; BA.2.75; BN.1; BQ.1; and BA.5; or
(x) an S-RBD from the following SARS-CoV-2 strains: BA.1; BQ.1.1; XBB.1.5, BN.1, XBB.1; BA.5; BA.2.75; BQ.1; and wild-type; or
(xi) virus-like particle (VLP) from enterovirus (EV)-D68; VLP from EV-71; F protein from metapneumovirus (MPV); pre-fusion F from RSV; HA proteins from Flu A/H1 (e.g., H1/Wisconsin 2019), Flu A/H3 (e.g., H3/Darwin 2021), and Flu B/Victoria (e.g., B/Austria 2021); capsid protein VP0 from rhinovirus C (RV-C); S protein from SARS-CoV-2 strain BA.5; F proteins from PIV1, PIV2, PIV3, and PIV4; and VP0 from parechovirus (PeV) A3,
optionally wherein:
the HA proteins from Flu A H1/Wisconsin, Flu A H3/Darwin, and Flu B Austria are in a same binding domain; and/or
the F proteins from PIV1, PIV2, PIV3, and PIV4 are in a same binding domain; or
(xii) HA from Flu A/H1 (e.g., H1/Wisconsin 2019); HA from Flu A/H3 (e.g., H3 Darwin 2021); HA from Flu B/Victoria (e.g., B/Austria 2021); F protein from PIV1; F protein from PIV2, F protein from PIV3; and F protein from PIV4,
(b) forming a binding complex in each binding domain, wherein the binding complex comprises the immobilized antigen and an antibody biomarker that binds to the immobilized antigen;
(c) contacting the binding complex in each binding domain with a detection reagent; and
(d) detecting the binding complex in each binding domain, thereby detecting the one or more antibody biomarkers in the sample.

6. The method of claim 5, wherein the detection reagent comprises a detection antibody, a detection antigen, or an ACE detection reagent.

7. The method of claim 5 or 6, wherein the detection reagent comprises an ECL label.

8. The method of any one of claims 5 to 7, wherein the surface comprises an electrode.

9. The method of any one of claims 5 to 8, wherein the surface comprises a well of a multi-well plate, and wherein each well comprises 1 to 10 binding domains.

10. The method of any one of claims 5 to 9, wherein the detection reagent comprises an ECL label, the surface comprises an electrode, and the detecting comprises applying a voltage to the surface and measuring an ECL signal generated from the ECL label on the detection reagent.
